(19) United States
(12) Patent Application Publication
(10) Pub. No.: US 2022/0177883 A1
(43) Pub. Date: Jun. 9, 2022

(54) Antisense Oligonucleotides Targeting ATXN3

(71) Applicant: Hoffmann-La Roche Inc., Little Falls, NJ (US)

(72) Inventors: Heidi Rye Hudlebusch, Bronshoj (DK); Lykke Pedersen, Kobenhavn (DK); Erik Funder, Hillerod (DK); Lukasz Kielpinski, Horsholm (DK); Christoffer Sondergaard, Capital (DK); Alexander Herbert Stephan, Basel-City (CH)

(21) Appl. No.: 17/540,569

(22) Filed: Dec. 2, 2021

(30) Foreign Application Priority Data
Dec. 3, 2020 (EP) EP20211618.2

(51) Int. Cl. C12N 15/113; A61K 31/7088; A61P 25/28

(52) U.S. Cl. CPC C12N 15/113; A61K 31/7088; A61P 25/28; C12N 2310/11; C12N 2310/14; C12N 2310/341; C12N 2310/314; C12N 2310/315; C12N 2310/321; C12N 2310/322; C12N 2310/3341; C12N 2310/3231

(57) ABSTRACT

The present invention relates to antisense LNA oligonucleotides (oligomers) complementary to ATXN3 pre-mRNA sequences, which are capable of inhibiting the expression of ATXN3 protein. Inhibition of ATXN3 expression is beneficial for the treatment of spinocerebellar ataxia.

### CROSS REFERENCE TO RELATED APPLICATIONS

This application claims benefit of and priority to the European Patent Application EP 20211618.2 filed on Dec. 3, 2020, all of which are incorporated by reference in their entireties where permissible.

### TECHNICAL FIELD OF THE INVENTION

The present invention relates to antisense LNA oligonucleotides (oligomers) complementary to ATXN3 pre-mRNA sequences, which are capable of inhibiting the expression of ATXN3. Inhibition of ATXN3 expression is beneficial for the treatment of spinocerebellar ataxia, such as spinocerebellar ataxia 3 (Machado-Joseph disease (MJD)).

### REFERENCE TO SEQUENCE LISTING

The instant application contains a Sequence Listing which has been submitted electronically in ASCII format and is hereby incorporated by reference in its entirety. Said ASCII copy, created on Dec. 3, 2020, is named 067211_015US1 SL.txt and is 614,400 bytes in size.

### BACKGROUND OF THE INVENTION

Spinocerebellar ataxia type 3 (SCA3), also known as Machado-Joseph disease (MJD), is one of nine polyglutamine expansion diseases and the most common dominantly inherited ataxia in the world. While certain symptoms in SCA3 may respond to symptomatic therapy, there is still no effective treatment for this relentlessly progressive and fatal neurodegenerative disease. The disease is caused by a CAG repeat expansion in the ATXN3 gene that encodes an abnormally long polyglutamine tract in the disease protein, ATXN3 (Ataxin 3). The toxic ataxin-3 protein is associated with aggregates which are frequently observed in the brain tissue of SCA3 patients.

Moore et al. reported that antisense oligonucleotides (ASOs) targeting ATXN3 were capable of reducing levels of the pathogenic ATXN3 protein both in human disease fibroblasts and in a mouse model expressing the full-length human mutant ATXN3 gene (Moore et al., Mol Ther Nucleic Acids. 2017; 7:200-210). Therefore, ASO-mediated targeting of ATXN3 was suggested as therapeutic approach for SCA3.

Swayze et al. (Nucleic Acids Res. 2007; 35(2):687-700. Epub 2006 Dec. 19), reports that antisense oligonucleotides containing locked nucleic acid have the potential to improve potency but cause significant toxicity in animals (hepatotoxicity).

Toonen et al. used antisense oligonucleotides to mask predicted exonic splicing signals of ATXN3, resulting in exon 10 skipping from ATXN3 pre-mRNA. The skipping of exon 10 led to formation of a truncated ataxin-3 protein lacking the toxic polyglutamine expansion, but retaining its ubiquitin binding and cleavage function (Toonen et al., Molecular Therapy—Nucleic Acids, 2017, Volume 8: 232-242).

WO2013/138353, WO2015/017675, WO2018/089805, WO2019/217708 and WO2020/172559 disclose antisense oligonucleotides targeting human ATXN3 mRNA for use in the treatment of SCA3.

### SUMMARY OF THE INVENTION

The present invention identifies regions of the ATXN3 transcript (ATXN3) for antisense inhibition in vitro or in vivo, and provides for antisense oligonucleotides, including LNA gapmer oligonucleotides, which target these regions of the ATXN3 pre-mRNA or mature mRNA. Particularly, the present invention identifies antisense oligonucleotides which target human ATXN3 pre-mRNA or mature mRNA more effectively than they target the human Potassium Voltage-Gated Channel Subfamily B Member 2 (KCNB2) pre-mRNA or mature mRNA. The present invention identifies oligonucleotides which inhibit human ATXN3 which are useful in the treatment of spinocerebellar ataxia.

The invention provides for an antisense oligonucleotide, 10-30 nucleotides in length, targeting a mammalian ATXN3 (Ataxin 3) target nucleic acid, wherein the antisense oligonucleotide is capable of inhibiting the expression of mammalian ATXN3 in a cell which is expressing mammalian ATXN3. The mammalian ATXN3 target nucleic acid may be, e.g., a human, monkey or mouse ATXN3 target nucleic acid.

The invention also provides for an LNA gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to SEQ ID NO:1, wherein the antisense oligonucleotide is capable of inhibiting the expression of human ATXN3 in a cell which is expressing human ATXN3.

In one aspect, the invention provides for an antisense oligonucleotide comprising a contiguous nucleotide sequence comprising the contiguous nucleotides present in SEQ ID NO:1122 except for one or more modified nucleosides and/or one or more modified internucleoside linkages, wherein the antisense oligonucleotide is capable of inhibiting the expression of human ATXN3 in a cell which is expressing human ATXN3; or a pharmaceutically acceptable salt thereof. In some embodiments, the antisense oligonucleotide is more capable of inhibiting the expression of human ATXN3 than human KCNB2 in a cell which is expressing human ATXN3 and human KCNB2. In some embodiments, the one or more modified nucleosides and/or one or more modified internucleoside linkages are, for each residue in SEQ ID NO:1122, independently selected from the options for that residue as shown in Table 15.

In some embodiments, the antisense oligonucleotide comprises a contiguous nucleotide sequence comprising at least 10, such as at least 12, such as at least 14, such as at least 16 contiguous nucleotides present in SEQ ID NO:1122. In some embodiments, the antisense oligonucleotide comprises the contiguous nucleotide sequence of SEQ ID NO: 1122.

In some embodiments, the antisense oligonucleotide comprises a contiguous nucleotide sequence comprising the base sequence of an antisense oligonucleotide selected from the group consisting of Compound ID Nos. 1122_82 to 1122_336, shown in Table 11.

In some embodiments, the antisense oligonucleotide comprises a contiguous nucleotide sequence comprising the nucleoside base sequence and, optionally, the sugar moiety modifications, of an antisense oligonucleotide selected from the group consisting of Compound ID Nos. 1122_91, 1122_107, 1122_154, 1122_155, 1122_156, 1122_157, 1122_158, 1122_167, 1122_172, 1122_175, 1122_294, and 1122_296, shown in Table 14.

In some embodiments, the antisense oligonucleotide is an LNA gapmer antisense oligonucleotide; or a pharmaceutically acceptable salt thereof. Typically, each LNA cytosine is an LNA 5-methyl cytosine.

In some embodiments, substantially all, or all, internucleoside linkages between the nucleosides are phosphorothioate internucleoside linkages. In some particular embodiments, one or more of the phosphothioate internucleoside linkages are stereodefined.

In some embodiments, one or more nucleosides are also or alternatively modified to a 2′-sugar-modified nucleoside.

In some embodiments, one or more cytosine nucleosides are also or alternatively modified to a 5-methyl cytosine nucleoside.

In some embodiments, one or more thymine nucleosides are modified to a uracil nucleoside.

In one aspect, the invention provides for an antisense oligonucleotide comprising a contiguous nucleotide sequence comprising the contiguous nucleotides present in SEQ ID NO:1816 except for one or more modified nucleosides and/or one or more modified internucleoside linkages, wherein the antisense oligonucleotide is capable of inhibiting the expression of human ATXN3 in a cell which is expressing human ATXN3; or a pharmaceutically acceptable salt thereof. In some embodiments, the antisense oligonucleotide is more capable of inhibiting the expression of human ATXN3 than human KCNB2 in a cell which is expressing human ATXN3 and human KCNB2. In some embodiments, the one or more modified nucleosides and/or one or more modified internucleoside linkages are, for each residue in SEQ ID NO:1816, independently selected from the options for that residue as shown in Table 16.

In some embodiments, the antisense oligonucleotide comprises a contiguous nucleotide sequence comprising at least 10, such as at least 12, such as at least 14, such as at least 16 contiguous nucleotides present in SEQ ID NO:1816. In some embodiments, the antisense oligonucleotide comprises the contiguous nucleotide sequence of SEQ ID NO: 1816.

In some embodiments, the antisense oligonucleotide comprises a contiguous nucleotide sequence comprising the base sequence of an antisense oligonucleotide selected from the group consisting of Compound ID Nos. 1816_2 to 1816_74, shown in Table 11.

In some embodiments, the antisense oligonucleotide comprises the nucleoside base sequence and, optionally, the sugar moiety modifications, of an antisense oligonucleotide selected from the group consisting of Compound ID Nos. 1816_13, 1816_15, 1816_28, 1816_41, 1816_42, 1816_43, 1816_60, 1816_61, 1816_64, 1816_65, and 1816_68, as shown in Table 14.

In some embodiments, the antisense oligonucleotide is an LNA gapmer antisense oligonucleotide; or a pharmaceutically acceptable salt thereof. Typically, each LNA cytosine is an LNA 5-methyl cytosine.

In some embodiments, substantially all, or all, internucleoside linkages between the nucleosides are phosphorothioate internucleoside linkages. In some particular embodiments, one or more of the phosphothioate internucleoside linkages are stereodefined.

In some embodiments, one or more nucleosides are also or alternatively modified to a 2′-sugar-modified nucleoside.

In some embodiments, one or more cytosine nucleosides are also or alternatively modified to a 5-methyl cytosine nucleoside.

In some embodiments, one or more thymine nucleosides are modified to a uracil nucleoside.

More details on these or other nucleoside modifications and/or internucleoside linkage modifications are provided in the present disclosure.

In one aspect, the invention provides for the antisense oligonucleotides disclosed herein, for example an antisense oligonucleotide selected from the group consisting of the compounds shown in the table in Example 13; or a pharmaceutically acceptable salt thereof.

In one aspect, the invention provides for the antisense oligonucleotide disclosed herein, for example an antisense oligonucleotide selected from the group consisting of the compounds shown in Table 11; or a pharmaceutically acceptable salt thereof.

In some embodiments, the antisense oligonucleotide is selected from the group consisting of the compounds shown in the table in Example 16.

In some embodiments, the antisense oligonucleotide is selected from the group consisting of the compounds shown in Table 14.

In one aspect, the invention particularly provides for an antisense oligonucleotide selected from the group consisting of Compound ID Nos. 1122_91, 1122_107, 1122_154, 1122_155, 1122_156, 1122_157, 1122_158, 1122_167, 1122_172, 1122_175, 1122_294, 1122_296, 1816_13, 1816_15, 1816_28, 1816_41, 1816_42, 1816_43, 1816_60, 1816_61, 1816_64, 1816_65, and 1816_68; or a pharmaceutically acceptable salt thereof.

In separate and specific aspects, the invention provides for an antisense oligonucleotide as shown in FIG. 12A, 12B, 12C, 12D, 12E, 12F, 12G, 12H, 12I, 12J, 12K, 12L, 12M, 12N, 12O, 12P, 12Q, 12R, 12S, 12T, 12U, 12V, or 12W; or a pharmaceutically acceptable salt thereof. A oligonucleotide of the invention as referred to or claimed herein may be in the form of a pharmaceutically acceptable salt, such as a sodium or potassium salt.

In one aspect, the invention provides for a conjugate comprising an oligonucleotide according to the invention, and at least one conjugate moiety covalently attached to said oligonucleotide.

In one aspect, the invention provides for a pharmaceutical composition comprising the oligonucleotide or conjugate of the invention and a pharmaceutically acceptable diluent, solvent, carrier, salt and/or adjuvant.

In one aspect, the invention provides for an in vivo or in vitro method for modulating ATXN3 expression in a target cell which is expressing ATXN3, said method comprising administering an oligonucleotide or conjugate or pharmaceutical composition of the invention in an effective amount to said cell.

In one aspect, the invention provides for a method for treating or preventing a disease comprising administering a therapeutically or prophylactically effective amount of an oligonucleotide, conjugate or the pharmaceutical composition of the invention to a subject suffering from or susceptible to the disease.

In some embodiments, the disease is spinocerebellar ataxia, such as spinocerebellar ataxia 3, such as Machado-Joseph disease (MJD).

In one aspect, the invention provides for the oligonucleotide, conjugate or the pharmaceutical composition of the invention for use in medicine.

In one aspect, the invention provides for the oligonucleotide, conjugate or the pharmaceutical composition of the invention for use in the treatment or prevention of spinocerebellar ataxia, such as spinocerebellar ataxia 3, such as Machado-Joseph disease (MJD).

In one aspect, the invention provides for the use of the oligonucleotide, conjugate or the pharmaceutical composition of the invention, for the preparation of a medicament for treatment or prevention of spinocerebellar ataxia, such as spinocerebellar ataxia 3 such as Machado-Joseph disease (MJD).

### BRIEF DESCRIPTION OF THE DRAWINGS

FIG. 1 displays a drawing of the compound 1122_67 (SEQ ID NO:1122).

FIG. 2 displays a drawing of the compound 1813_1 (SEQ ID NO:1813).

FIG. 3 displays a drawing of the compound 1856_1 (SEQ ID NO:1856).

FIG. 4 displays a drawing of the compound 1812_1 (SEQ ID NO:1812).

FIG. 5 displays a drawing of the compound 1809_2 (SEQ ID NO:1809).

FIG. 6 displays a drawing of the compound 1607_1 (SEQ ID NO:1607).

FIG. 7 displays a drawing of the compound 1122_62 (SEQ ID NO:1122).

FIG. 8 displays a drawing of the compound 1122_33 (SEQ ID NO:1122).

FIG. 9 portrays the stability of the compounds 1122_67 and 1813_1, and 5 reference compounds (i.e. compounds 1100673, 1101657, 1102130, 1103014, and 1102987) in a 24 hour SVPD assay.

FIG. 10A displays a WES analysis of GM06153 cells treated with different ASOs to obtain reduction of wild type Ataxin 3 (55 kDa) and polyQ extended Ataxin 3 (77 kDa). FIG. 10B displays an analysis of band intensity normalized to HPRT. Wild type Ataxin 3 is represented by the band at 55 kDa, and the polyQ extended Ataxin 3 is represented by the band at 77 kDa. Cells have been treated with 10 uM of ASO for 4 days prior to protein analysis. Data represents cells treated with ASOs in triplicates as mean+-SD. SC, scrambled control oligo.

FIG. 11 displays a drawing of the compound 1816_12.

FIGS. 12A-W display drawings of the compounds in Table 14 (Example 16). FIG. 12A displays a drawing of compound 1122_91. FIG. 12B displays a drawing of compound 1122_107. FIG. 12C displays a drawing of compound 1122_154. FIG. 12D displays a drawing of compound 1122_155. FIG. 12E displays a drawing of compound 1122_156. FIG. 12F displays a drawing of compound 1122_157. FIG. 12G displays a drawing of compound 1122_158. FIG. 12H displays a drawing of compound 1122_167. FIG. 12I displays a drawing of compound 1122_172. FIG. 12J displays a drawing of compound 1122_175. FIG. 12K displays a drawing of compound 1122_294. FIG. 12L displays a drawing of compound 1122_296. FIG. 12M displays a drawing of compound 1816_13. FIG. 12N displays a drawing of compound 1816_15. FIG. 12O displays a drawing of compound 1816_28. FIG. 12P displays a drawing of compound 1816_41. FIG. 12Q displays a drawing of compound 1816_42. FIG. 12R displays a drawing of compound 1816_43. FIG. 12S displays a drawing of compound 1816_60. FIG. 12T displays a drawing of compound 1816_61. FIG. 12U displays a drawing of compound 1816_64. FIG. 12V displays a drawing of compound 1816_65. FIG. 12W displays a drawing of compound 1816_68.

The chemical drawings show the protonated form of the antisense oligonucleotide, and it will be understood that each hydrogen on the sulphur atom in the phosphorothioate intemucleoside linkage may independently be present or absent. In a salt form, one or more more of the hydrogens may for example be replaced with a cation, such as a metal cation, such as a sodium cation or a potassium cation.

FIG. 13 displays an image showing raw results from the WES analysis of protein level. Included are compounds 1605_4, 1122_107, 1122_156 and a scrambled control oligo.

FIG. 14 displays an image showing raw results from the WES analysis of protein level. Included are compounds 1287095, 1102579, 1605_2 and a scrambled control oligo.

FIG. 15 displays an analysis of band intensity normalized to HPRT. Cells have been treated with 5 μM of ASO for 4 days prior to protein analysis. Data represents cells treated with ASOs in triplicates as mean+-SD. * p-value<0.05; ** p-value<0.01.

FIG. 16 displays a WES analysis of SK-N-AS cells treated with different ASOs to obtain reduction of wild type Ataxin 3 (55 kDa). The loading control used for normalization was HPRT.

FIG. 17 displays a WES analysis of SK-N-AS cells treated with different reference compound ASOs to obtain reduction of wild type Ataxin 3 (55 kDa). The loading control used for normalization was HPRT.

FIG. 18 displays an analysis of band intensity normalized to HPRT. Cells were treated with 5 or 15 uM of ASO for 4 days prior to protein analysis. Data represents cells treated with ASOs in triplicates as mean+-SD.

FIG. 19 displays results from ddPCR analysis showing remaining level of ATXN3 mRNA following treatment with the listed compounds.

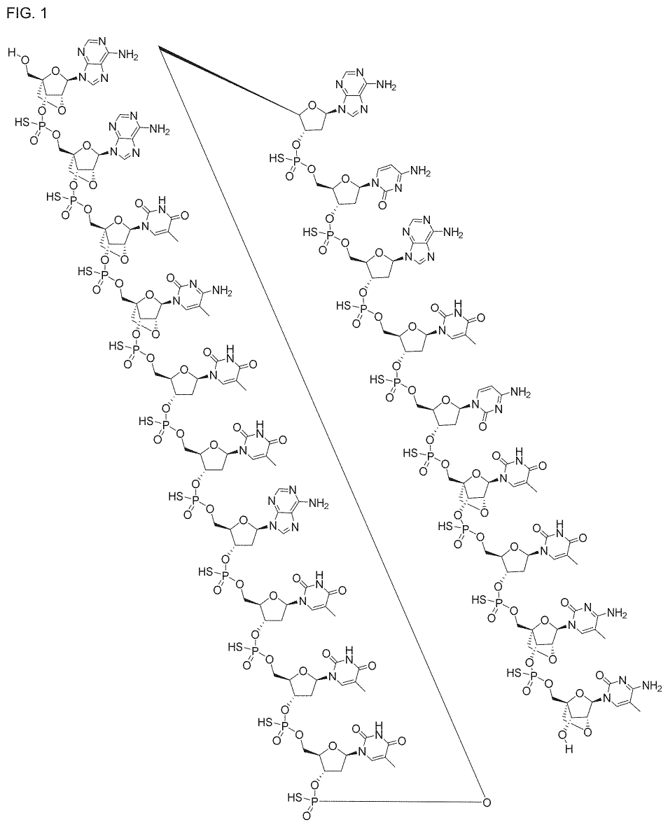

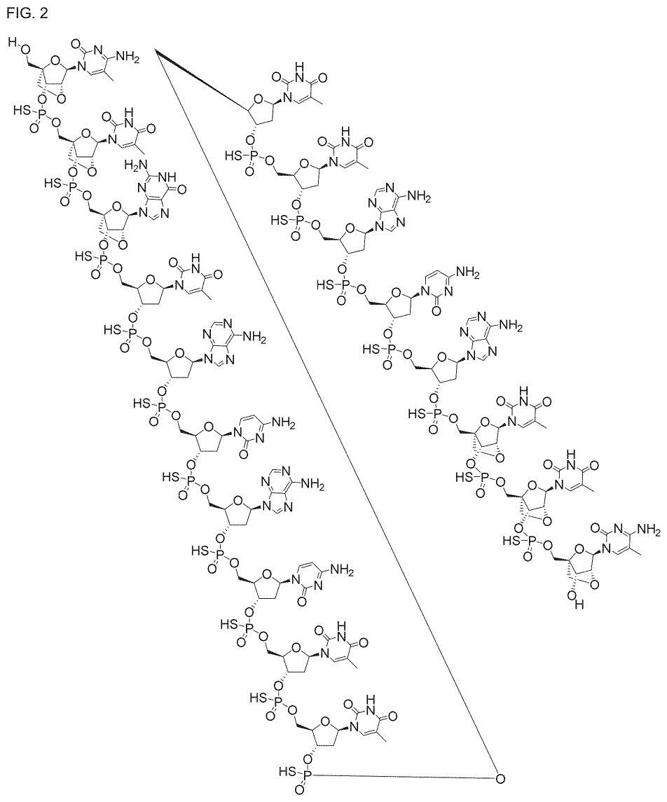

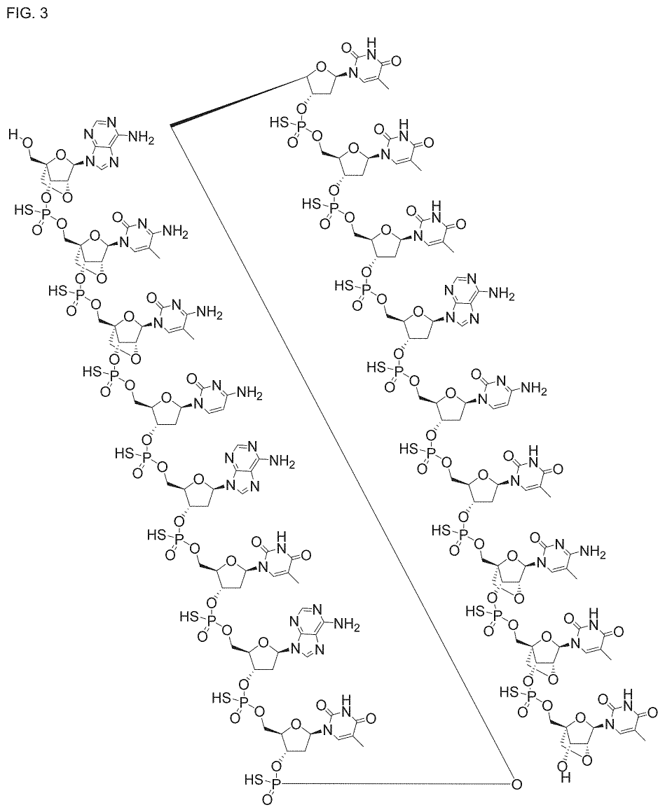

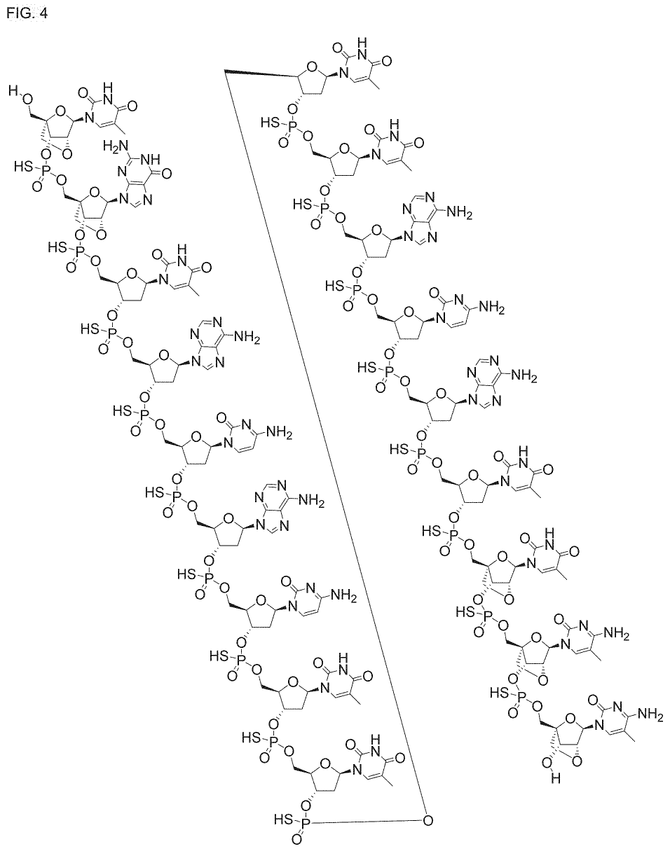

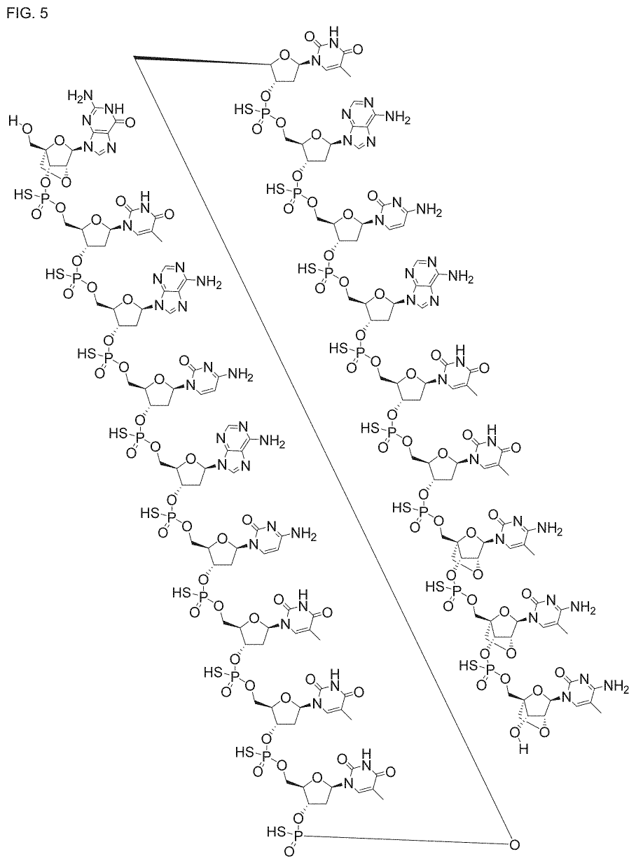

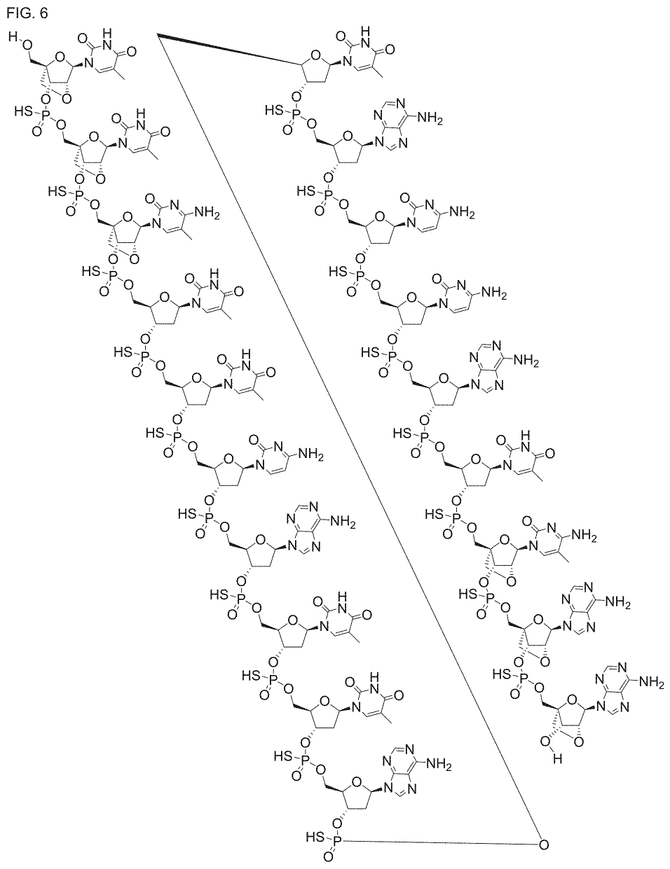

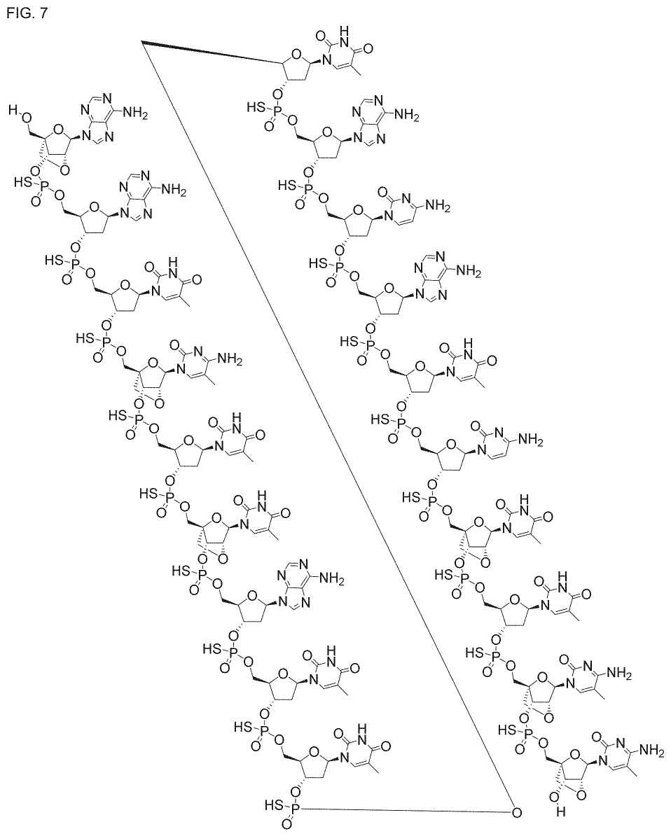

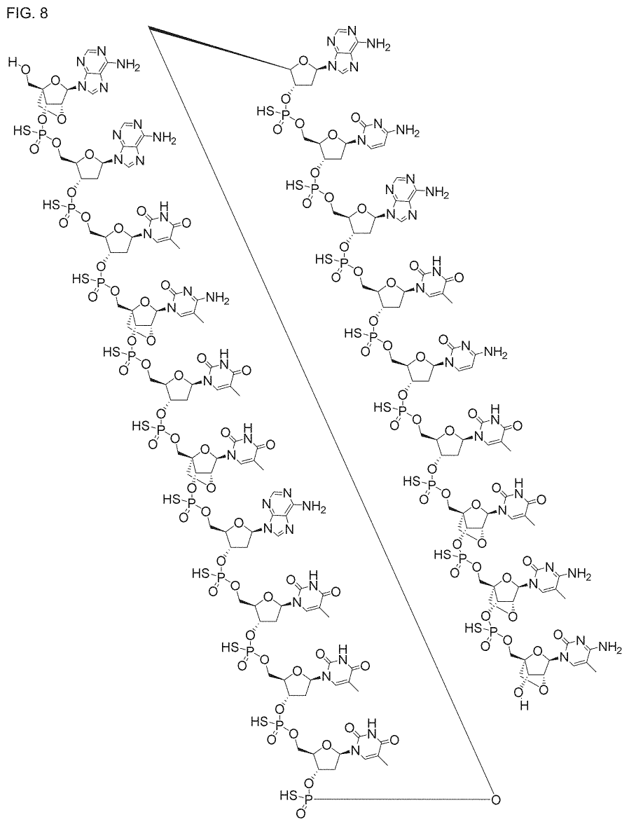

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “treat,” “treating,” “treatment” and “therapeutic use” refer to the elimination, reduction or amelioration of one or more symptoms of a disease or disorder. Specifically, the term “treatment” may refer to both treatment of an existing disease (e.g. a disease or disorder as herein referred to), or prevention of a disease (i.e. prophylaxis). It will therefore be recognized that treatment as referred to herein may, in some embodiments, be prophylactic. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease.

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence” also refered to as “motif sequence”. The “motif sequence” may also be referred to as the “Oligonucleotide Base Sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. Advantageously, the contiguous nucleotide sequence is 100% complementary to the target nucleic acid.

The term “modified oligonucleotide” describes an oligonucleotide comprising one or more modified nucleosides and/or modified internucleoside linkages. The term chimeric” oligonucleotide is a term that has been used in the literature to describe oligonucleotides with modified nucleosides.

The term “nucleotides” refers to the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

The term “nucleobase” refers to the purine (e.g. adenine and guanine) and pyrimidine (e.g. uracil, thymine and cytosine) moieties present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the “nucleobase moiety” is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. In a preferred embodiment, the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

As used herein, “sugar modifications” also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

As used herein, a “2′ sugar modified nucleoside” refers to a nucleoside which has a substituent other than H or OH at the 2′ position (2′ substituted nucleoside) or comprises a 2′ linked biradicle capable of forming a bridge between the 2′ carbon and a second carbon in the ribose ring, such as LNA (2′ 4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

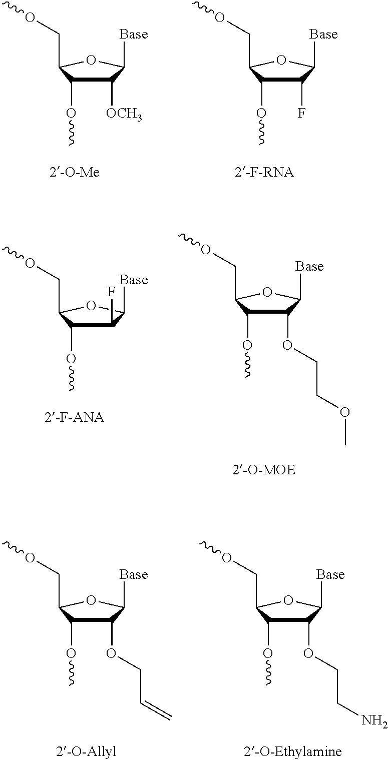

In relation to the present invention 2′ substituted does not include 2′ bridged molecules like LNA.

As used herein, a “Locked Nucleic Acid (LNA) nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1:

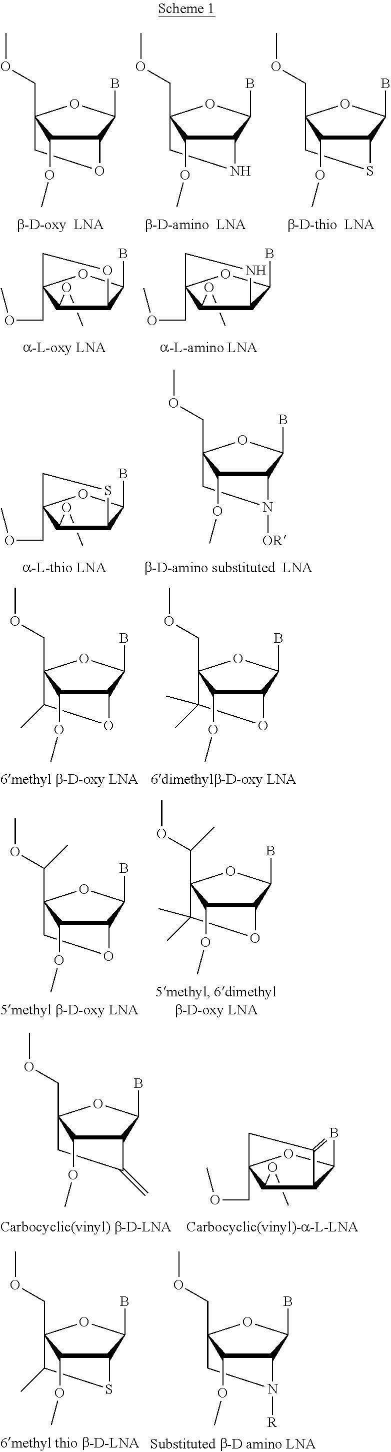

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

As used herein, the term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides.

Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′. In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate. Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate gap region.

As used herein, “phosphorothioate linkages” refer to internucleoside phosphate linkages where one of the non-bridging oxygens has been substituted with a sulfur. The substitution of one of the non-bridging oxygens with a sulfur introduces a chiral center, and as such within a single phosphorothioate oligonucleotide, each phosphorothioate internucleoside linkage will be either in the S (Sp) or R (Rp) stereoisoforms. Such internucleoside linkages are referred to as “chiral internucleoside linkages”. By comparison, phosphodiester internucleoside linkages are non-chiral as they have two non-terminal oxygen atoms.

The designation of the chirality of a stereocenter is determined by standard Cahn-Ingold-Prelog rules (CIP priority rules) first published in Cahn, R.S.; Ingold, C.K.; Prelog, V. (1966). “Specification of Molecular Chirality”. Angewandte Chemie International Edition. 5 (4): 385-415. doi:10.1002/anie.196603851.

During standard oligonucleotide synthesis, the stereoselectivity of the coupling and the following sulfurization is not controlled. For this reason, when producing an oligonucleotide by standard oligonucleotide synthetic methods, the stereoconfiguration of any specific phosphorothioate internucleoside linkage introduced may become either Sp or Rp. The resulting preparation of such an oligonucleotide may therefore contain as many as 2^{X} different individual phosphorothioate diastereoisomers, where X is the number of phosphorothioate internucleoside linkages. Such oligonucleotides are referred to as “stereorandom phosphorothioate oligonucleotides” herein, and do not contain any stereodefined internucleoside linkages. Stereorandom phosphorothioate oligonucleotides are therefore mixtures of individual diastereoisomers originating from the non-stereodefined synthesis. In this context the mixture is defined as up to 2′ different phosphorothioate diastereoisomers. A stereorandom phosphorothioate internucleoside linkage may also be referred to as a stereo-undefined phosphorothioate internucleoside linkage or, using HELM-annotations, [sP] or (abbreviated) “X”, herein (see Examples 13 and 16).

As used herein, a “stereodefined internucleoside linkage” refers to an internucleoside linkage which introduces a specific chiral center into the oligonucleotide, which exists in predominantly one stereoisomeric form, either R or S within a population of individual oligonucleotide molecules.

It should be recognized that stereoselective oligonucleotide synthesis methods used in the art typically provide at least about 90% or at least about 95% stereoselectivity at each internucleoside linkage stereocenter, and as such up to about 10%, such as about 5% of oligonucleotide molecules may have the alternative stereo isomeric form.

In some embodiments the stereoselectivity of each stereodefined phosphorothioate stereocenter is at least about 90%. In some embodiments the stereoselectivity of each stereodefined phosphorothioate stereocenter is at least about 95%.

As used herein, “stereodefined phosphorothioate linkages” refer to phosphorothioate linkages which have been chemically synthesized in either the Rp or Sp configuration within a population of individual oligonucleotide molecules, such as at least about 90% or at least about 95% stereoselectivity at each stereocenter (either Rp or Sp), and as such up to about 10%, such as about 5% of oligonucleotide molecules may have the alternative stereo isomeric form. The stereo configurations of the phosphorothioate internucleoside linkages are presented below

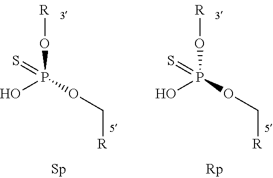

where the 3′ R group represents the 3′ position of the adjacent nucleoside (a 5′ nucleoside), and the 5′ R group represents the 5′ position of the adjacent nucleoside (a 3′ nucleoside).

Rp internucleoside linkages may also be represented as srP, and Sp internucleoside linkages may be represented as ssP herein. Using HELM annotations, a stereodefined Sp phosphorothioate internucleoside linkage may also be referred to as [ssP] or abbreviated as “S” herein (see Examples 13 and 16).

In some embodiments the stereoselectivity of each stereodefined phosphorothioate stereocenter is at least about 97%. In some embodiments the stereoselectivity of each stereodefined phosphorothioate stereocenter is at least about 98%. In some embodiments the stereoselectivity of each stereodefined phosphorothioate stereocenter is at least about 99%.

In some embodiments a stereoselective internucleoside linkage is in the same stereoisomeric form in at least 97%, such as at least 98%, such as at least 99%, or (essentially) all of the oligonucleotide molecules present in a population of the oligonucleotide molecule. Stereoselectivity can be measured in a model system only having an achiral backbone (i.e. phosphodiesters) it is possible to measure the stereoselectivity of each monomer by e.g. coupling a stereodefined monomer to the following model-system “5′ t-po-t-po-t-po 3”. The result of this will then give: 5′ DMTr-t-srp-t-po-t-po-t-po 3′ or 5′ DMTr-t-ssp-t-po-t-po-t-po 3′ which can be separated using HPLC. The stereoselectivity is determined by integrating the UV signal from the two possible compounds and giving a ratio of these e.g. 98:2, 99:1 or >99:1.

It will be understood that the stereo % purity of a specific single diastereoisomer (a single stereodefined oligonucleotide molecule) will be a function of the coupling selectivity for the defined stereocenter at each internucleoside position, and the number of stereodefined internucleoside linkages to be introduced. By way of example, if the coupling selectivity at each position is 97%, the resulting purity of the stereodefined oligonucleotide with 15 stereodefined internucleoside linkages will be 0.97¹⁵, i.e. 63% of the desired diastereoisomer as compared to 37% of the other diastereoisomers. The purity of the defined diastereoisomer may after synthesis be improved by purification, for example by HPLC, such as ion exchange chromatography or reverse phase chromatography.

In some embodiments, a stereodefined oligonucleotide refers to a population of an oligonucleotide wherein at least about 40%, such as at least about 50% of the population is of the desired diastereoisomer.

Alternatively stated, in some embodiments, a stereodefined oligonucleotide refers to a population of oligonucleotides wherein at least about 40%, such as at least about 50%, of the population consists of the desired (specific) stereodefined internucleoside linkage motif (also termed stereodefined motif).

For stereodefined oligonucleotides which comprise both stereorandom and stereodefined internucleoside stereocenters, the purity of the stereodefined oligonucleotide is determined with reference to the % of the population of the oligonucleotide which retains the defined stereodefined internucleoside linkage motif(s), the stereorandom linkages are disregarded in the calculation.

As used herein, a “stereodefined oligonucleotide” refers to an oligonucleotide wherein at least one of the internucleoside linkages is a stereodefined internucleoside linkage.

As used herein, a “stereodefined phosphorothioate oligonucleotide” refers to an oligonucleotide wherein at least one of the internucleoside linkages is a stereodefined phosphorothioate internucleoside linkage.

As used herein, the term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

As used herein, the term “% complementary” refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target nucleic acid or target sequence). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

As used herein, the term “fully complementary” refers to 100% complementarity.

As used herein, the term “identity” refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (e.g. in the contiguous nucleotide sequence of the compound of the invention and in the reference sequence), dividing that number by the total number of nucleotides in the aligned region and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

As used herein, the term “hybridizing” or “hybridizes” refers to two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

As used herein, the term “target nucleic acid” refers to the nucleic acid which encodes a mammalian ATXN3 protein and may for example be a gene, a ATXN3 RNA, a mRNA, a pre-mRNA, a mature mRNA or a cDNA sequence. The target may therefore be referred to as an “ATXN3 target nucleic acid”.

In some embodiments, the target nucleic acid encodes a human ATXN3 protein, such as the human ATXN3 gene encoding the pre-mRNA sequence provided herein as SEQ ID NO:1. Thus, the target nucleic acid may be SEQ ID NO:1.

In some embodiments, the target nucleic acid encodes a mouse ATXN3 protein. Suitably, the target nucleic acid encoding a mouse ATXN3 protein comprises a sequence as shown in SEQ ID NO: 3.

In some embodiments, the target nucleic acid encodes a cynomolgus monkey ATXN3 protein. Suitably, the target nucleic acid encoding a cynomolgus monkey ATXN3 protein comprises a sequence as shown in SEQ ID NO: 2.

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the ATXN3 target nucleic acid in a cell which is expressing the ATXN3 target nucleic acid. The contiguous sequence of nucleobases of the oligonucleotide of the invention is typically complementary to the ATXN3 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid is a messenger RNA, such as a mature mRNA or a pre-mRNA which encodes mammalian ATXN3 protein, such as human ATXN3, e.g. the human ATXN3 pre-mRNA sequence, such as that disclosed as SEQ ID NO:1, or ATXN3 mature mRNA. Further, the target nucleic acid may be a cynomolgus monkey ATXN3 pre-mRNA sequence, such as that disclosed as SEQ ID NO:1, or a cynomolgus monkey ATXN3 mature mRNA. Further, the target nucleic acid may be a mouse ATXN3 pre-mRNA sequence, such as that disclosed as SEQ ID NO:3, or mouse ATXN3 mature mRNA. SEQ ID NOS:1-3 are DNA sequences it will be understood that target RNA sequences have uracil (U) bases in place of the thymidine bases (T).

TABLE 1
Target nucleic acids

| Target Nucleic Acid | Sequence ID |
|---|---|
| ATXN3 Homo sapien spre-mRNA | SEQ ID NO: 1 |
| ATXN3 Macaca fascicularis pre-mRNA | SEQ ID NO: 2 |
| ATXN3 Mus musculus mRNA | SEQ ID NO: 3 |

In some embodiments, the oligonucleotide of the invention targets SEQ ID NO:1.

In some embodiments, the oligonucleotide of the invention targets SEQ ID NO:2.

In some embodiments, the oligonucleotide of the invention targets SEQ ID NO:3.

In some embodiments, the oligonucleotide of the invention targets SEQ ID NO:1 and SEQ ID NO:2.

In some embodiments, the oligonucleotide of the invention targets SEQ ID NO:1 and SEQ ID NO:3.

In some embodiments, the oligonucleotide of the invention targets SEQ ID NO:1, SEQ ID NO:2 and SEQ ID NO:3.

As used herein, the term “target sequence” refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention.

Herein are provided numerous target sequence regions, as defined by regions of the human ATXN3 pre-mRNA (using SEQ ID NO:1 as a reference) which may be targeted by the oligonucleotides of the invention.

In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence which are complementary to a target sequence present in the target nucleic acid molecule. The contiguous nucleotide sequence (and therefore the target sequence) comprises at least 10 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

As used herein, the term “target sequence region” refers to an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary to a region of SEQ ID NO:1. The region of SEQ ID NO:1 to which the antisense oligonucleotide of the invention is complementary to is referred to as the target sequence region.

In some embodiments the target sequence region is AAGAGTAAAATATGGGT (SEQ ID NO:1093).

In some embodiments the target sequence region is GAATGTAAAAGTGTACAG (SEQ ID NO:1094).

In some embodiments the target sequence region is GGAATGTAAAAGTGTACA (SEQ ID NO:1095).

In some embodiments the target sequence region is GGGAATGTAAAAGTGTAC (SEQ ID NO:1096).

In some embodiments the target sequence region is TTGATGGTATAATGAAGAA (SEQ ID NO:1097).

In some embodiments the target sequence region is GGAAGATGTAAATAAGATT (SEQ ID NO:1098).

In some embodiments, the target sequence region is AAGATGTAAATAAGATTC (SEQ ID NO:1992).

It is to be understood that target RNA sequences have uracil (U) bases in place of any thymidine (T) bases.

As used herein, the term “off-target sequence” refers to a sequence of nucleotides comprising a nucleobase sequence which may be partially complementary to an oligonucleotide of the invention but which is present in another nucleic acid than the target (ATXN3) nucleic acid.

As used herein, the term “target cell” refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell (e.g. a cynomolgus monkey cell) or a human cell.

In preferred embodiments the target cell expresses human ATXN3 mRNA, such as the ATXN3 pre-mRNA, e.g. SEQ ID NO:1, or ATXN3 mature mRNA. In some embodiments the target cell expresses monkey ATXN3 mRNA, such as the ATXN3 pre-mRNA, e.g. SEQ ID NO:2, or ATXN3 mature mRNA. In some embodiments the target cell expresses mouse ATXN3 mRNA, such as the ATXN3 pre-mRNA, e.g. SEQ ID NO:3, or ATXN3 mature mRNA. The poly A tail of ATXN3 mRNA is typically disregarded for antisense oligonucleotide targeting.

As used herein, the term “naturally occurring variant” refers to variants of ATXN3 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

The Homo sapiens ATXN3 gene is located at chromosome 14, 92058552..92106621, complement (NC 000014.9, Gene ID 4287).

In some embodiments, the naturally occurring variants have at least 95% such as at least 98% or at least 99% homology to a mammalian ATXN3 target nucleic acid, such as a target nucleic acid selected form the group consisting of SEQ ID NOS:1, 2 and 3. In some embodiments the naturally occurring variants have at least 99% homology to the human ATXN3 target nucleic acid of SEQ ID NO:1.

As used herein, the term “modulation of expression” refers to an overall term for an oligonucleotide's ability to alter the amount of ATXN3 protein or ATXN3 mRNA when compared to the amount of ATXN3 or ATXN3 mRNA prior to administration of the oligonucleotide. Alternatively modulation of expression may be determined by reference to a control experiment. It is generally understood that the control is an individual or target cell treated with a saline composition or an individual or target cell treated with a non-targeting oligonucleotide (mock).

One type of modulation is an oligonucleotide's ability to inhibit, down-regulate, reduce, suppress, remove, stop, block, prevent, lessen, lower, avoid or terminate expression of ATXN3, e.g. by degradation of ATXN3 mRNA.

As used herein, a “high affinity modified nucleoside” refers to a modified nucleoside which, when incorporated into the oligonucleotide enhances the affinity of the oligonucleotide for its complementary target, for example as measured by the melting temperature (Tₘ). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

As used herein, the term “RNase H activity” refers to the ability of an antisense oligonucleotide to recruit RNase H when in a duplex with a complementary RNA molecule. WO 01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

The antisense oligonucleotide of the invention, or contiguous nucleotide sequence thereof may be a gapmer. The antisense gapmers are commonly used to inhibit a target nucleic acid via RNase H mediated degradation.

As used herein, the term “gapmer oligonucleotide” refers to an oligonucleotide that comprises at least three distinct structural regions—a 5′-flank, a gap and a 3′-flank (F-G-F′)- in the ‘5→3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

As used herein, “region G (gap region)” of the gapmer refers to a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (^{me}C). In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages. Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Alternatively, there are numerous reports of the insertion of a modified nucleoside which confers a 3′ endo conformation into the gap region of gapmers, whilst retaining some RNaseH activity. Such gapmers with a gap region comprising one or more 3′ endo modified nucleosides are referred to as “gap-breaker” or “gap-disrupted” gapmers, see for example WO2013/022984.

As used herein, the term “gap-breaker” or “gap-disrupted” refers to oligonucleotides that retain sufficient region of DNA nucleosides within the gap region to allow for RNaseH recruitment. The ability of “gap-breaker” oligonucleotide design to recruit RNaseH is typically sequence or even compound specific see Rukov et al. 2015 Nucl. Acids Res. Vol. 43 pp. 8476-8487, which discloses “gap-breaker” oligonucleotides which recruit RNaseH which in some instances provide a more specific cleavage of the target RNA. Modified nucleosides used within the gap region of gap-breaker oligonucleotides may for example be modified nucleosides which confer a 3′ endo confirmation, such 2′ O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of “gap-breaker” or “gap-disrupted” gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include:

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

As used herein, “region F (flanking region)” of the gapmer refers to a region of nucleosides that is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

As used herein, “region F′ (flanking region)” of the gapmer refers to a region of nucleosides that is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

As used herein, the term “LNA gapmer” refers to a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

As used herein, the term “MOE gapmer” refers to a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

As used herein, the term “mixed wing gapmer” refers to an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

As used herein, the term “Alternating Flank Gapmer” refers to LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and there is at least one DNA nucleoside positioned between the 5′ and 3′ most LNA nucleosides of region F or F′ (or both region F and F′).

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as “region D′” and “region D″” herein.

The addition of “region D′” or “region D″” may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonuclease protection or for ease of synthesis or manufacture.

“Region D′” and “Region D″” can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

“Region D′” or “Region D″” may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

As used herein, the term “conjugate” refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

As used herein, the term “linkage” or “linker” refers to a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

As used herein, the term “Region B” refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to Si nuclease cleavage. DNA phosphodiester containing biocleavable linkers are described in more detail in WO 2014/076195 (hereby incorporated by reference) see also region D′ or D″ herein.

As used herein, the term “Region Y” refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B—Y—C, A-Y—B—C or A-Y-C. In some embodiments the linker (region Y) is an amino alkyl, such as a C2 to C36 amino alkyl group, including, for example C6 to C12 amino alkyl groups. In a preferred embodiment the linker (region Y) is a C6 amino alkyl group.

### II. Oligonucleotides for Inhibiting ATXN3

The invention relates to oligonucleotides, such as antisense oligonucleotides, targeting ATXN3 expression.

The oligonucleotides of the invention targeting ATXN3 are capable of hybridizing to and inhibiting the expression of a ATXN3 target nucleic acid in a cell which is expressing the ATXN3 target nucleic acid.

The ATXN3 target nucleic acid may be a mammalian ATXN3 mRNA or premRNA, such as a human, mouse or monkey ATXN3 mRNA or premRNA. In some embodiments, the ATXN3 target nucleic acid is ATXN3 mRNA or premRNA for example a premRNA or mRNA originating from the Homo sapiens Ataxin 3 (ATXN3), RefSeqGene on chromosome 14, exemplified by NCBI Reference Sequence NM 004993.5 (SEQ ID NO:1).

The human ATXN3 pre-mRNA is encoded on Homo sapiens Chromosome 14, NC 000014.9 (92058552..92106621, complement). GENE ID=4287 (ATXN3).

The oligonucleotides of the invention are capable of inhibiting the expression of ATXN3 target nucleic acid, such as the ATXN3 mRNA, in a cell which is expressing the target nucleic acid, such as the ATXN3 mRNA (e.g. a human, monkey or mouse cell).

In some embodiments, the oligonucleotides of the invention are capable of inhibiting the expression of ATXN3 target nucleic acid in a cell which is expressing the target nucleic acid, so to reduce the level of ATXN3 target nucleic acid (e.g. the mRNA) by at least 50%, at least 60%, at least 70%, at least 80%, or at least 90% inhibition compared to the expression level of the ATXN3 target nucleic acid (e.g. the mRNA) in the cell. Suitably the cell is selected from the group consisting of a human cell, a monkey cell and a mouse cell. In some embodiments, the cell is a SK-N-AS, A431, NCI-H23 or ARPE19 cell (for more information on these cells, see Examples). Example 1 provides a suitable assay for evaluating the ability of the oligonucleotides of the invention to inhibit the expression of the target nucleic acid. Suitably the evaluation of a compounds ability to inhibit the expression of the target nucleic acid is performed in vitro, such a gymnotic in vitro assay, for example as according to Example 1.

In some embodiments, an oligonucleotide of the invention is more capable in inhibiting the expression of ATXN3 target nucleic acid in a cell which is expressing the target nucleic acid than in inhibiting the expression of KCNB2 nucleic acid in a cell which is expressing the KCNB2 nucleic acid, providing for a higher selectivity in targeting the ATXN3 target nucleic acid. KCNB2 (Potassium Voltage-Gated Channel Subfamily B Member 2) nucleic acid was identified as containing a potential off-target sequence which may be annealed to certain oligonucleotides targeting SEQ ID NO:1098 and/or SEQ ID NO:1992. Information, including sequence information, about the KCNB2 gene and transcripts can be found in the public database Ensembl (release 101) at gene id ENSG00000182674.

Suitably, the capability of an oligonucleotide to inhibit the expression of ATXN3 and KCNB2 nucleic acids is tested in a cell expressing both nucleic acids. In some embodiments, an oligonucleotide of the invention is capable of inhibiting the expression of ATXN3 target nucleic acid so as to reduce the level of ATXN3 target nucleic acid (e.g. the mRNA) by a fraction which is at least 50%, at least 60%, at least 70%, at least 80%, or at least 90% inhibition compared to the expression level of the ATXN3 target nucleic acid (e.g. the mRNA) in the cell, but reduces the level of KCNB2 off-target nucleic acid (e.g., the mRNA) as compared to the expression level of the KCNB2 target nucleic acid (e.g. the mRNA) in the cell by a smaller fraction. The cell, may, for example, be selected from the group consisting of a human cell, a monkey cell and a mouse cell. In some embodiments, the cell is a neuronal cell, such as an iCell® Glutaneuron cell (for more information on these cells, see Table 2). Examples 5 and 14 provides suitable assays for evaluating the ability of the oligonucleotides of the invention to inhibit the expression of the target nucleic acid as compared to the off-target nucleic acid. Suitably the evaluation of the ability of an oligonucleotide to inhibit the expression of the target nucleic acid and the off-target nucleic acid is performed in vitro, such a gymnotic in vitro assay, for example as according to Example 14.

Advantageously, an oligonucleotide according to the invention has a low EC50 in inhibiting the expression of ATXN3 target nucleic acid in a cell which is expressing the target nucleic acid, providing for a high efficacy and/or potency in targeting the ATXN3 target nucleic acid. In some embodiments, the EC50 for inhibiting the expression of ATXN3 target nucleic acid is no more than about 1 μM, such as no more than about 500 nM, such as no more than about 300 nM, such as no more than about 200 nM, such as no more than about 180 nM, such as no more than about 170 nM, such as no more than about 160 nM, such as no more than about 150 nM, such as no more than about 140 nM, such as no more than about 130 nM, such as no more than about 120 nM, such as no more than about 110 nM, such as no more than about 100 nM, such as no more than about 90 nM, such as no more than about 80 nM, such as no more than about 70 nM, such as no more than about 60 nM, such as no more than about 50 nM. The cell, may, for example, be selected from the group consisting of a human cell, a monkey cell and a mouse cell. In some embodiments, the cell is a neuronal cell, such as a human neuronal cell, such as an iCell® GlutaNeuron cell (for more information on these cells, see Table 2). A particularly suitable assay is described in Example 16.

Preferably, an oligonucleotide according to the invention also or alternatively has a lower EC50 for inhibiting the expression of ATXN3 target nucleic acid (e.g., mRNA) in a cell than for inhibiting the expression of KCNB2 off-target nucleic acid (e.g., mRNA) in the cell, indicating that 50% inhibition of the expression of the nucleic acid is, for ATXN3 target nucleic acid, achieved at a lower oligonucleotide concentration, thereby providing for a higher selectivity. In some embodiments, the ratio between the EC50 for inhibiting the expression of KCNB2 off-target nucleic acid (e.g., mRNA) and the EC50 for inhibiting the expression of ATXN3 target nucleic acid is at least about 2, such as at least about 2.1, such as at least about 2.2, such as at least about 2.5, such as at least about 3, such as at least about 4, such as at least about 5, such as at least about 6, such as at least about 7, such as at least about 8, such as at least about 9, such as at least about 10, such as at least about 12, such as at least about 15, such as at least about 20, such as at least about 50, such as at least about 100, such as at least about 200, such as at least about 400, such as at least about 600, such as at least about 1000. The cell, may, for example, be selected from the group consisting of a human cell, a monkey cell and a mouse cell. In some embodiments, the cell is a neuronal cell, such as a human neuronal cell, such as an iCell® GlutaNeuron cell (for more information on these cells, see Table 2). Particularly suitable assays are described in Examples 14 and 16.

Typically, an oligonucleotide according to the invention also or alternatively has a low toxicity. Suitably, this can be tested in an in vitro assay, such as, e.g., in any one or more of the assays described in Example 7.

An aspect of the present invention relates to an antisense oligonucleotide, such as an LNA antisense oligonucleotide gapmer which comprises a contiguous nucleotide sequence of 10 to 30 nucleotides in length with at least 90% complementarity, such as is fully complementary to SEQ ID NO:1, 2 or 3.

In some embodiments, the oligonucleotide comprises a contiguous sequence of 10-30 nucleotides, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence. The sequences of suitable target nucleic acids are described herein above.

In some embodiments, the oligonucleotide of the invention comprises a contiguous nucleotides sequence of 12-24, such as 13, 14, 15, 16, 17, 18, 19, 20, 21, 22, or 23, contiguous nucleotides in length, wherein the contiguous nucleotide sequence is fully complementary to a target nucleic acid having a sequence as provided in the section “Taget sequence regions” above.

In some embodiments, the antisense oligonucleotide of the invention comprises a contiguous nucleotides sequence of 12-15, such as 13, or 14, 15 contiguous nucleotides in length, wherein the contiguous nucleotide sequence is fully complementary to a target nucleic acid having a sequence as provided in the section “Target sequence regions” above.

Typically, the antisense oligonucleotide of the invention or the contiguous nucleotide sequence thereof is a gapmer, such as an LNA gapmer, a mixed wing gapmer, or an alternating flank gapmer.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to a target sequence comprised in a sequence selected from SEQ ID NO:1098, SEQ ID NO:1992, or both.

In some embodiments, the target sequence region of an antisense oligonucleotide according to the invention comprises or consists of SEQ ID NO:1098.

In some embodiments, the target sequence region of an antisense oligonucleotide according to the invention comprises or consists of SEQ ID NO:1992.

In some embodiments the contiguous nucleotide sequence of the antisense oligonucleotide according to the invention is less than 20 nucleotides in length. In some embodiments the contiguous nucleotide sequence of the antisense oligonucleotide according to the invention is 12-24 nucleotides in length. In some embodiments the contiguous nucleotide sequence of the antisense oligonucleotide according to the invention is 12-22 nucleotides in length. In some embodiments the contiguous nucleotide sequence of the antisense oligonucleotide according to the invention is 12-20 nucleotides in length. In some embodiments the contiguous nucleotide sequence of the antisense oligonucleotide according to the invention is 12-18 nucleotides in length. In some embodiments the contiguous nucleotide sequence of the antisense oligonucleotide according to the invention is 12-16 nucleotides in length.

Advantageously, in some embodiments all of the internucleoside linkages between the nucleosides of the contiguous nucleotide sequence are phosphorothioate internucleoside linkages.

In some embodiments, the contiguous nucleotide sequence is fully complementary to a target nucleic acid.

The oligonucleotide compounds represent specific designs of a motif sequence. Typically, capital letters or the HELM-designation [LR] represent beta-D-oxy LNA nucleosides, lowercase letters or [dR] represent DNA nucleosides, all LNA C are 5-methyl cytosine, and 5-methyl DNA cytosines are presented by “e” or mc or [5meC], and all internucleoside linkages are, unless otherwise indicated, stereoundefined phosphorothioate internucleoside linkages [sP].

Design refers to the gapmer design, F-G-F′, where each number represents the number of consecutive modified nucleosides, e.g. 2′ modified nucleosides (first number=5′ flank), followed by the number of DNA nucleosides (second number=gap region), followed by the number of modified nucleosides, e.g. 2′ modified nucleosides (third number=3′ flank), optionally preceded by or followed by further repeated regions of DNA and LNA, which are not necessarily part of the contiguous nucleotide sequence that is complementary to the target nucleic acid.

Motif sequences represent the contiguous sequence of nucleobases present in the oligonucleotide, also referred to as the Oligonucleotide Base Sequence.

Typically, the antisense oligonucleotides is 12-24, such as 12-18, nucleosides in length wherein the antisense oligonucleotide comprises a contiguous nucleotide sequence comprising at least 12, such as at least 14, such as at least 15 contiguous nucleotides present in a sequence selected from SEQ ID NO:1122 and SEQ ID NO:1816, with one or more of the further modifications described herein.

In some embodiments, the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

In some embodiments, the sugar-modified nucleosides of region F and F′ are independently selected from the group consisting of 2′-O-alkyl-RNA, 2′-O-methyl-RNA, 2′-O-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

In some embodiments, region G comprises 5-16 contiguous DNA nucleosides.

In some embodiments, the antisense oligonucleotide is an LNA gapmer oligonucleotide comprising LNA nucleosides.

In some embodiments, the LNA nucleosides are beta-D-oxy LNA nucleosides.

In some embodiments, substantially all, or all of the internucleoside linkages between the contiguous nucleosides are phosphorothioate internucleoside linkages.

In some embodiments, substantially all, or all phosphorothioate internucleoside linkages between the contiguous nucleosides are stereo-undefined phosphorothioate internucleoside linkages.

In some embodiments, one or more internucleoside linkages between the contiguous nucleosides are stereodefined phosphorothioate internucleoside linkages.

Particular sequence motifs and antisense oligonucleotides of the present invention are shown in Table 11 of Example 13, wherein each compound represents a separate specific embodiment according to the invention.

In some embodiments, the antisense oligonucleotide comprises a contiguous nucleotide sequence comprising the base sequence of an antisense oligonucleotide selected from the group consisting of Compound ID Nos. 1122_82 to 1122_336, shown in Table 11.

In some embodiments, the antisense oligonucleotide comprises or consists of an antisense oligonucleotide selected from the group consisting of Compound ID Nos. 1122_82 to 1122_336, shown in Table 11.

In some embodiments, the antisense oligonucleotide comprises a contiguous nucleotide sequence comprising the nucleoside base sequence and, optionally, the sugar moiety modifications, of an antisense oligonucleotide selected from the group consisting of Compound ID Nos. 1122_91, 1122_107, 1122_154, 1122_155, 1122_156, 1122_157, 1122_158, 1122_167, 1122_172, 1122_175, 1122_294, and 1122_296, shown in Table 14.

In one aspect, the antisense oligonucleotide is an LNA gapmer antisense oligonucleotide comprising a contiguous nucleotide sequence comprising the contiguous nucleotides present in SEQ ID NO:1122 except for the modified nucleosides and modified intemucleoside linkages indicated in Table 15 for each residue in SEQ ID NO:1122. In a specific embodiment, the antisense oligonucleotide is more capable of inhibiting the expression of human ATXN3 than human KCNB2 in a cell which is expressing human ATXN3 and human KCNB2.

In one embodiment, the LNA gapmer antisense oligonucleotide comprises the contiguous nucleotides present in SEQ ID NO:1122, wherein all internucleoside linkages are stereo-undefined phosphorothioate internucleoside linkages, wherein 2, 3 or 4 of the nucleosides in each of region F and region F′ are beta-D-oxy LNA nucleosides [LR], typically wherein each beta-D-oxy LNA cytosine is 5-methyl cytosine [LR](5meC), except for:
(a) residue 10 being a 2′-O-methyl uracil nucleoside [mR](U) (e.g., Compound ID No. 1122_91);
(b) residue 3 being a 2′-O-methyl uracil nucleoside [mR](U) (e.g., Compound ID No. 1122_107);
(c) the intemucleoside linkage between residues 11 and 12 being a stereodefined phosphorothioate internucleoside linkage [ssP] (e.g., Compound ID No. 1122_154);
(d) the intemucleoside linkage between residues 12 and 13 being a stereodefined phosphorothioate internucleoside linkage [ssP] (e.g., Compound ID No. 1122_155);
(e) the intemucleoside linkage between residues 13 and 14 being a stereodefined phosphorothioate internucleoside linkage [ssP] (e.g., Compound ID No. 1122_156);
(f) the intemucleoside linkage between residues 14 and 15 being a stereodefined phosphorothioate internucleoside linkage [ssP] (e.g., Compound ID No. 1122_157);
(g) the intemucleoside linkage between residues 15 and 16 being a stereodefined phosphorothioate internucleoside linkage [ssP] (e.g., Compound ID No. 1122_158);
(h) residue 7 being a 2′-O-methoxyethyl adenine nucleoside [MOE](A) (e.g., Compound ID No. 1122_167);
(i) residue 15 being a 2′-O-methyl cytosine nucleoside [mR](C) (e.g., Compound ID NO. 1122_172);
(j) residue 11 being a 2′-O-methyl adenine nucleoside [mR](A) (e.g., Compound ID No. 1122_175);
(k) residue 18 being a 2′-fluoro cytosine nucleoside [fR](C) (e.g., Compound ID No. 1122_294);
(l) residue 4 being a 2′-O-methyl cytosine nucleoside [mR](C) (e.g., Compound ID No. 1122_296); or
(m) a combination of any two or more of (a) to (l).

In some embodiments, the one or more modified nucleosides and/or one or more modified intemucleoside linkages are, for each residue in SEQ ID NO:1122, independently selected from the options for that residue as shown in Table 15.

In one aspect, the antisense oligonucleotide is an LNA gapmer antisense oligonucleotide comprising a contiguous nucleotide sequence comprising the contiguous nucleotides present in SEQ ID NO:1816 except for the modified nucleosides and modified internucleoside linkages indicated in Table 16 for each residue in SEQ ID NO:1816. In a specific embodiment, the antisense oligonucleotide is more capable of inhibiting the expression of human ATXN3 than human KCNB2 in a cell which is expressing human ATXN3 and human KCNB2.

In one embodiment, the LNA gapmer antisense oligonucleotide comprises the contiguous nucleotides present in SEQ ID NO:1816, wherein all internucleoside linkages are stereo-undefined phosphorothioate internucleoside linkages, wherein 4, 5 or 6 of the nucleosides in the F-region and 2 or 3 of the nucleosides in the F′-region are beta-D-oxy LNA nucleosides [LR], typically wherein each beta-D-oxy LNA cytosine is 5-methyl cytosine [LR](5meC), except for:
(a) the internucleoside linkage between residues 12 and 13 being a stereodefined phosphorothioate internucleoside linkage [ssP] (e.g., Compound ID No. 1816_13);
(b) the internucleoside linkage between residues 14 and 15 being a stereodefined phosphorothioate internucleoside linkage [ssP] (e.g., Compound ID No. 1816_15);
(c) residue 8 being a 2′-fluoro adenine nucleoside [fR](A) (e.g., Compound ID No. 1816_28);
(d) residues 1, 2, 3, 5, 7, 8, 16, 17 and 18 being LNA beta-D-oxy LNA nucleosides [LR], wherein each beta-D-oxy LNA cytosine is 5-methyl cytosine [LR](5meC) (e.g, Compound ID No. 1816_41);
(e) residue 17 being a 2′-O-methoxyethyl thymine nucleoside [MOE](T) (e.g., Compound ID No. 1816_42);
(0 residue 16 being a 2′-O-methoxyethyl cytosine nucleoside [MOE](5meC) (e.g., Compound ID No. 1816_43);
(g) residue 8 being a 2′-O-methyl adenine nucleoside [mR](A) (e.g., Compound ID No. 1816_60);
(h) residue 3 being a 2′-O-methyl adenine nucleoside [mR](A) (e.g., Compound ID No. 1816_61);
(i) residue 16 being a 2′-O-fluoro cytosine nucleoside [fR](C) (e.g., Compound ID No. 1816_64);
(j) residue 16 being a 2′-O-methyl cytosine nucleoside [mR](C) (e.g., Compound ID No. 1816_65);
(k) residue 17 being a 2′-fluoro uracil nucleoside [fR](U) (e.g., Compound ID No. 1816_68); or
(l) a combination of any two or more of (a) to (k).

In some embodiments, the one or more modified nucleosides and/or one or more modified internucleoside linkages are, for each residue in SEQ ID NO:1816, independently selected from the options for that residue as shown in Table 16.

In some embodiments, the antisense oligonucleotide comprises or consists of Compound ID No. 1122_91, as shown in Table 11.

In some embodiments, the antisense oligonucleotide comprises or consists of Compound ID No. 1122_107, as shown in Table 11.

In some embodiments, the antisense oligonucleotide comprises or consists of Compound ID No. 1122_154, as shown in Table 11.

In some embodiments, the antisense oligonucleotide comprises or consists of Compound ID No. 1122_155, as shown in Table 11.

In some embodiments, the antisense oligonucleotide comprises or consists of Compound ID No. 1122_156, as shown in Table 11.

In some embodiments, the antisense oligonucleotide comprises or consists of Compound ID No. 1122_157, as shown in Table 11.

In some embodiments, the antisense oligonucleotide comprises or consists of Compound ID No. 1122_158, as shown in Table 11.

In some embodiments, the antisense oligonucleotide comprises or consists of Compound ID No. 1122_167, as shown in Table 11.

In some embodiments, the antisense oligonucleotide comprises or consists of Compound ID No. 1122_172, as shown in Table 11.

In some embodiments, the antisense oligonucleotide comprises or consists of Compound ID No. 1122_175, as shown in Table 11.

In some embodiments, the antisense oligonucleotide comprises or consists of Compound ID No. 1122_294, as shown in Table 11.

In some embodiments, the antisense oligonucleotide comprises or consists of Compound ID No. 1122_296, as shown in Table 11.

In some embodiments, the antisense oligonucleotide comprises or consists of Compound ID No. 1816_13, as shown in Table 11.

In some embodiments, the antisense oligonucleotide comprises or consists of Compound ID No. 1816_15, as shown in Table 11.

In some embodiments, the antisense oligonucleotide comprises or consists of Compound ID No. 1816_28, as shown in Table 11.

In some embodiments, the antisense oligonucleotide comprises or consists of Compound ID No. 1816_41, as shown in Table 11.

In some embodiments, the antisense oligonucleotide comprises or consists of Compound ID No. 1816_42, as shown in Table 11.

In some embodiments, the antisense oligonucleotide comprises or consists of Compound ID No. 1816_43, as shown in Table 11.

In some embodiments, the antisense oligonucleotide comprises or consists of Compound ID No. 1816_60, as shown in Table 11.

In some embodiments, the antisense oligonucleotide comprises or consists of Compound ID No. 1816_61, as shown in Table 11.

In some embodiments, the antisense oligonucleotide comprises or consists of Compound ID No. 1816_64, as shown in Table 11.

In some embodiments, the antisense oligonucleotide comprises or consists of Compound ID No. 1816_65, as shown in Table 11.

In some embodiments, the antisense oligonucleotide comprises or consists of Compound ID No. 1816_68, as shown in Table 11.

In some embodiments, the antisense oligonucleotide is an antisense oligonucleotide according to the following chemical annotation:
(a) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[m R]}U_{[sP]}.^{[dR]}(A)_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C)_{[sP]}.^{[LR][5me]}C (SEQ ID NO:1122, wherein residue 10 is U) (Compound ID No. 1122_91);
(b) ^{[LR]}A_{[sP]}.^{[LR]}A_{[sP]}.^{[mR]}U_{[sP]}.^{[LR][5me]}C_{[sP]}.^{[dR]}T_{[sP]}.^{[dR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[LR]}T_{[sP]}.^{[dR]}T_{[sP]}.^{[LR][5me]}C_{[sP]}.^{[LR][5me]}C (SEQ ID NO:1122, wherein residue 3 is U) (Compound ID No. 1122_107);
(c) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[ssP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_154);
(d) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[ssP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_155);
(e) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[ssP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_156);
(f) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[sP]}.^{[dR]}T_{[ssP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_157);
(g) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[sP]}.^{[dR]}A_{[sP]}.^{[dR]}T_{[sP]}.^{[dR]}C_{[ssP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_158);
(h) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[MOE]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_167);
(i) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[sP]}.^{[dR]}T_{[sP]}.^{[mR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_172);
(j) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_175);
(k) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[fR]}C_{[sP]}.^{[LR][5me]}C (SEQ ID NO:1122) (Compound ID No. 1122_294);
(l) ^{[LR]}A_{[sP]}.^{[dR]}A_{[sP]}.^{[dR]}T_{[sP]}.^{[mR]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[s P]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_296);
(m) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[ssP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_13);
(n) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[ssP]}.^{[dR]}T_{[sP]}.^{[LR][5me]}C_{[sP]}.^{[LR]}T_{[sP]}.^{[LR]}T (SEQ ID NO:1816) (Compound ID No. 1816_15);
(o) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[fR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_28);
(p) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_41);
(q) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[MOE]}T_{[sP]}.^{[LR]}T (SEQ ID NO:1816) (Compound ID No. 1816_42);
(r) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[MOE][5me]}C_{[sP]}.^{[LR]}T_{[sP]}.^{[LR]}T (SEQ ID NO:1816) (Compound ID No. 1816_43);
(s) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[mR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_60);
(t) ^{[LR]}G_{[sP]}.^{[LR]}A_{[sP]}.^{[mR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_61);
(u) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[fR]}C_{[sP]}.^{[LR]}T_{[sP]}.^{[LR]}T (SEQ ID NO:1816) (Compound ID No. 1816_64);
(v) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[mR]}C_{[sP]}.^{[LR]}T_{[sP]}.^{[LR]}T (SEQ ID NO:1816) (Compound ID No. 1816_65); or
(w) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[fR]}U_{[sP]}.^{[LR]}T (SEQ ID NO:1816, wherein residue 17 is U)(Compound ID No. 1816_68),
or is a pharmaceutically acceptable salt thereof, wherein
[LR] is a beta-D-oxy-LNA nucleoside,
[LR][5me]C is a beta-D-oxy-LNA 5-methyl cytosine nucleoside,
[dR] is a DNA nucleoside,
[sP] is a phosphorothioate internucleoside linkage (stereo undefined)
[ssP] is a stereodefined Sp phosphorothioate internucleoside linkage
[mR] is a 2′-O-methyl nucleoside,
[MOE] is a 2′-O-methoxyethyl nucleoside, and
[fR] is a 2′-fluoro nucleoside.

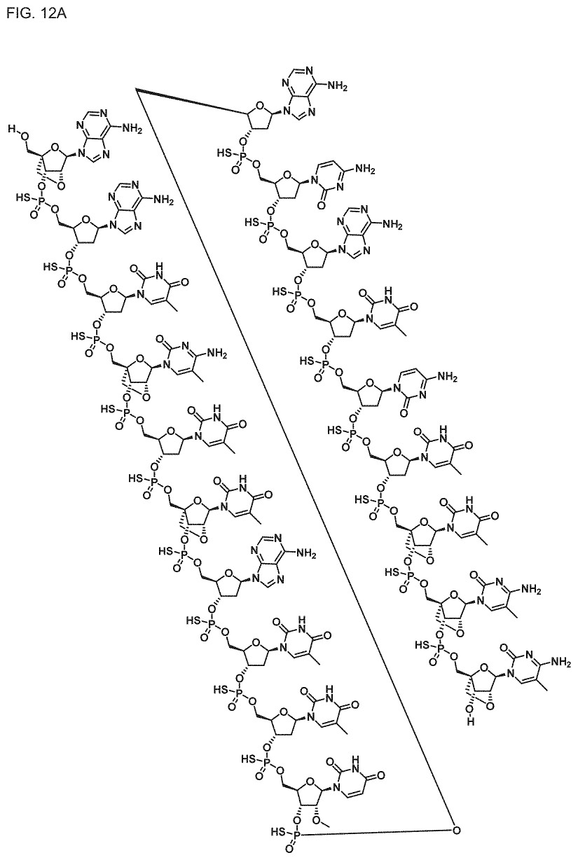

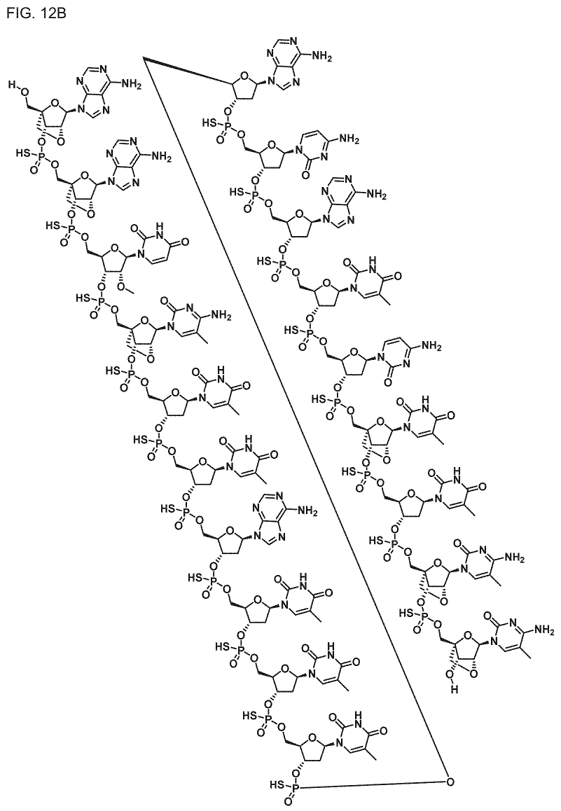

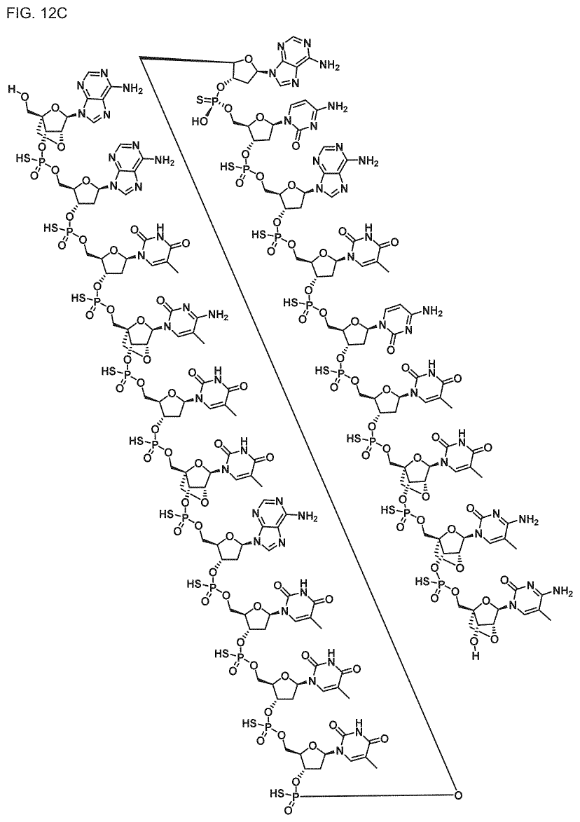

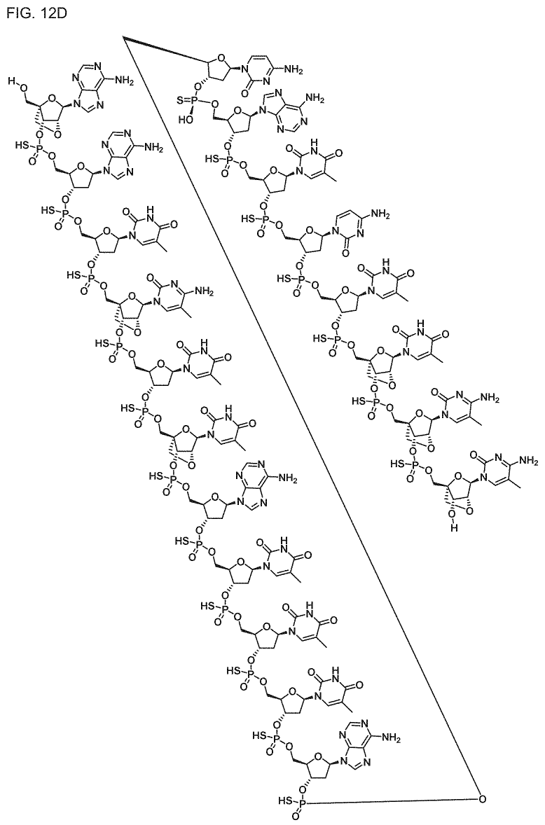

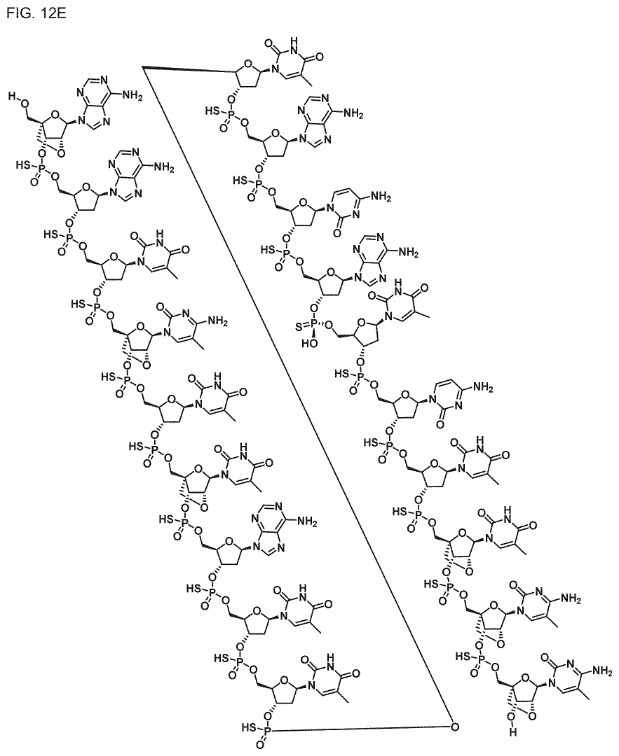

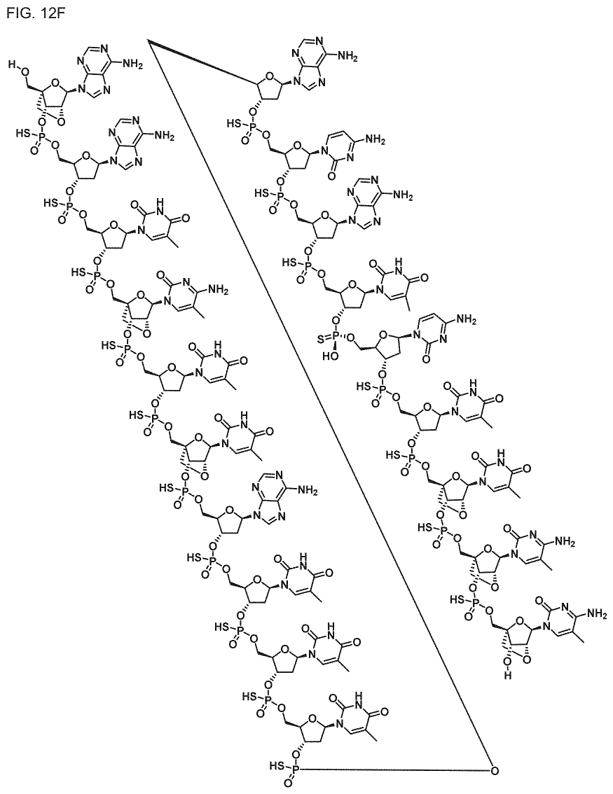

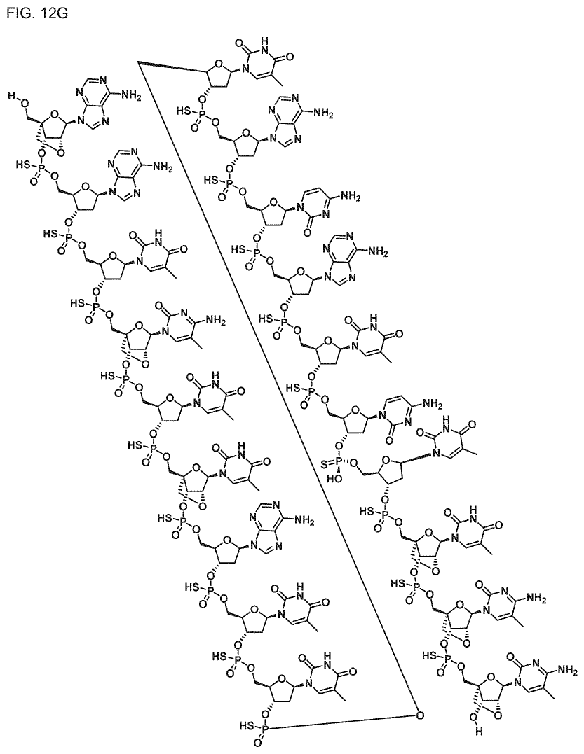

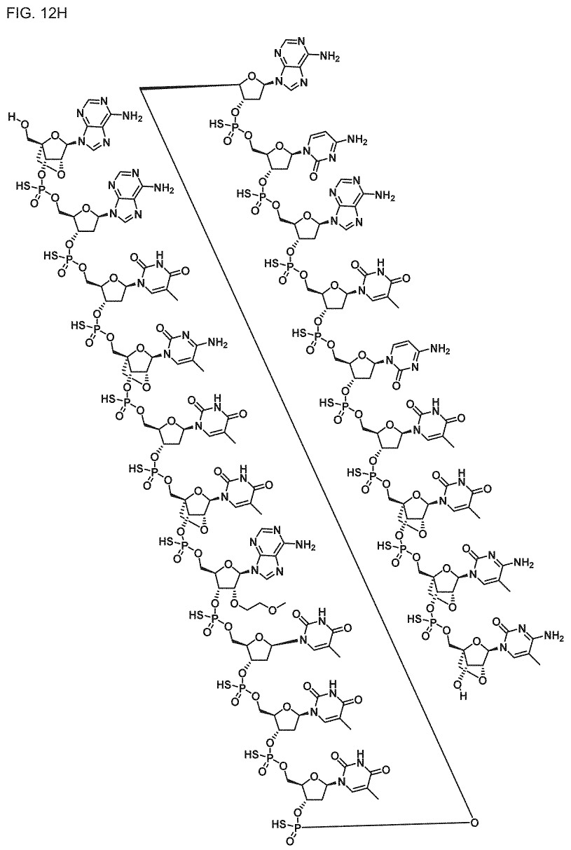

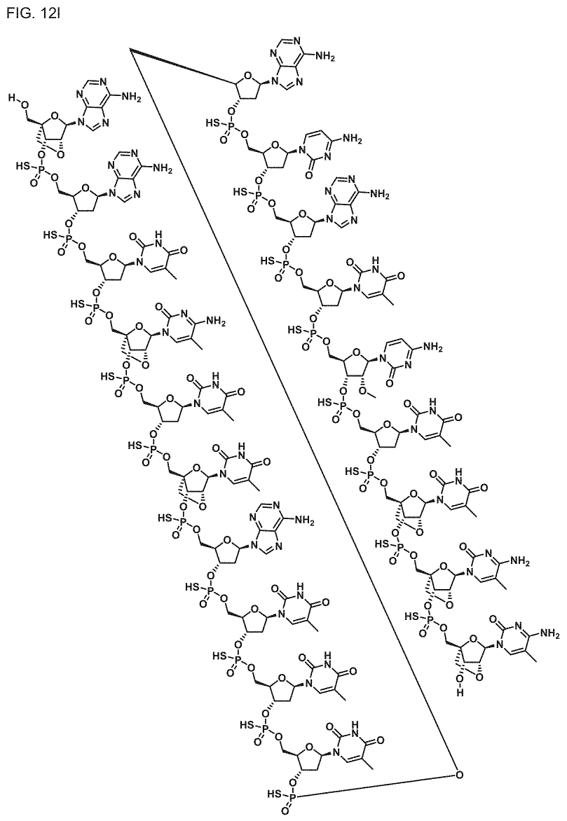

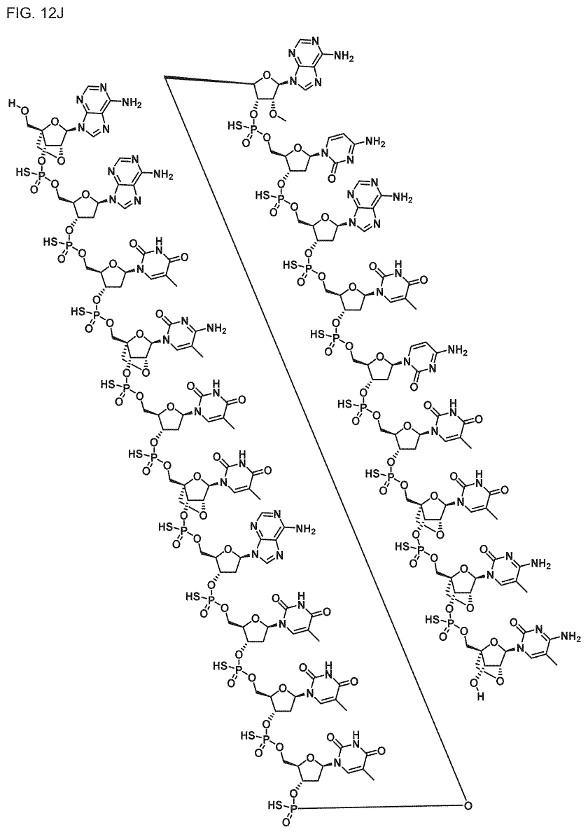

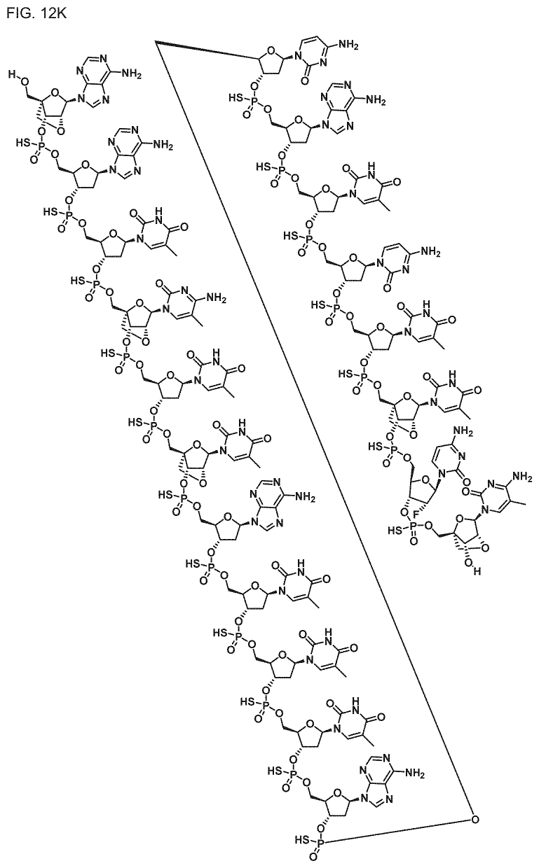

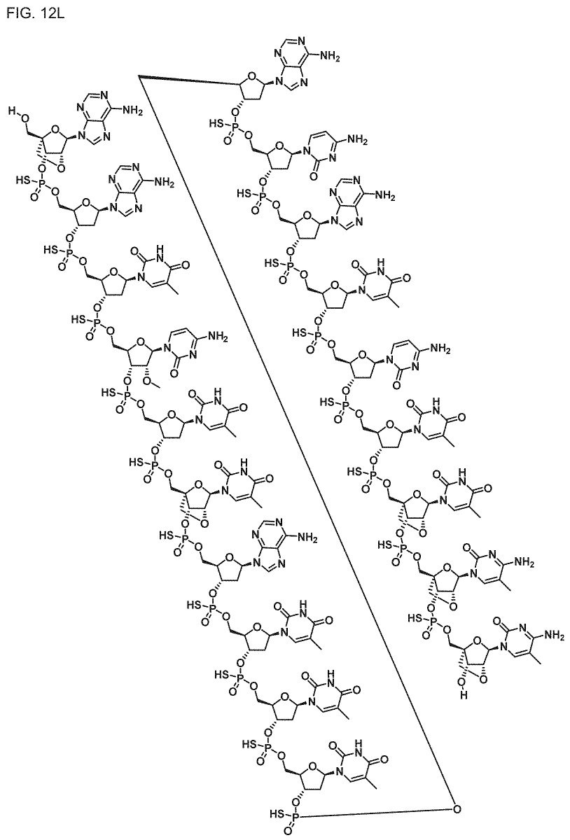

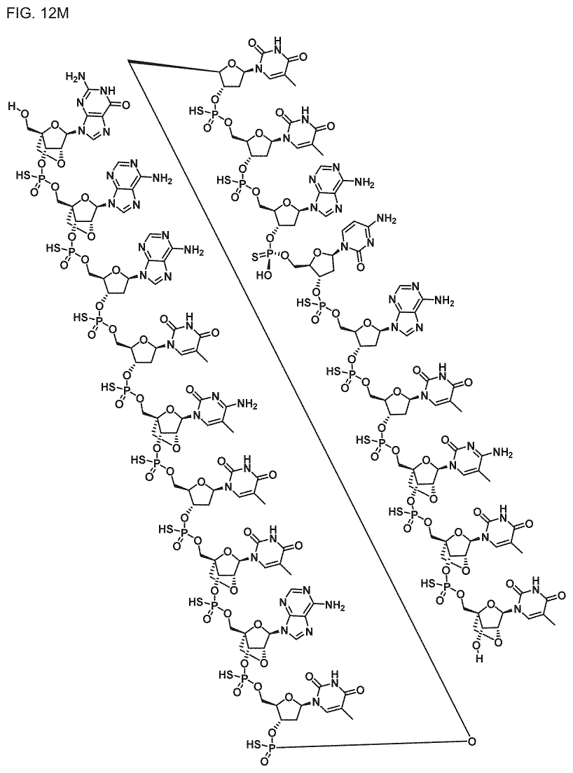

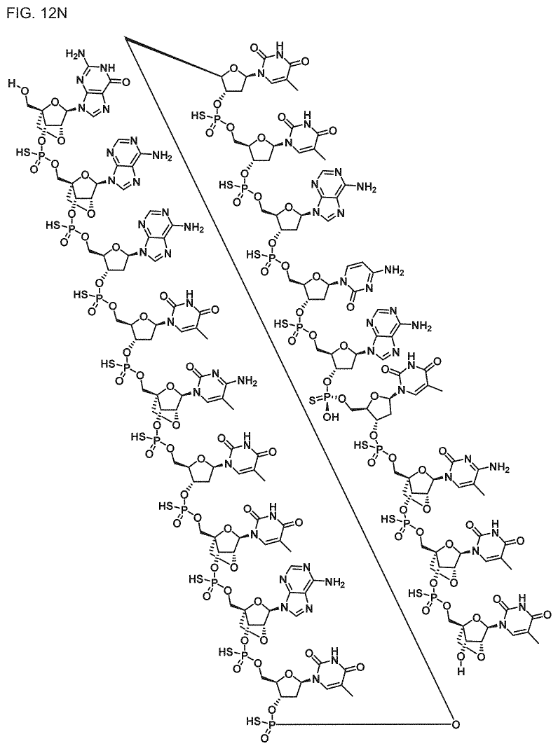

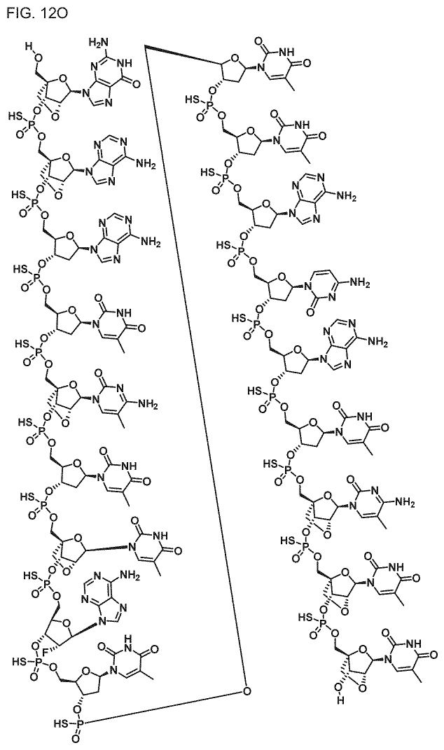

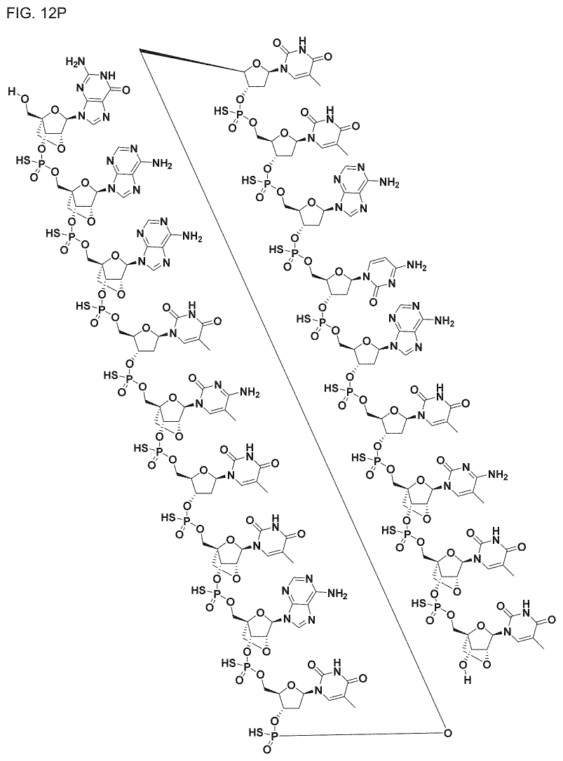

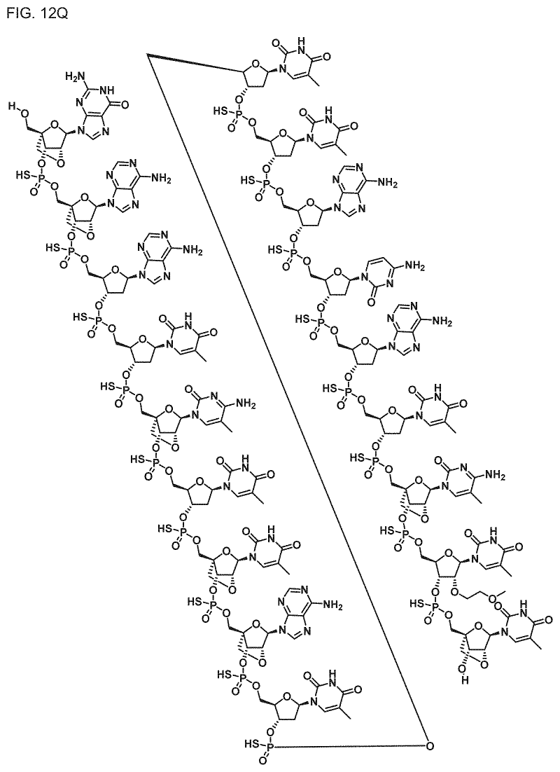

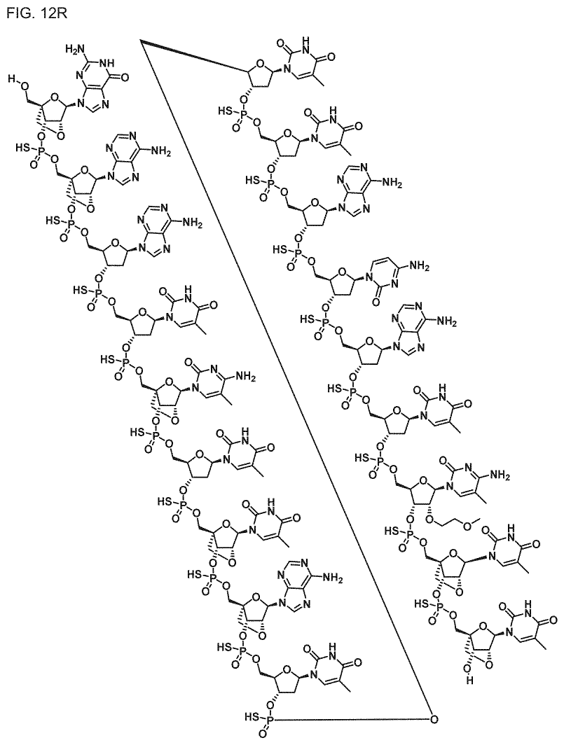

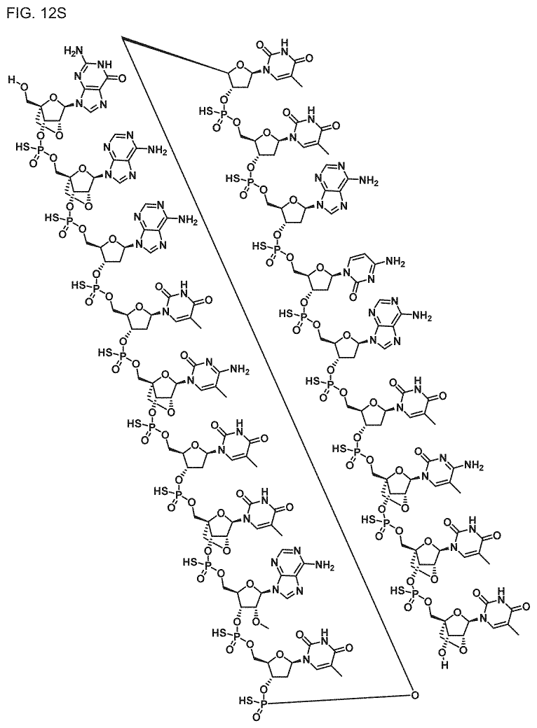

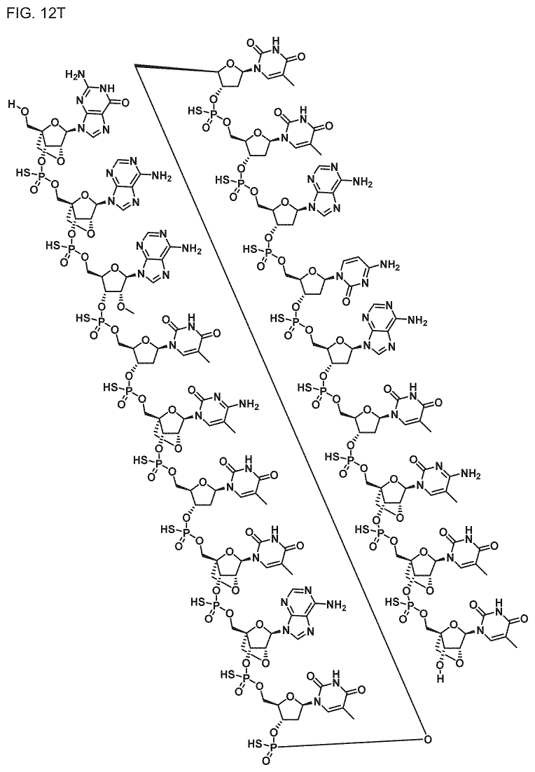

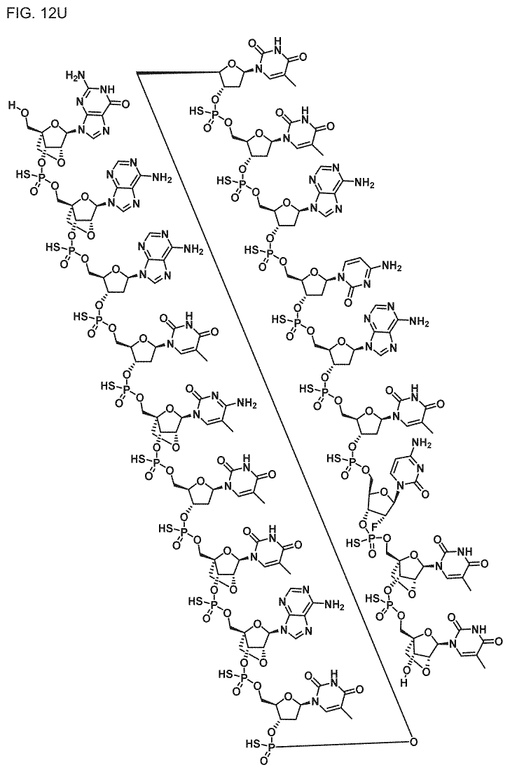

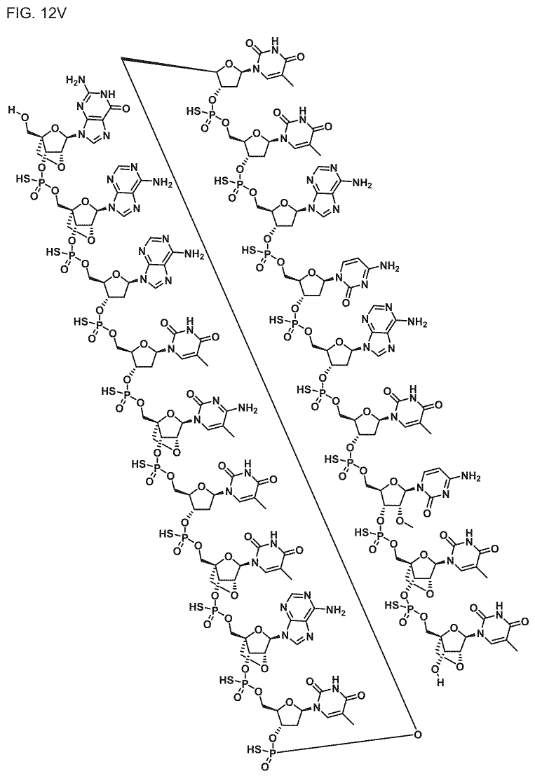

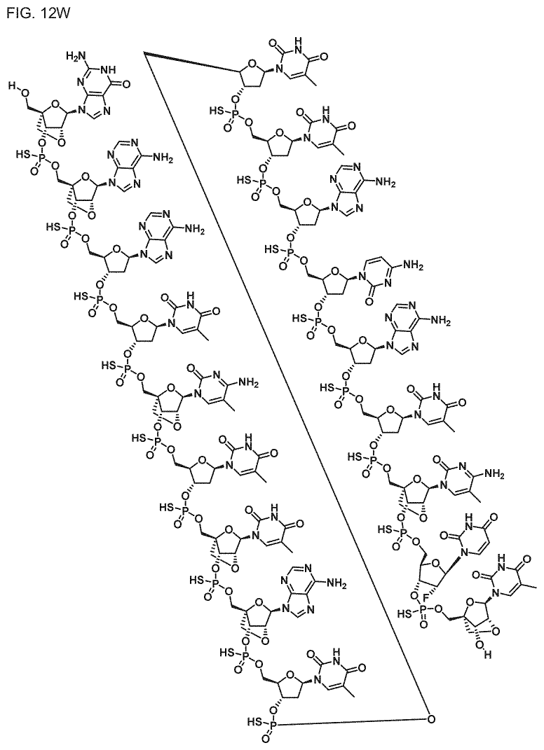

In one embodiment, the antisense oligonucleotide is the antisense oligonucleotide shown in FIG. 12A (Compound ID No. 1122_91); or a pharmaceutically acceptable salt thereof.

In one embodiment, the antisense oligonucleotide is the antisense oligonucleotide shown in FIG. 12B (Compound ID No. 1122_107); or a pharmaceutically acceptable salt thereof.

In one embodiment, the antisense oligonucleotide is the antisense oligonucleotide shown in FIG. 12C (Compound ID No. 1122_154); or a pharmaceutically acceptable salt thereof.

In one embodiment, the antisense oligonucleotide is the antisense oligonucleotide shown in FIG. 12D (Compound ID No. 1122_155); or a pharmaceutically acceptable salt thereof.

In one embodiment, the antisense oligonucleotide is the antisense oligonucleotide shown in FIG. 12E (Compound ID No. 1122_156); or a pharmaceutically acceptable salt thereof.

In one embodiment, the antisense oligonucleotide is the antisense oligonucleotide shown in FIG. 12F (Compound ID No. 1122_157); or a pharmaceutically acceptable salt thereof.

In one embodiment, the antisense oligonucleotide is the antisense oligonucleotide shown in FIG. 12G (Compound ID No. 1122_158); or a pharmaceutically acceptable salt thereof.

In one embodiment, the antisense oligonucleotide is the antisense oligonucleotide shown in FIG. 12H (Compound ID No. 1122_167); or a pharmaceutically acceptable salt thereof.

In one embodiment, the antisense oligonucleotide is the antisense oligonucleotide shown in FIG. 12I (Compound ID No. 1122_172); or a pharmaceutically acceptable salt thereof.

In one embodiment, the antisense oligonucleotide is the antisense oligonucleotide shown in FIG. 12J (Compound ID No. 1122_175); or a pharmaceutically acceptable salt thereof.

In one embodiment, the antisense oligonucleotide is the antisense oligonucleotide shown in FIG. 12K (Compound ID No. 1122_294); or a pharmaceutically acceptable salt thereof.

In one embodiment, the antisense oligonucleotide is the antisense oligonucleotide shown in FIG. 12L (Compound ID No. 1122_296); or a pharmaceutically acceptable salt thereof.

In one embodiment, the antisense oligonucleotide is the antisense oligonucleotide shown in FIG. 12M (Compound ID No. 1816_13); or a pharmaceutically acceptable salt thereof.

In one embodiment, the antisense oligonucleotide is the antisense oligonucleotide shown in FIG. 12N (Compound ID No. 1816_15); or a pharmaceutically acceptable salt thereof.

In one embodiment, the antisense oligonucleotide is the antisense oligonucleotide shown in FIG. 12O (Compound ID No. 1816_28); or a pharmaceutically acceptable salt thereof.

In one embodiment, the antisense oligonucleotide is the antisense oligonucleotide shown in FIG. 12P (Compound ID No. 1816_41); or a pharmaceutically acceptable salt thereof.

In one embodiment, the antisense oligonucleotide is the antisense oligonucleotide shown in FIG. 12Q (Compound ID No. 1816_42); or a pharmaceutically acceptable salt thereof.

In one embodiment, the antisense oligonucleotide is the antisense oligonucleotide shown in FIG. 12R (Compound ID No. 1816_43); or a pharmaceutically acceptable salt thereof.

In one embodiment, the antisense oligonucleotide is the antisense oligonucleotide shown in FIG. 12S (Compound ID No. 1816_60); or a pharmaceutically acceptable salt thereof.

In one embodiment, the antisense oligonucleotide is the antisense oligonucleotide shown in FIG. 12T (Compound ID No. 1816_61); or a pharmaceutically acceptable salt thereof.

In one embodiment, the antisense oligonucleotide is the antisense oligonucleotide shown in FIG. 12U (Compound ID No. 1816_64); or a pharmaceutically acceptable salt thereof.

In one embodiment, the antisense oligonucleotide is the antisense oligonucleotide shown in FIG. 12V (Compound ID No. 1816_65); or a pharmaceutically acceptable salt thereof.

In one embodiment, the antisense oligonucleotide is the antisense oligonucleotide shown in FIG. 12W (Compound ID No. 1816_68); or a pharmaceutically acceptable salt thereof.

### III. Compositions, Methods, and Applications for Inhibition of ATXN3 Expression

A. Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

B. Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant.

In a further aspect, the invention provides pharmaceutical compositions comprising any of the aforementioned oligonucleotides and/or oligonucleotide conjugates or salts thereof and a pharmaceutically acceptable diluent, carrier, salt or adjuvant.

A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

C. Applications

The oligonucleotides of the invention may be utilized as research reagents for, for example, diagnostics, therapeutics and prophylaxis.

In research, such oligonucleotides may be used to specifically modulate the synthesis of ATXN3 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating ATXN3 expression in a target cell which is expressing ATXN3, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal.

In diagnostics the oligonucleotides may be used to detect and quantitate ATXN3 expression in cell and tissues by northern blotting, in-situ hybridisation or similar techniques.

For therapeutics, an animal or a human, suspected of having a disease or disorder, which can be treated by modulating the expression of ATXN3

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of ATXN3. In some embodiments disease or disorder may be associated with a mutation in the ATXN3 gene. Therefore, in some embodiments, the target nucleic acid is a mutated form of the ATXN3 sequence.

The methods of the invention are preferably employed for treatment or prophylaxis against diseases caused by abnormal levels and/or activity of ATXN3.

The invention further relates to use of an oligonucleotide, oligonucleotide conjugate or a pharmaceutical composition as defined herein for the manufacture of a medicament for the treatment of abnormal levels and/or activity of ATXN3.

In one embodiment, the invention relates to oligonucleotides, oligonucleotide conjugates or pharmaceutical compositions for use in the treatment of spinocerebellar ataxia.

D. Administration

In some embodiments, the oligonucleotides or pharmaceutical compositions of the present invention may be administered oral. In further embodiments, the oligonucleotides or pharmaceutical compositions of the present invention may be administered topical or enteral or parenteral (such as, intravenous, subcutaneous, intra-muscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

E. Combination therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

### EXAMPLES

### Materials and Methods:

Oligonucleotide Synthesis:

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an MermMade 192 oligonucleotide synthesizer at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of The Oligonucleotide:

The coupling of 5′DMTr protected nucleoside O-cyanoethyl-phosphoramidites, including DNA-A(Bz), DNA-G(iBu), DNA-C(Bz), DNA-T, LNA-5-methyl-C(Bz), LNA-A(Bz), LNA-G(dmf), LNA-T, MOE-A(Bz), MOE-G(iBu), MOE-(T), MOE-(U), MOE-5-methyl-C(Bz), 2′F-A(Bz), 2′F(T), 2′F(U), 2′F—C(Ac), 2′F-G(iBu), 2′ OMe-A(Bz), 2′ OMe(U), 2′ OMe(T), 2′ OMe-C(Ac), 2′ OMe-G(iBu), 2′OMe-G(dmf) is performed by using a solution of 0.1 M of the 5′-O-DMT-protected amidite in acetonitrile and DCI (4,5-dicyanoimidazole) in acetonitrile (0.25 M) as activator.

Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

Abbreviations:
DCI: 4,5-Dicyanoimidazole
DCM: Dichloromethane
DMF: Dimethylformamidine
DMT: 4,4′-Dimethoxytrityl
THF: Tetrahydrofurane
Bz: Benzoyl
Ibu: Isobutyryl
RP-HPLC: Reverse phase high performance liquid chromatography

Tₘ Assay:

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

Cell Lines:

TABLE 2
Details in relation to the cell lines for assaying the compounds:

| | | | | | Hours of | |
|---|---|---|---|---|---|---|
| | | | | Cells/well | cell incubation | |
| Cell lines | | | | (96 well | prior to | Days of |
| Name | Vendor | Cat. no. | Cell medium | plate) | treatment | treatment |
| A431 | ECACC | 85090402 | EMEM (Cat. no. | 8000 | 24 | 3 |
| | | | M2279), 10% FBS | | | |
| | | | (Cat. no. F7524), | | | |
| | | | 2 mM Glutamine | | | |
| | | | (Cat. no. G8541), | | | |
| | | | 0.1 mM NEAA | | | |
| | | | (Cat. no. M7145), | | | |
| | | | 25 μg/ml Gentamicin | | | |
| | | | (Cat. no. G1397) | | | |
| NCI-H23 | ATCC | CRL-5800 | RPMI 1640 | 10000 | 24 | 3 |
| | | | (Cat. no. R2405), | | | |
| | | | 10% FBS (Cat. no. | | | |
| | | | F7524), 10 mM | | | |
| | | | Hepes (Cat. no. | | | |
| | | | H0887), 1 mM | | | |
| | | | Sodium Pyruvate | | | |
| | | | (Cat. no. S8636), | | | |
| | | | 25 μg/ml Gentamicin | | | |
| | | | (Cat. no. G1397) | | | |
| ARPE19 | ATCC | CRL-2302 | DMEM/F-12 HAM | 2000 | 0 | 4 |
| | | | (Cat. no. D8437), | | | |
| | | | 10% FBS (Cat. no. | | | |
| | | | F7524), 25 μg/ml | | | |
| | | | Gentamicin | | | |
| | | | (Cat. no. G1397) | | | |
| U251 | ECACC | 9063001 | EMEM (Cat. no. | 2000 | 0 | 4 |
| | | | M2279), 10% FBS | | | |
| | | | (Cat. no. F7524), | | | |
| | | | 2 mM Glutamine | | | |
| | | | (Cat. no. G8541), | | | |
| | | | 0.1 mM NEAA | | | |
| | | | (Cat. no. M7145), | | | |
| | | | 1 mM Sodium Pyruvate | | | |
| | | | (Cat. no. S8636), | | | |
| | | | 25 μg/ml Gentamicin | | | |
| | | | (Cat. no. G1397) | | | |
| U2-OS | ATCC | HTB-96 | MCCoy 5A medium | 7000 | 24 | 3 |
| | | | (Cat. no. M8403), | | | |
| | | | 10% FBS (Cat. no. | | | |
| | | | F7524), 1.5 mM | | | |
| | | | Glutamine (Cat. no. | | | |
| | | | G8541), 25 μg/ml | | | |
| | | | Gentamicin | | | |
| | | | (Cat. no. G1397) | | | |
| SK-N-AS | ATCC | CRL-2137 | Dulbecco's Modified | 9300 | 24 | 4 |
| | | | Eagle's Medium, | | | |
| | | | supplemented with | | | |
| | | | 0.1 mM Non-Essential | | | |
| | | | Amino Acids (NEAA) | | | |
| | | | and fetal bovine | | | |
| | | | serum to a final | | | |
| | | | concentration of 10% | | | |
| iCell ® | Stemcell | R1034 | BrainPhys Neuronal | 50.000-80.000 | 168 | 4 |
| GlutaNeurons | Technologies | | Medium (Cat. no. | | | |
| | | | 5790) supplemented | | | |
| | | | with iCell ® | | | |
| | | | GlutaNeurons Kit | | | |
| | | | (Stemcell Technologies. | | | |
| | | | no. R1034) according | | | |
| | | | to vendor), N-2 | | | |
| | | | (Thermo Fisher), | | | |
| | | | 1 μg/ml Laminin 512 | | | |
| | | | (BioLamina, no. LN521) | | | |

* All medium and additives are purchased from Sigma Aldrich unless otherweise stated.

### Example 1: Testing in vitro efficacy of LNA oligonucleotides in SK-N-AS, A431, NCI-H23 and ARPE19 cell lines at 25 and 5 μM

### Materials and Methods:

An oligonucleotide screen is performed in human cell lines using the LNA oligonucleotides in Table 3 (CMP ID NO: 4_1-1089_1, see column “oligonucleotide compounds”) targeting SEQ ID NO: 1. The human cell lines SK-N-AS, A341, NCI-H23 and ARPE19 are purchased from the vendors listed in Table 2, and are maintained as recommended by the supplier in a humidified incubator at 37° C. with 5% CO₂. For the screening assays, cells are seeded in 96 multi well plates in media recommended by the supplier (see Table 2 in the Materials and Methods section). The number of cells/well is optimized for each cell line (see Table 2 in the Materials and Methods section).

Cells are incubated between 0 and 24 hours before addition of the oligonucleotide in a concentration of 5 or 25 μM (dissolved in PBS). 3-4 days after addition of the oligonucleotide, the cells are harvested (The incubation times for each cell line are indicated in Table 2 in the Materials and Methods section).

RNA is extracted using the Qiagen RNeasy 96 kit (74182), according to the manufacturer's instructions). cDNA synthesis and qPCR is performed using qScript XLT one-step RT-qPCR ToughMix Low ROX, 95134-100 (Quanta Biosciences). Target transcript levels are quantified using FAM labeled TaqMan assays from Thermo Fisher Scientific in a multiplex reaction with a VIC labelled GUSB control. TaqMan primer assays for the target transcript of interest ATXN3 (see below) and a house keeping gene GUSB (4326320E VIC-MGB probe).

ATXN3 primer assay
(Assay ID: N/A Item Name Hs.PT.58.39355049):
Forward primer:
(SEQ ID NO: 1128)
GTTTCTAAAGACATGGTCACAGC
Reverse:
(SEQ ID NO: 1129)
CTATCAGGACAGAGTTCACATCC
Probe:
(SEQ ID NO: 1130)
56-FAM/AAAGGCCAG/ZEN/CCACCAGTTCAGG/3IABkFQ/

### Results:

The relative ATXN3 mRNA expression levels are determined as % of control (PBS-treated cells) i.e. the lower the value the larger the inhibition.

TABLE 3
Sequence Motifs and Compounds of Exemplary Compounds of the Invention

| | | | | | | Oligonucleotide |
|---|---|---|---|---|---|---|
| SEQ ID NO | motif sequence | start | end | design | CMP ID NO | compound |
| 4 | aagaaaccaaaccc | 743 | 756 | 2-10-2 | 4_1 | AAgaaaccaaacCC |
| 5 | aaagaaaccaaacc | 744 | 757 | 2-10-2 | 5_1 | AAagaaaccaaaCC |
| 6 | aaaagaaaccaaac | 745 | 758 | 2-10-2 | 6_1 | AAaagaaaccaaAC |
| 7 | caaaagaaaccaaa | 746 | 759 | 2-10-2 | 7_1 | CAaaagaaaccaAA |
| 8 | ccaaaagaaaccaa | 747 | 760 | 2-10-2 | 8_1 | CCaaaagaaaccAA |
| 9 | tccactcctaatac | 803 | 816 | 2-10-2 | 9_1 | TCcactcctaatAC |
| 10 | gtccactcctaata | 804 | 817 | 2-10-2 | 10_1 | GTccactcctaaTA |
| 11 | agtccactcctaat | 805 | 818 | 2-10-2 | 11_1 | AGtccactcctaAT |
| 12 | cagtccactcctaa | 806 | 819 | 2-10-2 | 12_1 | CAgtccactcctAA |
| 13 | ccagtccactccta | 807 | 820 | 2-10-2 | 13_1 | CCagtccactccTA |
| 14 | actctttccaaaca | 1012 | 1025 | 2-10-2 | 14_1 | ACtctttccaaaCA |
| 15 | aactctttccaaac | 1013 | 1026 | 2-10-2 | 15_1 | AActctttccaaAC |
| 16 | caactctttccaaa | 1014 | 1027 | 2-10-2 | 16_1 | CAactctttccaAA |
| 17 | gcaactctttccaa | 1015 | 1028 | 2-10-2 | 17_1 | GCaactctttccAA |
| 18 | agcaactctttcca | 1016 | 1029 | 2-10-2 | 18_1 | AGcaactctttcCA |
| 19 | cagcaactctttcc | 1017 | 1030 | 2-10-2 | 19_1 | CAgcaactctttCC |
| 20 | ccagcaactctttc | 1018 | 1031 | 2-10-2 | 20_1 | CCagcaactcttTC |
| 21 | accagcaactcttt | 1019 | 1032 | 2-10-2 | 21_1 | ACcagcaactctTT |
| 22 | ctcctattaaataa | 1040 | 1053 | 2-10-2 | 22_1 | CTcctattaaatAA |
| 23 | cctcctattaaata | 1041 | 1054 | 2-10-2 | 23_1 | CCtcctattaaaTA |
| 24 | tcctcctattaaat | 1042 | 1055 | 2-10-2 | 24_1 | TCctcctattaaAT |
| 25 | ctcctcctattaaa | 1043 | 1056 | 2-10-2 | 25_1 | CTcctcctattaAA |
| 26 | gctcctcctattaa | 1044 | 1057 | 2-10-2 | 26_1 | GCtcctcctattAA |
| 27 | tgctcctcctatta | 1045 | 1058 | 2-10-2 | 27_1 | TGctcctcctatTA |
| 28 | ttgctcctcctatt | 1046 | 1059 | 2-10-2 | 28_1 | TTgctcctcctaTT |
| 29 | tttgctcctcctat | 1047 | 1060 | 2-10-2 | 29_1 | TTtgctcctcctAT |
| 30 | ctttgctcctccta | 1048 | 1061 | 2-10-2 | 30_1 | CTttgctcctccTA |
| 31 | cctttgctcctcct | 1049 | 1062 | 2-10-2 | 31_1 | CctttgctcctcCT |
| 32 | ccctttgctcctcc | 1050 | 1063 | 2-10-2 | 32_1 | CCctttgctcctCC |
| 33 | accctttgctcctc | 1051 | 1064 | 2-10-2 | 33_1 | ACcctttgctccTC |
| 34 | aaccctttgctcct | 1052 | 1065 | 2-10-2 | 34_1 | AAccctttgctcCT |
| 35 | aaaccctttgctcc | 1053 | 1066 | 2-10-2 | 35_1 | AAaccctttgctCC |
| 36 | aaaaccctttgctc | 1054 | 1067 | 2-10-2 | 36_1 | AAaaccctttgcTC |
| 37 | aaaaaccctttgct | 1055 | 1068 | 2-10-2 | 37_1 | AAaaaccctttgCT |
| 38 | caaaaaccctttgc | 1056 | 1069 | 2-10-2 | 38_1 | CAaaaaccctttGC |
| 39 | acaaaaaccctttg | 1057 | 1070 | 2-10-2 | 39_1 | ACaaaaacccttTG |
| 40 | aacaaaaacccttt | 1058 | 1071 | 2-10-2 | 40_1 | AAcaaaaaccctTT |
| 41 | aaacaaaaaccctt | 1059 | 1072 | 2-10-2 | 41_1 | AAacaaaaacccTT |
| 42 | aaaacaaaaaccct | 1060 | 1073 | 2-10-2 | 42_1 | AAaacaaaaaccCT |
| 43 | taaaacaaaaaccc | 1061 | 1074 | 2-10-2 | 43_1 | TAaaacaaaaacCC |
| 44 | ataaaacaaaaacc | 1062 | 1075 | 2-10-2 | 44_1 | ATaaaacaaaaaCC |
| 45 | aataaaacaaaaac | 1063 | 1076 | 2-10-2 | 45_1 | AAtaaaacaaaaAC |
| 46 | taataaaacaaaaa | 1064 | 1077 | 2-10-2 | 46_1 | TAataaaacaaaAA |
| 47 | ttaataaaacaaaa | 1065 | 1078 | 2-10-2 | 47_1 | TTaataaaacaaAA |
| 48 | tttaataaaacaaa | 1066 | 1079 | 2-10-2 | 48_1 | TTtaataaaacaAA |
| 49 | atttaataaaacaa | 1067 | 1080 | 2-10-2 | 49_1 | ATttaataaaacAA |
| 50 | ttaaaataaaaatt | 1194 | 1207 | 2-10-2 | 50_1 | TTaaaataaaaaTT |
| 51 | tttaaaataaaaat | 1195 | 1208 | 2-10-2 | 51_1 | TTtaaaataaaaAT |
| 52 | ctttaaaataaaaa | 1196 | 1209 | 2-10-2 | 52_1 | CTttaaaataaaAA |
| 53 | tctttaaaataaaa | 1197 | 1210 | 2-10-2 | 53_1 | TCtttaaaataaAA |
| 54 | atctttaaaataaa | 1198 | 1211 | 2-10-2 | 54_1 | ATctttaaaataAA |
| 55 | catctttaaaataa | 1199 | 1212 | 2-10-2 | 55_1 | CAtctttaaaatAA |
| 56 | ccatctttaaaata | 1200 | 1213 | 2-10-2 | 56_1 | CCatctttaaaaTA |
| 57 | tctaacttaataaa | 2886 | 2899 | 2-10-2 | 57_1 | TCtaacttaataAA |
| 58 | ttctaacttaataa | 2887 | 2900 | 2-10-2 | 58_1 | TTctaacttaatAA |
| 59 | attctaacttaata | 2888 | 2901 | 2-10-2 | 59_1 | ATtctaacttaaTA |
| 60 | cattctaacttaat | 2889 | 2902 | 2-10-2 | 60_1 | CAttctaacttaAT |
| 61 | acattctaacttaa | 2890 | 2903 | 2-10-2 | 61_1 | ACattctaacttAA |
| 62 | tacattctaactta | 2891 | 2904 | 2-10-2 | 62_1 | TAcattctaactTA |
| 63 | ttacattctaactt | 2892 | 2905 | 2-10-2 | 63_1 | TTacattctaacTT |
| 64 | tttacattctaact | 2893 | 2906 | 2-10-2 | 64_1 | TTtacattctaaCT |
| 65 | ttttacattctaac | 2894 | 2907 | 2-10-2 | 65_1 | TTttacattctaAC |
| 66 | tttttacattctaa | 2895 | 2908 | 2-10-2 | 66_1 | TTtttacattctAA |
| 67 | gtttttacattcta | 2896 | 2909 | 2-10-2 | 67_1 | GTttttacattcTA |
| 68 | tgtttttacattct | 2897 | 2910 | 2-10-2 | 68_1 | TGtttttacattCT |
| 69 | ctgtttttacattc | 2898 | 2911 | 2-10-2 | 69_1 | CTgtttttacatTC |
| 70 | ttcaaatatttatt | 2969 | 2982 | 2-10-2 | 70_1 | TTcaaatatttaTT |
| 71 | attcaaatatttat | 2970 | 2983 | 2-10-2 | 71_1 | ATtcaaatatttAT |
| 72 | cattcaaatattta | 2971 | 2984 | 2-10-2 | 72_1 | CAttcaaatattTA |
| 73 | ccattcaaatattt | 2972 | 2985 | 2-10-2 | 73_1 | CCattcaaatatTT |
| 74 | cccattcaaatatt | 2973 | 2986 | 2-10-2 | 74_1 | CCcattcaaataTT |
| 75 | ccccattcaaatat | 2974 | 2987 | 2-10-2 | 75_1 | CCccattcaaatAT |
| 76 | gccccattcaaata | 2975 | 2988 | 2-10-2 | 76_1 | GCcccattcaaaTA |
| 77 | tatacatttttttc | 3173 | 3186 | 2-10-2 | 77_1 | TAtacattttttTC |
| 78 | atatacattttttt | 3174 | 3187 | 2-10-2 | 78_1 | ATatacatttttTT |
| 79 | tatatacatttttt | 3175 | 3188 | 2-10-2 | 79_1 | TAtatacattttTT |
| 80 | atatatacattttt | 3176 | 3189 | 2-10-2 | 80_1 | ATatatacatttTT |
| 81 | aatatatacatttt | 3177 | 3190 | 2-10-2 | 81_1 | AAtatatacattTT |
| 82 | aaatatatacattt | 3178 | 3191 | 2-10-2 | 82_1 | AAatatatacatTT |
| 83 | caaatatatacatt | 3179 | 3192 | 2-10-2 | 83_1 | CAaatatatacaTT |
| 84 | tcaaatatatacat | 3180 | 3193 | 2-10-2 | 84_1 | TCaaatatatacAT |
| 85 | ttcaaatatataca | 3181 | 3194 | 2-10-2 | 85_1 | TTcaaatatataCA |
| 86 | attcaaatatatac | 3182 | 3195 | 2-10-2 | 86_1 | ATtcaaatatatAC |
| 87 | cattcaaatatata | 3183 | 3196 | 2-10-2 | 87_1 | CAttcaaatataTA |
| 88 | ccattcaaatatat | 3184 | 3197 | 2-10-2 | 88_1 | CCattcaaatatAT |
| 89 | tccattcaaatata | 3185 | 3198 | 2-10-2 | 89_1 | TCcattcaaataTA |
| 90 | atccattcaaatat | 3186 | 3199 | 2-10-2 | 90_1 | ATccattcaaatAT |
| 91 | tatccattcaaata | 3187 | 3200 | 2-10-2 | 91_1 | TAtccattcaaaTA |
| 92 | ttatccattcaaat | 3188 | 3201 | 2-10-2 | 92_1 | TTatccattcaaAT |
| 93 | tttatccattcaaa | 3189 | 3202 | 2-10-2 | 93_1 | TTtatccattcaAA |
| 94 | ctttatccattcaa | 3190 | 3203 | 2-10-2 | 94_1 | CTttatccattcAA |
| 95 | tctttatccattca | 3191 | 3204 | 2-10-2 | 95_1 | TCtttatccattCA |
| 96 | ctctttatccattc | 3192 | 3205 | 2-10-2 | 96_1 | CTctttatccatTC |
| 97 | tctctttatccatt | 3193 | 3206 | 2-10-2 | 97_1 | TCtctttatccaTT |
| 98 | ccatatatatctca | 3221 | 3234 | 2-10-2 | 98_1 | CCatatatatctCA |
| 99 | accatatatatctc | 3222 | 3235 | 2-10-2 | 99_1 | ACcatatatatcTC |
| 100 | caccatatatatct | 3223 | 3236 | 2-10-2 | 100_1 | CAccatatatatCT |
| 101 | gcaccatatatatc | 3224 | 3237 | 2-10-2 | 101_1 | GCaccatatataTC |
| 102 | agcaccatatatat | 3225 | 3238 | 2-10-2 | 102_1 | AGcaccatatatAT |
| 103 | cagcaccatatata | 3226 | 3239 | 2-10-2 | 103_1 | CAgcaccatataTA |
| 104 | acagcaccatatat | 3227 | 3240 | 2-10-2 | 104_1 | ACagcaccatatAT |
| 105 | aacagcaccatata | 3228 | 3241 | 2-10-2 | 105_1 | AAcagcaccataTA |
| 106 | aaaacaaacaacaa | 3462 | 3475 | 2-10-2 | 106_1 | AAaacaaacaacAA |
| 107 | taaaacaaacaaca | 3463 | 3476 | 2-10-2 | 107_1 | TAaaacaaacaaCA |
| 108 | ctaaaacaaacaac | 3464 | 3477 | 2-10-2 | 108_1 | CTaaaacaaacaAC |
| 109 | actaaaacaaacaa | 3465 | 3478 | 2-10-2 | 109_1 | ACtaaaacaaacAA |
| 110 | aactaaaacaaaca | 3466 | 3479 | 2-10-2 | 110_1 | AActaaaacaaaCA |
| 111 | gaactaaaacaaac | 3467 | 3480 | 2-10-2 | 111_1 | GAactaaaacaaAC |
| 112 | agaactaaaacaaa | 3468 | 3481 | 2-10-2 | 112_1 | AGaactaaaacaAA |
| 113 | cagaactaaaacaa | 3469 | 3482 | 2-10-2 | 113_1 | CAgaactaaaacAA |
| 114 | ccagaactaaaaca | 3470 | 3483 | 2-10-2 | 114_1 | CCagaactaaaaCA |
| 115 | accagaactaaaac | 3471 | 3484 | 2-10-2 | 115_1 | ACcagaactaaaAC |
| 116 | atgttattatcccc | 3882 | 3895 | 2-10-2 | 116_1 | ATgttattatccCC |
| 117 | tatgttattatccc | 3883 | 3896 | 2-10-2 | 117_1 | TAtgttattatcCC |
| 118 | ctatgttattatcc | 3884 | 3897 | 2-10-2 | 118_1 | CTatgttattatCC |
| 119 | tctatgttattatc | 3885 | 3898 | 2-10-2 | 119_1 | TCtatgttattaTC |
| 120 | tacactctaactct | 3908 | 3921 | 2-10-2 | 120_1 | TAcactctaactCT |
| 121 | ctacactctaactc | 3909 | 3922 | 2-10-2 | 121_1 | CTacactctaacTC |
| 122 | tctacactctaact | 3910 | 3923 | 2-10-2 | 122_1 | TCtacactctaaCT |
| 123 | ctctacactctaac | 3911 | 3924 | 2-10-2 | 123_1 | CTctacactctaAC |
| 124 | tctctacactctaa | 3912 | 3925 | 2-10-2 | 124_1 | TCtctacactctAA |
| 125 | ttctctacactcta | 3913 | 3926 | 2-10-2 | 125_1 | TTctctacactcTA |
| 126 | cttctctacactct | 3914 | 3927 | 2-10-2 | 126_1 | CTtctctacactCT |
| 127 | ccttctctacactc | 3915 | 3928 | 2-10-2 | 127_1 | CCttctctacacTC |
| 128 | tacaacacaaatca | 4102 | 4115 | 2-10-2 | 128_1 | TAcaacacaaatCA |
| 129 | ctacaacacaaatc | 4103 | 4116 | 2-10-2 | 129_1 | CTacaacacaaaTC |
| 130 | actacaacacaaat | 4104 | 4117 | 2-10-2 | 130_1 | ACtacaacacaaAT |
| 131 | aactacaacacaaa | 4105 | 4118 | 2-10-2 | 131_1 | AActacaacacaAA |
| 132 | taactacaacacaa | 4106 | 4119 | 2-10-2 | 132_1 | TAactacaacacAA |
| 133 | ctaactacaacaca | 4107 | 4120 | 2-10-2 | 133_1 | CTaactacaacaCA |
| 134 | actaactacaacac | 4108 | 4121 | 2-10-2 | 134_1 | ACtaactacaacAC |
| 135 | tactaactacaaca | 4109 | 4122 | 2-10-2 | 135_1 | TActaactacaaCA |
| 136 | ctactaactacaac | 4110 | 4123 | 2-10-2 | 136_1 | CTactaactacaAC |
| 137 | actactaactacaa | 4111 | 4124 | 2-10-2 | 137_1 | ACtactaactacAA |
| 138 | cactactaactaca | 4112 | 4125 | 2-10-2 | 138_1 | CActactaactaCA |
| 139 | acactactaactac | 4113 | 4126 | 2-10-2 | 139_1 | ACactactaactAC |
| 140 | gacactactaacta | 4114 | 4127 | 2-10-2 | 140_1 | GAcactactaacTA |
| 141 | agacactactaact | 4115 | 4128 | 2-10-2 | 141_1 | AGacactactaaCT |
| 142 | tttacccccaacct | 4173 | 4186 | 2-10-2 | 142_1 | TTtacccccaacCT |
| 143 | atttacccccaacc | 4174 | 4187 | 2-10-2 | 143_1 | ATttacccccaaCC |
| 144 | catttacccccaac | 4175 | 4188 | 2-10-2 | 144_1 | CAtttacccccaAC |
| 145 | tcatttacccccaa | 4176 | 4189 | 2-10-2 | 145 1 | TCatttacccccAA |
| 146 | atcatttaccccca | 4177 | 4190 | 2-10-2 | 146_1 | ATcatttaccccCA |
| 147 | aatcatttaccccc | 4178 | 4191 | 2-10-2 | 147_1 | AAtcatttacccCC |
| 148 | aaatcatttacccc | 4179 | 4192 | 2-10-2 | 148_1 | AAatcatttaccCC |
| 149 | caaatcatttaccc | 4180 | 4193 | 2-10-2 | 149_1 | CAaatcatttacCC |
| 150 | ccaaatcatttacc | 4181 | 4194 | 2-10-2 | 150 1 | CCaaatcatttaCC |
| 151 | accaaatcatttac | 4182 | 4195 | 2-10-2 | 151_1 | ACcaaatcatttAC |
| 152 | taccaaatcattta | 4183 | 4196 | 2-10-2 | 152_1 | TAccaaatcattTA |
| 153 | ctaccaaatcattt | 4184 | 4197 | 2-10-2 | 153_1 | CTaccaaatcatTT |
| 154 | gctaccaaatcatt | 4185 | 4198 | 2-10-2 | 154_1 | GCtaccaaatcaTT |
| 155 | tgctaccaaatcat | 4186 | 4199 | 2-10-2 | 155_1 | TGctaccaaatcAT |
| 156 | ctgctaccaaatca | 4187 | 4200 | 2-10-2 | 156_1 | CTgctaccaaatCA |
| 157 | actgctaccaaatc | 4188 | 4201 | 2-10-2 | 157_1 | ACtgctaccaaaTC |
| 158 | aactgctaccaaat | 4189 | 4202 | 2-10-2 | 158_1 | AActgctaccaaAT |
| 159 | aagctttaatcaaa | 5102 | 5115 | 2-10-2 | 159_1 | AAgctttaatcaAA |
| 160 | caagctttaatcaa | 5103 | 5116 | 2-10-2 | 160_1 | CAagctttaatcAA |
| 161 | tcaagctttaatca | 5104 | 5117 | 2-10-2 | 161_1 | TCaagctttaatCA |
| 162 | atcaagctttaatc | 5105 | 5118 | 2-10-2 | 162_1 | ATcaagctttaaTC |
| 163 | catcaagctttaat | 5106 | 5119 | 2-10-2 | 163_1 | CAtcaagctttaAT |
| 164 | tcaaactatcccca | 5131 | 5144 | 2-10-2 | 164_1 | TCaaactatcccCA |
| 165 | ctcaaactatcccc | 5132 | 5145 | 2-10-2 | 165_1 | CTcaaactatccCC |
| 166 | tctcaaactatccc | 5133 | 5146 | 2-10-2 | 166_1 | TCtcaaactatcCC |
| 167 | atctcaaactatcc | 5134 | 5147 | 2-10-2 | 167_1 | ATctcaaactatCC |
| 168 | tatctcaaactatc | 5135 | 5148 | 2-10-2 | 168_1 | TAtctcaaactaTC |
| 169 | ttatctcaaactat | 5136 | 5149 | 2-10-2 | 169_1 | TTatctcaaactAT |
| 170 | cttatctcaaacta | 5137 | 5150 | 2-10-2 | 170_1 | CTtatctcaaacTA |
| 171 | ccttatctcaaact | 5138 | 5151 | 2-10-2 | 171_1 | CCttatctcaaaCT |
| 172 | cccttatctcaaac | 5139 | 5152 | 2-10-2 | 172_1 | CCcttatctcaaAC |
| 173 | gcccttatctcaaa | 5140 | 5153 | 2-10-2 | 173_1 | GCccttatctcaAA |
| 174 | tgcccttatctcaa | 5141 | 5154 | 2-10-2 | 174_1 | TGcccttatctcAA |
| 175 | caaacttcatcaaa | 5540 | 5553 | 2-10-2 | 175 1 | CAaacttcatcaAA |
| 176 | tcaaacttcatcaa | 5541 | 5554 | 2-10-2 | 176_1 | TCaaacttcatcAA |
| 177 | atcaaacttcatca | 5542 | 5555 | 2-10-2 | 177_1 | ATcaaacttcatCA |
| 178 | aatcaaacttcatc | 5543 | 5556 | 2-10-2 | 178_1 | AAtcaaacttcaTC |
| 179 | aaatcaaacttcat | 5544 | 5557 | 2-10-2 | 179_1 | AAatcaaacttcAT |
| 180 | gaaatcaaacttca | 5545 | 5558 | 2-10-2 | 180_1 | GAaatcaaacttCA |
| 181 | tgaaatcaaacttc | 5546 | 5559 | 2-10-2 | 181_1 | TGaaatcaaactTC |
| 182 | ttgaaatcaaactt | 5547 | 5560 | 2-10-2 | 182_1 | TTgaaatcaaacTT |
| 183 | aacacaaatttcct | 5693 | 5706 | 2-10-2 | 183_1 | AAcacaaatttcCT |
| 184 | taacacaaatttcc | 5694 | 5707 | 2-10-2 | 184_1 | TAacacaaatttCC |
| 185 | ctaacacaaatttc | 5695 | 5708 | 2-10-2 | 185_1 | CTaacacaaattTC |
| 186 | gctaacacaaattt | 5696 | 5709 | 2-10-2 | 186_1 | GCtaacacaaatTT |
| 187 | tgctaacacaaatt | 5697 | 5710 | 2-10-2 | 187_1 | TGctaacacaaaTT |
| 188 | ttgctaacacaaat | 5698 | 5711 | 2-10-2 | 188_1 | TTgctaacacaaAT |
| 189 | tttgctaacacaaa | 5699 | 5712 | 2-10-2 | 189_1 | TTtgctaacacaAA |
| 190 | ctttgctaacacaa | 5700 | 5713 | 2-10-2 | 190_1 | CTttgctaacacAA |
| 191 | cctttgctaacaca | 5701 | 5714 | 2-10-2 | 191_1 | CctttgctaacaCA |
| 192 | taactaataattat | 6417 | 6430 | 2-10-2 | 192_1 | TAactaataattAT |
| 193 | ataactaataatta | 6418 | 6431 | 2-10-2 | 193_1 | ATaactaataatTA |
| 194 | aataactaataatt | 6419 | 6432 | 2-10-2 | 194_1 | AAtaactaataaTT |
| 195 | taataactaataat | 6420 | 6433 | 2-10-2 | 195_1 | TAataactaataAT |
| 196 | ataataactaataa | 6421 | 6434 | 2-10-2 | 196_1 | ATaataactaatAA |
| 197 | aataataactaata | 6422 | 6435 | 2-10-2 | 197_1 | AAtaataactaaTA |
| 198 | caataataactaat | 6423 | 6436 | 2-10-2 | 198_1 | CAataataactaAT |
| 199 | ccaataataactaa | 6424 | 6437 | 2-10-2 | 199_1 | CCaataataactAA |
| 200 | accaataataacta | 6425 | 6438 | 2-10-2 | 200_1 | ACcaataataacTA |
| 201 | aaccaataataact | 6426 | 6439 | 2-10-2 | 201_1 | AAccaataataaCT |
| 202 | taaccaataataac | 6427 | 6440 | 2-10-2 | 202_1 | TAaccaataataAC |
| 203 | ataaccaataataa | 6428 | 6441 | 2-10-2 | 203_1 | ATaaccaataatAA |
| 204 | tataaccaataata | 6429 | 6442 | 2-10-2 | 204_1 | TAtaaccaataaTA |
| 205 | gtataaccaataat | 6430 | 6443 | 2-10-2 | 205_1 | GTataaccaataAT |
| 206 | acatcacacaattt | 7415 | 7428 | 2-10-2 | 206_1 | ACatcacacaatTT |
| 207 | gacatcacacaatt | 7416 | 7429 | 2-10-2 | 207_1 | GAcatcacacaaTT |
| 208 | tgacatcacacaat | 7417 | 7430 | 2-10-2 | 208_1 | TGacatcacacaAT |
| 209 | ctgacatcacacaa | 7418 | 7431 | 2-10-2 | 209_1 | CTgacatcacacAA |
| 210 | tctgacatcacaca | 7419 | 7432 | 2-10-2 | 210_1 | TCtgacatcacaCA |
| 211 | atctgacatcacac | 7420 | 7433 | 2-10-2 | 211 1 | ATctgacatcacAC |
| 212 | ttccttaacccaac | 7436 | 7449 | 2-10-2 | 212_1 | TTccttaacccaAC |
| 213 | attccttaacccaa | 7437 | 7450 | 2-10-2 | 213_1 | ATtccttaacccAA |
| 214 | tattccttaaccca | 7438 | 7451 | 2-10-2 | 214_1 | TAttccttaaccCA |
| 215 | ctattccttaaccc | 7439 | 7452 | 2-10-2 | 215_1 | CTattccttaacCC |
| 216 | tctattccttaacc | 7440 | 7453 | 2-10-2 | 216_1 | TCtattccttaaCC |
| 217 | gtctattccttaac | 7441 | 7454 | 2-10-2 | 217_1 | GTctattccttaAC |
| 218 | catcaaatctcata | 8609 | 8622 | 2-10-2 | 218_1 | CAtcaaatctcaTA |
| 219 | gcatcaaatctcat | 8610 | 8623 | 2-10-2 | 219_1 | GCatcaaatctcAT |
| 220 | tgcatcaaatctca | 8611 | 8624 | 2-10-2 | 220_1 | TGcatcaaatctCA |
| 221 | atgcatcaaatctc | 8612 | 8625 | 2-10-2 | 221_1 | ATgcatcaaatcTC |
| 222 | aatgcatcaaatct | 8613 | 8626 | 2-10-2 | 222_1 | AAtgcatcaaatCT |
| 223 | attttaaacaaaca | 8637 | 8650 | 2-10-2 | 223_1 | ATtttaaacaaaCA |
| 224 | tattttaaacaaac | 8638 | 8651 | 2-10-2 | 224_1 | TAttttaaacaaAC |
| 225 | ttattttaaacaaa | 8639 | 8652 | 2-10-2 | 225_1 | TTattttaaacaAA |
| 226 | attattttaaacaa | 8640 | 8653 | 2-10-2 | 226_1 | ATtattttaaacAA |
| 227 | aattattttaaaca | 8641 | 8654 | 2-10-2 | 227_1 | AAttattttaaaCA |
| 228 | gaattattttaaac | 8642 | 8655 | 2-10-2 | 228_1 | GAattattttaaAC |
| 229 | ttttacaaatctac | 8693 | 8706 | 2-10-2 | 229_1 | TTttacaaatctAC |
| 230 | attttacaaatcta | 8694 | 8707 | 2-10-2 | 230_1 | ATtttacaaatcTA |
| 231 | tattttacaaatct | 8695 | 8708 | 2-10-2 | 231_1 | TAttttacaaatCT |
| 232 | ttattttacaaatc | 8696 | 8709 | 2-10-2 | 232_1 | TTattttacaaaTC |
| 233 | tttattttacaaat | 8697 | 8710 | 2-10-2 | 233_1 | TTtattttacaaAT |
| 234 | atttattttacaaa | 8698 | 8711 | 2-10-2 | 234_1 | ATttattttacaAA |
| 235 | catttattttacaa | 8699 | 8712 | 2-10-2 | 235_1 | CAtttattttacAA |
| 236 | acatttattttaca | 8700 | 8713 | 2-10-2 | 236_1 | ACatttattttaCA |
| 237 | aacatttattttac | 8701 | 8714 | 2-10-2 | 237_1 | AAcatttattttAC |
| 238 | taacatttatttta | 8702 | 8715 | 2-10-2 | 238_1 | TAacatttatttTA |
| 239 | aatttaatcattaa | 9391 | 9404 | 2-10-2 | 239_1 | AAtttaatcattAA |
| 240 | taatttaatcatta | 9392 | 9405 | 2-10-2 | 240_1 | TAatttaatcatTA |
| 241 | ataatttaatcatt | 9393 | 9406 | 2-10-2 | 241_1 | ATaatttaatcaTT |
| 242 | aataatttaatcat | 9394 | 9407 | 2-10-2 | 242_1 | AAtaatttaatcAT |
| 243 | aaataatttaatca | 9395 | 9408 | 2-10-2 | 243_1 | AAataatttaatCA |
| 244 | taaataatttaatc | 9396 | 9409 | 2-10-2 | 244_1 | TAaataatttaaTC |
| 245 | ctaaataatttaat | 9397 | 9410 | 2-10-2 | 245_1 | CTaaataatttaAT |
| 246 | cctaaataatttaa | 9398 | 9411 | 2-10-2 | 246_1 | CCtaaataatttAA |
| 247 | ccctaaataattta | 9399 | 9412 | 2-10-2 | 247_1 | CCctaaataattTA |
| 248 | cccctaaataattt | 9400 | 9413 | 2-10-2 | 248_1 | CCcctaaataatTT |
| 249 | tcccctaaataatt | 9401 | 9414 | 2-10-2 | 249_1 | TCccctaaataaTT |
| 250 | tatataaaaatcta | 10958 | 10971 | 2-10-2 | 250_1 | TAtataaaaatcTA |
| 251 | ctatataaaaatct | 10959 | 10972 | 2-10-2 | 251_1 | CTatataaaaatCT |
| 252 | tctatataaaaatc | 10960 | 10973 | 2-10-2 | 252_1 | TCtatataaaaaTC |
| 253 | atctatataaaaat | 10961 | 10974 | 2-10-2 | 253_1 | ATctatataaaaAT |
| 254 | tatctatataaaaa | 10962 | 10975 | 2-10-2 | 254_1 | TAtctatataaaAA |
| 255 | ttatctatataaaa | 10963 | 10976 | 2-10-2 | 255_1 | TTatctatataaAA |
| 256 | tttatctatataaa | 10964 | 10977 | 2-10-2 | 256_1 | TTtatctatataAA |
| 257 | ccccactctaatat | 11001 | 11014 | 2-10-2 | 257_1 | CCccactctaatAT |
| 258 | gccccactctaata | 11002 | 11015 | 2-10-2 | 258_1 | GCcccactctaaTA |
| 259 | tgccccactctaat | 11003 | 11016 | 2-10-2 | 259_1 | TGccccactctaAT |
| 260 | atgccccactctaa | 11004 | 11017 | 2-10-2 | 260_1 | ATgccccactctAA |
| 261 | aatgccccactcta | 11005 | 11018 | 2-10-2 | 261_1 | AAtgccccactcTA |
| 262 | aaatgccccactct | 11006 | 11019 | 2-10-2 | 262_1 | AAatgccccactCT |
| 263 | taaatgccccactc | 11007 | 11020 | 2-10-2 | 263_1 | TAaatgccccacTC |
| 264 | ttaaatgccccact | 11008 | 11021 | 2-10-2 | 264_1 | TTaaatgccccaCT |
| 265 | atataaccaccaaa | 11546 | 11559 | 2-10-2 | 265_1 | ATataaccaccaAA |
| 266 | tatataaccaccaa | 11547 | 11560 | 2-10-2 | 266_1 | TAtataaccaccAA |
| 267 | atatataaccacca | 11548 | 11561 | 2-10-2 | 267_1 | ATatataaccacCA |
| 268 | tatatataaccacc | 11549 | 11562 | 2-10-2 | 268_1 | TAtatataaccaCC |
| 269 | atatatataaccac | 11550 | 11563 | 2-10-2 | 269_1 | ATatatataaccAC |
| 270 | aaaattcactatct | 11942 | 11955 | 2-10-2 | 270_1 | AAaattcactatCT |
| 271 | gaaaattcactatc | 11943 | 11956 | 2-10-2 | 271_1 | GAaaattcactaTC |
| 272 | tgaaaattcactat | 11944 | 11957 | 2-10-2 | 272_1 | TGaaaattcactAT |
| 273 | ctgaaaattcacta | 11945 | 11958 | 2-10-2 | 273_1 | CTgaaaattcacTA |
| 274 | tctgaaaattcact | 11946 | 11959 | 2-10-2 | 274_1 | TCtgaaaattcaCT |
| 275 | tactatatacatct | 12176 | 12189 | 2-10-2 | 275_1 | TActatatacatCT |
| 276 | ctactatatacatc | 12177 | 12190 | 2-10-2 | 276_1 | CTactatatacaTC |
| 277 | tctactatatacat | 12178 | 12191 | 2-10-2 | 277_1 | TCtactatatacAT |
| 278 | gtctactatataca | 12179 | 12192 | 2-10-2 | 278_1 | GTctactatataCA |
| 279 | agtctactatatac | 12180 | 12193 | 2-10-2 | 279_1 | AGtctactatatAC |
| 280 | tagtctactatata | 12181 | 12194 | 2-10-2 | 280_1 | TAgtctactataTA |
| 281 | ctagtctactatat | 12182 | 12195 | 2-10-2 | 281_1 | CTagtctactatAT |
| 282 | actagtctactata | 12183 | 12196 | 2-10-2 | 282_1 | ACtagtctactaTA |
| 283 | aactagtctactat | 12184 | 12197 | 2-10-2 | 283_1 | AActagtctactAT |
| 284 | tattctacccataa | 12211 | 12224 | 2-10-2 | 284_1 | TAttctacccatAA |
| 285 | atattctacccata | 12212 | 12225 | 2-10-2 | 285_1 | ATattctacccaTA |
| 286 | tatattctacccat | 12213 | 12226 | 2-10-2 | 286_1 | TAtattctacccAT |
| 287 | gtatattctaccca | 12214 | 12227 | 2-10-2 | 287_1 | GTatattctaccCA |
| 288 | tgtatattctaccc | 12215 | 12228 | 2-10-2 | 288_1 | TGtatattctacCC |
| 289 | atgtatattctacc | 12216 | 12229 | 2-10-2 | 289_1 | ATgtatattctaCC |
| 290 | ccacacaattccta | 12254 | 12267 | 2-10-2 | 290_1 | CCacacaattccTA |
| 291 | accacacaattcct | 12255 | 12268 | 2-10-2 | 291_1 | ACcacacaattcCT |
| 292 | aaccacacaattcc | 12256 | 12269 | 2-10-2 | 292_1 | AAccacacaattCC |
| 293 | aaaccacacaattc | 12257 | 12270 | 2-10-2 | 293_1 | AAaccacacaatTC |
| 294 | aaaaccacacaatt | 12258 | 12271 | 2-10-2 | 294_1 | AAaaccacacaaTT |
| 295 | gaaaaccacacaat | 12259 | 12272 | 2-10-2 | 295_1 | GAaaaccacacaAT |
| 296 | agaaaaccacacaa | 12260 | 12273 | 2-10-2 | 296_1 | AGaaaaccacacAA |
| 297 | cagaaaaccacaca | 12261 | 12274 | 2-10-2 | 297_1 | CAgaaaaccacaCA |
| 298 | ccagaaaaccacac | 12262 | 12275 | 2-10-2 | 298_1 | CCagaaaaccacAC |
| 299 | tccagaaaaccaca | 12263 | 12276 | 2-10-2 | 299_1 | TCcagaaaaccaCA |
| 300 | aaatccataaaaaa | 12327 | 12340 | 2-10-2 | 300_1 | AAatccataaaaAA |
| 301 | taaatccataaaaa | 12328 | 12341 | 2-10-2 | 301 1 | TAaatccataaaAA |
| 302 | ctaaatccataaaa | 12329 | 12342 | 2-10-2 | 302_1 | CTaaatccataaAA |
| 303 | actaaatccataaa | 12330 | 12343 | 2-10-2 | 303_1 | ACtaaatccataAA |
| 304 | cactaaatccataa | 12331 | 12344 | 2-10-2 | 304_1 | CActaaatccatAA |
| 305 | tcactaaatccata | 12332 | 12345 | 2-10-2 | 305_1 | TCactaaatccaTA |
| 306 | atcactaaatccat | 12333 | 12346 | 2-10-2 | 306_1 | ATcactaaatccAT |
| 307 | tatcactaaatcca | 12334 | 12347 | 2-10-2 | 307_1 | TAtcactaaatcCA |
| 308 | atatcactaaatcc | 12335 | 12348 | 2-10-2 | 308_1 | ATatcactaaatCC |
| 309 | tatatcactaaatc | 12336 | 12349 | 2-10-2 | 309_1 | TAtatcactaaaTC |
| 310 | atatatcactaaat | 12337 | 12350 | 2-10-2 | 310_1 | ATatatcactaaAT |
| 311 | gatatatcactaaa | 12338 | 12351 | 2-10-2 | 311_1 | GAtatatcactaAA |
| 312 | agatatatcactaa | 12339 | 12352 | 2-10-2 | 312_1 | AGatatatcactAA |
| 313 | tagatatatcacta | 12340 | 12353 | 2-10-2 | 313_1 | TAgatatatcacTA |
| 314 | tataaatttctcta | 12690 | 12703 | 2-10-2 | 314_1 | TAtaaatttctcTA |
| 315 | atataaatttctct | 12691 | 12704 | 2-10-2 | 315_1 | ATataaatttctCT |
| 316 | tatataaatttctc | 12692 | 12705 | 2-10-2 | 316_1 | TAtataaatttcTC |
| 317 | atatataaatttct | 12693 | 12706 | 2-10-2 | 317_1 | ATatataaatttCT |
| 318 | catatataaatttc | 12694 | 12707 | 2-10-2 | 318_1 | CAtatataaattTC |
| 319 | tcatatataaattt | 12695 | 12708 | 2-10-2 | 319_1 | TCatatataaatTT |
| 320 | ctccattccaaatt | 12739 | 12752 | 2-10-2 | 320_1 | CTccattccaaaTT |
| 321 | actccattccaaat | 12740 | 12753 | 2-10-2 | 321_1 | ACtccattccaaAT |
| 322 | cactccattccaaa | 12741 | 12754 | 2-10-2 | 322_1 | CActccattccaAA |
| 323 | ccactccattccaa | 12742 | 12755 | 2-10-2 | 323_1 | CCactccattccAA |
| 324 | accactccattcca | 12743 | 12756 | 2-10-2 | 324_1 | ACcactccattcCA |
| 325 | aaccactccattcc | 12744 | 12757 | 2-10-2 | 325_1 | AAccactccattCC |
| 326 | aaaccactccattc | 12745 | 12758 | 2-10-2 | 326_1 | AAaccactccatTC |
| 327 | tcacacaaccatat | 13155 | 13168 | 2-10-2 | 327_1 | TCacacaaccatAT |
| 328 | atcacacaaccata | 13156 | 13169 | 2-10-2 | 328_1 | ATcacacaaccaTA |
| 329 | gatcacacaaccat | 13157 | 13170 | 2-10-2 | 329_1 | GAtcacacaaccAT |
| 330 | agatcacacaacca | 13158 | 13171 | 2-10-2 | 330_1 | AGatcacacaacCA |
| 331 | aagatcacacaacc | 13159 | 13172 | 2-10-2 | 331_1 | AAgatcacacaaCC |
| 332 | aaagatcacacaac | 13160 | 13173 | 2-10-2 | 332_1 | AAagatcacacaAC |
| 333 | aaaagatcacacaa | 13161 | 13174 | 2-10-2 | 333_1 | AAaagatcacacAA |
| 334 | taaaagatcacaca | 13162 | 13175 | 2-10-2 | 334_1 | TAaaagatcacaCA |
| 335 | ttcatttctaaaaa | 13297 | 13310 | 2-10-2 | 335_1 | TTcatttctaaaAA |
| 336 | tttcatttctaaaa | 13298 | 13311 | 2-10-2 | 336_1 | TTtcatttctaaAA |
| 337 | ctttcatttctaaa | 13299 | 13312 | 2-10-2 | 337_1 | CTttcatttctaAA |
| 338 | tctttcatttctaa | 13300 | 13313 | 2-10-2 | 338_1 | TCtttcatttctAA |
| 339 | atctttcatttcta | 13301 | 13314 | 2-10-2 | 339_1 | ATctttcatttcTA |
| 340 | gatctttcatttct | 13302 | 13315 | 2-10-2 | 340_1 | GAtctttcatttCT |
| 341 | tgatctttcatttc | 13303 | 13316 | 2-10-2 | 341_1 | TGatctttcattTC |
| 342 | atgatctttcattt | 13304 | 13317 | 2-10-2 | 342_1 | ATgatctttcatTT |
| 343 | ataaaaacccactt | 13990 | 14003 | 2-10-2 | 343_1 | ATaaaaacccacTT |
| 344 | cataaaaacccact | 13991 | 14004 | 2-10-2 | 344_1 | CAtaaaaacccaCT |
| 345 | acataaaaacccac | 13992 | 14005 | 2-10-2 | 345_1 | ACataaaaacccAC |
| 346 | cacataaaaaccca | 13993 | 14006 | 2-10-2 | 346_1 | CAcataaaaaccCA |
| 347 | tcacataaaaaccc | 13994 | 14007 | 2-10-2 | 347_1 | TCacataaaaacCC |
| 348 | atcacataaaaacc | 13995 | 14008 | 2-10-2 | 348_1 | ATcacataaaaaCC |
| 349 | catcacataaaaac | 13996 | 14009 | 2-10-2 | 349_1 | CAtcacataaaaAC |
| 350 | tcatcacataaaaa | 13997 | 14010 | 2-10-2 | 350_1 | TCatcacataaaAA |
| 351 | gtcatcacataaaa | 13998 | 14011 | 2-10-2 | 351_1 | GTcatcacataaAA |
| 352 | agtcatcacataaa | 13999 | 14012 | 2-10-2 | 352_1 | AGtcatcacataAA |
| 353 | tagtcatcacataa | 14000 | 14013 | 2-10-2 | 353_1 | TAgtcatcacatAA |
| 354 | atagtcatcacata | 14001 | 14014 | 2-10-2 | 354_1 | ATagtcatcacaTA |
| 355 | catagtcatcacat | 14002 | 14015 | 2-10-2 | 355_1 | CAtagtcatcacAT |
| 356 | taaatacaaatcta | 14041 | 14054 | 2-10-2 | 356_1 | TAaatacaaatcTA |
| 357 | ctaaatacaaatct | 14042 | 14055 | 2-10-2 | 357_1 | CTaaatacaaatCT |
| 358 | gctaaatacaaatc | 14043 | 14056 | 2-10-2 | 358_1 | GCtaaatacaaaTC |
| 359 | tgctaaatacaaat | 14044 | 14057 | 2-10-2 | 359_1 | TGctaaatacaaAT |
| 360 | atgctaaatacaaa | 14045 | 14058 | 2-10-2 | 360_1 | ATgctaaatacaAA |
| 361 | tatgctaaatacaa | 14046 | 14059 | 2-10-2 | 361_1 | TAtgctaaatacAA |
| 362 | aatcttacactaaa | 14119 | 14132 | 2-10-2 | 362_1 | AAtcttacactaAA |
| 363 | taatcttacactaa | 14120 | 14133 | 2-10-2 | 363_1 | TAatcttacactAA |
| 364 | ataatcttacacta | 14121 | 14134 | 2-10-2 | 364_1 | ATaatcttacacTA |
| 365 | aataatcttacact | 14122 | 14135 | 2-10-2 | 365_1 | AAtaatcttacaCT |
| 366 | gaataatcttacac | 14123 | 14136 | 2-10-2 | 366_1 | GAataatcttacAC |
| 367 | tgaataatcttaca | 14124 | 14137 | 2-10-2 | 367_1 | TGaataatcttaCA |
| 368 | atgaataatcttac | 14125 | 14138 | 2-10-2 | 368_1 | ATgaataatcttAC |
| 369 | caaaattctaataa | 14257 | 14270 | 2-10-2 | 369_1 | CAaaattctaatAA |
| 370 | tcaaaattctaata | 14258 | 14271 | 2-10-2 | 370_1 | TCaaaattctaaTA |
| 371 | ttcaaaattctaat | 14259 | 14272 | 2-10-2 | 371_1 | TTcaaaattctaAT |
| 372 | attcaaaattctaa | 14260 | 14273 | 2-10-2 | 372_1 | ATtcaaaattctAA |
| 373 | gattcaaaattcta | 14261 | 14274 | 2-10-2 | 373_1 | GAttcaaaattcTA |
| 374 | agattcaaaattct | 14262 | 14275 | 2-10-2 | 374_1 | AGattcaaaattCT |
| 375 | attactacaaccaa | 14570 | 14583 | 2-10-2 | 375_1 | ATtactacaaccAA |
| 376 | cattactacaacca | 14571 | 14584 | 2-10-2 | 376_1 | CAttactacaacCA |
| 377 | ccattactacaacc | 14572 | 14585 | 2-10-2 | 377_1 | CCattactacaaCC |
| 378 | accattactacaac | 14573 | 14586 | 2-10-2 | 378_1 | ACcattactacaAC |
| 379 | aaccattactacaa | 14574 | 14587 | 2-10-2 | 379_1 | AAccattactacAA |
| 380 | aaaccattactaca | 14575 | 14588 | 2-10-2 | 380_1 | AAaccattactaCA |
| 381 | gaaaccattactac | 14576 | 14589 | 2-10-2 | 381_1 | GAaaccattactAC |
| 382 | tgaaaccattacta | 14577 | 14590 | 2-10-2 | 382_1 | TGaaaccattacTA |
| 383 | atgaaaccattact | 14578 | 14591 | 2-10-2 | 383_1 | ATgaaaccattaCT |
| 384 | atttttaaaaacac | 15778 | 15791 | 2-10-2 | 384_1 | ATttttaaaaacAC |
| 385 | aatttttaaaaaca | 15779 | 15792 | 2-10-2 | 385_1 | AAtttttaaaaaCA |
| 386 | taatttttaaaaac | 15780 | 15793 | 2-10-2 | 386_1 | TAatttttaaaaAC |
| 387 | ataatttttaaaaa | 15781 | 15794 | 2-10-2 | 387_1 | ATaatttttaaaAA |
| 388 | cataatttttaaaa | 15782 | 15795 | 2-10-2 | 388_1 | CAtaatttttaaAA |
| 389 | tcataatttttaaa | 15783 | 15796 | 2-10-2 | 389_1 | TCataatttttaAA |
| 390 | atcataatttttaa | 15784 | 15797 | 2-10-2 | 390_1 | ATcataatttttAA |
| 391 | ctttatacaaaaaa | 15814 | 15827 | 2-10-2 | 391_1 | CTttatacaaaaAA |
| 392 | actttatacaaaaa | 15815 | 15828 | 2-10-2 | 392_1 | ACtttatacaaaAA |
| 393 | tactttatacaaaa | 15816 | 15829 | 2-10-2 | 393_1 | TActttatacaaAA |
| 394 | ttactttatacaaa | 15817 | 15830 | 2-10-2 | 394_1 | TTactttatacaAA |
| 395 | cttactttatacaa | 15818 | 15831 | 2-10-2 | 395_1 | CTtactttatacAA |
| 396 | gcttactttataca | 15819 | 15832 | 2-10-2 | 396_1 | GCttactttataCA |
| 397 | tgcttactttatac | 15820 | 15833 | 2-10-2 | 397_1 | TGcttactttatAC |
| 398 | tctcaaaataataa | 15877 | 15890 | 2-10-2 | 398_1 | TCtcaaaataatAA |
| 399 | ctctcaaaataata | 15878 | 15891 | 2-10-2 | 399_1 | CTctcaaaataaTA |
| 400 | tctctcaaaataat | 15879 | 15892 | 2-10-2 | 400_1 | TCtctcaaaataAT |
| 401 | atctctcaaaataa | 15880 | 15893 | 2-10-2 | 401_1 | ATctctcaaaatAA |
| 402 | aatctctcaaaata | 15881 | 15894 | 2-10-2 | 402_1 | AAtctctcaaaaTA |
| 403 | aaatctctcaaaat | 15882 | 15895 | 2-10-2 | 403_1 | AAatctctcaaaAT |
| 404 | taaatctctcaaaa | 15883 | 15896 | 2-10-2 | 404_1 | TAaatctctcaaAA |
| 405 | ttaaatctctcaaa | 15884 | 15897 | 2-10-2 | 405_1 | TTaaatctctcaAA |
| 406 | tttaaatctctcaa | 15885 | 15898 | 2-10-2 | 406_1 | TTtaaatctctcAA |
| 407 | ttttaaatctctca | 15886 | 15899 | 2-10-2 | 407_1 | TTttaaatctctCA |
| 408 | taatactttttcca | 16080 | 16093 | 2-10-2 | 408_1 | TAatactttttcCA |
| 409 | ttaatactttttcc | 16081 | 16094 | 2-10-2 | 409_1 | TTaatactttttCC |
| 410 | gttaatactttttc | 16082 | 16095 | 2-10-2 | 410_1 | GTtaatacttttTC |
| 411 | tgttaatacttttt | 16083 | 16096 | 2-10-2 | 411_1 | TGttaatactttTT |
| 412 | atgttaatactttt | 16084 | 16097 | 2-10-2 | 412_1 | ATgttaatacttTT |
| 413 | ttatcactaccaca | 16187 | 16200 | 2-10-2 | 413_1 | TTatcactaccaCA |
| 414 | tttatcactaccac | 16188 | 16201 | 2-10-2 | 414_1 | TTtatcactaccAC |
| 415 | atttatcactacca | 16189 | 16202 | 2-10-2 | 415_1 | ATttatcactacCA |
| 416 | catttatcactacc | 16190 | 16203 | 2-10-2 | 416_1 | CAtttatcactaCC |
| 417 | tcatttatcactac | 16191 | 16204 | 2-10-2 | 417_1 | TCatttatcactAC |
| 418 | atcatttatcacta | 16192 | 16205 | 2-10-2 | 418_1 | ATcatttatcacTA |
| 419 | catcatttatcact | 16193 | 16206 | 2-10-2 | 419_1 | CAtcatttatcaCT |
| 420 | acatcatttatcac | 16194 | 16207 | 2-10-2 | 420_1 | ACatcatttatcAC |
| 421 | aacatcatttatca | 16195 | 16208 | 2-10-2 | 421_1 | AAcatcatttatCA |
| 422 | taacatcatttatc | 16196 | 16209 | 2-10-2 | 422_1 | TAacatcatttaTC |
| 423 | ttaacatcatttat | 16197 | 16210 | 2-10-2 | 423_1 | TTaacatcatttAT |
| 424 | attaacatcattta | 16198 | 16211 | 2-10-2 | 424_1 | ATtaacatcattTA |
| 425 | aattaacatcattt | 16199 | 16212 | 2-10-2 | 425_1 | AAttaacatcatTT |
| 426 | taattaacatcatt | 16200 | 16213 | 2-10-2 | 426_1 | TAattaacatcaTT |
| 427 | ctaattaacatcat | 16201 | 16214 | 2-10-2 | 427_1 | CTaattaacatcAT |
| 428 | cctaattaacatca | 16202 | 16215 | 2-10-2 | 428_1 | CCtaattaacatCA |
| 429 | ccctaattaacatc | 16203 | 16216 | 2-10-2 | 429_1 | CCctaattaacaTC |
| 430 | gccctaattaacat | 16204 | 16217 | 2-10-2 | 430_1 | GCcctaattaacAT |
| 431 | ggccctaattaaca | 16205 | 16218 | 2-10-2 | 431_1 | GGccctaattaaCA |
| 432 | cggccctaattaac | 16206 | 16219 | 2-10-2 | 432_1 | CGgccctaattaAC |
| 433 | aaacacattttttt | 16494 | 16507 | 2-10-2 | 433_1 | AAacacatttttTT |
| 434 | taaacacatttttt | 16495 | 16508 | 2-10-2 | 434_1 | TAaacacattttTT |
| 435 | ataaacacattttt | 16496 | 16509 | 2-10-2 | 435_1 | ATaaacacatttTT |
| 436 | tataaacacatttt | 16497 | 16510 | 2-10-2 | 436_1 | TAtaaacacattTT |
| 437 | atataaacacattt | 16498 | 16511 | 2-10-2 | 437_1 | ATataaacacatTT |
| 438 | catataaacacatt | 16499 | 16512 | 2-10-2 | 438_1 | CAtataaacacaTT |
| 439 | acatataaacacat | 16500 | 16513 | 2-10-2 | 439_1 | ACatataaacacAT |
| 440 | aacatataaacaca | 16501 | 16514 | 2-10-2 | 440_1 | AAcatataaacaCA |
| 441 | taacatataaacac | 16502 | 16515 | 2-10-2 | 441_1 | TAacatataaacAC |
| 442 | ataacatataaaca | 16503 | 16516 | 2-10-2 | 442_1 | ATaacatataaaCA |
| 443 | tataacatataaac | 16504 | 16517 | 2-10-2 | 443_1 | TAtaacatataaAC |
| 444 | atataacatataaa | 16505 | 16518 | 2-10-2 | 444_1 | ATataacatataAA |
| 445 | catataacatataa | 16506 | 16519 | 2-10-2 | 445_1 | CAtataacatatAA |
| 446 | acatataacatata | 16507 | 16520 | 2-10-2 | 446_1 | ACatataacataTA |
| 447 | cacatataacatat | 16508 | 16521 | 2-10-2 | 447_1 | CAcatataacatAT |
| 448 | tcacatataacata | 16509 | 16522 | 2-10-2 | 448_1 | TCacatataacaTA |
| 449 | atcacatataacat | 16510 | 16523 | 2-10-2 | 449_1 | ATcacatataacAT |
| 450 | tatcacatataaca | 16511 | 16524 | 2-10-2 | 450_1 | TAtcacatataaCA |
| 451 | ctatcacatataac | 16512 | 16525 | 2-10-2 | 451_1 | CTatcacatataAC |
| 452 | actatcacatataa | 16513 | 16526 | 2-10-2 | 452_1 | ACtatcacatatAA |
| 453 | cactatcacatata | 16514 | 16527 | 2-10-2 | 453_1 | CActatcacataTA |
| 454 | gtccaacataactc | 16834 | 16847 | 2-10-2 | 454_1 | GTccaacataacTC |
| 455 | agtccaacataact | 16835 | 16848 | 2-10-2 | 455_1 | AGtccaacataaCT |
| 456 | cagtccaacataac | 16836 | 16849 | 2-10-2 | 456_1 | CAgtccaacataAC |
| 457 | tcagtccaacataa | 16837 | 16850 | 2-10-2 | 457_1 | TCagtccaacatAA |
| 458 | atcagtccaacata | 16838 | 16851 | 2-10-2 | 458_1 | ATcagtccaacaTA |
| 459 | tatcagtccaacat | 16839 | 16852 | 2-10-2 | 459_1 | TAtcagtccaacAT |
| 460 | aaaccctcccaaaa | 16921 | 16934 | 2-10-2 | 460_1 | AAaccctcccaaAA |
| 461 | taaaccctcccaaa | 16922 | 16935 | 2-10-2 | 461_1 | TAaaccctcccaAA |
| 462 | ttaaaccctcccaa | 16923 | 16936 | 2-10-2 | 462_1 | TTaaaccctcccAA |
| 463 | attaaaccctccca | 16924 | 16937 | 2-10-2 | 463_1 | ATtaaaccctccCA |
| 464 | cattaaaccctccc | 16925 | 16938 | 2-10-2 | 464_1 | CAttaaaccctcCC |
| 465 | acattaaaccctcc | 16926 | 16939 | 2-10-2 | 465_1 | ACattaaaccctCC |
| 466 | aacattaaaccctc | 16927 | 16940 | 2-10-2 | 466_1 | AAcattaaacccTC |
| 467 | aaacattaaaccct | 16928 | 16941 | 2-10-2 | 467_1 | AAacattaaaccCT |
| 468 | taaacattaaaccc | 16929 | 16942 | 2-10-2 | 468_1 | TAaacattaaacCC |
| 469 | ataaacattaaacc | 16930 | 16943 | 2-10-2 | 469_1 | ATaaacattaaaCC |
| 470 | tataaacattaaac | 16931 | 16944 | 2-10-2 | 470_1 | TAtaaacattaaAC |
| 471 | ctataaacattaaa | 16932 | 16945 | 2-10-2 | 471_1 | CTataaacattaAA |
| 472 | actataaacattaa | 16933 | 16946 | 2-10-2 | 472_1 | ACtataaacattAA |
| 473 | aactataaacatta | 16934 | 16947 | 2-10-2 | 473_1 | AActataaacatTA |
| 474 | aaactataaacatt | 16935 | 16948 | 2-10-2 | 474_1 | AAactataaacaTT |
| 475 | taaactataaacat | 16936 | 16949 | 2-10-2 | 475_1 | TAaactataaacAT |
| 476 | ttaaactataaaca | 16937 | 16950 | 2-10-2 | 476_1 | TTaaactataaaCA |
| 477 | tttaaactataaac | 16938 | 16951 | 2-10-2 | 477_1 | TTtaaactataaAC |
| 478 | ctttaaactataaa | 16939 | 16952 | 2-10-2 | 478_1 | CTttaaactataAA |
| 479 | gctttaaactataa | 16940 | 16953 | 2-10-2 | 479_1 | GCtttaaactatAA |
| 480 | tgctttaaactata | 16941 | 16954 | 2-10-2 | 480_1 | TGctttaaactaTA |
| 481 | cagcctatcaccac | 18018 | 18031 | 2-10-2 | 481_1 | CAgcctatcaccAC |
| 482 | acagcctatcacca | 18019 | 18032 | 2-10-2 | 482_1 | ACagcctatcacCA |
| 483 | cacagcctatcacc | 18020 | 18033 | 2-10-2 | 483_1 | CAcagcctatcaCC |
| 484 | tcacagcctatcac | 18021 | 18034 | 2-10-2 | 484_1 | TCacagcctatcAC |
| 485 | atcacagcctatca | 18022 | 18035 | 2-10-2 | 485_1 | ATcacagcctatCA |
| 486 | aatcacagcctatc | 18023 | 18036 | 2-10-2 | 486_1 | AAtcacagcctaTC |
| 487 | aaatcacagcctat | 18024 | 18037 | 2-10-2 | 487_1 | AAatcacagcctAT |
| 488 | caaatcacagccta | 18025 | 18038 | 2-10-2 | 488_1 | CAaatcacagccTA |
| 489 | ccaaatcacagcct | 18026 | 18039 | 2-10-2 | 489_1 | CCaaatcacagcCT |
| 490 | cccaaatcacagcc | 18027 | 18040 | 2-10-2 | 490_1 | CCcaaatcacagCC |
| 491 | acccaaatcacagc | 18028 | 18041 | 2-10-2 | 491_1 | ACccaaatcacaGC |
| 492 | cacccaaatcacag | 18029 | 18042 | 2-10-2 | 492_1 | CAcccaaatcacAG |
| 493 | tcacccaaatcaca | 18030 | 18043 | 2-10-2 | 493_1 | TCacccaaatcaCA |
| 494 | gtcacccaaatcac | 18031 | 18044 | 2-10-2 | 494_1 | GTcacccaaatcAC |
| 495 | cgtcacccaaatca | 18032 | 18045 | 2-10-2 | 495_1 | CGtcacccaaatCA |
| 496 | gcgtcacccaaatc | 18033 | 18046 | 2-10-2 | 496_1 | GCgtcacccaaaTC |
| 497 | agcgtcacccaaat | 18034 | 18047 | 2-10-2 | 497_1 | AGcgtcacccaaAT |
| 498 | atcctaaaatcact | 18630 | 18643 | 2-10-2 | 498_1 | ATcctaaaatcaCT |
| 499 | gatcctaaaatcac | 18631 | 18644 | 2-10-2 | 499_1 | GAtcctaaaatcAC |
| 500 | agatcctaaaatca | 18632 | 18645 | 2-10-2 | 500_1 | AGatcctaaaatCA |
| 501 | cagatcctaaaatc | 18633 | 18646 | 2-10-2 | 501_1 | CAgatcctaaaaTC |
| 502 | tcagatcctaaaat | 18634 | 18647 | 2-10-2 | 502_1 | TCagatcctaaaAT |
| 503 | aaaccaatcatcat | 19107 | 19120 | 2-10-2 | 503_1 | AAaccaatcatcAT |
| 504 | aaaaccaatcatca | 19108 | 19121 | 2-10-2 | 504_1 | AAaaccaatcatCA |
| 505 | taaaaccaatcatc | 19109 | 19122 | 2-10-2 | 505_1 | TAaaaccaatcaTC |
| 506 | gtaaaaccaatcat | 19110 | 19123 | 2-10-2 | 506_1 | GTaaaaccaatcAT |
| 507 | agtaaaaccaatca | 19111 | 19124 | 2-10-2 | 507_1 | AGtaaaaccaatCA |
| 508 | aagtaaaaccaatc | 19112 | 19125 | 2-10-2 | 508_1 | AAgtaaaaccaaTC |
| 509 | aaagtaaaaccaat | 19113 | 19126 | 2-10-2 | 509_1 | AAagtaaaaccaAT |
| 510 | catctctactaaaa | 20214 | 20227 | 2-10-2 | 510_1 | CAtctctactaaAA |
| 511 | ccatctctactaaa | 20215 | 20228 | 2-10-2 | 511_1 | CCatctctactaAA |
| 512 | tccatctctactaa | 20216 | 20229 | 2-10-2 | 512_1 | TCcatctctactAA |
| 513 | ttccatctctacta | 20217 | 20230 | 2-10-2 | 513_1 | TTccatctctacTA |
| 514 | cttccatctctact | 20218 | 20231 | 2-10-2 | 514_1 | CTtccatctctaCT |
| 515 | ccttccatctctac | 20219 | 20232 | 2-10-2 | 515_1 | CCttccatctctAC |
| 516 | cccttccatctcta | 20220 | 20233 | 2-10-2 | 516_1 | CCcttccatctcTA |
| 517 | acataacaaaccca | 20555 | 20568 | 2-10-2 | 517_1 | ACataacaaaccCA |
| 518 | tacataacaaaccc | 20556 | 20569 | 2-10-2 | 518_1 | TAcataacaaacCC |
| 519 | ctacataacaaacc | 20557 | 20570 | 2-10-2 | 519_1 | CTacataacaaaCC |
| 520 | actacataacaaac | 20558 | 20571 | 2-10-2 | 520_1 | ACtacataacaaAC |
| 521 | aactacataacaaa | 20559 | 20572 | 2-10-2 | 521_1 | AActacataacaAA |
| 522 | taactacataacaa | 20560 | 20573 | 2-10-2 | 522_1 | TAactacataacAA |
| 523 | ataactacataaca | 20561 | 20574 | 2-10-2 | 523_1 | ATaactacataaCA |
| 524 | aataactacataac | 20562 | 20575 | 2-10-2 | 524_1 | AAtaactacataAC |
| 525 | caataactacataa | 20563 | 20576 | 2-10-2 | 525_1 | CAataactacatAA |
| 526 | acaataactacata | 20564 | 20577 | 2-10-2 | 526_1 | ACaataactacaTA |
| 527 | cacaataactacat | 20565 | 20578 | 2-10-2 | 527_1 | CAcaataactacAT |
| 528 | tcacaataactaca | 20566 | 20579 | 2-10-2 | 528_1 | TCacaataactaCA |
| 529 | ttcacaataactac | 20567 | 20580 | 2-10-2 | 529_1 | TTcacaataactAC |
| 530 | attcacaataacta | 20568 | 20581 | 2-10-2 | 530_1 | ATtcacaataacTA |
| 531 | aattcacaataact | 20569 | 20582 | 2-10-2 | 531_1 | AAttcacaataaCT |
| 532 | gaattcacaataac | 20570 | 20583 | 2-10-2 | 532_1 | GAattcacaataAC |
| 533 | tgaattcacaataa | 20571 | 20584 | 2-10-2 | 533_1 | TGaattcacaatAA |
| 534 | ctaaaacaatctaa | 22073 | 22086 | 2-10-2 | 534_1 | CTaaaacaatctAA |
| 535 | cctaaaacaatcta | 22074 | 22087 | 2-10-2 | 535_1 | CCtaaaacaatcTA |
| 536 | acctaaaacaatct | 22075 | 22088 | 2-10-2 | 536_1 | ACctaaaacaatCT |
| 537 | tacctaaaacaatc | 22076 | 22089 | 2-10-2 | 537_1 | TAcctaaaacaaTC |
| 538 | atacctaaaacaat | 22077 | 22090 | 2-10-2 | 538_1 | ATacctaaaacaAT |
| 539 | tatacctaaaacaa | 22078 | 22091 | 2-10-2 | 539_1 | TAtacctaaaacAA |
| 540 | ctatacctaaaaca | 22079 | 22092 | 2-10-2 | 540_1 | CTatacctaaaaCA |
| 541 | gctatacctaaaac | 22080 | 22093 | 2-10-2 | 541_1 | GCtatacctaaaAC |
| 542 | ttgtaactaaaaat | 22254 | 22267 | 2-10-2 | 542_1 | TTgtaactaaaaAT |
| 543 | cttgtaactaaaaa | 22255 | 22268 | 2-10-2 | 543_1 | CTtgtaactaaaAA |
| 544 | ccttgtaactaaaa | 22256 | 22269 | 2-10-2 | 544_1 | CCttgtaactaaAA |
| 545 | cccttgtaactaaa | 22257 | 22270 | 2-10-2 | 545_1 | CCcttgtaactaAA |
| 546 | ccccttgtaactaa | 22258 | 22271 | 2-10-2 | 546_1 | CCccttgtaactAA |
| 547 | accccttgtaacta | 22259 | 22272 | 2-10-2 | 547_1 | ACcccttgtaacTA |
| 548 | caccccttgtaact | 22260 | 22273 | 2-10-2 | 548_1 | CAccccttgtaaCT |
| 549 | acaccccttgtaac | 22261 | 22274 | 2-10-2 | 549_1 | ACaccccttgtaAC |
| 550 | ttcatatatacatc | 22424 | 22437 | 2-10-2 | 550_1 | TTcatatatacaTC |
| 551 | cttcatatatacat | 22425 | 22438 | 2-10-2 | 551_1 | CTtcatatatacAT |
| 552 | ccttcatatataca | 22426 | 22439 | 2-10-2 | 552_1 | CCttcatatataCA |
| 553 | cccttcatatatac | 22427 | 22440 | 2-10-2 | 553_1 | CCcttcatatatAC |
| 554 | acccttcatatata | 22428 | 22441 | 2-10-2 | 554_1 | ACccttcatataTA |
| 555 | tacccttcatatat | 22429 | 22442 | 2-10-2 | 555_1 | TAcccttcatatAT |
| 556 | ttacccttcatata | 22430 | 22443 | 2-10-2 | 556_1 | TTacccttcataTA |
| 557 | attacccttcatat | 22431 | 22444 | 2-10-2 | 557_1 | ATtacccttcatAT |
| 558 | cattacccttcata | 22432 | 22445 | 2-10-2 | 558_1 | CAttacccttcaTA |
| 559 | acattacccttcat | 22433 | 22446 | 2-10-2 | 559_1 | ACattacccttcAT |
| 560 | tacattacccttca | 22434 | 22447 | 2-10-2 | 560_1 | TAcattacccttCA |
| 561 | tcttatacttacta | 23204 | 23217 | 2-10-2 | 561_1 | TCttatacttacTA |
| 562 | ttcttatacttact | 23205 | 23218 | 2-10-2 | 562_1 | TTcttatacttaCT |
| 563 | attcttatacttac | 23206 | 23219 | 2-10-2 | 563_1 | ATtcttatacttAC |
| 564 | gattcttatactta | 23207 | 23220 | 2-10-2 | 564_1 | GAttcttatactTA |
| 565 | tgattcttatactt | 23208 | 23221 | 2-10-2 | 565_1 | TGattcttatacTT |
| 566 | atgattcttatact | 23209 | 23222 | 2-10-2 | 566_1 | ATgattcttataCT |
| 567 | aacttcactaaaat | 23616 | 23629 | 2-10-2 | 567_1 | AActtcactaaaAT |
| 568 | aaacttcactaaaa | 23617 | 23630 | 2-10-2 | 568_1 | AAacttcactaaAA |
| 569 | taaacttcactaaa | 23618 | 23631 | 2-10-2 | 569_1 | TAaacttcactaAA |
| 570 | ataaacttcactaa | 23619 | 23632 | 2-10-2 | 570_1 | ATaaacttcactAA |
| 571 | aataaacttcacta | 23620 | 23633 | 2-10-2 | 571_1 | AAtaaacttcacTA |
| 572 | taataaacttcact | 23621 | 23634 | 2-10-2 | 572_1 | TAataaacttcaCT |
| 573 | ctaataaacttcac | 23622 | 23635 | 2-10-2 | 573_1 | CTaataaacttcAC |
| 574 | actaataaacttca | 23623 | 23636 | 2-10-2 | 574_1 | ACtaataaacttCA |
| 575 | aactaataaacttc | 23624 | 23637 | 2-10-2 | 575_1 | AActaataaactTC |
| 576 | aatcttctatttta | 24108 | 24121 | 2-10-2 | 576_1 | AAtcttctatttTA |
| 577 | caatcttctatttt | 24109 | 24122 | 2-10-2 | 577_1 | CAatcttctattTT |
| 578 | ccaatcttctattt | 24110 | 24123 | 2-10-2 | 578_1 | CCaatcttctatTT |
| 579 | accaatcttctatt | 24111 | 24124 | 2-10-2 | 579_1 | ACcaatcttctaTT |
| 580 | aaccaatcttctat | 24112 | 24125 | 2-10-2 | 580_1 | AAccaatcttctAT |
| 581 | caaccaatcttcta | 24113 | 24126 | 2-10-2 | 581_1 | CAaccaatcttcTA |
| 582 | gcaaccaatcttct | 24114 | 24127 | 2-10-2 | 582_1 | GCaaccaatcttCT |
| 583 | tgcaaccaatcttc | 24115 | 24128 | 2-10-2 | 583_1 | TGcaaccaatctTC |
| 584 | ctgcaaccaatctt | 24116 | 24129 | 2-10-2 | 584_1 | CTgcaaccaatcTT |
| 585 | actgcaaccaatct | 24117 | 24130 | 2-10-2 | 585_1 | ACtgcaaccaatCT |
| 586 | aactgcaaccaatc | 24118 | 24131 | 2-10-2 | 586_1 | AActgcaaccaaTC |
| 587 | taactgcaaccaat | 24119 | 24132 | 2-10-2 | 587_1 | TAactgcaaccaAT |
| 588 | tacaacacacatca | 24335 | 24348 | 2-10-2 | 588_1 | TAcaacacacatCA |
| 589 | atacaacacacatc | 24336 | 24349 | 2-10-2 | 589_1 | ATacaacacacaTC |
| 590 | aatacaacacacat | 24337 | 24350 | 2-10-2 | 590_1 | AAtacaacacacAT |
| 591 | gaatacaacacaca | 24338 | 24351 | 2-10-2 | 591_1 | GAatacaacacaCA |
| 592 | tgaatacaacacac | 24339 | 24352 | 2-10-2 | 592_1 | TGaatacaacacAC |
| 593 | atgaatacaacaca | 24340 | 24353 | 2-10-2 | 593_1 | ATgaatacaacaCA |
| 594 | cctaataaaatata | 24499 | 24512 | 2-10-2 | 594_1 | CCtaataaaataTA |
| 595 | tcctaataaaatat | 24500 | 24513 | 2-10-2 | 595_1 | TCctaataaaatAT |
| 596 | ctcctaataaaata | 24501 | 24514 | 2-10-2 | 596_1 | CTcctaataaaaTA |
| 597 | actcctaataaaat | 24502 | 24515 | 2-10-2 | 597_1 | ACtcctaataaaAT |
| 598 | tactcctaataaaa | 24503 | 24516 | 2-10-2 | 598_1 | TActcctaataaAA |
| 599 | ctactcctaataaa | 24504 | 24517 | 2-10-2 | 599_1 | CTactcctaataAA |
| 600 | actactcctaataa | 24505 | 24518 | 2-10-2 | 600_1 | ACtactcctaatAA |
| 601 | aactactcctaata | 24506 | 24519 | 2-10-2 | 601_1 | AActactcctaaTA |
| 602 | taactactcctaat | 24507 | 24520 | 2-10-2 | 602_1 | TAactactcctaAT |
| 603 | ataactactcctaa | 24508 | 24521 | 2-10-2 | 603_1 | ATaactactcctAA |
| 604 | tataactactccta | 24509 | 24522 | 2-10-2 | 604_1 | TAtaactactccTA |
| 605 | atataactactcct | 24510 | 24523 | 2-10-2 | 605_1 | ATataactactcCT |
| 606 | aatataactactcc | 24511 | 24524 | 2-10-2 | 606_1 | AAtataactactCC |
| 607 | aaatataactactc | 24512 | 24525 | 2-10-2 | 607_1 | AAatataactacTC |
| 608 | aaaatataactact | 24513 | 24526 | 2-10-2 | 608_1 | AAaatataactaCT |
| 609 | aaaaatataactac | 24514 | 24527 | 2-10-2 | 609_1 | AAaaatataactAC |
| 610 | taaaaatataacta | 24515 | 24528 | 2-10-2 | 610_1 | TAaaaatataacTA |
| 611 | gtaaaaatataact | 24516 | 24529 | 2-10-2 | 611_1 | GTaaaaatataaCT |
| 612 | agtaaaaatataac | 24517 | 24530 | 2-10-2 | 612_1 | AGtaaaaatataAC |
| 613 | actgatacccacaa | 24593 | 24606 | 2-10-2 | 613_1 | ACtgatacccacAA |
| 614 | aactgatacccaca | 24594 | 24607 | 2-10-2 | 614_1 | AActgatacccaCA |
| 615 | caactgatacccac | 24595 | 24608 | 2-10-2 | 615_1 | CAactgatacccAC |
| 616 | tcaactgataccca | 24596 | 24609 | 2-10-2 | 616_1 | TCaactgataccCA |
| 617 | atcactaaaaaact | 24752 | 24765 | 2-10-2 | 617_1 | ATcactaaaaaaCT |
| 618 | tatcactaaaaaac | 24753 | 24766 | 2-10-2 | 618_1 | TAtcactaaaaaAC |
| 619 | atatcactaaaaaa | 24754 | 24767 | 2-10-2 | 619_1 | ATatcactaaaaAA |
| 620 | tatatcactaaaaa | 24755 | 24768 | 2-10-2 | 620_1 | TAtatcactaaaAA |
| 621 | ttatatcactaaaa | 24756 | 24769 | 2-10-2 | 621_1 | TTatatcactaaAA |
| 622 | tttatatcactaaa | 24757 | 24770 | 2-10-2 | 622_1 | TTtatatcactaAA |
| 623 | gtttatatcactaa | 24758 | 24771 | 2-10-2 | 623_1 | GTttatatcactAA |
| 624 | aaacttttaattaa | 24850 | 24863 | 2-10-2 | 624_1 | AAacttttaattAA |
| 625 | caaacttttaatta | 24851 | 24864 | 2-10-2 | 625_1 | CAaacttttaatTA |
| 626 | tcaaacttttaatt | 24852 | 24865 | 2-10-2 | 626_1 | TCaaacttttaaTT |
| 627 | ttcaaacttttaat | 24853 | 24866 | 2-10-2 | 627_1 | TTcaaacttttaAT |
| 628 | cttcaaacttttaa | 24854 | 24867 | 2-10-2 | 628_1 | CTtcaaacttttAA |
| 629 | acttcaaactttta | 24855 | 24868 | 2-10-2 | 629_1 | ACttcaaactttTA |
| 630 | cacttcaaactttt | 24856 | 24869 | 2-10-2 | 630_1 | CActtcaaacttTT |
| 631 | ccacttcaaacttt | 24857 | 24870 | 2-10-2 | 631_1 | CCacttcaaactTT |
| 632 | cccacttcaaactt | 24858 | 24871 | 2-10-2 | 632_1 | CCcacttcaaacTT |
| 633 | acccacttcaaact | 24859 | 24872 | 2-10-2 | 633_1 | ACccacttcaaaCT |
| 634 | aacccacttcaaac | 24860 | 24873 | 2-10-2 | 634_1 | AAcccacttcaaAC |
| 635 | aaacccacttcaaa | 24861 | 24874 | 2-10-2 | 635_1 | AAacccacttcaAA |
| 636 | aaaacccacttcaa | 24862 | 24875 | 2-10-2 | 636_1 | AAaacccacttcAA |
| 637 | aaaaacccacttca | 24863 | 24876 | 2-10-2 | 637_1 | AAaaacccacttCA |
| 638 | aaaaaacccacttc | 24864 | 24877 | 2-10-2 | 638_1 | AAaaaacccactTC |
| 639 | caaaaaacccactt | 24865 | 24878 | 2-10-2 | 639_1 | CAaaaaacccacTT |
| 640 | acaaaaaacccact | 24866 | 24879 | 2-10-2 | 640_1 | ACaaaaaacccaCT |
| 641 | aacaaaaaacccac | 24867 | 24880 | 2-10-2 | 641_1 | AAcaaaaaacccAC |
| 642 | aaacaaaaaaccca | 24868 | 24881 | 2-10-2 | 642_1 | AAacaaaaaaccCA |
| 643 | aaaacaaaaaaccc | 24869 | 24882 | 2-10-2 | 643_1 | AAaacaaaaaacCC |
| 644 | atcttcccattaat | 24976 | 24989 | 2-10-2 | 644_1 | ATcttcccattaAT |
| 645 | aatcttcccattaa | 24977 | 24990 | 2-10-2 | 645_1 | AAtcttcccattAA |
| 646 | taatcttcccatta | 24978 | 24991 | 2-10-2 | 646_1 | TAatcttcccatTA |
| 647 | ataatcttcccatt | 24979 | 24992 | 2-10-2 | 647_1 | ATaatcttcccaTT |
| 648 | aataatcttcccat | 24980 | 24993 | 2-10-2 | 648_1 | AAtaatcttcccAT |
| 649 | aaataatcttccca | 24981 | 24994 | 2-10-2 | 649_1 | AAataatcttccCA |
| 650 | aaaataatcttccc | 24982 | 24995 | 2-10-2 | 650_1 | AAaataatcttcCC |
| 651 | tattaatcaaaaat | 25057 | 25070 | 2-10-2 | 651_1 | TAttaatcaaaaAT |
| 652 | ctattaatcaaaaa | 25058 | 25071 | 2-10-2 | 652_1 | CTattaatcaaaAA |
| 653 | tctattaatcaaaa | 25059 | 25072 | 2-10-2 | 653_1 | TCtattaatcaaAA |
| 654 | ctctattaatcaaa | 25060 | 25073 | 2-10-2 | 654_1 | CTctattaatcaAA |
| 655 | actctattaatcaa | 25061 | 25074 | 2-10-2 | 655_1 | ACtctattaatcAA |
| 656 | gactctattaatca | 25062 | 25075 | 2-10-2 | 656_1 | GActctattaatCA |
| 657 | tattctactcttct | 25433 | 25446 | 2-10-2 | 657_1 | TAttctactcttCT |
| 658 | atattctactcttc | 25434 | 25447 | 2-10-2 | 658_1 | ATattctactctTC |
| 659 | aatattctactctt | 25435 | 25448 | 2-10-2 | 659_1 | AAtattctactcTT |
| 660 | gaatattctactct | 25436 | 25449 | 2-10-2 | 660_1 | GAatattctactCT |
| 661 | agaatattctactc | 25437 | 25450 | 2-10-2 | 661_1 | AGaatattctacTC |
| 662 | atttaccaattcaa | 25508 | 25521 | 2-10-2 | 662_1 | ATttaccaattcAA |
| 663 | tatttaccaattca | 25509 | 25522 | 2-10-2 | 663_1 | TAtttaccaattCA |
| 664 | gtatttaccaattc | 25510 | 25523 | 2-10-2 | 664_1 | GTatttaccaatTC |
| 665 | tgtatttaccaatt | 25511 | 25524 | 2-10-2 | 665_1 | TGtatttaccaaTT |
| 666 | ctgtatttaccaat | 25512 | 25525 | 2-10-2 | 666_1 | CTgtatttaccaAT |
| 667 | actgtatttaccaa | 25513 | 25526 | 2-10-2 | 667_1 | ACtgtatttaccAA |
| 668 | ttataccatcaaat | 27100 | 27113 | 2-10-2 | 668_1 | TTataccatcaaAT |
| 669 | attataccatcaaa | 27101 | 27114 | 2-10-2 | 669_1 | ATtataccatcaAA |
| 670 | cattataccatcaa | 27102 | 27115 | 2-10-2 | 670_1 | CAttataccatcAA |
| 671 | tcattataccatca | 27103 | 27116 | 2-10-2 | 671_1 | TCattataccatCA |
| 672 | ttcattataccatc | 27104 | 27117 | 2-10-2 | 672_1 | TTcattataccaTC |
| 673 | cttcattataccat | 27105 | 27118 | 2-10-2 | 673_1 | CTtcattataccAT |
| 674 | tcttcattatacca | 27106 | 27119 | 2-10-2 | 674_1 | TCttcattatacCA |
| 675 | ttcttcattatacc | 27107 | 27120 | 2-10-2 | 675_1 | TTcttcattataCC |
| 676 | tttcttcattatac | 27108 | 27121 | 2-10-2 | 676_1 | TTtcttcattatAC |
| 677 | ttttcttcattata | 27109 | 27122 | 2-10-2 | 677_1 | TTttcttcattaTA |
| 678 | attttcttcattat | 27110 | 27123 | 2-10-2 | 678_1 | ATtttcttcattAT |
| 679 | tattttcttcatta | 27111 | 27124 | 2-10-2 | 679_1 | TAttttcttcatTA |
| 680 | atattttcttcatt | 27112 | 27125 | 2-10-2 | 680_1 | ATattttcttcaTT |
| 681 | aatattttcttcat | 27113 | 27126 | 2-10-2 | 681_1 | AAattttcttcaAT |
| 682 | aaatattttcttca | 27114 | 27127 | 2-10-2 | 682_1 | AAatattttcttCA |
| 683 | taaatattttcttc | 27115 | 27128 | 2-10-2 | 683_1 | TAaatattttctTC |
| 684 | aataatccaaactt | 27772 | 27785 | 2-10-2 | 684_1 | AAtaatccaaacTT |
| 685 | aaataatccaaact | 27773 | 27786 | 2-10-2 | 685_1 | AAataatccaaaCT |
| 686 | aaaataatccaaac | 27774 | 27787 | 2-10-2 | 686_1 | AAaataatccaaAC |
| 687 | caaaataatccaaa | 27775 | 27788 | 2-10-2 | 687_1 | CAaaataatccaAA |
| 688 | acaaaataatccaa | 27776 | 27789 | 2-10-2 | 688_1 | ACaaaataatccAA |
| 689 | tacaaaataatcca | 27777 | 27790 | 2-10-2 | 689_1 | TAcaaaataatcCA |
| 690 | ttacaaaataatcc | 27778 | 27791 | 2-10-2 | 690_1 | TTacaaaataatCC |
| 691 | gttacaaaataatc | 27779 | 27792 | 2-10-2 | 691_1 | GTtacaaaataaTC |
| 692 | tgttacaaaataat | 27780 | 27793 | 2-10-2 | 692_1 | TGttacaaaataAT |
| 693 | ttttacattaacta | 27935 | 27948 | 2-10-2 | 693_1 | TTttacattaacTA |
| 694 | tttttacattaact | 27936 | 27949 | 2-10-2 | 694_1 | TTtttacattaaCT |
| 695 | ttttttacattaac | 27937 | 27950 | 2-10-2 | 695_1 | TTttttacattaAC |
| 696 | attttttacattaa | 27938 | 27951 | 2-10-2 | 696_1 | ATtttttacattAA |
| 697 | tattttttacatta | 27939 | 27952 | 2-10-2 | 697_1 | TAttttttacatTA |
| 698 | ttattttttacatt | 27940 | 27953 | 2-10-2 | 698_1 | TTattttttacaTT |
| 699 | aaatactaacatca | 29299 | 29312 | 2-10-2 | 699_1 | AAatactaacatCA |
| 700 | aaaatactaacatc | 29300 | 29313 | 2-10-2 | 700_1 | AAaatactaacaTC |
| 701 | caaaatactaacat | 29301 | 29314 | 2-10-2 | 701_1 | CAaaatactaacAT |
| 702 | ccaaaatactaaca | 29302 | 29315 | 2-10-2 | 702_1 | CCaaaatactaaCA |
| 703 | gccaaaatactaac | 29303 | 29316 | 2-10-2 | 703_1 | GCcaaaatactaAC |
| 704 | tgccaaaatactaa | 29304 | 29317 | 2-10-2 | 704_1 | TGccaaaatactAA |
| 705 | tccattcattttat | 29415 | 29428 | 2-10-2 | 705_1 | TCcattcattttAT |
| 706 | atccattcatttta | 29416 | 29429 | 2-10-2 | 706_1 | ATccattcatttTA |
| 707 | catccattcatttt | 29417 | 29430 | 2-10-2 | 707_1 | CAtccattcattTT |
| 708 | acatccattcattt | 29418 | 29431 | 2-10-2 | 708_1 | ACatccattcatTT |
| 709 | cacatccattcatt | 29419 | 29432 | 2-10-2 | 709_1 | CAcatccattcaTT |
| 710 | ccacatccattcat | 29420 | 29433 | 2-10-2 | 710_1 | CCacatccattcAT |
| 711 | gccacatccattca | 29421 | 29434 | 2-10-2 | 711_1 | GCcacatccattCA |
| 712 | tgccacatccattc | 29422 | 29435 | 2-10-2 | 712_1 | TGccacatccatTC |
| 713 | atgccacatccatt | 29423 | 29436 | 2-10-2 | 713_1 | ATgccacatccaTT |
| 714 | tatgccacatccat | 29424 | 29437 | 2-10-2 | 714_1 | TAtgccacatccAT |
| 715 | ttatgccacatcca | 29425 | 29438 | 2-10-2 | 715_1 | TTatgccacatcCA |
| 716 | attatgccacatcc | 29426 | 29439 | 2-10-2 | 716_1 | ATtatgccacatCC |
| 717 | tcttaactcttctc | 30753 | 30766 | 2-10-2 | 717_1 | TCttaactcttcTC |
| 718 | ttcttaactcttct | 30754 | 30767 | 2-10-2 | 718_1 | TTcttaactcttCT |
| 719 | gttcttaactcttc | 30755 | 30768 | 2-10-2 | 719_1 | GTtcttaactctTC |
| 720 | agttcttaactctt | 30756 | 30769 | 2-10-2 | 720_1 | AGttcttaactcTT |
| 721 | tagttcttaactct | 30757 | 30770 | 2-10-2 | 721_1 | TAgttcttaactCT |
| 722 | caaatactcaaaaa | 31029 | 31042 | 2-10-2 | 722_1 | CAaatactcaaaAA |
| 723 | tcaaatactcaaaa | 31030 | 31043 | 2-10-2 | 723_1 | TCaaatactcaaAA |
| 724 | ttcaaatactcaaa | 31031 | 31044 | 2-10-2 | 724_1 | TTcaaatactcaAA |
| 725 | cttcaaatactcaa | 31032 | 31045 | 2-10-2 | 725_1 | CTtcaaatactcAA |
| 726 | gcttcaaatactca | 31033 | 31046 | 2-10-2 | 726_1 | GCttcaaatactCA |
| 727 | agcttcaaatactc | 31034 | 31047 | 2-10-2 | 727_1 | AGcttcaaatacTC |
| 728 | aagcttcaaatact | 31035 | 31048 | 2-10-2 | 728_1 | AAgcttcaaataCT |
| 729 | cctcattacccatt | 32059 | 32072 | 2-10-2 | 729_1 | CCtcattacccaTT |
| 730 | tcctcattacccat | 32060 | 32073 | 2-10-2 | 730_1 | TCctcattacccAT |
| 731 | atcctcattaccca | 32061 | 32074 | 2-10-2 | 731_1 | ATcctcattaccCA |
| 732 | tatcctcattaccc | 32062 | 32075 | 2-10-2 | 732_1 | TAtcctcattacCC |
| 733 | atatcctcattacc | 32063 | 32076 | 2-10-2 | 733_1 | ATatcctcattaCC |
| 734 | aatatcctcattac | 32064 | 32077 | 2-10-2 | 734_1 | AAtatcctcattAC |
| 735 | taatatcctcatta | 32065 | 32078 | 2-10-2 | 735_1 | TAatatcctcatTA |
| 736 | ttaatatcctcatt | 32066 | 32079 | 2-10-2 | 736_1 | TTaatatcctcaTT |
| 737 | tttaatatcctcat | 32067 | 32080 | 2-10-2 | 737_1 | TTtaatatcctcAT |
| 738 | atttaatatcctca | 32068 | 32081 | 2-10-2 | 738_1 | ATttaatatcctCA |
| 739 | aatttaatatcctc | 32069 | 32082 | 2-10-2 | 739_1 | AAtttaatatccTC |
| 740 | aaatttaatatcct | 32070 | 32083 | 2-10-2 | 740_1 | AAatttaatatcCT |
| 741 | taaatttaatatcc | 32071 | 32084 | 2-10-2 | 741_1 | TAaatttaatatCC |
| 742 | ttaaatttaatatc | 32072 | 32085 | 2-10-2 | 742_1 | TTaaatttaataTC |
| 743 | cttaaatttaatat | 32073 | 32086 | 2-10-2 | 743_1 | CTtaaatttaatAT |
| 744 | tcttaaatttaata | 32074 | 32087 | 2-10-2 | 744_1 | TCttaaatttaaTA |
| 745 | ttcttaaatttaat | 32075 | 32088 | 2-10-2 | 745_1 | TTcttaaatttaAT |
| 746 | gttcttaaatttaa | 32076 | 32089 | 2-10-2 | 746_1 | GTtcttaaatttAA |
| 747 | ttattctactttta | 33431 | 33444 | 2-10-2 | 747_1 | TTattctactttTA |
| 748 | tttattctactttt | 33432 | 33445 | 2-10-2 | 748_1 | TTtattctacttTT |
| 749 | ctttattctacttt | 33433 | 33446 | 2-10-2 | 749_1 | CTttattctactTT |
| 750 | cctttattctactt | 33434 | 33447 | 2-10-2 | 750_1 | CCtttattctacTT |
| 751 | gcctttattctact | 33435 | 33448 | 2-10-2 | 751_1 | GCctttattctaCT |
| 752 | aacaattattaata | 33797 | 33810 | 2-10-2 | 752_1 | AAcaattattaaTA |
| 753 | caacaattattaat | 33798 | 33811 | 2-10-2 | 753_1 | CAacaattattaAT |
| 754 | gcaacaattattaa | 33799 | 33812 | 2-10-2 | 754_1 | GCaacaattattAA |
| 755 | agcaacaattatta | 33800 | 33813 | 2-10-2 | 755_1 | AGcaacaattatTA |
| 756 | cagcaacaattatt | 33801 | 33814 | 2-10-2 | 756_1 | CAgcaacaattaTT |
| 757 | ccagcaacaattat | 33802 | 33815 | 2-10-2 | 757_1 | CCagcaacaattAT |
| 758 | accagcaacaatta | 33803 | 33816 | 2-10-2 | 758_1 | ACcagcaacaatTA |
| 759 | aaaccaaaacttac | 33963 | 33976 | 2-10-2 | 759_1 | AAaccaaaacttAC |
| 760 | aaaaccaaaactta | 33964 | 33977 | 2-10-2 | 760_1 | AAaaccaaaactTA |
| 761 | aaaaaccaaaactt | 33965 | 33978 | 2-10-2 | 761_1 | AAaaaccaaaacTT |
| 762 | caaaaaccaaaact | 33966 | 33979 | 2-10-2 | 762_1 | CAaaaaccaaaaCT |
| 763 | ccaaaaaccaaaac | 33967 | 33980 | 2-10-2 | 763_1 | CCaaaaaccaaaAC |
| 764 | accaaaaaccaaaa | 33968 | 33981 | 2-10-2 | 764_1 | ACcaaaaaccaaAA |
| 765 | aaccaaaaaccaaa | 33969 | 33982 | 2-10-2 | 765_1 | AAccaaaaaccaAA |
| 766 | aaaccaaaaaccaa | 33970 | 33983 | 2-10-2 | 766_1 | AAaccaaaaaccAA |
| 767 | atctaaaacacttc | 34050 | 34063 | 2-10-2 | 767_1 | ATctaaaacactTC |
| 768 | aatctaaaacactt | 34051 | 34064 | 2-10-2 | 768_1 | AAtctaaaacacTT |
| 769 | aaatctaaaacact | 34052 | 34065 | 2-10-2 | 769_1 | AAatctaaaacaCT |
| 770 | caaatctaaaacac | 34053 | 34066 | 2-10-2 | 770_1 | CAaatctaaaacAC |
| 771 | ccaaatctaaaaca | 34054 | 34067 | 2-10-2 | 771_1 | CCaaatctaaaaCA |
| 772 | cccaaatctaaaac | 34055 | 34068 | 2-10-2 | 772_1 | CCcaaatctaaaAC |
| 773 | ccccaaatctaaaa | 34056 | 34069 | 2-10-2 | 773_1 | CCccaaatctaaAA |
| 774 | accccaaatctaaa | 34057 | 34070 | 2-10-2 | 774_1 | ACcccaaatctaAA |
| 775 | aaccccaaatctaa | 34058 | 34071 | 2-10-2 | 775_1 | AAccccaaatctAA |
| 776 | aaaccccaaatcta | 34059 | 34072 | 2-10-2 | 776_1 | AAaccccaaatcTA |
| 777 | attcacaaatccta | 34075 | 34088 | 2-10-2 | 777_1 | ATtcacaaatccTA |
| 778 | tattcacaaatcct | 34076 | 34089 | 2-10-2 | 778_1 | TAttcacaaatcCT |
| 779 | atattcacaaatcc | 34077 | 34090 | 2-10-2 | 779_1 | ATattcacaaatCC |
| 780 | aatattcacaaatc | 34078 | 34091 | 2-10-2 | 780_1 | AAtattcacaaaTC |
| 781 | aaatattcacaaat | 34079 | 34092 | 2-10-2 | 781_1 | AAatattcacaaAT |
| 782 | caaatattcacaaa | 34080 | 34093 | 2-10-2 | 782_1 | CAaatattcacaAA |
| 783 | gcaaatattcacaa | 34081 | 34094 | 2-10-2 | 783_1 | GCaaatattcacAA |
| 784 | aacacacattatca | 34537 | 34550 | 2-10-2 | 784_1 | AAcacacattatCA |
| 785 | taacacacattatc | 34538 | 34551 | 2-10-2 | 785_1 | TAacacacattaTC |
| 786 | ttaacacacattat | 34539 | 34552 | 2-10-2 | 786_1 | TTaacacacattAT |
| 787 | tttaacacacatta | 34540 | 34553 | 2-10-2 | 787_1 | TTtaacacacatTA |
| 788 | atttaacacacatt | 34541 | 34554 | 2-10-2 | 788_1 | ATttaacacacaTT |
| 789 | tatttaacacacat | 34542 | 34555 | 2-10-2 | 789_1 | TAtttaacacacAT |
| 790 | ctatttaacacaca | 34543 | 34556 | 2-10-2 | 790_1 | CTatttaacacaCA |
| 791 | actatttaacacac | 34544 | 34557 | 2-10-2 | 791_1 | ACtatttaacacAC |
| 792 | tactatttaacaca | 34545 | 34558 | 2-10-2 | 792_1 | TActatttaacaCA |
| 793 | ctactatttaacac | 34546 | 34559 | 2-10-2 | 793_1 | CTactatttaacAC |
| 794 | actactatttaaca | 34547 | 34560 | 2-10-2 | 794_1 | ACtactatttaaCA |
| 795 | aactactatttaac | 34548 | 34561 | 2-10-2 | 795_1 | AActactatttaAC |
| 796 | aaactactatttaa | 34549 | 34562 | 2-10-2 | 796_1 | AAactactatttAA |
| 797 | aaaactactattta | 34550 | 34563 | 2-10-2 | 797_1 | AAaactactattTA |
| 798 | gaaaactactattt | 34551 | 34564 | 2-10-2 | 798_1 | GAaaactactatTT |
| 799 | tgaaaactactatt | 34552 | 34565 | 2-10-2 | 799_1 | TGaaaactactaTT |
| 800 | aaataacctatcat | 35309 | 35322 | 2-10-2 | 800_1 | AAataacctatcAT |
| 801 | aaaataacctatca | 35310 | 35323 | 2-10-2 | 801_1 | AAaataacctatCA |
| 802 | caaaataacctatc | 35311 | 35324 | 2-10-2 | 802_1 | CAaaataacctaTC |
| 803 | acaaaataacctat | 35312 | 35325 | 2-10-2 | 803_1 | ACaaaataacctAT |
| 804 | cacaaaataaccta | 35313 | 35326 | 2-10-2 | 804_1 | CAcaaaataaccTA |
| 805 | tcacaaaataacct | 35314 | 35327 | 2-10-2 | 805_1 | TCacaaaataacCT |
| 806 | atcacaaaataacc | 35315 | 35328 | 2-10-2 | 806_1 | ATcacaaaataaCC |
| 807 | catcacaaaataac | 35316 | 35329 | 2-10-2 | 807_1 | CAtcacaaaataAC |
| 808 | tcatcacaaaataa | 35317 | 35330 | 2-10-2 | 808_1 | TCatcacaaaatAA |
| 809 | ttcatcacaaaata | 35318 | 35331 | 2-10-2 | 809_1 | TTcatcacaaaaTA |
| 810 | tttcatcacaaaat | 35319 | 35332 | 2-10-2 | 810_1 | TTtcatcacaaaAT |
| 811 | ttttcatcacaaaa | 35320 | 35333 | 2-10-2 | 811_1 | TTttcatcacaaAA |
| 812 | attttcatcacaaa | 35321 | 35334 | 2-10-2 | 812_1 | ATtttcatcacaAA |
| 813 | tattttcatcacaa | 35322 | 35335 | 2-10-2 | 813_1 | TAttttcatcacAA |
| 814 | gtattttcatcaca | 35323 | 35336 | 2-10-2 | 814_1 | GTattttcatcaCA |
| 815 | atttaaatttatca | 35354 | 35367 | 2-10-2 | 815_1 | ATttaaatttatCA |
| 816 | aatttaaatttatc | 35355 | 35368 | 2-10-2 | 816_1 | AAtttaaatttaTC |
| 817 | aaatttaaatttat | 35356 | 35369 | 2-10-2 | 817_1 | AAatttaaatttAT |
| 818 | aaaatttaaattta | 35357 | 35370 | 2-10-2 | 818_1 | AAaatttaaattTA |
| 819 | taaaatttaaattt | 35358 | 35371 | 2-10-2 | 819_1 | TAaaatttaaatTT |
| 820 | ataaaatttaaatt | 35359 | 35372 | 2-10-2 | 820_1 | ATaaaatttaaaTT |
| 821 | cataaaatttaaat | 35360 | 35373 | 2-10-2 | 821_1 | CAtaaaatttaaAT |
| 822 | acataaaatttaaa | 35361 | 35374 | 2-10-2 | 822_1 | ACataaaatttaAA |
| 823 | ctactaatattcat | 36332 | 36345 | 2-10-2 | 823_1 | CTactaatattcAT |
| 824 | cctactaatattca | 36333 | 36346 | 2-10-2 | 824_1 | CCtactaatattCA |
| 825 | acctactaatattc | 36334 | 36347 | 2-10-2 | 825_1 | ACctactaatatTC |
| 826 | cacctactaatatt | 36335 | 36348 | 2-10-2 | 826_1 | CAcctactaataTT |
| 827 | tcacctactaatat | 36336 | 36349 | 2-10-2 | 827_1 | TCacctactaatAT |
| 828 | ttcacctactaata | 36337 | 36350 | 2-10-2 | 828_1 | TTcacctactaaTA |
| 829 | tttcacctactaat | 36338 | 36351 | 2-10-2 | 829_1 | TTtcacctactaAT |
| 830 | ttttcacctactaa | 36339 | 36352 | 2-10-2 | 830_1 | TTttcacctactAA |
| 831 | tttttcacctacta | 36340 | 36353 | 2-10-2 | 831_1 | TTtttcacctacTA |
| 832 | atttttcacctact | 36341 | 36354 | 2-10-2 | 832_1 | ATttttcacctaCT |
| 833 | tatttttcacctac | 36342 | 36355 | 2-10-2 | 833_1 | TAtttttcacctAC |
| 834 | ttatttttcaccta | 36343 | 36356 | 2-10-2 | 834_1 | TTatttttcaccTA |
| 835 | tttatttttcacct | 36344 | 36357 | 2-10-2 | 835_1 | TTtatttttcacCT |
| 836 | ttctactactaatt | 36468 | 36481 | 2-10-2 | 836_1 | TTctactactaaTT |
| 837 | cttctactactaat | 36469 | 36482 | 2-10-2 | 837_1 | CTtctactactaAT |
| 838 | acttctactactaa | 36470 | 36483 | 2-10-2 | 838_1 | ACttctactactAA |
| 839 | aacttctactacta | 36471 | 36484 | 2-10-2 | 839_1 | AActtctactacTA |
| 840 | caacttctactact | 36472 | 36485 | 2-10-2 | 840_1 | CAacttctactaCT |
| 841 | tcaacttctactac | 36473 | 36486 | 2-10-2 | 841_1 | TCaacttctactAC |
| 842 | ctcaacttctacta | 36474 | 36487 | 2-10-2 | 842_1 | CTcaacttctacTA |
| 843 | tctcaacttctact | 36475 | 36488 | 2-10-2 | 843_1 | TCtcaacttctaCT |
| 844 | ctctcaacttctac | 36476 | 36489 | 2-10-2 | 844_1 | CTctcaacttctAC |
| 845 | tctctcaacttcta | 36477 | 36490 | 2-10-2 | 845_1 | TCtctcaacttcTA |
| 846 | ttctctcaacttct | 36478 | 36491 | 2-10-2 | 846_1 | TTctctcaacttCT |
| 847 | tttctctcaacttc | 36479 | 36492 | 2-10-2 | 847_1 | TTtctctcaactTC |
| 848 | ttttctctcaactt | 36480 | 36493 | 2-10-2 | 848_1 | TTttctctcaacTT |
| 849 | tttttctctcaact | 36481 | 36494 | 2-10-2 | 849_1 | TTtttctctcaaCT |
| 850 | ctttttctctcaac | 36482 | 36495 | 2-10-2 | 850_1 | CTttttctctcaAC |
| 851 | actttttctctcaa | 36483 | 36496 | 2-10-2 | 851_1 | ACtttttctctcAA |
| 852 | tactttttctctca | 36484 | 36497 | 2-10-2 | 852_1 | TActttttctctCA |
| 853 | ttactttttctctc | 36485 | 36498 | 2-10-2 | 853_1 | TTactttttctcTC |
| 854 | gttactttttctct | 36486 | 36499 | 2-10-2 | 854_1 | GTtactttttctCT |
| 855 | agttactttttctc | 36487 | 36500 | 2-10-2 | 855_1 | AGttac11111cTC |
| 856 | cattcccattaaca | 36788 | 36801 | 2-10-2 | 856_1 | CAttcccattaaCA |
| 857 | acattcccattaac | 36789 | 36802 | 2-10-2 | 857_1 | ACattcccattaAC |
| 858 | tacattcccattaa | 36790 | 36803 | 2-10-2 | 858_1 | TAcattcccattAA |
| 859 | ttacattcccatta | 36791 | 36804 | 2-10-2 | 859_1 | TTacattcccatTA |
| 860 | tttacattcccatt | 36792 | 36805 | 2-10-2 | 860_1 | TTtacattcccaTT |
| 861 | ttttacattcccat | 36793 | 36806 | 2-10-2 | 861_1 | TTttacattcccAT |
| 862 | cttttacattccca | 36794 | 36807 | 2-10-2 | 862_1 | CTtttacattccCA |
| 863 | acttttacattccc | 36795 | 36808 | 2-10-2 | 863_1 | ACttttacattcCC |
| 864 | cacttttacattcc | 36796 | 36809 | 2-10-2 | 864_1 | CActtttacattCC |
| 865 | acacttttacattc | 36797 | 36810 | 2-10-2 | 865_1 | ACacttttacatTC |
| 866 | tacacttttacatt | 36798 | 36811 | 2-10-2 | 866_1 | TAcacttttacaTT |
| 867 | gtacacttttacat | 36799 | 36812 | 2-10-2 | 867_1 | GTacacttttacAT |
| 868 | tgtacacttttaca | 36800 | 36813 | 2-10-2 | 868_1 | TGtacacttttaCA |
| 869 | tttatcaaaaaaat | 36834 | 36847 | 2-10-2 | 869_1 | TTtatcaaaaaaAT |
| 870 | atttatcaaaaaaa | 36835 | 36848 | 2-10-2 | 870_1 | ATttatcaaaaaAA |
| 871 | catttatcaaaaaa | 36836 | 36849 | 2-10-2 | 871_1 | CAtttatcaaaaAA |
| 872 | acatttatcaaaaa | 36837 | 36850 | 2-10-2 | 872_1 | ACatttatcaaaAA |
| 873 | tacatttatcaaaa | 36838 | 36851 | 2-10-2 | 873_1 | TAcatttatcaaAA |
| 874 | atacatttatcaaa | 36839 | 36852 | 2-10-2 | 874_1 | ATacatttatcaAA |
| 875 | tatacatttatcaa | 36840 | 36853 | 2-10-2 | 875_1 | TAtacatttatcAA |
| 876 | acatcttccaattt | 38848 | 38861 | 2-10-2 | 876_1 | ACatcttccaatTT |
| 877 | tacatcttccaatt | 38849 | 38862 | 2-10-2 | 877_1 | TAcatcttccaaTT |
| 878 | ttacatcttccaat | 38850 | 38863 | 2-10-2 | 878_1 | TTacatcttccaAT |
| 879 | tttacatcttccaa | 38851 | 38864 | 2-10-2 | 879_1 | TTtacatcttccAA |
| 880 | atttacatcttcca | 38852 | 38865 | 2-10-2 | 880_1 | ATttacatcttcCA |
| 881 | tatttacatcttcc | 38853 | 38866 | 2-10-2 | 881_1 | TAtttacatcttCC |
| 882 | ttatttacatcttc | 38854 | 38867 | 2-10-2 | 882_1 | TTatttacatctTC |
| 883 | cttatttacatctt | 38855 | 38868 | 2-10-2 | 883_1 | CTtatttacatcTT |
| 884 | tcttatttacatct | 38856 | 38869 | 2-10-2 | 884_1 | TCttatttacatCT |
| 885 | atcttatttacatc | 38857 | 38870 | 2-10-2 | 885_1 | ATcttatttacaTC |
| 886 | aatcttatttacat | 38858 | 38871 | 2-10-2 | 886_1 | AAtcttatttacAT |
| 887 | gaatcttatttaca | 38859 | 38872 | 2-10-2 | 887_1 | GAatcttatttaCA |
| 888 | tgaatcttatttac | 38860 | 38873 | 2-10-2 | 888_1 | TGaatcttatttAC |
| 889 | ttcccttcactcct | 40071 | 40084 | 2-10-2 | 889_1 | TTcccttcactcCT |
| 890 | tttcccttcactcc | 40072 | 40085 | 2-10-2 | 890_1 | TTtcccttcactCC |
| 891 | ttttcccttcactc | 40073 | 40086 | 2-10-2 | 891_1 | TTttcccttcacTC |
| 892 | attttcccttcact | 40074 | 40087 | 2-10-2 | 892_1 | ATtttcccttcaCT |
| 893 | aattttcccttcac | 40075 | 40088 | 2-10-2 | 893_1 | AAttttcccttcAC |
| 894 | taattttcccttca | 40076 | 40089 | 2-10-2 | 894_1 | TAattttcccttCA |
| 895 | ttaattttcccttc | 40077 | 40090 | 2-10-2 | 895_1 | TTaattttccctTC |
| 896 | gttaattttccctt | 40078 | 40091 | 2-10-2 | 896_1 | GTtaattttcccTT |
| 897 | tttatcatttcttt | 40150 | 40163 | 2-10-2 | 897_1 | TTtatcatttctTT |
| 898 | ttttatcatttctt | 40151 | 40164 | 2-10-2 | 898_1 | TTttatcatttcTT |
| 899 | cttttatcatttct | 40152 | 40165 | 2-10-2 | 899_1 | CTtttatcatttCT |
| 900 | tcttttatcatttc | 40153 | 40166 | 2-10-2 | 900_1 | TCttttatcattTC |
| 901 | ttcttttatcattt | 40154 | 40167 | 2-10-2 | 901_1 | TTcttttatcatTT |
| 902 | cttcttttatcatt | 40155 | 40168 | 2-10-2 | 902_1 | CTtcttttatcaTT |
| 903 | acttcttttatcat | 40156 | 40169 | 2-10-2 | 903_1 | ACttcttttatcAT |
| 904 | tacttcttttatca | 40157 | 40170 | 2-10-2 | 904_1 | TActtcttttatCA |
| 905 | ttacttcttttatc | 40158 | 40171 | 2-10-2 | 905_1 | TTacttcttttaTC |
| 906 | attacttcttttat | 40159 | 40172 | 2-10-2 | 906_1 | ATtacttcttttAT |
| 907 | aattacttctttta | 40160 | 40173 | 2-10-2 | 907_1 | AAttacttctttTA |
| 908 | aaattacttctttt | 40161 | 40174 | 2-10-2 | 908_1 | AAattacttcttTT |
| 909 | aaaattacttcttt | 40162 | 40175 | 2-10-2 | 909_1 | AAaattacttctTT |
| 910 | caaaattacttctt | 40163 | 40176 | 2-10-2 | 910_1 | CAaaattacttcTT |
| 911 | ccaaaattacttct | 40164 | 40177 | 2-10-2 | 911_1 | CCaaaattacttCT |
| 912 | tccaaaattacttc | 40165 | 40178 | 2-10-2 | 912_1 | TCcaaaattactTC |
| 913 | ttccaaaattactt | 40166 | 40179 | 2-10-2 | 913_1 | TTccaaaattacTT |
| 914 | gttccaaaattact | 40167 | 40180 | 2-10-2 | 914_1 | GTtccaaaattaCT |
| 915 | tgttccaaaattac | 40168 | 40181 | 2-10-2 | 915_1 | TGttccaaaattAC |
| 916 | atgttccaaaatta | 40169 | 40182 | 2-10-2 | 916_1 | ATgttccaaaatTA |
| 917 | ttactctttttatt | 40201 | 40214 | 2-10-2 | 917_1 | TTactctttttaTT |
| 918 | tttactctttttat | 40202 | 40215 | 2-10-2 | 918_1 | TTtactctttttAT |
| 919 | ttttactcttttta | 40203 | 40216 | 2-10-2 | 919_1 | TTttactcttttTA |
| 920 | attttactcttttt | 40204 | 40217 | 2-10-2 | 920_1 | ATtttactctttTT |
| 921 | tattttactctttt | 40205 | 40218 | 2-10-2 | 921_1 | TAttttactcttTT |
| 922 | atattttactcttt | 40206 | 40219 | 2-10-2 | 922_1 | ATattttactctTT |
| 923 | catattttactctt | 40207 | 40220 | 2-10-2 | 923_1 | CAtattttactcTT |
| 924 | ccatattttactct | 40208 | 40221 | 2-10-2 | 924_1 | CCatattttactCT |
| 925 | cccatattttactc | 40209 | 40222 | 2-10-2 | 925_1 | CCcatattttacTC |
| 926 | acccatattttact | 40210 | 40223 | 2-10-2 | 926_1 | ACccatattttaCT |
| 927 | tacccatattttac | 40211 | 40224 | 2-10-2 | 927_1 | TAcccatattttAC |
| 928 | ttacccatatttta | 40212 | 40225 | 2-10-2 | 928_1 | TTacccatatttTA |
| 929 | tttacccatatttt | 40213 | 40226 | 2-10-2 | 929_1 | TTtacccatattTT |
| 930 | gtttacccatattt | 40214 | 40227 | 2-10-2 | 930_1 | GTttacccatatTT |
| 931 | tgtttacccatatt | 40215 | 40228 | 2-10-2 | 931_1 | TGtttacccataTT |
| 932 | gttacctcccttta | 40368 | 40381 | 2-10-2 | 932_1 | GTtacctcccttTA |
| 933 | ggttacctcccttt | 40369 | 40382 | 2-10-2 | 933_1 | GGttacctccctTT |
| 934 | aggttacctccctt | 40370 | 40383 | 2-10-2 | 934_1 | AGgttacctcccTT |
| 935 | caaactaaaaccta | 41659 | 41672 | 2-10-2 | 935_1 | CAaactaaaaccTA |
| 936 | tcaaactaaaacct | 41660 | 41673 | 2-10-2 | 936_1 | TCaaactaaaacCT |
| 937 | atcaaactaaaacc | 41661 | 41674 | 2-10-2 | 937_1 | ATcaaactaaaaCC |
| 938 | gatcaaactaaaac | 41662 | 41675 | 2-10-2 | 938_1 | GAtcaaactaaaAC |
| 939 | agatcaaactaaaa | 41663 | 41676 | 2-10-2 | 939_1 | AGatcaaactaaAA |
| 940 | aagatcaaactaaa | 41664 | 41677 | 2-10-2 | 940_1 | AAgatcaaactaAA |
| 941 | ccaatttcacccaa | 41699 | 41712 | 2-10-2 | 941_1 | CCaatttcacccAA |
| 942 | cccaatttcaccca | 41700 | 41713 | 2-10-2 | 942_1 | CCcaatttcaccCA |
| 943 | gcccaatttcaccc | 41701 | 41714 | 2-10-2 | 943_1 | GCccaatttcacCC |
| 944 | tgcccaatttcacc | 41702 | 41715 | 2-10-2 | 944_1 | TGcccaatttcaCC |
| 945 | ttgcccaatttcac | 41703 | 41716 | 2-10-2 | 945_1 | TTgcccaatttcAC |
| 946 | caactttctatttt | 41777 | 41790 | 2-10-2 | 946_1 | CAactttctattTT |
| 947 | ccaactttctattt | 41778 | 41791 | 2-10-2 | 947_1 | CCaactttctatTT |
| 948 | cccaactttctatt | 41779 | 41792 | 2-10-2 | 948_1 | CCcaactttctaTT |
| 949 | acccaactttctat | 41780 | 41793 | 2-10-2 | 949_1 | ACccaactttctAT |
| 950 | aacccaactttcta | 41781 | 41794 | 2-10-2 | 950_1 | AAcccaactttcTA |
| 951 | aaacccaactttct | 41782 | 41795 | 2-10-2 | 951_1 | AAacccaactttCT |
| 952 | aaaacccaactttc | 41783 | 41796 | 2-10-2 | 952_1 | AAaacccaacttTC |
| 953 | aaaaacccaacttt | 41784 | 41797 | 2-10-2 | 953_1 | AAaaacccaactTT |
| 954 | caaaaacccaactt | 41785 | 41798 | 2-10-2 | 954_1 | CAaaaacccaacTT |
| 955 | acaaaaacccaact | 41786 | 41799 | 2-10-2 | 955_1 | ACaaaaacccaaCT |
| 956 | ctttaaaatttcca | 42170 | 42183 | 2-10-2 | 956_1 | CTttaaaatttcCA |
| 957 | tctttaaaatttcc | 42171 | 42184 | 2-10-2 | 957_1 | TCtttaaaatttCC |
| 958 | ttctttaaaatttc | 42172 | 42185 | 2-10-2 | 958_1 | TTctttaaaattTC |
| 959 | tttctttaaaattt | 42173 | 42186 | 2-10-2 | 959_1 | TTtctttaaaatTT |
| 960 | atttctttaaaatt | 42174 | 42187 | 2-10-2 | 960_1 | ATttctttaaaaTT |
| 961 | catttctttaaaat | 42175 | 42188 | 2-10-2 | 961_1 | CAtttctttaaaAT |
| 962 | acatttctttaaaa | 42176 | 42189 | 2-10-2 | 962_1 | ACatttctttaaAA |
| 963 | cacatttctttaaa | 42177 | 42190 | 2-10-2 | 963_1 | CAcatttctttaAA |
| 964 | ccacatttctttaa | 42178 | 42191 | 2-10-2 | 964_1 | CCacatttctttAA |
| 965 | accacatttcttta | 42179 | 42192 | 2-10-2 | 965_1 | ACcacatttcttTA |
| 966 | aaccacatttcttt | 42180 | 42193 | 2-10-2 | 966_1 | AAccacatttctTT |
| 967 | aaaccacatttctt | 42181 | 42194 | 2-10-2 | 967_1 | AAaccacatttcTT |
| 968 | aaaaccacatttct | 42182 | 42195 | 2-10-2 | 968_1 | AAaaccacatttCT |
| 969 | caaaaccacatttc | 42183 | 42196 | 2-10-2 | 969_1 | CAaaaccacattTC |
| 970 | ttcttctcttttca | 43831 | 43844 | 2-10-2 | 970_1 | TTcttctcttttCA |
| 971 | tttcttctcttttc | 43832 | 43845 | 2-10-2 | 971_1 | TTtcttctctttTC |
| 972 | ttttcttctctttt | 43833 | 43846 | 2-10-2 | 972_1 | TTttcttctcttTT |
| 973 | tttttcttctcttt | 43834 | 43847 | 2-10-2 | 973_1 | TTtttcttctctTT |
| 974 | ttttttcttctctt | 43835 | 43848 | 2-10-2 | 974_1 | TTttttcttctcTT |
| 975 | attttttcttctct | 43836 | 43849 | 2-10-2 | 975_1 | ATtttttcttctCT |
| 976 | tattttttcttctc | 43837 | 43850 | 2-10-2 | 976_1 | TAttttttcttcTC |
| 977 | aacttaatattaaa | 45488 | 45501 | 2-10-2 | 977_1 | AActtaatattaAA |
| 978 | caacttaatattaa | 45489 | 45502 | 2-10-2 | 978_1 | CAacttaatattAA |
| 979 | tcaacttaatatta | 45490 | 45503 | 2-10-2 | 979_1 | TCaacttaatatTA |
| 980 | ttcaacttaatatt | 45491 | 45504 | 2-10-2 | 980_1 | TTcaacttaataTT |
| 981 | attcaacttaatat | 45492 | 45505 | 2-10-2 | 981_1 | ATtcaacttaatAT |
| 982 | tattcaacttaata | 45493 | 45506 | 2-10-2 | 982_1 | TAttcaacttaaTA |
| 983 | ttattcaacttaat | 45494 | 45507 | 2-10-2 | 983_1 | TTattcaacttaAT |
| 984 | tttattcaacttaa | 45495 | 45508 | 2-10-2 | 984_1 | TTtattcaacttAA |
| 985 | caaattaaaaaaca | 47397 | 47410 | 2-10-2 | 985_1 | CAaattaaaaaaCA |
| 986 | tcaaattaaaaaac | 47398 | 47411 | 2-10-2 | 986_1 | TCaaattaaaaaAC |
| 987 | ttcaaattaaaaaa | 47399 | 47412 | 2-10-2 | 987_1 | TTcaaattaaaaAA |
| 988 | cttcaaattaaaaa | 47400 | 47413 | 2-10-2 | 988_1 | CTtcaaattaaaAA |
| 989 | tcttcaaattaaaa | 47401 | 47414 | 2-10-2 | 989_1 | TCttcaaattaaAA |
| 990 | ttcttcaaattaaa | 47402 | 47415 | 2-10-2 | 990_1 | TTcttcaaattaAA |
| 991 | tttcttcaaattaa | 47403 | 47416 | 2-10-2 | 991_1 | TTtcttcaaattAA |
| 992 | aacacaaattcaaa | 48077 | 48090 | 2-10-2 | 992_1 | AAcacaaattcaAA |
| 993 | aaacacaaattcaa | 48078 | 48091 | 2-10-2 | 993_1 | AAacacaaattcAA |
| 994 | taaacacaaattca | 48079 | 48092 | 2-10-2 | 994_1 | TAaacacaaattCA |
| 995 | ataaacacaaattc | 48080 | 48093 | 2-10-2 | 995_1 | ATaaacacaaatTC |
| 996 | aataaacacaaatt | 48081 | 48094 | 2-10-2 | 996_1 | AAtaaacacaaaTT |
| 997 | caataaacacaaat | 48082 | 48095 | 2-10-2 | 997_1 | CAataaacacaaAT |
| 998 | acaataaacacaaa | 48083 | 48096 | 2-10-2 | 998_1 | ACaataaacacaAA |
| 999 | aacaataaacacaa | 48084 | 48097 | 2-10-2 | 999_1 | AAcaataaacacAA |
| 1000 | taacaataaacaca | 48085 | 48098 | 2-10-2 | 1000_1 | TAacaataaacaCA |
| 1001 | ttaacaataaacac | 48086 | 48099 | 2-10-2 | 1001_1 | TTaacaataaacAC |
| 1002 | attaacaataaaca | 48087 | 48100 | 2-10-2 | 1002_1 | ATtaacaataaaCA |
| 1003 | aattaacaataaac | 48088 | 48101 | 2-10-2 | 1003_1 | AAttaacaataaAC |
| 1004 | gaattaacaataaa | 48089 | 48102 | 2-10-2 | 1004_1 | GAattaacaataAA |
| 1005 | tgaattaacaataa | 48090 | 48103 | 2-10-2 | 1005_1 | TGaattaacaatAA |
| 1006 | atattcctcaatca | 48905 | 48918 | 2-10-2 | 1006_1 | ATattcctcaatCA |
| 1007 | tatattcctcaatc | 48906 | 48919 | 2-10-2 | 1007_1 | TAtattcctcaaTC |
| 1008 | atatattcctcaat | 48907 | 48920 | 2-10-2 | 1008_1 | ATatattcctcaAT |
| 1009 | aatatattcctcaa | 48908 | 48921 | 2-10-2 | 1009_1 | AAtatattcctcAA |
| 1010 | caatatattcctca | 48909 | 48922 | 2-10-2 | 1010_1 | CAatatattcctCA |
| 1011 | acaatatattcctc | 48910 | 48923 | 2-10-2 | 1011_1 | ACaatatattccTC |
| 1012 | gacaatatattcct | 48911 | 48924 | 2-10-2 | 1012_1 | GAcaatatattcCT |
| 1013 | caatcctaattaaa | 48960 | 48973 | 2-10-2 | 1013_1 | CAatcctaattaAA |
| 1014 | ccaatcctaattaa | 48961 | 48974 | 2-10-2 | 1014_1 | CCaatcctaattAA |
| 1015 | cccaatcctaatta | 48962 | 48975 | 2-10-2 | 1015_1 | CCcaatcctaatTA |
| 1016 | gcccaatcctaatt | 48963 | 48976 | 2-10-2 | 1016_1 | GCccaatcctaaTT |
| 1017 | tgcccaatcctaat | 48964 | 48977 | 2-10-2 | 1017_1 | TGcccaatcctaAT |
| 1018 | accctacaaatact | 50093 | 50106 | 2-10-2 | 1018_1 | ACcctacaaataCT |
| 1019 | aaccctacaaatac | 50094 | 50107 | 2-10-2 | 1019_1 | AAccctacaaatAC |
| 1020 | aaaccctacaaata | 50095 | 50108 | 2-10-2 | 1020_1 | AAaccctacaaaTA |
| 1021 | aaaaccctacaaat | 50096 | 50109 | 2-10-2 | 1021_1 | AAaaccctacaaAT |
| 1022 | aaaaaccctacaaa | 50097 | 50110 | 2-10-2 | 1022_1 | AAaaaccctacaAA |
| 1023 | aaaaaaccctacaa | 50098 | 50111 | 2-10-2 | 1023_1 | AAaaaaccctacAA |
| 1024 | aaaaaaaccctaca | 50099 | 50112 | 2-10-2 | 1024_1 | AAaaaaaccctaCA |
| 1025 | tatacactattaat | 51008 | 51021 | 2-10-2 | 1025_1 | TAtacactattaAT |
| 1026 | ttatacactattaa | 51009 | 51022 | 2-10-2 | 1026_1 | TTatacactattAA |
| 1027 | attatacactatta | 51010 | 51023 | 2-10-2 | 1027_1 | ATtatacactatTA |
| 1028 | aattatacactatt | 51011 | 51024 | 2-10-2 | 1028_1 | AAttatacactaTT |
| 1029 | gaattatacactat | 51012 | 51025 | 2-10-2 | 1029_1 | GAattatacactAT |
| 1030 | gtaacaattataca | 51866 | 51879 | 2-10-2 | 1030_1 | GTaacaattataCA |
| 1031 | tgtaacaattatac | 51867 | 51880 | 2-10-2 | 1031_1 | TGtaacaattatAC |
| 1032 | ctgtaacaattata | 51868 | 51881 | 2-10-2 | 1032_1 | CTgtaacaattaTA |
| 1033 | cctgtaacaattat | 51869 | 51882 | 2-10-2 | 1033_1 | CCtgtaacaattAT |
| 1034 | tcctgtaacaatta | 51870 | 51883 | 2-10-2 | 1034_1 | TCctgtaacaatTA |
| 1035 | ataaaaaccacctt | 53263 | 53276 | 2-10-2 | 1035_1 | ATaaaaaccaccTT |
| 1036 | aataaaaaccacct | 53264 | 53277 | 2-10-2 | 1036_1 | AAtaaaaaccacCT |
| 1037 | gaataaaaaccacc | 53265 | 53278 | 2-10-2 | 1037_1 | GAataaaaaccaCC |
| 1038 | agaataaaaaccac | 53266 | 53279 | 2-10-2 | 1038_1 | AGaataaaaaccAC |
| 1039 | cagaataaaaacca | 53267 | 53280 | 2-10-2 | 1039_1 | CAgaataaaaacCA |
| 1040 | ccagaataaaaacc | 53268 | 53281 | 2-10-2 | 1040_1 | CCagaataaaaaCC |
| 1041 | cccagaataaaaac | 53269 | 53282 | 2-10-2 | 1041_1 | CCcagaataaaaAC |
| 1042 | acccagaataaaaa | 53270 | 53283 | 2-10-2 | 1042_1 | ACccagaataaaAA |
| 1043 | tttcttactcccct | 53699 | 53712 | 2-10-2 | 1043_1 | TTtcttactcccCT |
| 1044 | ctttcttactcccc | 53700 | 53713 | 2-10-2 | 1044_1 | CTttcttactccCC |
| 1045 | actttcttactccc | 53701 | 53714 | 2-10-2 | 1045_1 | ACtttcttactcCC |
| 1046 | cactttcttactcc | 53702 | 53715 | 2-10-2 | 1046_1 | CActttcttactCC |
| 1047 | ccactttcttactc | 53703 | 53716 | 2-10-2 | 1047_1 | CCactttcttacTC |
| 1048 | cctttaccactttt | 53948 | 53961 | 2-10-2 | 1048_1 | CCtttaccacttTT |
| 1049 | ccctttaccacttt | 53949 | 53962 | 2-10-2 | 1049_1 | CCctttaccactTT |
| 1050 | tccctttaccactt | 53950 | 53963 | 2-10-2 | 1050_1 | TCcctttaccacTT |
| 1051 | atccctttaccact | 53951 | 53964 | 2-10-2 | 1051_1 | ATccctttaccaCT |
| 1052 | catccctttaccac | 53952 | 53965 | 2-10-2 | 1052_1 | CAtccctttaccAC |
| 1053 | ctacatctaacccc | 54550 | 54563 | 2-10-2 | 1053_1 | CTacatctaaccCC |
| 1054 | tctacatctaaccc | 54551 | 54564 | 2-10-2 | 1054_1 | TCtacatctaacCC |
| 1055 | gtctacatctaacc | 54552 | 54565 | 2-10-2 | 1055_1 | GTctacatctaaCC |
| 1056 | agtctacatctaac | 54553 | 54566 | 2-10-2 | 1056_1 | AGtctacatctaAC |
| 1057 | cagtctacatctaa | 54554 | 54567 | 2-10-2 | 1057_1 | CAgtctacatctAA |
| 1058 | tcagtctacatcta | 54555 | 54568 | 2-10-2 | 1058_1 | TCagtctacatcTA |
| 1059 | ttcagtctacatct | 54556 | 54569 | 2-10-2 | 1059_1 | TTcagtctacatCT |
| 1060 | taaccacacctcct | 54573 | 54586 | 2-10-2 | 1060_1 | TAaccacacctcCT |
| 1061 | ttaaccacacctcc | 54574 | 54587 | 2-10-2 | 1061_1 | TTaaccacacctCC |
| 1062 | tttaaccacacctc | 54575 | 54588 | 2-10-2 | 1062_1 | TTtaaccacaccTC |
| 1063 | ttttaaccacacct | 54576 | 54589 | 2-10-2 | 1063_1 | TTttaaccacacCT |
| 1064 | gttttaaccacacc | 54577 | 54590 | 2-10-2 | 1064_1 | GTtttaaccacaCC |
| 1065 | agttttaaccacac | 54578 | 54591 | 2-10-2 | 1065_1 | AGttttaaccacAC |
| 1066 | caacaaaacatcaa | 55228 | 55241 | 2-10-2 | 1066_1 | CAacaaaacatcAA |
| 1067 | tcaacaaaacatca | 55229 | 55242 | 2-10-2 | 1067_1 | TCaacaaaacatCA |
| 1068 | ttcaacaaaacatc | 55230 | 55243 | 2-10-2 | 1068_1 | TTcaacaaaacaTC |
| 1069 | tttcaacaaaacat | 55231 | 55244 | 2-10-2 | 1069_1 | TTtcaacaaaacAT |
| 1070 | ttttcaacaaaaca | 55232 | 55245 | 2-10-2 | 1070_1 | TTttcaacaaaaCA |
| 1071 | gttttcaacaaaac | 55233 | 55246 | 2-10-2 | 1071_1 | GTtttcaacaaaAC |
| 1072 | tgttttcaacaaaa | 55234 | 55247 | 2-10-2 | 1072_1 | TGttttcaacaaAA |
| 1073 | ttctaaaacttacc | 55269 | 55282 | 2-10-2 | 1073_1 | TTctaaaacttaCC |
| 1074 | tttctaaaacttac | 55270 | 55283 | 2-10-2 | 1074_1 | TTtctaaaacttAC |
| 1075 | ctttctaaaactta | 55271 | 55284 | 2-10-2 | 1075_1 | CTttctaaaactTA |
| 1076 | tctttctaaaactt | 55272 | 55285 | 2-10-2 | 1076_1 | TCtttctaaaacTT |
| 1077 | atctttctaaaact | 55273 | 55286 | 2-10-2 | 1077_1 | ATctttctaaaaCT |
| 1078 | aatctttctaaaac | 55274 | 55287 | 2-10-2 | 1078_1 | AAtctttctaaaAC |
| 1079 | gaatctttctaaaa | 55275 | 55288 | 2-10-2 | 1079_1 | GAatctttctaaAA |
| 1080 | agaatctttctaaa | 55276 | 55289 | 2-10-2 | 1080_1 | AGaatctttctaAA |
| 1081 | cagaatctttctaa | 55277 | 55290 | 2-10-2 | 1081_1 | CAgaatctttctAA |
| 1082 | cctttatttccctt | 55494 | 55507 | 2-10-2 | 1082_1 | CCtttatttcccTT |
| 1083 | ccctttatttccct | 55495 | 55508 | 2-10-2 | 1083_1 | CCctttatttccCT |
| 1084 | tccctttatttccc | 55496 | 55509 | 2-10-2 | 1084_1 | TCcctttatttcCC |
| 1085 | ttccctttatttcc | 55497 | 55510 | 2-10-2 | 1085_1 | TTccctttatttCC |
| 1086 | tttccctttatttc | 55498 | 55511 | 2-10-2 | 1086_1 | TTtccctttattTC |
| 1087 | atttccctttattt | 55499 | 55512 | 2-10-2 | 1087_1 | ATttccctttatTT |
| 1088 | tatttccctttatt | 55500 | 55513 | 2-10-2 | 1088_1 | TAtttccctttaTT |
| 1089 | gtatttccctttat | 55501 | 55514 | 2-10-2 | 1089_1 | GTatttccctttAT |

### Example 2: In vitro reduction of ATXN3 in SK-N-AS human cell line using further LNA gapmer oligonucleotides targeting ATNX3

### Materials and Methods:

LNA modified oligonucleotides targeting human ATXN3 were tested for their ability to reduce ATXN3 mRNA expression in human SK-N-AS neuroblastoma cells acquired from ECACC Cat: 94092302. The cells were cultured according to the vendor guidelines in Dulbecco's Modified Eagle's Medium, supplemented with 0.1 mM Non-Essential Amino Acids (NEAA) and fetal bovine serum to a final concentration of 10%. Cells were cultured at 37° C., 5% CO2 and 95% humidity in an active evaporation incubator (Thermo C10). Cells were seeded at a density of 9000 cells per well (96-well plate) in 190 ul of SK-N-AS cell culture medium. The cells were hereafter added 10 μl of oligo suspension or PBS (controls) to a final concentration of 5 μM from pre-made 96-well dilution plates. The cell culture plates were incubated for 72 hours in the incubator.

After incubation, cells were harvested by removal of media followed by cell lysis and RNA purification using QIAGEN RNeasy 96 Kit (cat 74181), following manufacturers protocol. RNA was diluted 2 fold in water prior to the one-step qPCR reaction. For one-step qPCR reaction qPCR-mix (qScriptTM XLT One-Step RT-qPCR ToughMix® Low ROX from QuantaBio, cat.no 95134-500) and QPCR was run as duplex QPCR using assays from Integrated DNA technologies for ATXN3 (Hs.PT.58.39355049) and TBP (Hs.PT.58v.39858774)

Hs.PT.58.39355049 - Primer Sequences
Probe:
(SEQ ID NO: 1130)
5′-/56-FAM/AAAGGCCAG/ZEN/CCACCAGTTCAGG/3IABkFQ/-3′
Primer 1:
(SEQ ID NO: 1129)
5′-CTATCAGGACAGAGTTCACATCC-3′
Primer 2:
(SEQ ID NO: 1128)
5′-GTTTCTAAAGACATGGTCACAGC-3′
Hs.PT.58v.39858774 - Primer Sequences
Probe:
(SEQ ID NO: 1131)
5′-/5HEX/TGA TCT TTG/ZEN/CAG TGA CCC AGC ATC A/
3IABkFQ/-3′
Primer 1:
(SEQ ID NO: 1132)
5′-GCT GTT TAA CTT CGC TTC CG-3′
Primer 2:
(SEQ ID NO: 1133)
5′-CAG CAA CTT CCT CAA TTC CTT G-3′

The reactions were then mixed in a qPCR plate (MICROAMP®optical 384 well, 4309849). After sealing, the plate was given a quick spin, 1000 g for 1 minute at RT, and transferred to a Viia™ 7 system (Applied Biosystems, Thermo), and the following PCR conditions used: 50° C. for 15 minutes; 95° C. for 3 minutes; 40 cycles of: 95° C. for 5 sec followed by a temperature decrease of 1.6° C./sec followed by 60° C. for 45 sec. The data was analyzed using the QuantStudio™ Real time PCR Software and quantity calculated by the delta delta Ct method (Quantity=2\hat{ }(−Ct)*1000000000). Quantity is normalized to the calculated quantity for the housekeeping gene assay (TBP) run in the same well. Relative Target Quantity=QUANTITY_target/QUANTITY_housekeeping (RNA knockdown) was calculated for each well by division with the mean of all PBS-treated wells on the same plate. Normalised Target Quantity=(Relative Target Quantity/[mean] Relative Target Quantity]_pbs_wells)*100.

Compounds targeting selected target sequence regions of SEQ ID NO:1 were evaluated in the above assay.

### Results:

The target knock-down data is presented in the following Compound and Data Table:

In the Compound table, motif sequences represent the contiguous sequence of nucleobases present in the oligonucleotide.

Oligonucleotide compound represent specific designs of a motif sequence. Capital letters represent beta-D-oxy LNA nucleosides, lowercase letters represent DNA nucleosides, all LNA C are 5-methyl cytosine, all internucleoside linkages are phosphorothioate internucleoside linkages.

TABLE 4
Compound and Data Table

| | | | | % of |
|---|---|---|---|---|
| | | | | ATXN3 |
| | | Oligonucleotide | Oligonucleotide | mRNA |
| SEQID | CMPID | Base Sequence | compound | remaining |
| 1099 | 1099_1 | CCAAAAGAAACCAAACCC | CCAAaagaaaccaaacCC | 90.6 |
| 1100 | 1100_1 | CCCCATTCAAATATTTATT | CCccattcaaatatttATT | 90.5 |
| 1101 | 1101_1 | AATCATTTACCCCCAAC | AAtcatttaccccCAAC | 92 |
| 1102 | 1102_1 | TATCTCAAACTATCCCCA | TAtctcaaactatcccCA | 93 |
| 1103 | 1103_1 | TCTATTCCTTAACCCAAC | TCTattccttaacccAAC | 76.6 |
| 1104 | 1104_1 | TCCCCTAAATAATTTAATCA | TCccctaaataatttaATCA | 79.3 |
| 1105 | 1105_1 | AAACCACTCCATTCCAA | AaaccactccattCCAA | 57.7 |
| 1106 | 1106_1 | TCTAAACCCCAAACTTTCA | TCtaaaccccaaactttCA | 74.3 |
| 1107 | 1107_1 | TTCTAAACCCCAAACTTTC | TTCtaaaccccaaacttTC | 61.8 |
| 1108 | 1108_1 | AGTTCTAAACCCCAAACT | AGttctaaaccccaaACT | 73.7 |
| 1109 | 1109_1 | TGAAACCATTACTACAACC | TGaaaccattactacAACC | 24.9 |
| 1110 | 1110_1 | ACATCATTTATCACTACCAC | ACAtcatttatcactaccAC | 71.9 |
| 1111 | 1111_1 | AACATTAAACCCTCCCA | AacattaaaccctcCCA | 80.2 |
| 1112 | 1112_1 | TCAGATCCTAAAATCACT | TCAgatcctaaaatcACT | 79.5 |
| 1113 | 1113_1 | CTATACCTAAAACAATCTA | CTAtacctaaaacaatCTA | 99.1 |
| 1114 | 1114_1 | TGATTCTTATACTTACTA | TGAttcttatacttaCTA | 72.1 |
| 1115 | 1115_1 | TAAAAATATAACTACTCCTA | TAaaaatataactactCCTA | 93.7 |
| 1116 | 1116_1 | TCTTCATTATACCATCAAAT | TCTtcattataccatcaAAT | 51.5 |
| 1117 | 1117_1 | GTTTCATATTTTTAATCC | GTTtcatatttttaaTCC | 37.7 |
| 1118 | 1118_1 | TAATATCCTCATTACCCATT | TAatatcctcattacccaTT | 84 |
| 1119 | 1119_1 | CAAATATTCACAAATCCTA | CAaatattcacaaatCCTA | 73.3 |
| 1120 | 1120_1 | CATCACAAAATAACCTATAC | CATcacaaaataacctaTCA | 79.9 |
| 1121 | 1121_1 | CTCTCAACTTCTACTACTAA | CTCtcaacttctactactAA | 59.6 |
| 1122 | 1122_1 | AATCTTATTTACATCTTCC | AATcttatttacatctTCC | 20.7 |
| 1123 | 1123_1 | CCAAAATTACTTCTTTTATC | CCAaaattacttcttttATC | 56.5 |
| 1124 | 1124_1 | AACCCAACTTTCTATTTT | AACCcaactttctattTT | 52.7 |
| 1125 | 1125_1 | ACAATATATTCCTCAATCA | ACAatatattcctcaaTCA | 86.8 |
| 1126 | 1126_1 | CCTGTAACAATTATACA | CCTgtaacaattatACA | 92.3 |
| 1127 | 1127_1 | CATCCCTTTACCACTTT | CAtccctttaccactTT | 94.5 |

In the oligonucleotide compound column, capital letters represent beta-D-oxy LNA nucleosides, LNA cytosines are 5-methyl cytosine, lower case letters are DNA nucleosides, and all internucleoside linkages are phosphorothioate.

As can be seen, most of the above compounds targeting the listed target sequence regions are capable of inhibiting the expression of the human ataxin 3 transcript and that compounds targeting the target sequence region complementary to SEQ ID NOS:1122 and 1109 are particularly effective in inhibiting the human ataxin 3 transcript. Other effective target sites for ATXN3 can be determined from the above table.

### Example 3

### Materials and Methods:

The screening assay described in Example 2 was performed using a series of further oligonucleotide targeting human ATXN3 pre-mRNA using the qpCR: (ATXN3 exon 8-9(1) PrimeTime® XL qPCR Assay (IDT).

qPCR probe and primers set 2:
Probe:
(SEQ ID NO: 1134)
5′-/56-FAM/CTCCGCAGG/ZEN/GCT ATTCAGCT AAGT/
31ABkFQ/-3′
Primer 1:
(SEQ ID NO: 1135)
5′-AGT AAGATTTGT ACCTGATGTCTGT-3′
Primer 2:
(SEQ ID NO: 1136)
5′-CATGGAAGATGAGGAAGCAGAT-3′

### Results:

The results are shown in the following table:

TABLE 5

| | | | | % of |
|---|---|---|---|---|
| | | | | ATXN3 |
| | | Oligonucleotide | Oligonucleotide | mRNA |
| SEQID | CMPID | Base Sequence | compound | remaining |
| 1137 | 1137_1 | CCTACTTCACTTCCTAA | CctacttcacttcCTAA | 68.9 |
| 1138 | 1138_1 | TTTCCTACTTCACTTCCTA | TttcctacttcacttccTA | 95.1 |
| 1139 | 1139_1 | TTCCTACTTCACTTCCTA | TtcctacttcacttcCTA | 85 |
| 1140 | 1140_1 | TTTCCTACTTCACTTCCT | TTtcctacttcacttcCT | 88.1 |
| 1141 | 1141_1 | TTTCCTACTTCACTTCC | TttcctacttcactTCC | 83.1 |
| 1142 | 1142_1 | GTTTCCTACTTCACTTC | GTTtcctacttcactTC | 60.2 |
| 1143 | 1143_1 | ACCAAACCCAAACATCCC | AccaaacccaaacatcCC | 88 |
| 1144 | 1144_1 | AGAAACCAAACCCAAACATC | AgaaaccaaacccaaaCATC | 91.3 |
| 1145 | 1145_1 | AGAAACCAAACCCAAACAT | AGaaaccaaacccaaACAT | 93.5 |
| 1146 | 1146_1 | CTCCTAATACCTAAAAACAAA | CTCCtaatacctaaaaacaAA | 100 |
| 1147 | 1147_1 | CTCCTAATACCTAAAAACA | CTCCtaatacctaaaaaCA | 94.2 |
| 1148 | 1148_1 | ACTCCTAATACCTAAAAACA | ACTCctaatacctaaaaaCA | 81 |
| 1149 | 1149_1 | CACTCCTAATACCTAAAAACA | CACtcctaatacctaaaaACA | 90.4 |
| 1150 | 1150_1 | CCACTCCTAATACCTAAAAA | CCACtcctaatacctaaaAA | 63 |
| 1151 | 1151_1 | TCCACTCCTAATACCTAAAAA | TCCactcctaatacctaaaAA | 54 |
| 1152 | 1152_1 | CCACTCCTAATACCTAAAA | CCACtcctaatacctaaAA | 73.7 |
| 1153 | 1153_1 | TCCACTCCTAATACCTAAAA | TCCactcctaatacctaAAA | 59 |
| 1154 | 1154_1 | CCACTCCTAATACCTAAA | CCACtcctaatacctaAA | 65.2 |
| 1155 | 1155_1 | GTCCACTCCTAATACCTAAA | GtccactcctaataccTAAA | 86.8 |
| 1156 | 1156_1 | CCACTCCTAATACCTAA | CCactcctaatacCTAA | 52.3 |
| 1157 | 1157_1 | TCCACTCCTAATACCTAA | TCcactcctaatacCTAA | 64.3 |
| 1157 | 1157_2 | TCCACTCCTAATACCTAA | TCCActcctaatacctAA | 66 |
| 1158 | 1158_1 | GTCCACTCCTAATACCTAA | GtccactcctaataccTAA | 85.5 |
| 1159 | 1159_1 | AGTCCACTCCTAATACCTA | AgtccactcctaataccTA | 87.4 |
| 1160 | 1160_1 | TCCACTCCTAATACCTA | TCcactcctaatacCTA | 70.1 |
| 1161 | 1161_1 | AGTCCACTCCTAATACCT | AgtccactcctaatacCT | 84.2 |
| 1162 | 1162_1 | GTCCACTCCTAATACC | GTCcactcctaataCC | 57.8 |
| 1163 | 1163_1 | AGTCCACTCCTAATACC | AGtccactcctaataCC | 77.1 |
| 1164 | 1164_1 | CAGTCCACTCCTAATACC | CagtccactcctaatACC | 86.7 |
| 1162 | 1162_2 | GTCCACTCCTAATACC | GTCcactcctaatACC | 67.8 |
| 1165 | 1165_1 | CCAGTCCACTCCTAATAC | CcagtccactcctaaTAC | 85.4 |
| 1166 | 1166_1 | CAGTCCACTCCTAATAC | CAgtccactcctaaTAC | 60.7 |
| 1167 | 1167_1 | AGTCCACTCCTAATAC | AGTCcactcctaatAC | 78.9 |
| 1168 | 1168_1 | CAGTCCACTCCTAATA | CAGtccactcctaaTA | 44.5 |
| 1169 | 1169_1 | CCAGTCCACTCCTAATA | CCagtccactcctaaTA | 33.8 |
| 1170 | 1170_1 | GCAACTCTTTCCAAACA | GCAActctttccaaaCA | 36 |
| 1171 | 1171_1 | AGCAACTCTTTCCAAACA | AGCaactctttccaaaCA | 35.3 |
| 1172 | 1172_1 | CAGCAACTCTTTCCAAACA | CAgcaactctttccaaACA | 58.3 |
| 1173 | 1173_1 | CCAGCAACTCTTTCCAAA | CcagcaactctttcCAAA | 69.7 |
| 1174 | 1174_1 | CCAGCAACTCTTTCCAA | CCagcaactctttcCAA | 42.1 |
| 1175 | 1175_1 | ACCAGCAACTCTTTCCAA | ACcagcaactctttcCAA | 65 |
| 1176 | 1176_1 | TTACCAGCAACTCTTTC | TTACcagcaactcttTC | 53 |
| 1177 | 1177_1 | TGCTCCTCCTATTAAATAA | TGCtcctcctattaaatAA | 76.3 |
| 1178 | 1178_1 | GCTCCTCCTATTAAATAA | GCtcctcctattaaATAA | 61.8 |
| 1179 | 1179_1 | GCTCCTCCTATTAAATA | GCtcctcctattaaATA | 60.2 |
| 1180 | 1180_1 | TGCTCCTCCTATTAAATA | TGctcctcctattaaATA | 70.2 |
| 1181 | 1181_1 | TGCTCCTCCTATTAAAT | TGCtcctcctattaaAT | 80.2 |
| 1182 | 1182_1 | TTGCTCCTCCTATTAAAT | TTGCtcctcctattaaAT | 79 |
| 1183 | 1183_1 | ATTTAATAAAACAAAAACCCT | ATttaataaaacaaaaaCCCT | 97.2 |
| 1184 | 1184_1 | GCCCAAAAAACTAAATT | GCCCaaaaaactaaaTT | 95.5 |
| 1185 | 1185_1 | GTTTTTACATTCTAACTT | GTTtttacattctaaCTT | 54.1 |
| 1186 | 1186_1 | TGTTTTTACATTCTAACT | TGTTtttacattctaaCT | 63.8 |
| 1187 | 1187_1 | CTGTTTTTACATTCTAAC | CTGTttttacattctaAC | 62.5 |
| 1188 | 1188_1 | CCCCATTCAAATATTTAT | CCCcattcaaatattTAT | 64.9 |
| 1189 | 1189_1 | GCCCCATTCAAATATTTAT | GCcccattcaaatattTAT | 86.2 |
| 1188 | 1188_2 | CCCCATTCAAATATTTAT | CCCCattcaaatatttAT | 96.2 |
| 1190 | 1190_1 | GCCCCATTCAAATATTTA | GCcccattcaaatatTTA | 82.2 |
| 1191 | 1191_1 | CCATTCAAATATATACATTTT | CCATtcaaatatatacattTT | 72 |
| 1191 | 1191_2 | CCATTCAAATATATACATTTT | CCATtcaaataTatacattTT | 37.7 |
| 1192 | 1192_1 | TCCATTCAAATATATACATTT | TCCAttcaaatatatacatTT | 56.8 |
| 1193 | 1193_1 | ATCCATTCAAATATATACATT | ATCCattcaaaTatatacaTT | 48 |
| 1194 | 1194_1 | TCCATTCAAATATATACATT | TCCAttcaaatatatacaTT | 53.7 |
| 1193 | 1193_2 | ATCCATTCAAATATATACATT | ATCCattcaaatatatacaTT | 54.7 |
| 1195 | 1195_1 | TATCCATTCAAATATATACAT | TATccattcaaatatataCAT | 80.1 |
| 1196 | 1196_1 | TCCATTCAAATATATACAT | TCCattcaaatatataCAT | 43.1 |
| 1197 | 1197_1 | ATCCATTCAAATATATACA | ATCCattcaaatatataCA | 53.9 |
| 1198 | 1198_1 | TTATCCATTCAAATATATACA | TTatccattcaaatataTACA | 69.4 |
| 1199 | 1199_1 | TCCATTCAAATATATACA | TCCAttcaaatatataCA | 54.7 |
| 1200 | 1200_1 | TATCCATTCAAATATATACA | TATCcattcaaatatataCA | 53.3 |
| 1201 | 1201_1 | CTTTATCCATTCAAATATATA | CTttatccattcaaataTATA | 85.5 |
| 1202 | 1202_1 | TCTTTATCCATTCAAATATAT | TCTttatccattcaaataTAT | 62.6 |
| 1203 | 1203_1 | CTCTTTATCCATTCAAATATA | CTCtttatccattcaaatATA | 38.4 |
| 1204 | 1204_1 | TCTTTATCCATTCAAATATA | TCtttatccattcaaaTATA | 70.9 |
| 1203 | 1203_2 | CTCTTTATCCATTCAAATATA | CTCTttatccattcaaataTA | 33.6 |
| 1205 | 1205_1 | CTTTATCCATTCAAATATA | CTttatccattcaaaTATA | 78.4 |
| 1206 | 1206_1 | TCTCTTTATCCATTCAAATAT | TCtctttatccattcaaaTAT | 82 |
| 1207 | 1207_1 | CTCTTTATCCATTCAAATAT | CTCtttatccattcaaaTAT | 39.8 |
| 1208 | 1208_1 | TCTTTATCCATTCAAATAT | TCTttatccattcaaaTAT | 63.1 |
| 1209 | 1209_1 | TCTCTTTATCCATTCAAATA | TCtctttatccattcaAATA | 65.2 |
| 1210 | 1210_1 | CTCTTTATCCATTCAAATA | CTCTttatccattcaaaTA | 32.2 |
| 1211 | 1211_1 | TCTCTTTATCCATTCAAAT | TCTCtttatccattcaaAT | 42 |
| 1212 | 1212_1 | TCTCTTTATCCATTCAAA | TCTCtttatccattcaAA | 42.5 |
| 1213 | 1213_1 | AGCACCATATATATCTCA | AgcaccatatatatCTCA | 16 |
| 1214 | 1214_1 | GCACCATATATATCTCA | GCaccatatatatcTCA | 16 |
| 1215 | 1215_1 | CAGCACCATATATATCTCA | CagcaccatatatatCTCA | 19.2 |
| 1215 | 1215_2 | CAGCACCATATATATCTCA | CAgcaccatatatatcTCA | 24.1 |
| 1216 | 1216_1 | AGCACCATATATATCTC | AGCaccatatatatcTC | 19.9 |
| 1217 | 1217_1 | GCACCATATATATCTC | GCACcatatatatcTC | 82.7 |
| 1218 | 1218_1 | CAGCACCATATATATCTC | CAgcaccatatatatCTC | 21.1 |
| 1219 | 1219_1 | CAGCACCATATATATCT | CAGcaccatatataTCT | 28.9 |
| 1220 | 1220_1 | ACAGCACCATATATATCT | ACAGcaccatatatatCT | 21.9 |
| 1221 | 1221_1 | ACAGCACCATATATATC | ACAGcaccatatataTC | 25.4 |
| 1222 | 1222_1 | CTATGTTATTATCCCCA | CTAtgttattatcccCA | 56.1 |
| 1223 | 1223_1 | TCTATGTTATTATCCCC | TctatgttattatcCCC | 47.7 |
| 1224 | 1224_1 | CTCTACACTCTAACTCT | CtctacactctaaCTCT | 79.3 |
| 1225 | 1225_1 | TCTCTACACTCTAACTCT | TctctacactctaaCTCT | 79.6 |
| 1226 | 1226_1 | TTCTCTACACTCTAACTCT | TTCtctacactctaactCT | 86.9 |
| 1227 | 1227_1 | CTTCTCTACACTCTAACTCT | CTtctctacactctaactCT | 97 |
| 1227 | 1227_2 | CTTCTCTACACTCTAACTCT | CttctctacactctaacTCT | 84.5 |
| 1228 | 1228_1 | TTCTCTACACTCTAACTC | TTCtctacactctaacTC | 81.4 |
| 1229 | 1229_1 | CTTCTCTACACTCTAACTC | CttctctacactctaaCTC | 89.1 |
| 1230 | 1230_1 | TCTCTACACTCTAACTC | TCtctacactctaaCTC | 87.3 |
| 1231 | 1231_1 | CCTTCTCTACACTCTAACTC | CcttctctacactctaacTC | 98.3 |
| 1232 | 1232_1 | TTCTCTACACTCTAACT | TTCTctacactctaaCT | 80.1 |
| 1233 | 1233_1 | CTTCTCTACACTCTAACT | CTTctctacactctaaCT | 73.6 |
| 1234 | 1234_1 | CCTTCTCTACACTCTAACT | CcttctctacactctAACT | 77.7 |
| 1235 | 1235_1 | CCTTCTCTACACTCTAAC | CCTtctctacactctaAC | 82.4 |
| 1236 | 1236_1 | CTTCTCTACACTCTAAC | CTtctctacactcTAAC | 90.6 |
| 1237 | 1237_1 | AGCCTTCTCTACACTCTAA | AgccttctctacactcTAA | 80 |
| 1238 | 1238_1 | CCTTCTCTACACTCTAA | CCttctctacactcTAA | 72.2 |
| 1239 | 1239_1 | GCCTTCTCTACACTCTAA | GCcttctctacactctAA | 62.9 |
| 1240 | 1240_1 | AGCCTTCTCTACACTCTA | AgccttctctacactcTA | 85.2 |
| 1241 | 1241_1 | TACTAACTACAACACAAATCA | TACtaactacaacacaaaTCA | 91.3 |
| 1241 | 1241_2 | TACTAACTACAACACAAATCA | TACtaactacAacacaaaTCA | 81.1 |
| 1242 | 1242_1 | CTACTAACTACAACACAAATC | CTACtaactacaacacaaaTC | 108 |
| 1243 | 1243_1 | CACTACTAACTACAACACAAA | CACTactaactacaacacaAA | 74 |
| 1244 | 1244_1 | CACTACTAACTACAACACAA | CACtactaactacaacaCAA | 87.4 |
| 1245 | 1245_1 | ACACTACTAACTACAACACAA | ACActactaactacaacaCAA | 84.1 |
| 1246 | 1246_1 | CACTACTAACTACAACACA | CACTactaactacaacaCA | 83.5 |
| 1247 | 1247_1 | ACACTACTAACTACAACACA | ACACtactaactacaacaCA | 81.3 |
| 1248 | 1248_1 | GACACTACTAACTACAACAC | GACactactaactacaaCAC | 51.6 |
| 1249 | 1249_1 | GACACTACTAACTACAACA | GACActactaactacaaCA | 51 |
| 1250 | 1250_1 | AGACACTACTAACTACAA | AGAcactactaactaCAA | 57.2 |
| 1251 | 1251_1 | AGACACTACTAACTACA | AGACactactaactaCA | 34.7 |
| 1252 | 1252_1 | ATCATTTACCCCCAACCT | AtcatttacccccaacCT | 96 |
| 1253 | 1253_1 | ATCATTTACCCCCAACC | AtcatttacccccAACC | 89.1 |
| 1254 | 1254_1 | CAAATCATTTACCCCCAA | CaaatcatttacccCCAA | 92 |
| 1255 | 1255_1 | CCAAATCATTTACCCCCAA | CcaaatcatttaccccCAA | 91 |
| 1256 | 1256_1 | ACCAAATCATTTACCCCCA | AccaaatcatttaccccCA | 89.9 |
| 1257 | 1257_1 | TACCAAATCATTTACCCCC | TaccaaatcatttacccCC | 84 |
| 1258 | 1258_1 | ACCAAATCATTTACCCC | ACcaaatcatttacCCC | 69.9 |
| 1259 | 1259_1 | TACCAAATCATTTACCCC | TACcaaatcatttaccCC | 56.3 |
| 1260 | 1260_1 | CTACCAAATCATTTACCCC | CtaccaaatcatttaccCC | 94 |
| 1261 | 1261_1 | TACCAAATCATTTACCC | TAccaaatcatttACCC | 68.9 |
| 1262 | 1262_1 | CTACCAAATCATTTACC | CTACcaaatcatttaCC | 70.3 |
| 1263 | 1263_1 | TGCTACCAAATCATTTACC | TgctaccaaatcattTACC | 79 |
| 1264 | 1264_1 | GCTACCAAATCATTTACC | GCtaccaaatcatttACC | 83.6 |
| 1265 | 1265_1 | TGCTACCAAATCATTTAC | TGCTaccaaatcatttAC | 88.3 |
| 1266 | 1266_1 | GCTACCAAATCATTTAC | GCTaccaaatcattTAC | 71.4 |
| 1267 | 1267_1 | TGCTACCAAATCATTTA | TGCtaccaaatcatTTA | 79.8 |
| 1268 | 1268_1 | CTGCTACCAAATCATTTA | CTGctaccaaatcatTTA | 75.3 |
| 1269 | 1269_1 | ACTGCTACCAAATCATTT | ACTGctaccaaatcatTT | 83.4 |
| 1270 | 1270_1 | CTGCTACCAAATCATTT | CTGCtaccaaatcatTT | 83 |
| 1271 | 1271_1 | ACTGCTACCAAATCATT | ACTGctaccaaatcaTT | 71.1 |
| 1272 | 1272_1 | CACTTTGCCATAATCAA | CActttgccataaTCAA | 26 |
| 1273 | 1273_1 | TTATCTCAAACTATCCCCA | TTAtctcaaactatcccCA | 92.9 |
| 1274 | 1274_1 | ATCTCAAACTATCCCCA | ATctcaaactatccCCA | 72.3 |
| 1275 | 1275_1 | CTTATCTCAAACTATCCCCA | CttatctcaaactatcccCA | 85.5 |
| 1276 | 1276_1 | TATCTCAAACTATCCCC | TatctcaaactatcCCC | 79.8 |
| 1277 | 1277_1 | CTTATCTCAAACTATCCCC | CTtatctcaaactatccCC | 84 |
| 1278 | 1278_1 | TTATCTCAAACTATCCCC | TTAtctcaaactatccCC | 89.7 |
| 1279 | 1279_1 | CTTATCTCAAACTATCCC | CttatctcaaactaTCCC | 83.5 |
| 1280 | 1280_1 | CCTTATCTCAAACTATCCC | CcttatctcaaactatcCC | 87.6 |
| 1279 | 1279_2 | CTTATCTCAAACTATCCC | CTtatctcaaactatCCC | 76.9 |
| 1281 | 1281_1 | TTATCTCAAACTATCCC | TtatctcaaactaTCCC | 84.7 |
| 1282 | 1282_1 | CTTATCTCAAACTATCC | CTTatctcaaactaTCC | 78.3 |
| 1283 | 1283_1 | CCCTTATCTCAAACTATCC | CccttatctcaaactaTCC | 76.4 |
| 1284 | 1284_1 | CCTTATCTCAAACTATCC | CCTtatctcaaactatCC | 69.3 |
| 1285 | 1285_1 | CCTTATCTCAAACTATC | CCttatctcaaacTATC | 75.9 |
| 1286 | 1286_1 | GCCCTTATCTCAAACTATC | GCccttatctcaaactaTC | 76.6 |
| 1287 | 1287_1 | CCCTTATCTCAAACTATC | CCcttatctcaaacTATC | 67.2 |
| 1288 | 1288_1 | TGCCCTTATCTCAAACTAT | TgcccttatctcaaacTAT | 90.5 |
| 1289 | 1289_1 | GCCCTTATCTCAAACTAT | GCccttatctcaaacTAT | 71.9 |
| 1290 | 1290_1 | CCCTTATCTCAAACTAT | CCCTtatctcaaactAT | 77.7 |
| 1291 | 1291_1 | GCCCTTATCTCAAACTA | GCccttatctcaaacTA | 68.4 |
| 1292 | 1292_1 | TGCCCTTATCTCAAACTA | TgcccttatctcaaaCTA | 81.5 |
| 1293 | 1293_1 | TGCCCTTATCTCAAACT | TGcccttatctcaaaCT | 75.7 |
| 1294 | 1294_1 | TTGCCCTTATCTCAAAC | TTGCccttatctcaaAC | 89 |
| 1295 | 1295_1 | CTTGCCCTTATCTCAA | CTtgcccttatcTCAA | 48.2 |
| 1296 | 1296_1 | TGAAATCAAACTTCATCA | TGAaatcaaacttcaTCA | 66.5 |
| 1297 | 1297_1 | GGTCACCATACTTAAT | GGTCaccatacttaAT | 89.7 |
| 1298 | 1298_1 | TGCTAACACAAATTTCCT | TGctaacacaaattTCCT | 47.3 |
| 1299 | 1299_1 | GCTAACACAAATTTCCT | GCTaacacaaatttCCT | 48.9 |
| 1299 | 1299_2 | GCTAACACAAATTTCCT | GCtaacacaaattTCCT | 45.7 |
| 1300 | 1300_1 | TTGCTAACACAAATTTCC | TTGCtaacacaaatttCC | 60.7 |
| 1301 | 1301_1 | TGCTAACACAAATTTCC | TGCTaacacaaatttCC | 62.6 |
| 1302 | 1302_1 | TTGCTAACACAAATTTC | TTGCtaacacaaattTC | 72.4 |
| 1303 | 1303_1 | CCTTTGCTAACACAAAT | CCTTtgctaacacaaAT | 48.1 |
| 1304 | 1304_1 | GTATAACCAATAATAACTA | GTAtaaccaataataaCTA | 86.1 |
| 1305 | 1305_1 | TCTGACATCACACAATTT | TCTGacatcacacaatTT | 67.8 |
| 1306 | 1306_1 | TCTGACATCACACAATT | TCTGacatcacacaaTT | 70.2 |
| 1307 | 1307_1 | TATCTGACATCACACAA | TATctgacatcacaCAA | 69.8 |
| 1308 | 1308_1 | CTATTCCTTAACCCAAC | CTattccttaaccCAAC | 77.7 |
| 1309 | 1309_1 | GTCTATTCCTTAACCCAAC | GtctattccttaaccCAAC | 86.2 |
| 1310 | 1310_1 | GTCTATTCCTTAACCCAA | GtctattccttaacCCAA | 60.4 |
| 1311 | 1311_1 | TCTATTCCTTAACCCAA | TCTattccttaaccCAA | 51 |
| 1312 | 1312_1 | GTCTATTCCTTAACCCA | GtctattccttaacCCA | 67.3 |
| 1313 | 1313_1 | GTCTATTCCTTAACCC | GtctattccttaaCCC | 77.4 |
| 1314 | 1314_1 | GGTCTATTCCTTAACC | GGtctattccttaaCC | 83.2 |
| 1315 | 1315_1 | AGAACATTTCCTTCTCCT | AgaacatttccttctcCT | 84.2 |
| 1316 | 1316_1 | AACTGTCCCAAACAACC | AACtgtcccaaacaaCC | 75 |
| 1317 | 1317_1 | TTAGTCTCCCTCATTTTC | TtagtctccctcattTTC | 72.4 |
| 1318 | 1318_1 | ATTTAGTCTCCCTCATT | ATttagtctccctCATT | 48 |
| 1319 | 1319_1 | ATGCATCAAATCTCATA | ATGCatcaaatctcaTA | 83.7 |
| 1320 | 1320_1 | CCTAAATAATTTAATCATTAA | CCTaaataatttaatcatTAA | 94 |
| 1321 | 1321_1 | CCCTAAATAATTTAATCATTA | CCCTaaataatttaatcatTA | 88.8 |
| 1322 | 1322_1 | CCCCTAAATAATTTAATCATT | CCCctaaataatttaatcATT | 80.7 |
| 1323 | 1323_1 | CCCTAAATAATTTAATCATT | CCCTaaataatttaatcaTT | 82.2 |
| 1324 | 1324_1 | CCCTAAATAATTTAATCAT | CCCtaaataatttaatCAT | 87.1 |
| 1325 | 1325_1 | CCCCTAAATAATTTAATCA | CCCctaaataatttaaTCA | 79.9 |
| 1326 | 1326_1 | CCCTAAATAATTTAATCA | CCCtaaataatttaaTCA | 82.5 |
| 1327 | 1327_1 | CCCCTAAATAATTTAATC | CCCCtaaataatttaaTC | 116 |
| 1328 | 1328_1 | TCCCCTAAATAATTTAATC | TCCCctaaataatttaaTC | 109 |
| 1329 | 1329_1 | TTGCTAATATTTCCAAAA | TTGCtaatatttccaaAA | 84.2 |
| 1330 | 1330_1 | CTTGCTAATATTTCCAA | CTtgctaatatttCCAA | 66.1 |
| 1331 | 1331_1 | ACTGTCATCCATATTTCC | ActgtcatccatattTCC | 66.2 |
| 1332 | 1332_1 | ACTGTCATCCATATTTC | ACTgtcatccatatTTC | 48.1 |
| 1333 | 1333_1 | AATGCCCCACTCTAATAT | AATGccccactctaatAT | 36.9 |
| 1334 | 1334_1 | TGCCCCACTCTAATAT | TGCcccactctaatAT | 52.8 |
| 1335 | 1335_1 | ATGCCCCACTCTAATAT | ATgccccactctaaTAT | 43.7 |
| 1336 | 1336_1 | AAATGCCCCACTCTAATA | AAATgccccactctaaTA | 25.7 |
| 1337 | 1337_1 | ATGCCCCACTCTAATA | ATGccccactctaaTA | 28.6 |
| 1338 | 1338_1 | AATGCCCCACTCTAATA | AATGccccactctaaTA | 29.9 |
| 1339 | 1339_1 | TTAAATGCCCCACTCTA | TtaaatgccccactCTA | 51.3 |
| 1340 | 1340_1 | TCTGAAAATTCACTATCT | TCTGaaaattcactatCT | 35.7 |
| 1341 | 1341_1 | GTCTACTATATACATCT | GTCtactatatacaTCT | 30.6 |
| 1342 | 1342_1 | AGTCTACTATATACATCT | AGTCtactatatacatCT | 45.3 |
| 1343 | 1343_1 | AGTCTACTATATACATC | AGTCtactatatacaTC | 57 |
| 1344 | 1344_1 | GTCTACTATATACATC | GTCTactatatacaTC | 46.5 |
| 1345 | 1345_1 | TAGTCTACTATATACATC | TAgtctactatataCATC | 68.3 |
| 1346 | 1346_1 | TAGTCTACTATATACAT | TAGtctactatataCAT | 89 |
| 1347 | 1347_1 | CTAGTCTACTATATACAT | CTAgtctactatataCAT | 86.6 |
| 1348 | 1348_1 | CTAGTCTACTATATACA | CTAGtctactatataCA | 88.5 |
| 1349 | 1349_1 | ACTAGTCTACTATATAC | ACTagtctactataTAC | 85.1 |
| 1350 | 1350_1 | CTAGTCTACTATATAC | CTAgtctactataTAC | 85.3 |
| 1351 | 1351_1 | GTATATTCTACCCATAA | GTAtattctacccaTAA | 51.3 |
| 1352 | 1352_1 | TGTATATTCTACCCATAA | TGTatattctacccaTAA | 48.4 |
| 1353 | 1353_1 | TGTATATTCTACCCATA | TGtatattctaccCATA | 45.6 |
| 1354 | 1354_1 | ATGTATATTCTACCCATA | ATgtatattctaccCATA | 90.2 |
| 1355 | 1355_1 | ATGTATATTCTACCCAT | ATgtatattctacCCAT | 51.1 |
| 1356 | 1356_1 | GAAAACCACACAATTCCTA | GaaaaccacacaattCCTA | 58.9 |
| 1357 | 1357_1 | GAAAACCACACAATTCCT | GAaaaccacacaatTCCT | 56.4 |
| 1358 | 1358_1 | AGAAAACCACACAATTCCT | AGaaaaccacacaattCCT | 58.4 |
| 1359 | 1359_1 | CAGAAAACCACACAATTCC | CAGaaaaccacacaatTCC | 43.3 |
| 1360 | 1360_1 | AGAAAACCACACAATTCC | AGAaaaccacacaatTCC | 47.6 |
| 1361 | 1361_1 | CCAGAAAACCACACAATTC | CCAGaaaaccacacaatTC | 26.3 |
| 1362 | 1362_1 | CCAGAAAACCACACAATT | CCAGaaaaccacacaaTT | 21 |
| 1363 | 1363_1 | TCCAGAAAACCACACAAT | TCCAgaaaaccacacaAT | 47.1 |
| 1364 | 1364_1 | TTCCAGAAAACCACACAA | TTCCagaaaaccacacAA | 49.8 |
| 1364 | 1364_2 | TTCCAGAAAACCACACAA | TTCcagaaaaccacaCAA | 45.8 |
| 1365 | 1365_1 | GATATATCACTAAATCCAT | GAtatatcactaaatCCAT | 27.4 |
| 1366 | 1366_1 | GATATATCACTAAATCCA | GAtatatcactaaaTCCA | 43.7 |
| 1367 | 1367_1 | AGATATATCACTAAATCCA | AGatatatcactaaaTCCA | 37.4 |
| 1368 | 1368_1 | AGATATATCACTAAATCC | AGAtatatcactaaaTCC | 33.6 |
| 1369 | 1369_1 | TCATATATAAATTTCTCTA | TCAtatataaatttctCTA | 78 |
| 1369 | 1369_2 | TCATATATAAATTTCTCTA | TCATatataaatttctcTA | 73.1 |
| 1370 | 1370_1 | TCATATATAAATTTCTCT | TCatatataaatttCTCT | 27.9 |
| 1370 | 1370_2 | TCATATATAAATTTCTCT | TCATatataaatttctCT | 60.2 |
| 1371 | 1371_1 | AAGATCACACAACCATA | AAGAtcacacaaccaTA | 19.8 |
| 1372 | 1372_1 | TAAAAGATCACACAACCA | TAaaagatcacacaACCA | 47.5 |
| 1373 | 1373_1 | CATCACATAAAAACCCACTT | CATcacataaaaacccaCTT | 45.4 |
| 1374 | 1374_1 | CATCACATAAAAACCCACT | CAtcacataaaaaccCACT | 57.9 |
| 1375 | 1375_1 | TCATCACATAAAAACCCACT | TCatcacataaaaaccCACT | 30.1 |
| 1376 | 1376_1 | CATCACATAAAAACCCAC | CAtcacataaaaacCCAC | 61.6 |
| 1377 | 1377_1 | TCATCACATAAAAACCCAC | TCatcacataaaaacCCAC | 30.6 |
| 1378 | 1378_1 | GTCATCACATAAAAACCCAC | GTCatcacataaaaaccCAC | 24.9 |
| 1379 | 1379_1 | GTCATCACATAAAAACCCA | GTCatcacataaaaacCCA | 28.7 |
| 1380 | 1380_1 | TCATCACATAAAAACCCA | TCatcacataaaaaCCCA | 43.9 |
| 1381 | 1381_1 | CATCACATAAAAACCCA | CAtcacataaaaaCCCA | 71.5 |
| 1382 | 1382_1 | TCATCACATAAAAACCC | TCAtcacataaaaaCCC | 42.9 |
| 1383 | 1383_1 | GTCATCACATAAAAACCC | GTCatcacataaaaaCCC | 24.9 |
| 1384 | 1384_1 | AGTCATCACATAAAAACCC | AGtcatcacataaaaACCC | 35.8 |
| 1384 | 1384_2 | AGTCATCACATAAAAACCC | AGTCatcacataaaaacCC | 23 |
| 1385 | 1385_1 | TAGTCATCACATAAAAACC | TAGTcatcacataaaaaCC | 36.3 |
| 1386 | 1386_1 | AGTCATCACATAAAAACC | AGTCatcacataaaaaCC | 34.9 |
| 1387 | 1387_1 | ATGCTAAATACAAATCT | ATGCtaaatacaaatCT | 81 |
| 1388 | 1388_1 | GAAACCATTACTACAACCAA | GAaaccattactacaaCCAA | 20.1 |
| 1389 | 1389_1 | GAAACCATTACTACAACCA | GAaaccattactacaACCA | 15.9 |
| 1390 | 1390_1 | ATGAAACCATTACTACAAC | ATGAaaccattactacaAC | 45.6 |
| 1391 | 1391_1 | CATGAAACCATTACTACA | CATGaaaccattactaCA | 55.9 |
| 1392 | 1392_1 | CCATGAAACCATTACTAC | CCatgaaaccattaCTAC | 29.5 |
| 1393 | 1393_1 | CTCCCATGAAACCATTA | CTCCcatgaaaccatTA | 73.7 |
| 1394 | 1394_1 | TGCTTACTTTATACAAAA | TGCTtactttatacaaAA | 55.9 |
| 1395 | 1395_1 | ATGTTAATACTTTTTCCA | ATGttaatactttttCCA | 92.9 |
| 1396 | 1396_1 | CCTAATTTAACCCACAA | CCTaatttaacccaCAA | 32.2 |
| 1397 | 1397_1 | ATCCTAATTTAACCCACAA | ATCctaatttaacccaCAA | 38.1 |
| 1398 | 1398_1 | TCCTAATTTAACCCACAA | TCCtaatttaacccaCAA | 39.9 |
| 1399 | 1399_1 | TAATCCTAATTTAACCCACAA | TAAtcctaatttaacccaCAA | 72.8 |
| 1400 | 1400_1 | TAATCCTAATTTAACCCACA | TAatcctaatttaaccCACA | 45 |
| 1401 | 1401_1 | AATCCTAATTTAACCCACA | AATCctaatttaacccaCA | 41.2 |
| 1402 | 1402_1 | TCCTAATTTAACCCACA | TCCtaatttaacccACA | 38.3 |
| 1403 | 1403_1 | TAATCCTAATTTAACCCAC | TAatcctaatttaacCCAC | 37.5 |
| 1404 | 1404_1 | ATCCTAATTTAACCCAC | ATcctaatttaacCCAC | 34.4 |
| 1405 | 1405_1 | AATCCTAATTTAACCCAC | AAtcctaatttaacCCAC | 48.2 |
| 1406 | 1406_1 | TAATCCTAATTTAACCCA | TAatcctaatttaaCCCA | 56.5 |
| 1407 | 1407_1 | AATCCTAATTTAACCCA | AAtcctaatttaaCCCA | 71.7 |
| 1408 | 1408_1 | GTAATCCTAATTTAACCCA | GtaatcctaatttaaCCCA | 63.6 |
| 1409 | 1409_1 | TAATCCTAATTTAACCC | TAatcctaatttaACCC | 56.5 |
| 1410 | 1410_1 | GTAATCCTAATTTAACCC | GTaatcctaatttaACCC | 44 |
| 1411 | 1411_1 | AGTAATCCTAATTTAACCC | AGtaatcctaatttaaCCC | 66.2 |
| 1410 | 1410_2 | GTAATCCTAATTTAACCC | GTAatcctaatttaaCCC | 34.2 |
| 1412 | 1412_1 | AGTAATCCTAATTTAACC | AGTAatcctaatttaaCC | 42.7 |
| 1413 | 1413_1 | TCATTTATCACTACCACA | TCAtttatcactaccACA | 26.5 |
| 1414 | 1414_1 | CATCATTTATCACTACCACA | CatcatttatcactacCACA | 46 |
| 1415 | 1415_1 | CATTTATCACTACCACA | CAtttatcactacCACA | 19.4 |
| 1416 | 1416_1 | ATCATTTATCACTACCACA | ATcatttatcactacCACA | 16.8 |
| 1416 | 1416_2 | ATCATTTATCACTACCACA | ATCAtttatcactaccaCA | 14.1 |
| 1417 | 1417_1 | ACATCATTTATCACTACCACA | ACatcatttatcactaccACA | 53.4 |
| 1418 | 1418_1 | TCATTTATCACTACCAC | TCAtttatcactacCAC | 18.9 |
| 1419 | 1419_1 | ATCATTTATCACTACCAC | ATcatttatcactaCCAC | 21.8 |
| 1420 | 1420_1 | CATCATTTATCACTACCAC | CATcatttatcactacCAC | 25.1 |
| 1421 | 1421_1 | AACATCATTTATCACTACCAC | AACatcatttatcactacCAC | 30.5 |
| 1421 | 1421_2 | AACATCATTTATCACTACCAC | AacatcatttatcactaCCAC | 40.4 |
| 1420 | 1420_2 | CATCATTTATCACTACCAC | CatcatttatcactaCCAC | 34.3 |
| 1422 | 1422_1 | AACATCATTTATCACTACCA | AAcatcatttatcactACCA | 34 |
| 1423 | 1423_1 | CATCATTTATCACTACCA | CATCatttatcactacCA | 11.3 |
| 1424 | 1424_1 | TAACATCATTTATCACTACCA | TAacatcatttatcactaCCA | 63.1 |
| 1425 | 1425_1 | ACATCATTTATCACTACCA | ACATcatttatcactacCA | 19 |
| 1422 | 1422_2 | AACATCATTTATCACTACCA | AACatcatttatcactaCCA | 25 |
| 1424 | 1424_2 | TAACATCATTTATCACTACCA | TaacatcatttatcactACCA | 61.3 |
| 1425 | 1425_2 | ACATCATTTATCACTACCA | ACatcatttatcactACCA | 23.5 |
| 1426 | 1426_1 | TAACATCATTTATCACTACC | TAACatcatttatcactaCC | 33.6 |
| 1427 | 1427_1 | ACATCATTTATCACTACC | ACatcatttatcacTACC | 32.3 |
| 1428 | 1428_1 | TTAACATCATTTATCACTACC | TTAacatcatttatcactaCC | 75.5 |
| 1429 | 1429_1 | AACATCATTTATCACTACC | AACAtcatttatcactaCC | 37.3 |
| 1430 | 1430_1 | TTAACATCATTTATCACTAC | TTaacatcatttatcaCTAC | 69.1 |
| 1431 | 1431_1 | TAACATCATTTATCACTAC | TAacatcatttatcaCTAC | 66.6 |
| 1432 | 1432_1 | ATTAACATCATTTATCACTAC | ATtaacatcatttatcaCTAC | 84.2 |
| 1432 | 1432_2 | ATTAACATCATTTATCACTAC | ATtaacatcAtttatcaCTAC | 62.8 |
| 1433 | 1433_1 | ATTAACATCATTTATCACTA | ATTaacatcatttatcaCTA | 81.3 |
| 1434 | 1434_1 | TTAACATCATTTATCACTA | TTAacatcatttatcaCTA | 74.5 |
| 1435 | 1435_1 | TAATTAACATCATTTATCACT | TAattaacatcatttatCACT | 84.3 |
| 1435 | 1435_2 | TAATTAACATCATTTATCACT | TAattaacaTcatttatCACT | 43.3 |
| 1436 | 1436_1 | CTAATTAACATCATTTATCAC | CTaattaacatcatttaTCAC | 81.4 |
| 1436 | 1436_2 | CTAATTAACATCATTTATCAC | CTaattaacAtcatttaTCAC | 46.7 |
| 1437 | 1437_1 | CCTAATTAACATCATTTATCA | CCtaattaacatcatttaTCA | 93.8 |
| 1438 | 1438_1 | CTAATTAACATCATTTATCA | CTAattaacatcatttaTCA | 89.6 |
| 1439 | 1439_1 | CCTAATTAACATCATTTATC | CCTAattaacatcatttaTC | 69.4 |
| 1440 | 1440_1 | CCCTAATTAACATCATTTATC | CCctaattaacatcatttATC | 86.3 |
| 1441 | 1441_1 | CCTAATTAACATCATTTAT | CCTaattaacatcattTAT | 87.4 |
| 1442 | 1442_1 | CCTAATTAACATCATTTA | CCTAattaacatcattTA | 66 |
| 1443 | 1443_1 | CCCTAATTAACATCATTTA | CCCtaattaacatcattTA | 88.7 |
| 1444 | 1444_1 | GCCCTAATTAACATCATTT | GCCctaattaacatcatTT | 87.9 |
| 1445 | 1445_1 | CCCTAATTAACATCATTT | CCCTaattaacatcatTT | 75.6 |
| 1446 | 1446_1 | CGGCCCTAATTAACAT | CGGCcctaattaacAT | 103 |
| 1447 | 1447_1 | CTCGGCCCTAATTAA | CTCggccctaatTAA | 57.4 |
| 1448 | 1448_1 | CACATATAACATATAAACACA | CACAtataacatataaacaCA | 61.7 |
| 1449 | 1449_1 | TCACATATAACATATAAACAC | TCAcatataacatataaaCAC | 43.6 |
| 1450 | 1450_1 | ACTATCACATATAACATATA | ACTAtcacatataacataTA | 58.5 |
| 1451 | 1451_1 | CACTATCACATATAACATATA | CACTatcacatataacataTA | 28.1 |
| 1452 | 1452_1 | CACTATCACATATAACATAT | CACtatcacatataacaTAT | 52 |
| 1453 | 1453_1 | CACTATCACATATAACATA | CACTatcacatataacaTA | 24.3 |
| 1454 | 1454_1 | CACTATCACATATAACAT | CACtatcacatataaCAT | 40.1 |
| 1455 | 1455_1 | CACTATCACATATAACA | CACTatcacatataaCA | 27 |
| 1456 | 1456_1 | CAAAGTTTTCCCATTAC | CAaagttttcccaTTAC | 21 |
| 1457 | 1457_1 | ACAAAGTTTTCCCATTA | ACAaagttttcccaTTA | 20.5 |
| 1458 | 1458_1 | TCAGTCCAACATAACTC | TCAGtccaacataacTC | 15.2 |
| 1459 | 1459_1 | CAGTCCAACATAACTC | CAGtccaacataaCTC | 23.5 |
| 1460 | 1460_1 | ATCAGTCCAACATAACTC | ATCAgtccaacataacTC | 13.7 |
| 1461 | 1461_1 | ATCAGTCCAACATAACT | ATCAgtccaacataaCT | 15.9 |
| 1462 | 1462_1 | TAAACATTAAACCCTCCCAAA | TAaacattaaaccctccCAAA | 87 |
| 1463 | 1463_1 | AACATTAAACCCTCCCAA | AAcattaaaccctcCCAA | 68.4 |
| 1464 | 1464_1 | TAAACATTAAACCCTCCCAA | TaaacattaaaccctcCCAA | 79.2 |
| 1465 | 1465_1 | AAACATTAAACCCTCCCAA | AAacattaaaccctcCCAA | 70.8 |
| 1466 | 1466_1 | TATAAACATTAAACCCTCCCA | TAtaaacattaaaccctccCA | 94 |
| 1467 | 1467_1 | AACATTAAACCCTCCC | AAcattaaacccTCCC | 78.3 |
| 1468 | 1468_1 | AAACATTAAACCCTCCC | AAacattaaacccTCCC | 89.4 |
| 1469 | 1469_1 | TAAACATTAAACCCTCCC | TAAacattaaaccctCCC | 72.9 |
| 1470 | 1470_1 | ATAAACATTAAACCCTCCC | AtaaacattaaacccTCCC | 86 |
| 1471 | 1471_1 | TATAAACATTAAACCCTCC | TAtaaacattaaaccCTCC | 91.1 |
| 1472 | 1472_1 | TAAACATTAAACCCTCC | TAaacattaaaccCTCC | 82.2 |
| 1473 | 1473_1 | ACTATAAACATTAAACCCTCC | ActataaacattaaaccCTCC | 86.5 |
| 1474 | 1474_1 | ATAAACATTAAACCCTCC | ATaaacattaaaccCTCC | 88.4 |
| 1475 | 1475_1 | AACTATAAACATTAAACCCTC | AActataaacattaaacCCTC | 92.6 |
| 1476 | 1476_1 | CTATAAACATTAAACCCTC | CTataaacattaaacCCTC | 82.9 |
| 1477 | 1477_1 | ACTATAAACATTAAACCCTC | ACtataaacattaaacCCTC | 89.8 |
| 1478 | 1478_1 | AAACTATAAACATTAAACCCT | AAactataaacattaaaCCCT | 98.9 |
| 1479 | 1479_1 | CTATAAACATTAAACCCT | CTataaacattaaaCCCT | 82.2 |
| 1480 | 1480_1 | ACTATAAACATTAAACCCT | ACtataaacattaaaCCCT | 86.6 |
| 1481 | 1481_1 | AACTATAAACATTAAACCCT | AActataaacattaaaCCCT | 89.5 |
| 1482 | 1482_1 | GCTTTAAACTATAAACATT | GCtttaaactataaaCATT | 58.2 |
| 1483 | 1483_1 | TGCTTTAAACTATAAACA | TGCTttaaactataaaCA | 57.2 |
| 1484 | 1484_1 | CAGATTTATCACTATTA | CAGAtttatcactatTA | 15.4 |
| 1485 | 1485_1 | TCACAGCCTATCACCAC | TCacagcctatcacCAC | 47.3 |
| 1485 | 1485_2 | TCACAGCCTATCACCAC | TCAcagcctatcaccAC | 46.3 |
| 1486 | 1486_1 | ATCACAGCCTATCACCA | AtcacagcctatcACCA | 56.9 |
| 1486 | 1486_2 | ATCACAGCCTATCACCA | ATCacagcctatcacCA | 23.7 |
| 1487 | 1487_1 | AATCACAGCCTATCACC | AATCacagcctatcaCC | 32.9 |
| 1487 | 1487_2 | AATCACAGCCTATCACC | AatcacagcctatCACC | 52.2 |
| 1488 | 1488_1 | ATCACAGCCTATCACC | AtcacagcctatCACC | 60.1 |
| 1489 | 1489_1 | GCGTCACCCAAATCAC | GCgtcacccaaatCAC | 11 |
| 1490 | 1490_1 | AGCGTCACCCAAATCA | AGmcgtcacccaaaTCA | 17.4 |
| 1491 | 1491_1 | AGCGTCACCCAAATC | AGmcgtcacccaAATC | 18.8 |
| 1492 | 1492_1 | CAGATCCTAAAATCACT | CAGAtcctaaaatcaCT | 71.8 |
| 1493 | 1493_1 | TCAGATCCTAAAATCAC | TCAgatcctaaaatCAC | 66.2 |
| 1494 | 1494_1 | AGTAAAACCAATCATCAT | AGTaaaaccaatcatCAT | 30.8 |
| 1495 | 1495_1 | AGTAAAACCAATCATCA | AGTaaaaccaatcaTCA | 24.2 |
| 1496 | 1496_1 | CCCTTCCATCTCTACTAAAA | CccttccatctctactaaAA | 89.7 |
| 1497 | 1497_1 | ATAACTACATAACAAACCCA | ATaactacataacaaaCCCA | 69.1 |
| 1498 | 1498_1 | AATAACTACATAACAAACCCA | AAtaactacataacaaaCCCA | 77.8 |
| 1499 | 1499_1 | AACTACATAACAAACCCA | AActacataacaaaCCCA | 62.9 |
| 1500 | 1500_1 | TAACTACATAACAAACCCA | TAactacataacaaaCCCA | 65 |
| 1501 | 1501_1 | ACTACATAACAAACCCA | ACtacataacaaaCCCA | 60.4 |
| 1502 | 1502_1 | CAATAACTACATAACAAACCC | CAAtaactacataacaaaCCC | 72.6 |
| 1503 | 1503_1 | ATAACTACATAACAAACCC | ATAactacataacaaaCCC | 60.2 |
| 1504 | 1504_1 | ACAATAACTACATAACAAACC | ACAataactacataacaaACC | 78.5 |
| 1504 | 1504_2 | ACAATAACTACATAACAAACC | ACAAtaactacataacaaaCC | 80.9 |
| 1505 | 1505_1 | TGAATTCACAATAACTACA | TGaattcacaataacTACA | 38.1 |
| 1506 | 1506_1 | GCACATTTTTCTTAAACT | GCACatttttcttaaaCT | 62.2 |
| 1507 | 1507_1 | GCTATACCTAAAACAATCT | GCTatacctaaaacaaTCT | 62.2 |
| 1508 | 1508_1 | GCTATACCTAAAACAATC | GCTAtacctaaaacaaTC | 68.9 |
| 1509 | 1509_1 | CCCTTGTAACTAAAAAT | CCCTtgtaactaaaaAT | 100 |
| 1510 | 1510_1 | CCCCTTGTAACTAAAAA | CCCCttgtaactaaaAA | 86.1 |
| 1511 | 1511_1 | CCCCTTGTAACTAAAA | CCCCttgtaactaaAA | 101 |
| 1512 | 1512_1 | ACCCCTTGTAACTAAA | ACCCcttgtaactaAA | 88.8 |
| 1513 | 1513_1 | CACCCCTTGTAACTAA | CAccccttgtaaCTAA | 80.4 |
| 1514 | 1514_1 | ACACCCCTTGTAACTA | ACACcccttgtaacTA | 72.4 |
| 1515 | 1515_1 | GCTAAAACTAATCATCT | GCTaaaactaatcaTCT | 72.2 |
| 1516 | 1516_1 | GGCTAAAACTAATCAT | GGCtaaaactaatCAT | 70.8 |
| 1517 | 1517_1 | TTACCCTTCATATATACATCT | TtacccttcatatatacaTCT | 89.4 |
| 1518 | 1518_1 | ATTACCCTTCATATATACATC | AttacccttcatatataCATC | 82.4 |
| 1519 | 1519_1 | TTACCCTTCATATATACATC | TTAcccttcatatatacATC | 56.3 |
| 1520 | 1520_1 | CATTACCCTTCATATATACAT | CAttacccttcatatataCAT | 84.2 |
| 1521 | 1521_1 | TTACCCTTCATATATACAT | TTAcccttcatatataCAT | 55.3 |
| 1522 | 1522_1 | ATTACCCTTCATATATACAT | ATTacccttcatatataCAT | 49.3 |
| 1523 | 1523_1 | ACATTACCCTTCATATATACA | ACAttacccttcatatataCA | 55.2 |
| 1523 | 1523_2 | ACATTACCCTTCATATATACA | AcattacccttcatataTACA | 63.4 |
| 1524 | 1524_1 | TTACCCTTCATATATACA | TTACccttcatatataCA | 46.9 |
| 1525 | 1525_1 | CATTACCCTTCATATATACA | CattacccttcatataTACA | 66 |
| 1526 | 1526_1 | ATTACCCTTCATATATACA | ATTAcccttcatatataCA | 36.7 |
| 1527 | 1527_1 | ATTACCCTTCATATATAC | ATTacccttcatataTAC | 46.6 |
| 1528 | 1528_1 | TTACCCTTCATATATAC | TTAcccttcatataTAC | 56.9 |
| 1529 | 1529_1 | CATTACCCTTCATATATAC | CATtacccttcatataTAC | 63.4 |
| 1530 | 1530_1 | ACATTACCCTTCATATATAC | ACAttacccttcatataTAC | 34.5 |
| 1531 | 1531_1 | TACATTACCCTTCATATATAC | TAcattacccttcatataTAC | 76.9 |
| 1532 | 1532_1 | CATTACCCTTCATATATA | CAttacccttcataTATA | 76.5 |
| 1533 | 1533_1 | TACATTACCCTTCATATATA | TACattacccttcatatATA | 36.5 |
| 1534 | 1534_1 | ATTACCCTTCATATATA | ATtacccttcataTATA | 78 |
| 1535 | 1535_1 | ACATTACCCTTCATATATA | ACattacccttcataTATA | 59.5 |
| 1536 | 1536_1 | CATTACCCTTCATATAT | CATtacccttcataTAT | 73.7 |
| 1537 | 1537_1 | ACATTACCCTTCATATAT | ACAttacccttcataTAT | 46.1 |
| 1538 | 1538_1 | TACATTACCCTTCATATAT | TACattacccttcataTAT | 36.9 |
| 1539 | 1539_1 | ACATTACCCTTCATATA | ACattacccttcaTATA | 54.2 |
| 1540 | 1540_1 | TACATTACCCTTCATATA | TAcattacccttcaTATA | 71.5 |
| 1541 | 1541_1 | TACATTACCCTTCATAT | TACattacccttcaTAT | 34.5 |
| 1542 | 1542_1 | GATTCTTATACTTACTA | GATtcttatacttaCTA | 46.2 |
| 1543 | 1543_1 | TGATTCTTATACTTACT | TGattcttatactTACT | 45.7 |
| 1544 | 1544_1 | ATGATTCTTATACTTACT | ATGAttcttatacttaCT | 54 |
| 1545 | 1545_1 | GCCTCATTTTTACCTTT | GCctcalllllaccTTT | 82.6 |
| 1546 | 1546_1 | ACCAATCTTCTATTTTA | ACCAatcttctatttTA | 94.8 |
| 1547 | 1547_1 | CAACCAATCTTCTATTTTA | CAACcaatcttctatttTA | 90.3 |
| 1548 | 1548_1 | GCAACCAATCTTCTATTTT | GCAAccaatcttctattTT | 88.3 |
| 1549 | 1549_1 | GCAACCAATCTTCTATTT | GCAaccaatcttctaTTT | 85 |
| 1550 | 1550_1 | GCAACCAATCTTCTATT | GCaaccaatcttcTATT | 87.3 |
| 1551 | 1551_1 | TGCAACCAATCTTCTATT | TGCaaccaatcttctaTT | 90.2 |
| 1552 | 1552_1 | TAACTGCAACCAATCTT | TAactgcaaccaaTCTT | 88.2 |
| 1553 | 1553_1 | TGAATACAACACACATCA | TGAatacaacacacaTCA | 97.4 |
| 1554 | 1554_1 | ATGAATACAACACACATCA | ATGAatacaacacacatCA | 84.4 |
| 1555 | 1555_1 | TAAAAATATAACTACTCCT | TAaaaatataactacTCCT | 99.8 |
| 1556 | 1556_1 | GTAAAAATATAACTACTCC | GTaaaaatataactaCTCC | 93.7 |
| 1557 | 1557_1 | TCAACTGATACCCACAA | TCAactgatacccaCAA | 57.7 |
| 1558 | 1558_1 | TGTCTTAACATTTTTCTT | TGTCttaacatttttcTT | 63.1 |
| 1559 | 1559_1 | CCACTTCAAACTTTTAATTAA | CCAcftcaaacttttaatTAA | 85 |
| 1560 | 1560_1 | CCACTTCAAACTTTTAATTA | CCACttcaaacttttaatTA | 84.9 |
| 1561 | 1561_1 | CCCACTTCAAACTTTTAATTA | CCcacttcaaacttttaaTTA | 88.7 |
| 1562 | 1562_1 | CCACTTCAAACTTTTAATT | CCACftcaaacttttaaTT | 79.1 |
| 1563 | 1563_1 | CCCACTTCAAACTTTTAATT | CCCacftcaaacttttaaTT | 86.2 |
| 1564 | 1564_1 | ACCCACTTCAAACTTTTAATT | ACCcacftcaaacttttaaTT | 100 |
| 1565 | 1565_1 | CCACTTCAAACTTTTAAT | CCACttcaaacttttaAT | 85.3 |
| 1566 | 1566_1 | ACCCACTTCAAACTTTTAAT | ACCcacftcaaacttttAAT | 88.8 |
| 1567 | 1567_1 | AACCCACTTCAAACTTTTAAT | AACCcacttcaaacttttaAT | 92.3 |
| 1568 | 1568_1 | CCCACTTCAAACTTTTAA | CCCacttcaaactttTAA | 79.9 |
| 1569 | 1569_1 | ACCCACTTCAAACTTTTAA | ACCcacttcaaactaTAA | 82.5 |
| 1570 | 1570_1 | CCCACTTCAAACTTTTA | CCCacttcaaactttTA | 79.6 |
| 1571 | 1571_1 | ACCCACTTCAAACTTTTA | ACCcacttcaaacttTTA | 77.2 |
| 1572 | 1572_1 | AACCCACTTCAAACTTTTA | AACCcacttcaaactttTA | 86.2 |
| 1573 | 1573_1 | ACCCACTTCAAACTTTT | ACCCacttcaaacttTT | 93.3 |
| 1574 | 1574_1 | AACCCACTTCAAACTTTT | AACCcacttcaaacttTT | 82.7 |
| 1575 | 1575_1 | AACCCACTTCAAACTTT | AACCcacttcaaactTT | 85.8 |
| 1576 | 1576_1 | GGACTCTATTAATCAA | GGActctattaatCAA | 91.7 |
| 1577 | 1577_1 | GAATATTCTACTCTTCT | GAatattctactcTTCT | 95.3 |
| 1578 | 1578_1 | CTGTATTTACCAATTCAA | CTGtatttaccaattCAA | 90.8 |
| 1579 | 1579_1 | CTGTATTTACCAATTCA | CTGTatttaccaattCA | 88.7 |
| 1580 | 1580_1 | ACTGTATTTACCAATTCA | ACTGtatttaccaattCA | 97.3 |
| 1581 | 1581_1 | ACTGTATTTACCAATTC | ACTGtatttaccaatTC | 104 |
| 1582 | 1582_1 | CACTGTATTTACCAATT | CACTgtatttaccaaTT | 91.1 |
| 1583 | 1583_1 | TCACTGTATTTACCAAT | TCACtgtatttaccaAT | 98.6 |
| 1584 | 1584_1 | CCAACTACTTTACTTTTCAAA | CCaactactttacttttCAAA | 84.3 |
| 1585 | 1585_1 | CCAACTACTTTACTTTTCAA | CCaactactttactttTCAA | 80 |
| 1586 | 1586_1 | ACCAACTACTTTACTTTTCAA | ACcaactactttactttTCAA | 85.1 |
| 1585 | 1585_2 | CCAACTACTTTACTTTTCAA | CCAactactttacttttCAA | 75.2 |
| 1587 | 1587_1 | CCAACTACTTTACTTTTCA | CCAactactttacttttCA | 71.9 |
| 1588 | 1588_1 | TACCAACTACTTTACTTTTCA | TaccaactactttacttTTCA | 82.8 |
| 1587 | 1587_2 | CCAACTACTTTACTTTTCA | CCAactactttactttTCA | 67.7 |
| 1589 | 1589_1 | ACCAACTACTTTACTTTTCA | ACcaactactttactttTCA | 84 |
| 1590 | 1590_1 | TACCAACTACTTTACTTTTC | TACcaactactttacttTTC | 75.3 |
| 1591 | 1591_1 | GTACCAACTACTTTACTTT | GTACcaactactttactTT | 75.8 |
| 1592 | 1592_1 | GTACCAACTACTTTACTT | GTAccaactactttaCTT | 65.7 |
| 1593 | 1593_1 | GTACCAACTACTTTACT | GTACcaactactttaCT | 74.5 |
| 1594 | 1594_1 | TGTACCAACTACTTTACT | TGtaccaactacttTACT | 87.1 |
| 1595 | 1595_1 | TTGTACCAACTACTTTAC | TTGtaccaactacttTAC | 73.3 |
| 1596 | 1596_1 | GTACCAACTACTTTAC | GTAccaactacttTAC | 72.5 |
| 1597 | 1597_1 | TGTACCAACTACTTTAC | TGTaccaactacttTAC | 66 |
| 1598 | 1598_1 | TTGTACCAACTACTTTA | TTGTaccaactacttTA | 49.3 |
| 1599 | 1599_1 | ATTTCATTTTTCTTTTAATA | ATTtcatttttcttttaATA | 98.6 |
| 1599 | 1599_2 | ATTTCATTTTTCTTTTAATA | ATTTcatttttcttttaaTA | 90.7 |
| 1600 | 1600_1 | CCTAATTTCATTTTTCTTTT | CCtaatttcatttttcTTTT | 69.2 |
| 1601 | 1601_1 | TCCTAATTTCATTTTTCTTT | TCctaatttcatttttCTTT | 47 |
| 1602 | 1602_1 | TTCTTCATTATACCATCAAAT | TTCTtcattataccatcaaAT | 29.4 |
| 1603 | 1603_1 | TTTCTTCATTATACCATCAAA | TTTCttcattataccatcaAA | 24.1 |
| 1604 | 1604_1 | TTTTCTTCATTATACCATCAA | TTttcttcattataccaTCAA | 14.3 |
| 1605 | 1605_1 | TCTTCATTATACCATCAA | TCttcattataccaTCAA | 5.02 |
| 1606 | 1606_1 | TTTCTTCATTATACCATCAA | TTtcttcattataccaTCAA | 21.2 |
| 1607 | 1607_1 | TTCTTCATTATACCATCAA | TTCttcattataccatCAA | 5.83 |
| 1608 | 1608_1 | ATATTTTCTTCATTATACCAT | AtattttcttcattataCCAT | 76.1 |
| 1609 | 1609_1 | ATATTTTCTTCATTATACCA | ATattttcttcattataCCA | 40.2 |
| 1610 | 1610_1 | AATATTTTCTTCATTATACCA | AATattacttcattataCCA | 37 |
| 1611 | 1611_1 | AAATATTTTCTTCATTATACC | AAatattttcttcattaTACC | 23.4 |
| 1612 | 1612_1 | ATATTTTCTTCATTATACC | ATattttcttcattaTACC | 14.2 |
| 1613 | 1613_1 | AATATTTTCTTCATTATACC | AATAttttcttcattataCC | 68 |
| 1614 | 1614_1 | TAAATATTTTCTTCATTATA | TAaatattttcttcatTATA | 96.8 |
| 1615 | 1615_1 | TTTTCCTTCATCTACTTCT | TTTtccttcatctacttCT | 42.8 |
| 1616 | 1616_1 | ATTTTCCTTCATCTACTTCT | ATtttccttcatctacttCT | 76 |
| 1617 | 1617_1 | AATTTTCCTTCATCTACTTC | AATTttccttcatctactTC | 54.9 |
| 1618 | 1618_1 | AGAATTTTCCTTCATCTA | AgaattttccttcaTCTA | 58 |
| 1619 | 1619_1 | CAGAATTTTCCTTCATCT | CAgaattttccttcATCT | 23.5 |
| 1620 | 1620_1 | TCAGAATTTTCCTTCATC | TCAgaattttccttcaTC | 29.7 |
| 1621 | 1621_1 | CTAGAAATATCTCACATT | CTAGaaatatctcacaTT | 64.6 |
| 1622 | 1622_1 | CTAGAAATATCTCACAT | CTAgaaatatctcaCAT | 75.5 |
| 1623 | 1623_1 | ACTAGAAATATCTCACA | ACTAgaaatatctcaCA | 53.2 |
| 1624 | 1624_1 | ATTAGCCATTAATCTAT | ATtagccattaatCTAT | 71.9 |
| 1625 | 1625_1 | TTGTTACAAAATAATCCA | TTgttacaaaataaTCCA | 12 |
| 1625 | 1625_2 | TTGTTACAAAATAATCCA | TTGttacaaaataatCCA | 23.8 |
| 1626 | 1626_1 | TTATTTTTTACATTAACTA | TTAttttttacattaaCTA | 92.1 |
| 1627 | 1627_1 | TGCCAAAATACTAACATCA | TGCcaaaatactaacaTCA | 32 |
| 1628 | 1628_1 | GCCAAAATACTAACATCA | GCCaaaatactaacaTCA | 27.8 |
| 1629 | 1629_1 | TGCCAAAATACTAACATC | TGCCaaaatactaacaTC | 61.5 |
| 1630 | 1630_1 | GAGTACAACACTTACA | GAGTacaacacttaCA | 31.8 |
| 1631 | 1631_1 | CACATCCATTCATTTTAT | CACatccattcatttTAT | 30.6 |
| 1632 | 1632_1 | CCACATCCATTCATTTTAT | CCAcatccattcattttAT | 21.7 |
| 1633 | 1633_1 | CCACATCCATTCATTTTA | CCacatccattcattTTA | 20 |
| 1634 | 1634_1 | TATGCCACATCCATTCAT | TatgccacatccattCAT | 47 |
| 1635 | 1635_1 | TTATGCCACATCCATTCA | TtatgccacatccaTTCA | 20.7 |
| 1636 | 1636_1 | TATGCCACATCCATTCA | TAtgccacatccattCA | 43.3 |
| 1637 | 1637_1 | TTATGCCACATCCATTC | TtatgccacatccATTC | 19.5 |
| 1638 | 1638_1 | ATTATGCCACATCCATT | ATtatgccacatcCATT | 25.1 |
| 1639 | 1639_1 | AGTTTCATATTTTTAATC | AGTttcatatttttaATC | 65.9 |
| 1640 | 1640_1 | ATCACTGCACACTTTCC | ATCactgcacactttCC | 12.9 |
| 1641 | 1641_1 | AAGCTCTTTCCAAATTCT | AAGCtctttccaaattCT | 34.6 |
| 1642 | 1642_1 | TAGTTCTTAACTCTTCTC | TagttcttaactctTCTC | 19.2 |
| 1643 | 1643_1 | TTAGTTCTTAACTCTTC | TTAGttcttaactctTC | 18 |
| 1644 | 1644_1 | AGCTTCAAATACTCAAA | AGCTtcaaatactcaAA | 74.5 |
| 1645 | 1645_1 | TTTCAAAGCCACACCTA | TttcaaagccacaCCTA | 66.9 |
| 1646 | 1646_1 | AATATCCTCATTACCCATT | AATAtcctcattacccaTT | 52.3 |
| 1647 | 1647_1 | TATCCTCATTACCCATT | TAtcctcattaccCATT | 53.4 |
| 1647 | 1647_2 | TATCCTCATTACCCATT | TATCctcattacccaTT | 22.3 |
| 1648 | 1648_1 | ATATCCTCATTACCCATT | ATAtcctcattacccATT | 55.8 |
| 1649 | 1649_1 | AATATCCTCATTACCCAT | AAtatcctcattacCCAT | 46.1 |
| 1650 | 1650_1 | TAATATCCTCATTACCCAT | TAAtatcctcattaccCAT | 58.3 |
| 1651 | 1651_1 | TTAATATCCTCATTACCCAT | TTaatatcctcattaccCAT | 61.8 |
| 1652 | 1652_1 | ATATCCTCATTACCCAT | ATAtcctcattaccCAT | 56.2 |
| 1653 | 1653_1 | AATATCCTCATTACCCA | AAtatcctcattaCCCA | 49.7 |
| 1654 | 1654_1 | TAATATCCTCATTACCCA | TAATatcctcattaccCA | 45.6 |
| 1655 | 1655_1 | TTTAATATCCTCATTACCCA | TttaatatcctcattacCCA | 67.5 |
| 1656 | 1656_1 | TTAATATCCTCATTACCCA | TTaatatcctcattacCCA | 36 |
| 1656 | 1656_2 | TTAATATCCTCATTACCCA | TTAAtatcctcattaccCA | 57.9 |
| 1654 | 1654_2 | TAATATCCTCATTACCCA | TAAtatcctcattacCCA | 40 |
| 1653 | 1653_2 | AATATCCTCATTACCCA | AATatcctcattacCCA | 44.8 |
| 1657 | 1657_1 | ATTTAATATCCTCATTACCC | AtttaatatcctcattaCCC | 59.9 |
| 1658 | 1658_1 | TAATATCCTCATTACCC | TAATatcctcattacCC | 32.9 |
| 1659 | 1659_1 | TTAATATCCTCATTACCC | TTAAtatcctcattacCC | 42 |
| 1660 | 1660_1 | TTTAATATCCTCATTACCC | TttaatatcctcattACCC | 41.1 |
| 1661 | 1661_1 | AATTTAATATCCTCATTACCC | AatttaatatcctcattaCCC | 61 |
| 1662 | 1662_1 | TTTAATATCCTCATTACC | TTTAatatcctcattaCC | 60.6 |
| 1663 | 1663_1 | AATTTAATATCCTCATTACC | AAtttaatatcctcatTACC | 58.8 |
| 1664 | 1664_1 | TTAATATCCTCATTACC | TTaatatcctcatTACC | 42.3 |
| 1665 | 1665_1 | AAATTTAATATCCTCATTACC | AAatttaatatcctcatTACC | 55.9 |
| 1666 | 1666_1 | ATTTAATATCCTCATTACC | ATttaatatcctcatTACC | 55.5 |
| 1667 | 1667_1 | TAAATTTAATATCCTCATTAC | TAaatttaatatcctcaTTAC | 78 |
| 1668 | 1668_1 | TTAAATTTAATATCCTCATTA | TTAaatttaatatcctcaTTA | 95.2 |
| 1669 | 1669_1 | CTTAAATTTAATATCCTCATT | CTtaaatttaatatcctCATT | 73.2 |
| 1670 | 1670_1 | TCTTAAATTTAATATCCTCAT | TCttaaatttaatatccTCAT | 46.8 |
| 1671 | 1671_1 | TCTTAAATTTAATATCCTCA | TCttaaatttaatatcCTCA | 29.8 |
| 1672 | 1672_1 | TTCTTAAATTTAATATCCTCA | TTCttaaatttaatatccTCA | 35 |
| 1673 | 1673_1 | TTCTTAAATTTAATATCCTC | TTcttaaatttaatatCCTC | 36.2 |
| 1674 | 1674_1 | TCTTAAATTTAATATCCTC | TCttaaatttaatatCCTC | 25.1 |
| 1675 | 1675_1 | TTCTTAAATTTAATATCCT | TTCttaaatttaatatCCT | 46.9 |
| 1676 | 1676_1 | TCTTAAATTTAATATCCT | TCttaaatttaataTCCT | 50.9 |
| 1677 | 1677_1 | AATAGCCTTTATTCTAC | AAtagcctttattCTAC | 33.6 |
| 1678 | 1678_1 | CAGCAACAATTATTAATA | CAGCaacaattattaaTA | 70.5 |
| 1679 | 1679_1 | CCAGCAACAATTATTAAT | CCAGcaacaattattaAT | 64.2 |
| 1680 | 1680_1 | ACCAGCAACAATTATTAA | ACCagcaacaattatTAA | 20.5 |
| 1680 | 1680_2 | ACCAGCAACAATTATTAA | ACCAgcaacaattattAA | 39.7 |
| 1681 | 1681_1 | ACCAGCAACAATTATTA | ACCAgcaacaattatTA | 39.4 |
| 1682 | 1682_1 | TACCAGCAACAATTATT | TACCagcaacaattaTT | 26.4 |
| 1683 | 1683_1 | CCCCAAATCTAAAACACTTC | CCccaaatctaaaacacTTC | 79.4 |
| 1684 | 1684_1 | AACCCCAAATCTAAAACACTT | AACCccaaatctaaaacacTT | 82 |
| 1685 | 1685_1 | CCCCAAATCTAAAACACTT | CCCcaaatctaaaacacTT | 86.4 |
| 1686 | 1686_1 | AACCCCAAATCTAAAACACT | AACCccaaatctaaaacaCT | 75.2 |
| 1687 | 1687_1 | ACCCCAAATCTAAAACACT | ACcccaaatctaaaaCACT | 72.5 |
| 1688 | 1688_1 | ACCCCAAATCTAAAACAC | ACCccaaatctaaaaCAC | 80.9 |
| 1689 | 1689_1 | GCAAATATTCACAAATCCT | GCAaatattcacaaatCCT | 20.7 |
| 1689 | 1689_2 | GCAAATATTCACAAATCCT | GCaaatattcacaaaTCCT | 29.3 |
| 1690 | 1690_1 | ACTATTTAACACACATTATCA | ACTatttaacacacattaTCA | 36.6 |
| 1691 | 1691_1 | CTATTTAACACACATTATCA | CTAtttaacacacattaTCA | 49.6 |
| 1692 | 1692_1 | TACTATTTAACACACATTATC | TACTatttaacacacattaTC | 52.4 |
| 1693 | 1693_1 | ACTATTTAACACACATTATC | ACTAtttaacacacattaTC | 51.8 |
| 1694 | 1694_1 | TACTATTTAACACACATTAT | TACtatttaacacacatTAT | 91.1 |
| 1695 | 1695_1 | CTACTATTTAACACACATTAT | CTActatttaacacacatTAT | 72.7 |
| 1696 | 1696_1 | CTACTATTTAACACACATTA | CTACtatttaacacacatTA | 47.4 |
| 1697 | 1697_1 | ACTACTATTTAACACACATTA | ACTActatttaacacacatTA | 38.3 |
| 1698 | 1698_1 | CTACTATTTAACACACATT | CTACtatttaacacacaTT | 41.6 |
| 1699 | 1699_1 | ACTACTATTTAACACACATT | ACtactatttaacacaCATT | 40.3 |
| 1700 | 1700_1 | ACTACTATTTAACACACAT | ACTactatttaacacaCAT | 36.8 |
| 1701 | 1701_1 | CTACTATTTAACACACA | CTACtatttaacacaCA | 45.9 |
| 1702 | 1702_1 | ACTACTATTTAACACACA | ACTActatttaacacaCA | 32.6 |
| 1703 | 1703_1 | TATAGACCCTTAATATT | TATAgacccttaataTT | 41.4 |
| 1704 | 1704_1 | TTATAGACCCTTAATAT | TTAtagacccttaaTAT | 68.5 |
| 1705 | 1705_1 | CATCACAAAATAACCTATCAT | CAtcacaaaataacctaTCAT | 86.8 |
| 1706 | 1706_1 | TCATCACAAAATAACCTATCA | TCAtcacaaaataacctaTCA | 67.4 |
| 1707 | 1707_1 | TTCATCACAAAATAACCTATC | TTCAtcacaaaataacctaTC | 49 |
| 1708 | 1708_1 | TTCATCACAAAATAACCTA | TTcatcacaaaataaCCTA | 76.4 |
| 1709 | 1709_1 | TTTCATCACAAAATAACCTA | TTtcatcacaaaataaCCTA | 88.6 |
| 1710 | 1710_1 | TCATCACAAAATAACCTA | TCatcacaaaataaCCTA | 59.2 |
| 1711 | 1711_1 | TTTTCATCACAAAATAACCTA | TTttcatcacaaaataaCCTA | 86.1 |
| 1712 | 1712_1 | ATTTTCATCACAAAATAACCT | ATTttcatcacaaaataaCCT | 64.8 |
| 1713 | 1713_1 | TATTTTCATCACAAAATAACC | TATTttcatcacaaaataaCC | 76.9 |
| 1713 | 1713_2 | TATTTTCATCACAAAATAACC | TATTttcatcaCaaaataaCC | 56 |
| 1714 | 1714_1 | GTATTTTCATCACAAAATA | GTAT1f1catcacaaaaTA | 47 |
| 1715 | 1715_1 | TTACCTAGATCACTCC | TtacctagatcaCTCC | 73.1 |
| 1716 | 1716_1 | CTTACCTAGATCACTC | CTTacctagatcaCTC | 81.5 |
| 1717 | 1717_1 | CCTTACCTAGATCACT | CCTtacctagatcaCT | 95.9 |
| 1718 | 1718_1 | TAACTGCTCCTTAATCC | TAActgctccttaatCC | 34.8 |
| 1719 | 1719_1 | TCTAGCAATCCTCTCCT | TCtagcaatcctctcCT | 64.2 |
| 1720 | 1720_1 | TTCTAGCAATCCTCTCC | TtctagcaatcctcTCC | 70.4 |
| 1721 | 1721_1 | TTTTCACCTACTAATATTCAT | TTttcacctactaatatTCAT | 55.3 |
| 1722 | 1722_1 | TTTCACCTACTAATATTCAT | TTtcacctactaatatTCAT | 66.2 |
| 1723 | 1723_1 | TTCACCTACTAATATTCAT | TTCacctactaatattCAT | 17.2 |
| 1724 | 1724_1 | TCACCTACTAATATTCAT | TCAcctactaatattCAT | 23.5 |
| 1725 | 1725_1 | TCACCTACTAATATTCA | TCAcctactaatatTCA | 21.1 |
| 1726 | 1726_1 | TTTCACCTACTAATATTCA | TTTCacctactaatattCA | 16.7 |
| 1727 | 1727_1 | TTTTCACCTACTAATATTCA | TTttcacctactaataTTCA | 31.3 |
| 1728 | 1728_1 | TTTTTCACCTACTAATATTCA | TTtttcacctactaataTTCA | 45.3 |
| 1729 | 1729_1 | TTCACCTACTAATATTCA | TTCAcctactaatattCA | 24.7 |
| 1730 | 1730_1 | ATTTTTCACCTACTAATATTC | ATTMcacctactaataTTC | 48.5 |
| 1731 | 1731_1 | TTTTTCACCTACTAATATTC | TTTttcacctactaataTTC | 31.5 |
| 1732 | 1732_1 | TATTTTTCACCTACTAATATT | TAtttttcacctactaaTATT | 90.2 |
| 1733 | 1733_1 | TATTTTTCACCTACTAATAT | TATttttcacctactaaTAT | 89.1 |
| 1734 | 1734_1 | TTATTTTTCACCTACTAATAT | TTAtttttcacctactaaTAT | 86.1 |
| 1735 | 1735_1 | TTATTTTTCACCTACTAATA | TTATttttcacctactaaTA | 52.9 |
| 1736 | 1736_1 | TATTTTTCACCTACTAATA | TATTtttcacctactaaTA | 54.9 |
| 1737 | 1737_1 | TTTATTTTTCACCTACTAATA | TTTAtttttcacctactaaTA | 52 |
| 1738 | 1738_1 | TTTATTTTTCACCTACTAA | TTtatttttcacctaCTAA | 51.2 |
| 1739 | 1739_1 | TTTATTTTTCACCTACTA | TTTatttttcacctaCTA | 19 |
| 1740 | 1740_1 | CTCAACTTCTACTACTAATT | CTCAacttctactactaaTT | 19.7 |
| 1741 | 1741_1 | TCTCAACTTCTACTACTAATT | TCTCaacttctactactaaTT | 25.8 |
| 1742 | 1742_1 | CTCTCAACTTCTACTACTAAT | CTCtcaacttctactactAAT | 43 |
| 1743 | 1743_1 | CTCAACTTCTACTACTAAT | CTCAacttctactactaAT | 20.1 |
| 1744 | 1744_1 | TCTCAACTTCTACTACTAAT | TCTCaacttctactactaAT | 22.8 |
| 1745 | 1745_1 | TCTCTCAACTTCTACTACTAA | TCtctcaacttctactacTAA | 58.4 |
| 1746 | 1746_1 | CTCAACTTCTACTACTAA | CTcaacttctactaCTAA | 47.3 |
| 1747 | 1747_1 | TCTCAACTTCTACTACTAA | TCtcaacttctactaCTAA | 56.3 |
| 1748 | 1748_1 | CTCAACTTCTACTACTA | CTCaacttctactaCTA | 10.7 |
| 1749 | 1749_1 | TTCTCTCAACTTCTACTACTA | TtctctcaacttctactaCTA | 79.1 |
| 1750 | 1750_1 | TCTCTCAACTTCTACTACTA | TCtctcaacttctactacTA | 61.2 |
| 1751 | 1751_1 | TCTCAACTTCTACTACTA | TCtcaacttctactaCTA | 66.8 |
| 1752 | 1752_1 | CTCTCAACTTCTACTACTA | CtctcaacttctactACTA | 61.7 |
| 1753 | 1753_1 | CTCTCAACTTCTACTACT | CTCtcaacttctactaCT | 37.9 |
| 1754 | 1754_1 | TCTCAACTTCTACTACT | TCtcaacttctacTACT | 51.1 |
| 1755 | 1755_1 | TCTCTCAACTTCTACTACT | TCtctcaacttctactACT | 44.2 |
| 1756 | 1756_1 | TTTCTCTCAACTTCTACTACT | TTtctctcaacttctactACT | 65.7 |
| 1757 | 1757_1 | TTCTCTCAACTTCTACTACT | TTCtctcaacttctactaCT | 33.5 |
| 1758 | 1758_1 | TTTCTCTCAACTTCTACTAC | TTtctctcaacttctacTAC | 67.9 |
| 1759 | 1759_1 | CTCTCAACTTCTACTAC | CTCtcaacttctacTAC | 34.1 |
| 1760 | 1760_1 | TTCTCTCAACTTCTACTAC | TtctctcaacttctaCTAC | 63.8 |
| 1761 | 1761_1 | TTTTCTCTCAACTTCTACTAC | TTTTctctcaacttctactAC | 20.6 |
| 1762 | 1762_1 | TCTCTCAACTTCTACTAC | TCtctcaacttctacTAC | 49.7 |
| 1763 | 1763_1 | TTTCTCTCAACTTCTACTA | TTtctctcaacttctaCTA | 60.2 |
| 1764 | 1764_1 | TTTTCTCTCAACTTCTACTA | TtttctctcaacttctACTA | 52.2 |
| 1765 | 1765_1 | TTTTTCTCTCAACTTCTACTA | TTTttctctcaacttctacTA | 40.2 |
| 1766 | 1766_1 | TCTCTCAACTTCTACTA | TCtctcaacttctaCTA | 47.5 |
| 1767 | 1767_1 | TTCTCTCAACTTCTACTA | TTCtctcaacttctacTA | 35.1 |
| 1768 | 1768_1 | TTTCTCTCAACTTCTACT | TTTCtctcaacttctaCT | 28.6 |
| 1769 | 1769_1 | TTTTCTCTCAACTTCTACT | TTTtctctcaacttctaCT | 44.1 |
| 1770 | 1770_1 | CTTTTTCTCTCAACTTCTACT | CtttttctctcaacttctaCT | 99.8 |
| 1771 | 1771_1 | TTTTTCTCTCAACTTCTACT | TTTttctctcaacttctaCT | 43.7 |
| 1772 | 1772_1 | CTTTTTCTCTCAACTTCTAC | CTTtttctctcaacttctAC | 36.2 |
| 1773 | 1773_1 | ACTTTTTCTCTCAACTTCTAC | ACTttttctctcaacttctAC | 35.6 |
| 1774 | 1774_1 | TTTTCTCTCAACTTCTAC | TtttctctcaacttCTAC | 38.6 |
| 1775 | 1775_1 | TTTTTCTCTCAACTTCTAC | TttttctctcaacttCTAC | 42.1 |
| 1776 | 1776_1 | CTTTTTCTCTCAACTTCTA | CTttttctctcaacttCTA | 41.2 |
| 1777 | 1777_1 | TACTTTTTCTCTCAACTTCTA | TactttttctctcaacttCTA | 69.4 |
| 1778 | 1778_1 | ACTTTTTCTCTCAACTTCTA | ActttttctctcaacttCTA | 66.2 |
| 1779 | 1779_1 | TTTTTCTCTCAACTTCTA | TttttctctcaactTCTA | 35.5 |
| 1780 | 1780_1 | TACTTTTTCTCTCAACTTCT | TActttttctctcaacttCT | 65 |
| 1781 | 1781_1 | TTACTTTTTCTCTCAACTTCT | TtactttttctctcaactTCT | 62.1 |
| 1782 | 1782_1 | TTACTTTTTCTCTCAACTTC | TTActttttctctcaactTC | 38.9 |
| 1783 | 1783_1 | TACTTTTTCTCTCAACTTC | TACtttttctctcaactTC | 34 |
| 1784 | 1784_1 | ACTTTTTCTCTCAACTTC | ActttttctctcaaCTTC | 19.7 |
| 1785 | 1785_1 | TTACTTTTTCTCTCAACTT | TTActttttctctcaaCTT | 22 |
| 1786 | 1786_1 | TACTTTTTCTCTCAACTT | TACtttttctctcaaCTT | 22.3 |
| 1787 | 1787_1 | TTACTTTTTCTCTCAACT | TTACtttttctctcaaCT | 11.6 |
| 1788 | 1788_1 | GTTACTTTTTCTCTCAACT | GTtactttttctctcAACT | 43.2 |
| 1789 | 1789_1 | GTTACTTTTTCTCTCAAC | GTtactttttctctCAAC | 29 |
| 1790 | 1790_1 | GTTACTTTTTCTCTCAA | GTtactttttctcTCAA | 5.53 |
| 1791 | 1791_1 | AGTTACTTTTTCTCTCAA | AGTtactttttctctCAA | 6.5 |
| 1792 | 1792_1 | CTTTTACATTCCCATTAACA | CTTTtacattcccattaaCA | 24.5 |
| 1793 | 1793_1 | CACTTTTACATTCCCATTAAC | CACttttacattcccattaAC | 25.3 |
| 1794 | 1794_1 | CTTTTACATTCCCATTAAC | CTtttacattcccatTAAC | 21.5 |
| 1795 | 1795_1 | ACTTTTACATTCCCATTAAC | ACttttacattcccatTAAC | 23 |
| 1796 | 1796_1 | ACTTTTACATTCCCATTAA | ACttttacattcccaTTAA | 30 |
| 1797 | 1797_1 | CTTTTACATTCCCATTAA | CTtttacattcccaTTAA | 27.4 |
| 1798 | 1798_1 | CACTTTTACATTCCCATTAA | CActtttacattcccaTTAA | 28 |
| 1798 | 1798_2 | CACTTTTACATTCCCATTAA | CACttttacattcccatTAA | 15.9 |
| 1799 | 1799_1 | TACACTTTTACATTCCCATTA | TAcacttttacattcccatTA | 52.2 |
| 1800 | 1800_1 | ACTTTTACATTCCCATTA | ACTtttacattcccaTTA | 13.1 |
| 1801 | 1801_1 | CACTTTTACATTCCCATTA | CActtttacattcccATTA | 15.7 |
| 1802 | 1802_1 | ACACTTTTACATTCCCATTA | ACacttttacattcccaTTA | 19.1 |
| 1802 | 1802_2 | ACACTTTTACATTCCCATTA | ACActtttacattcccatTA | 9.66 |
| 1803 | 1803_1 | CACTTTTACATTCCCATT | CActtttacattccCATT | 10.2 |
| 1804 | 1804_1 | TACACTTTTACATTCCCATT | TACacttttacattcccaTT | 10.3 |
| 1805 | 1805_1 | ACACTTTTACATTCCCATT | ACACttttacattcccaTT | 4.51 |
| 1805 | 1805_2 | ACACTTTTACATTCCCATT | ACacttttacattccCATT | 6.8 |
| 1806 | 1806_1 | TACACTTTTACATTCCCAT | TACacttttacattccCAT | 3.53 |
| 1806 | 1806_2 | TACACTTTTACATTCCCAT | TACActtttacattcccAT | 4.79 |
| 1807 | 1807_1 | TACACTTTTACATTCCCA | TACacttttacattccCA | 6.35 |
| 1808 | 1808_1 | GTACACTTTTACATTCCCA | GtacacttttacattcCCA | 3 |
| 1808 | 1808_2 | GTACACTTTTACATTCCCA | GTacacttttacattccCA | 16.3 |
| 1809 | 1809_1 | GTACACTTTTACATTCCC | GTAcacttttacattcCC | 4.33 |
| 1810 | 1810_1 | TACACTTTTACATTCCC | TACacttttacattcCC | 3.26 |
| 1811 | 1811_1 | TGTACACTTTTACATTCCC | TGtacacttttacattcCC | 12.3 |
| 1809 | 1809_2 | GTACACTTTTACATTCCC | GtacacttttacattCCC | 2.49 |
| 1812 | 1812_1 | TGTACACTTTTACATTCC | TGtacacttttacatTCC | 2.47 |
| 1813 | 1813_1 | CTGTACACTTTTACATTC | CTGtacacttttacaTTC | 1.89 |
| 1814 | 1814_1 | ATCTTATTTACATCTTCC | ATcttatttacatcTTCC | 5.41 |
| 1815 | 1815_1 | GAATCTTATTTACATCTTC | GAatcttatttacatCTTC | 25.8 |
| 1816 | 1816_1 | GAATCTTATTTACATCTT | GAatcttatttacaTCTT | 19.1 |
| 1817 | 1817_1 | TGAATCTTATTTACATCT | TGAatcttatttacaTCT | 41.3 |
| 1818 | 1818_1 | ATTCAGCTTTTTCAATC | ATTCagctttttcaaTC | 16.8 |
| 1819 | 1819_1 | TTAATTTTCCCTTCACTCCT | TtaattttcccttcactcCT | 85.8 |
| 1820 | 1820_1 | TTAATTTTCCCTTCACTCC | TtaattttcccttcactCC | 85.8 |
| 1821 | 1821_1 | TTAATTTTCCCTTCACTC | TtaattttcccttcACTC | 51 |
| 1822 | 1822_1 | GTTAATTTTCCCTTCACTC | GttaattttcccttcACTC | 27.2 |
| 1823 | 1823_1 | CAAAATTACTTCTTTTATCAT | CAaaattacttcttttaTCAT | 86.7 |
| 1823 | 1823_2 | CAAAATTACTTCTTTTATCAT | CAaaattacTtcttttaTCAT | 51.5 |
| 1824 | 1824_1 | CCAAAATTACTTCTTTTATCA | CCAaaattacttcttttaTCA | 31.3 |
| 1824 | 1824_2 | CCAAAATTACTTCTTTTATCA | CCaaaattacttcttttATCA | 36 |
| 1825 | 1825_1 | TCCAAAATTACTTCTTTTATC | TCcaaaattacttctttTATC | 40.9 |
| 1826 | 1826_1 | TCCAAAATTACTTCTTTTAT | TCCaaaattacttctttTAT | 50.2 |
| 1827 | 1827_1 | CCAAAATTACTTCTTTTAT | CCAaaattacttctttTAT | 70 |
| 1828 | 1828_1 | TTCCAAAATTACTTCTTTTAT | TTCcaaaattacttctttTAT | 64.9 |
| 1829 | 1829_1 | TCCAAAATTACTTCTTTTA | TCCAaaattacttctttTA | 36.9 |
| 1830 | 1830_1 | TTCCAAAATTACTTCTTTTA | TTCCaaaattacttctttTA | 52.2 |
| 1831 | 1831_1 | GTTCCAAAATTACTTCTTT | GTTCcaaaattacttctTT | 54.8 |
| 1832 | 1832_1 | GTTCCAAAATTACTTCTT | GTtccaaaattactTCTT | 12.5 |
| 1833 | 1833_1 | TGTTCCAAAATTACTTCT | TGTtccaaaattactTCT | 20.1 |
| 1834 | 1834_1 | ATGTTCCAAAATTACTTC | ATGTtccaaaattactTC | 23.8 |
| 1835 | 1835_1 | CATATTTTACTCTTTTTATT | CATAttttactctttttaTT | 90.6 |
| 1836 | 1836_1 | CCATATTTTACTCTTTTTAT | CCATattttactctttttAT | 35.4 |
| 1836 | 1836_2 | CCATATTTTACTCTTTTTAT | CCAtattttactcttaTAT | 60.8 |
| 1837 | 1837_1 | CCCATATTTTACTCTTTTTAT | CccatattttactctttTTAT | 75.8 |
| 1838 | 1838_1 | CATATTTTACTCTTTTTAT | CATattttactcttttTAT | 83.2 |
| 1839 | 1839_1 | CCCATATTTTACTCTTTTTA | CCcatattttactcttttTA | 81.1 |
| 1840 | 1840_1 | CCATATTTTACTCTTTTTA | CCatattttactcttTTTA | 24.7 |
| 1841 | 1841_1 | ACCCATATTTTACTCTTTTTA | AcccatattttactcttTTTA | 59 |
| 1842 | 1842_1 | CCATATTTTACTCTTTTT | CCATattttactctttTT | 21.6 |
| 1843 | 1843_1 | CCCATATTTTACTCTTTTT | CCcatattttactcttTTT | 77.2 |
| 1844 | 1844_1 | ACCCATATTTTACTCTTTTT | ACccatattttactctTTTT | 97.4 |
| 1845 | 1845_1 | TACCCATATTTTACTCTTTTT | TAcccatattttactcttTTT | 58.6 |
| 1846 | 1846_1 | TACCCATATTTTACTCTTTT | TACccatattttactctTTT | 20.4 |
| 1847 | 1847_1 | CCCATATTTTACTCTTTT | CCCatattttactcttTT | 93.2 |
| 1848 | 1848_1 | ACCCATATTTTACTCTTTT | ACCcatattttactcttTT | 21.8 |
| 1846 | 1846_2 | TACCCATATTTTACTCTTTT | TAcccatattttactcTTTT | 22.5 |
| 1849 | 1849_1 | TTACCCATATTTTACTCTTTT | TTAcccatattttactcttTT | 41.4 |
| 1850 | 1850_1 | TACCCATATTTTACTCTTT | TAcccatattttactCTTT | 18.9 |
| 1851 | 1851_1 | ACCCATATTTTACTCTTT | ACCcatattttactcTTT | 13.4 |
| 1852 | 1852_1 | TTACCCATATTTTACTCTTT | TTacccatattttactCTTT | 14.5 |
| 1853 | 1853_1 | TTTACCCATATTTTACTCTTT | TTTacccatattttactcTTT | 22.2 |
| 1852 | 1852_2 | TTACCCATATTTTACTCTTT | TTACccatattttactctTT | 16.7 |
| 1853 | 1853_2 | TTTACCCATATTTTACTCTTT | TTTAcccataactctTT | 16 |
| 1854 | 1854_1 | TTACCCATATTTTACTCTT | TTAcccatattttactCTT | 14 |
| 1855 | 1855_1 | TTTACCCATATTTTACTCTT | TTtacccatattttacTCTT | 14.9 |
| 1856 | 1856_1 | ACCCATATTTTACTCTT | ACCcatattttactCTT | 8.02 |
| 1857 | 1857_1 | TACCCATATTTTACTCTT | TACccatattttactCTT | 16.7 |
| 1858 | 1858_1 | TACCCATATTTTACTCT | TACccatattttacTCT | 22.3 |
| 1859 | 1859_1 | TTACCCATATTTTACTCT | TTACccatattttactCT | 15.2 |
| 1860 | 1860_1 | TTTACCCATATTTTACTCT | TTTAcccatattttactCT | 11.8 |
| 1861 | 1861_1 | TTACCCATATTTTACTC | TTAcccatattttaCTC | 24.4 |
| 1862 | 1862_1 | TTTACCCATATTTTACTC | TTTacccatattttaCTC | 14 |
| 1863 | 1863_1 | GTTTACCCATATTTTACTC | GTttacccatattttaCTC | 12.2 |
| 1864 | 1864_1 | GTTTACCCATATTTTACT | GTttacccatatttTACT | 24.9 |
| 1865 | 1865_1 | TGTTTACCCATATTTTAC | TGTttacccatatttTAC | 13.1 |
| 1866 | 1866_1 | GTTTACCCATATTTTAC | GTttacccatattTTAC | 13.2 |
| 1867 | 1867_1 | TGTTTACCCATATTTTA | TGTttacccatattTTA | 6.69 |
| 1868 | 1868_1 | TTCTTGCTTCAACCATC | TtcttgcttcaacCATC | 13.6 |
| 1869 | 1869_1 | GTTACCTCCCTTTATAT | GTtacctccctttatAT | 60.9 |
| 1870 | 1870_1 | GGTTACCTCCCTTTAT | GgttacctccctTTAT | 39 |
| 1871 | 1871_1 | AGGTTACCTCCCTTTA | AggttacctcccTTTA | 35.4 |
| 1872 | 1872_1 | ATGTTCTCTATCTCTATA | ATGttctctatctctATA | 53.3 |
| 1873 | 1873_1 | TATGTTCTCTATCTCTA | TAtgttctctatctCTA | 73.4 |
| 1874 | 1874_1 | AGATCAAACTAAAACCT | AGAtcaaactaaaaCCT | 88.7 |
| 1875 | 1875_1 | TGCCCAATTTCACCCAA | TGcccaatttcacccAA | 30.3 |
| 1876 | 1876_1 | TTTGCCCAATTTCACCC | TttgcccaatttcacCC | 53.3 |
| 1877 | 1877_1 | TTTTGCCCAATTTCACC | TTttgcccaatttcaCC | 57.8 |
| 1878 | 1878_1 | TGTATATCAACAATTCAT | TGTatatcaacaattCAT | 20.8 |
| 1879 | 1879_1 | ACATTTCTTTAAAATTTCCA | ACatttctttaaaattTCCA | 96.4 |
| 1879 | 1879_2 | ACATTTCTTTAAAATTTCCA | ACAtttctttaaaatttCCA | 96.6 |
| 1880 | 1880_1 | CACATTTCTTTAAAATTTCCA | CACAtttctttaaaatttcCA | 95.5 |
| 1879 | 1879_3 | ACATTTCTTTAAAATTTCCA | AcatttctttaaaattTCCA | 98.1 |
| 1879 | 1879_4 | ACATTTCTTTAAAATTTCCA | ACATttctttaaaatttcCA | 98 |
| 1881 | 1881_1 | CCACATTTCTTTAAAATTTCC | CcacatttctttaaaatTTCC | 90 |
| 1882 | 1882_1 | CACATTTCTTTAAAATTTCC | CAcatttctttaaaattTCC | 94.8 |
| 1882 | 1882_2 | CACATTTCTTTAAAATTTCC | CAcatttctttaaaatTTCC | 89.1 |
| 1882 | 1882_3 | CACATTTCTTTAAAATTTCC | CACAtttctttaaaatttCC | 94.4 |
| 1883 | 1883_1 | ACATTTCTTTAAAATTTCC | ACAtttctttaaaattTCC | 91.9 |
| 1882 | 1882_4 | CACATTTCTTTAAAATTTCC | CACatttctttaaaattTCC | 92.4 |
| 1884 | 1884_1 | CCACATTTCTTTAAAATTTC | CCACatttctttaaaattTC | 98.3 |
| 1885 | 1885_1 | ACCACATTTCTTTAAAATTTC | ACCAcatttctttaaaattTC | 97.5 |
| 1884 | 1884_2 | CCACATTTCTTTAAAATTTC | CCAcatttctttaaaattTC | 102 |
| 1884 | 1884_3 | CCACATTTCTTTAAAATTTC | CCacatttctttaaaaTTTC | 94.9 |
| 1884 | 1884_4 | CCACATTTCTTTAAAATTTC | CCAcatttctttaaaatTTC | 87.2 |
| 1886 | 1886_1 | ACCACATTTCTTTAAAATTT | ACCAcatttctttaaaatTT | 94.8 |
| 1887 | 1887_1 | ACAAAACCACATTTCTTTAA | ACAaaaccacatttcttTAA | 97.4 |
| 1888 | 1888_1 | CTGTTTTCAAATCATTTC | CTGTtttcaaatcattTC | 15.8 |
| 1889 | 1889_1 | GAACCATTACTATTATCAA | GAaccattactattaTCAA | 27.3 |
| 1890 | 1890_1 | AGAACCATTACTATTATCA | AGAaccattactattaTCA | 19.8 |
| 1891 | 1891_1 | AGAACCATTACTATTATC | AGaaccattactatTATC | 17.9 |
| 1892 | 1892_1 | CTAGAACCATTACTATTA | CTAGaaccattactatTA | 35.3 |
| 1893 | 1893_1 | TAGAACCATTACTATTA | TAGAaccattactatTA | 13.2 |
| 1894 | 1894_1 | CTAGAACCATTACTATT | CTAGaaccattactaTT | 32.1 |
| 1895 | 1895_1 | AGATTACCATCTTTCAAAA | AGATtaccatctttcaaAA | 59.5 |
| 1895 | 1895_2 | AGATTACCATCTTTCAAAA | AGAttaccatctttcaAAA | 54.1 |
| 1896 | 1896_1 | AGATTACCATCTTTCAAA | AGATtaccatctttcaAA | 50.6 |
| 1896 | 1896_2 | AGATTACCATCTTTCAAA | AGattaccatattCAAA | 42.3 |
| 1897 | 1897_1 | AGATTACCATCTTTCAA | AGAttaccatctttCAA | 32.4 |
| 1898 | 1898_1 | AAGATTACCATCTTTCA | AAGAttaccatattCA | 47.9 |
| 1899 | 1899_1 | CATGCTCACACATTTTAA | CATgctcacacatttTAA | 60.5 |
| 1899 | 1899_2 | CATGCTCACACATTTTAA | CAtgctcacacattTTAA | 70.3 |
| 1899 | 1899_3 | CATGCTCACACATTTTAA | CAtgctcacacatttTAA | 69.8 |
| 1899 | 1899_4 | CATGCTCACACATTTTAA | CATGctcacacattttAA | 55.9 |
| 1900 | 1900_1 | CTTAAGCTATCTAAACA | CTTAagctatctaaaCA | 82.6 |
| 1901 | 1901_1 | TGAACAATTCAACATTCA | TGAacaattcaacatTCA | 67.7 |
| 1902 | 1902_1 | GATCAAAAAACTTTCCCT | GAtcaaaaaactttCCCT | 76.1 |
| 1903 | 1903_1 | AGATCAAAAAACTTTCCCT | AGatcaaaaaactttCCCT | 70.4 |
| 1904 | 1904_1 | AGATCAAAAAACTTTCCC | AGAtcaaaaaactaCCC | 73.6 |
| 1905 | 1905_1 | TCCTAGATCAAAAAACT | TCCTagatcaaaaaaCT | 69.9 |
| 1906 | 1906_1 | ATTTTTTCTTCTCTTTTCA | ATTTtttcttcttttCA | 8.98 |
| 1907 | 1907_1 | TATTTTTTCTTCTCTTTTCA | TATtttttcttctcttttCA | 63.8 |
| 1908 | 1908_1 | ATATTTTTTCTTCTCTTTTC | ATattttttcttctctTTTC | 16.1 |
| 1909 | 1909_1 | TCTGCTTTAAAAACTCTC | TCtgctttaaaaacTCTC | 34.3 |
| 1910 | 1910_1 | CTCTGCTTTAAAAACTC | CTCtgctttaaaaaCTC | 51.6 |
| 1911 | 1911_1 | ACTACACAAACACATTCAA | ACtacacaaacacatTCAA | 37.6 |
| 1912 | 1912_1 | CAAACTACACAAACACATTCA | CAaactacacaaacacaTTCA | 41.2 |
| 1913 | 1913_1 | ACAAACTACACAAACACATTC | ACAaactacacaaacacaTTC | 63.1 |
| 1914 | 1914_1 | CAACAAACTACACAAACACAT | CAAcaaactacacaaacaCAT | 86.1 |
| 1915 | 1915_1 | CACAACAAACTACACAAACAC | CACaacaaactacacaaaCAC | 62.1 |
| 1916 | 1916_1 | TCACAACAAACTACACAAACA | TCACaacaaactacacaaaCA | 48.6 |
| 1917 | 1917_1 | TTCACAACAAACTACACAAAC | TTCAcaacaaactacacaaAC | 58.8 |
| 1918 | 1918_1 | ATTTCACAACAAACTACACAA | ATTtcacaacaaactacaCAA | 76.8 |
| 1919 | 1919_1 | CAATTTCACAACAAACTACAC | CAAtttcacaacaaactaCAC | 70.7 |
| 1920 | 1920_1 | TGTAACAATTTCACAACAA | TGTaacaatttcacaaCAA | 59.5 |
| 1921 | 1921_1 | TGTAACAATTTCACAACA | TGTAacaatttcacaaCA | 28.7 |
| 1922 | 1922_1 | TTAAGCCAACCCCACCA | TtaagccaaccccacCA | 83.1 |
| 1923 | 1923_1 | TTTAAGCCAACCCCACC | TttaagccaaccccACC | 69.2 |
| 1924 | 1924_1 | ATTTAAGCCAACCCCAC | AtttaagccaaccCCAC | 60.6 |
| 1925 | 1925_1 | CCAGTAATACAAATTATA | CCAGtaatacaaattaTA | 69.5 |
| 1926 | 1926_1 | CCCAGTAATACAAATTA | CCCAgtaatacaaatTA | 55.9 |
| 1927 | 1927_1 | TCCCAGTAATACAAATT | TCCCagtaatacaaaTT | 64.9 |
| 1928 | 1928_1 | ATCCCAGTAATACAAAT | ATCCcagtaatacaaAT | 65.9 |
| 1929 | 1929_1 | CTACTAGCATCACCACT | CtactagcatcacCACT | 19.8 |
| 1930 | 1930_1 | TTCTACTAGCATCACC | TtctactagcatCACC | 21.8 |
| 1931 | 1931_1 | CTTCTACTAGCATCAC | CTtctactagcaTCAC | 33.2 |
| 1932 | 1932_1 | TAAATTACTCATTAAATCCAT | TAaattactcattaaatCCAT | 77.8 |
| 1933 | 1933_1 | ATAAATTACTCATTAAATCCA | ATaaattactcattaaaTCCA | 52.4 |
| 1934 | 1934_1 | TAAATTACTCATTAAATCCA | TAaattactcattaaaTCCA | 51.6 |
| 1935 | 1935_1 | CATAAATTACTCATTAAATCC | CATaaattactcattaaaTCC | 58.5 |
| 1935 | 1935_2 | CATAAATTACTCATTAAATCC | CATaaattacTcattaaaTCC | 22.3 |
| 1936 | 1936_1 | GATTTATTTTTCTACTTA | GAtttatwtctaCTTA | 66 |
| 1937 | 1937_1 | ATACAACAAACAATTCACTTT | ATacaacaaacaattcaCTTT | 53.2 |
| 1937 | 1937_2 | ATACAACAAACAATTCACTTT | ATACaacaaacaattcactTT | 48.1 |
| 1938 | 1938_1 | CGATACAACAAACAATTCA | CGATacaacaaacaattCA | 23 |
| 1939 | 1939_1 | GAACATCCACACTAACAACA | GAACatccacactaacaaCA | 43.6 |
| 1940 | 1940_1 | ACATCCACACTAACAACA | ACAtccacactaacaACA | 65 |
| 1939 | 1939_2 | GAACATCCACACTAACAACA | GAAcatccacactaacaACA | 52 |
| 1939 | 1939_3 | GAACATCCACACTAACAACA | GAacatccacactaacAACA | 58.1 |
| 1941 | 1941_1 | GAACATCCACACTAACAAC | GAACatccacactaacaAC | 51.3 |
| 1941 | 1941_2 | GAACATCCACACTAACAAC | GAacatccacactaaCAAC | 63.3 |
| 1942 | 1942_1 | TGAACATCCACACTAACAA | TGAacatccacactaaCAA | 57.8 |
| 1943 | 1943_1 | TTGAACATCCACACTAACA | TTGAacatccacactaaCA | 60.3 |
| 1944 | 1944_1 | TGAACATCCACACTAACA | TGAAcatccacactaaCA | 42.6 |
| 1945 | 1945_1 | CATTGAACATCCACACTA | CATtgaacatccacaCTA | 59.4 |
| 1946 | 1946_1 | ATTGAACATCCACACTA | ATTgaacatccacaCTA | 50 |
| 1947 | 1947_1 | CATTGAACATCCACACT | CAttgaacatccaCACT | 43 |
| 1948 | 1948_1 | ACTCATTGAACATCCAC | ACtcattgaacatCCAC | 46.8 |
| 1949 | 1949_1 | TATCTTTATTTAATAATCTT | TATCtttatttaataatcTT | 93.4 |
| 1949 | 1949_2 | TATCTTTATTTAATAATCTT | TAtctttatttaataaTCTT | 96.9 |
| 1950 | 1950_1 | TCTCAAGCTTCACTCTA | TCtcaagcttcactcTA | 78.6 |
| 1951 | 1951_1 | GACAATATATTCCTCAATC | GACAatatattcctcaaTC | 73 |
| 1952 | 1952_1 | GACAATATATTCCTCAAT | GACAatatattcctcaAT | 82 |
| 1952 | 1952_2 | GACAATATATTCCTCAAT | GAcaatatattcctCAAT | 76.8 |
| 1953 | 1953_1 | TCCTGTAACAATTATAC | TCCtgtaacaattaTAC | 95.4 |
| 1954 | 1954_1 | ACCCAGAATAAAAACCAC | ACccagaataaaaaCCAC | 95.5 |
| 1955 | 1955_1 | TTCCACTTTCTTACTCCC | TtccactttcttactcCC | 96.6 |
| 1956 | 1956_1 | TTCCACTTTCTTACTCC | TtccactttcttacTCC | 86.3 |
| 1957 | 1957_1 | TTTCCACTTTCTTACTCC | TttccactttcttacTCC | 89.2 |
| 1958 | 1958_1 | TTTCCACTTTCTTACTC | TTTCcactttcttacTC | 89.2 |
| 1959 | 1959_1 | ATCCCTTTACCACTTTT | ATCcctttaccactTTT | 101 |
| 1960 | 1960_1 | CATCCCTTTACCACTTTT | CAtccctttaccactTTT | 98 |
| 1961 | 1961_1 | TCATCCCTTTACCACTTT | TCatccctttaccactTT | 101 |
| 1962 | 1962_1 | TCATCCCTTTACCACTT | TCAtccctttaccacTT | 96.9 |
| 1963 | 1963_1 | CTCATCCCTTTACCACTT | CtcatccctttaccacTT | 97.7 |
| 1964 | 1964_1 | GTCTACATCTAACCCC | GtctacatctaacCCC | 97 |
| 1965 | 1965_1 | AGTCTACATCTAACCCC | AGtctacatctaaccCC | 99.6 |
| 1966 | 1966_1 | CAGTCTACATCTAACCCC | CagtctacatctaaccCC | 97.4 |
| 1967 | 1967_1 | CAGTCTACATCTAACCC | CagtctacatctaaCCC | 99.5 |
| 1968 | 1968_1 | TCAGTCTACATCTAACCC | TCagtctacatctaacCC | 98.9 |
| 1969 | 1969_1 | AGTCTACATCTAACCC | AGTctacatctaacCC | 98.2 |
| 1970 | 1970_1 | TCAGTCTACATCTAACC | TCagtctacatctAACC | 98.3 |
| 1971 | 1971_1 | TTCAGTCTACATCTAACC | TTCagtctacatctaaCC | 98 |
| 1972 | 1972_1 | TTCAGTCTACATCTAAC | TTCAgtctacatctaAC | 98.7 |
| 1973 | 1973_1 | TTTCAGTCTACATCTAA | TTtcagtctacatCTAA | 90.1 |
| 1974 | 1974_1 | AGTTTTAACCACACCTCCT | AgttttaaccacacctcCT | 102 |
| 1975 | 1975_1 | GTTTTAACCACACCTCC | GTTttaaccacacctCC | 93.7 |
| 1976 | 1976_1 | AGTTTTAACCACACCTCC | AgttttaaccacaccTCC | 95 |
| 1977 | 1977_1 | AGTTTTAACCACACCTC | AGttttaaccacacCTC | 88.7 |
| 1978 | 1978_1 | GAGTTTTAACCACACC | GAGMtaaccacACC | 94.7 |
| 1979 | 1979_1 | CAGATCTTCTCTTTATTT | CAGatcttctctttaTTT | 96.3 |
| 1980 | 1980_1 | TGTTTTCAACAAAACATCA | TGTtttcaacaaaacaTCA | 89.9 |
| 1981 | 1981_1 | TGTTTTCAACAAAACATC | TGttacaacaaaaCATC | 97.5 |
| 1982 | 1982_1 | CTGTTTTCAACAAAACAT | CTGttttcaacaaaaCAT | 102 |
| 1983 | 1983_1 | TCTGTTTTCAACAAAACA | TCTGttacaacaaaaCA | 98 |
| 1984 | 1984_1 | ATCTTTCTAAAACTTACC | ATCTttctaaaacttaCC | 96.3 |
| 1985 | 1985_1 | CAGAATCTTTCTAAAACT | CAGAatctttctaaaaCT | 91.7 |
| 1986 | 1986_1 | CTACAGAATCTTTCTAA | CTacagaatctttCTAA | 97.6 |
| 1986 | 1986_2 | CTACAGAATCTTTCTAA | CTAcagaatctttcTAA | 95.6 |
| 1987 | 1987_1 | ATTTCCCTTTATTTCCCTT | AtttccctttatttccCTT | 92 |
| 1988 | 1988_1 | GTATTTCCCTTTATTTCC | GtatttccctttattTCC | 99.5 |

In the oligonucleotide compound column, capital letters represent beta-D-oxy LNA nucleosides, LNA cytosines are 5-methyl cytosine, lower case letters are DNA nucleosides, and all internucleoside linkages are phosphorothioate. ^{m}c represent 5-methyl cytosine DNA nucleosides (used in compounds 1490_1 and 1491_1).

### Example 4

### Materials and Methods:

The screening assay described in Example 2 was performed using a series of further oligonucleotide targeting human ATXN3 pre-mRNA using the qpCR: (ATXN3 exon 8-9(1) PrimeTime® XL qPCR Assay (IDT).

qPCR probe and primers set 2:
(SEQ ID NO: 1134)
Probe: 5′-/56-FAM/CTCCGCAGG/ZEN/GCT ATTCAGCT AAGT/
31ABkFQ/-3′
(SEQ ID NO: 1135)
Primer 1: 5′-AGT AAGATTTGT ACCTGATGTCTGT-3′
(SEQ ID NO: 1136)
Primer 2: 5′-CATGGAAGATGAGGAAGCAGAT-3′

### Results:

TABLE 6

| | | | | % of |
|---|---|---|---|---|
| | | | | ATXN3 |
| | | Oligonucleotide | Oligonucleotide | mRNA |
| SEQID | CMPID | Base Sequence | compound | remaining |
| 1110 | 1110_2 | ACATCATTTATCACTACCAC | ACatcatttatcactacCAC | 44 |
| 1102 | 1102_2 | TATCTCAAACTATCCCCA | TatctcaaactatccCCA | 74 |
| 1104 | 1104_2 | TCCCCTAAATAATTTAATCA | TCCcctaaataatttaaTCA | 78 |
| 1116 | 1116_2 | TCTTCATTATACCATCAAAT | TCTTcattataccatcaaAT | 12 |
| 1121 | 1121_2 | CTCTCAACTTCTACTACTAA | CtctcaacttctactaCTAA | 68 |
| 1114 | 1114_2 | TGATTCTTATACTTACTA | TGATtcttatacttacTA | 64 |
| 1120 | 1120_2 | CATCACAAAATAACCTATCA | CATCacaaaataacctatCA | 38 |
| 1100 | 1100_2 | CCCCATTCAAATATTTATT | CCCcattcaaatatttATT | 79 |
| 1112 | 1112_2 | TCAGATCCTAAAATCACT | TCAGatcctaaaatcaCT | 65 |
| 1123 | 1123_2 | CCAAAATTACTTCTTTTATC | CCaaaattacttctttTATC | 37 |
| 1117 | 1117_2 | GTTTCATATTTTTAATCC | GTftcatatttttaATCC | 10 |
| 1099 | 1099_2 | CCAAAAGAAACCAAACCC | CCaaaagaaaccaaACCC | 88 |
| 1109 | 1109_2 | TGAAACCATTACTACAACC | TGAaaccattactacaACC | 22 |
| 1113 | 1113_2 | CTATACCTAAAACAATCTA | CTatacctaaaacaaTCTA | 86 |
| 1119 | 1119_2 | CAAATATTCACAAATCCTA | CaaatattcacaaatCCTA | 78 |
| 1125 | 1125_2 | ACAATATATTCCTCAATCA | ACaatatattcctcaATCA | 74 |
| 1127 | 1127_2 | CATCCCTTTACCACTTT | CatccctttaccaCTTT | 97 |
| 1118 | 1118_2 | TAATATCCTCATTACCCATT | TaatatcctcattaccCATT | 97 |
| 1103 | 1103_2 | TCTATTCCTTAACCCAAC | TCtattccttaaccCAAC | 81 |
| 1122 | 1122_2 | AATCTTATTTACATCTTCC | AATCttatttacatcttCC | 11 |
| 1126 | 1126 2 | CCTGTAACAATTATACA | CCTGtaacaattataCA | 93 |
| 1122 | 1122_3 | AATCTTATTTACATCTTCC | AatcttatttacaTCtTCC | 54 |
| 1122 | 1122_4 | AATCTTATTTACATCTTCC | AAtcTtatttacAtCttCC | 17 |
| 1122 | 1122_5 | AATCTTATTTACATCTTCC | AAtcttatttacAtCttCC | 21 |
| 1122 | 1122_6 | AATCTTATTTACATCTTCC | AatctTatttacaTCttCC | 12 |
| 1122 | 1122_7 | AATCTTATTTACATCTTCC | AatcttatttacAtCttCC | 28 |
| 1122 | 1122_8 | AATCTTATTTACATCTTCC | AAtcttatttacAtcTTCC | 28 |
| 1122 | 1122_9 | AATCTTATTTACATCTTCC | AAtcTtatttacAtctTCC | 11 |
| 1122 | 1122_10 | AATCTTATTTACATCTTCC | AatctTatttacAtctTCC | 9 |
| 1122 | 1122_11 | AATCTTATTTACATCTTCC | AatcTtatttacatcTTCC | 10 |
| 1122 | 1122_12 | AATCTTATTTACATCTTCC | AATcTtatttacAtcTtCC | 10 |
| 1122 | 1122_13 | AATCTTATTTACATCTTCC | AatCTtatttacAtcttCC | 10 |
| 1122 | 1122_14 | AATCTTATTTACATCTTCC | AatCttatttacatctTCC | 7 |
| 1122 | 1122_15 | AATCTTATTTACATCTTCC | AatcttatttacaTCttCC | 32 |
| 1122 | 1122_16 | AATCTTATTTACATCTTCC | AatCttatttacatcTTCC | 4 |
| 1122 | 1122_17 | AATCTTATTTACATCTTCC | AAtCttatttacatcTtCC | 5 |
| 1122 | 1122_18 | AATCTTATTTACATCTTCC | AaTcTtatttacaTcTtCC | 9 |
| 1122 | 1122_19 | AATCTTATTTACATCTTCC | AatcTTatttacatcTtCC | 5 |
| 1122 | 1122_20 | AATCTTATTTACATCTTCC | AatcTtatttacatCttCC | 13 |
| 1122 | 1122_21 | AATCTTATTTACATCTTCC | AAtcttatttacatCttCC | 23 |
| 1122 | 1122_22 | AATCTTATTTACATCTTCC | AatctTatttacatCttCC | 8 |
| 1122 | 1122_23 | AATCTTATTTACATCTTCC | AatcTTatttacatCttCC | 4 |
| 1122 | 1122_24 | AATCTTATTTACATCTTCC | AatctTatttacatcTTCC | 8 |
| 1122 | 1122_25 | AATCTTATTTACATCTTCC | AATcTTatttacatcTtCC | 5 |
| 1122 | 1122_26 | AATCTTATTTACATCTTCC | AAtctTatttacatcTtCC | 12 |
| 1122 | 1122_27 | AATCTTATTTACATCTTCC | AaTCTtatttacatcTtCC | 3 |
| 1122 | 1122_28 | AATCTTATTTACATCTTCC | AaTcTTatttacatcTtCC | 3 |
| 1122 | 1122_29 | AATCTTATTTACATCTTCC | AatCTTatttacatcTtCC | 3 |
| 1122 | 1122_30 | AATCTTATTTACATCTTCC | AAtcTTatttacatctTCC | 5 |
| 1122 | 1122_31 | AATCTTATTTACATCTTCC | AAtcTtatttacatctTCC | 12 |
| 1122 | 1122_32 | AATCTTATTTACATCTTCC | AAtcttatttacatctTCC | 33 |
| 1122 | 1122_33 | AATCTTATTTACATCTTCC | AatCtTatttacatctTCC | 3 |
| 1122 | 1122_34 | AATCTTATTTACATCTTCC | AatcTTatttacatctTCC | 6 |
| 1122 | 1122_35 | AATCTTATTTACATCTTCC | AatcTtatttacatctTCC | 16 |
| 1122 | 1122_36 | AATCTTATTTACATCTTCC | AATCtTatttacatcttCC | 8 |
| 1122 | 1122_37 | AATCTTATTTACATCTTCC | AAtCTTatttacatcttCC | 5 |
| 1122 | 1122_38 | AATCTTATTTACATCTTCC | AAtCttatttacatcttCC | 16 |
| 1122 | 1122_39 | AATCTTATTTACATCTTCC | AaTCTtatttacatcttCC | 7 |
| 1122 | 1122_40 | AATCTTATTTACATCTTCC | AaTCtTatttacatcttCC | 5 |
| 1122 | 1122_41 | AATCTTATTTACATCTTCC | AatCTTatttacatcttCC | 5 |
| 1122 | 1122_42 | AATCTTATTTACATCTTCC | AatCTtatttacatcttCC | 13 |
| 1122 | 1122_43 | AATCTTATTTACATCTTCC | AatcTTatttacatcttCC | 17 |
| 1109 | 1109_3 | TGAAACCATTACTACAACC | TgaaaccattacTAcaaCC | 58 |
| 1109 | 1109_4 | TGAAACCATTACTACAACC | TgaaaccattacTAcAaCC | 20 |
| 1109 | 1109_5 | TGAAACCATTACTACAACC | TgaAAccattacTacAaCC | 23 |
| 1109 | 1109_6 | TGAAACCATTACTACAACC | TgAaAccattactAcaaCC | 50 |
| 1109 | 1109_7 | TGAAACCATTACTACAACC | TgAaaCcattactAcaaCC | 46 |
| 1109 | 1109_8 | TGAAACCATTACTACAACC | TgaAAccattacTacaaCC | 48 |
| 1109 | 1109_9 | TGAAACCATTACTACAACC | TgaaaccattactaCAaCC | 25 |
| 1109 | 1109_10 | TGAAACCATTACTACAACC | TgaaAccattacTaCaACC | 24 |
| 1109 | 1109_11 | TGAAACCATTACTACAACC | TGaaAccattactaCaaCC | 36 |
| 1109 | 1109_12 | TGAAACCATTACTACAACC | TgAAAccattactaCaaCC | 20 |
| 1109 | 1109_13 | TGAAACCATTACTACAACC | TgAAaCcattactaCaaCC | 26 |
| 1109 | 1109_14 | TGAAACCATTACTACAACC | TgAaaccattactaCaaCC | 27 |
| 1109 | 1109_15 | TGAAACCATTACTACAACC | TGaAaccattacTacAaCC | 14 |
| 1109 | 1109_16 | TGAAACCATTACTACAACC | TgAaaCcattactacAACC | 12 |
| 1109 | 1109_17 | TGAAACCATTACTACAACC | TgaaaCcattacTacAaCC | 36 |
| 1109 | 1109_18 | TGAAACCATTACTACAACC | TgaaaCcattacTacaaCC | 62 |
| 1109 | 1109_19 | TGAAACCATTACTACAACC | TGaaAccattactacaaCC | 47 |
| 1109 | 1109_20 | TGAAACCATTACTACAACC | TgaAaccattactaCAaCC | 19 |
| 1109 | 1109_21 | TGAAACCATTACTACAACC | TgaAaccattactACaACC | 16 |
| 1109 | 1109_22 | TGAAACCATTACTACAACC | TgAAaccattactACaACC | 9 |
| 1109 | 1109_23 | TGAAACCATTACTACAACC | TgAaAccattactAcaACC | 29 |
| 1109 | 1109_24 | TGAAACCATTACTACAACC | TgaaaCcattactAcaACC | 41 |
| 1109 | 1109_25 | TGAAACCATTACTACAACC | TgaAACcattactAcaaCC | 34 |
| 1109 | 1109_26 | TGAAACCATTACTACAACC | TgaAaCcattactaCaaCC | 28 |
| 1109 | 1109_27 | TGAAACCATTACTACAACC | TGaAaCcattactacAACC | 10 |
| 1109 | 1109_28 | TGAAACCATTACTACAACC | TgAAaCcattactAcAACC | 52 |
| 1109 | 1109_29 | TGAAACCATTACTACAACC | TGaAAccattactacaACC | 16 |
| 1109 | 1109_30 | TGAAACCATTACTACAACC | TGAaaccattactacaaCC | 36 |
| 1109 | 1109_31 | TGAAACCATTACTACAACC | TgaaaCcattactaCaACC | 21 |
| 1109 | 1109_32 | TGAAACCATTACTACAACC | TgAAAccattactacAACC | 9 |
| 1109 | 1109_33 | TGAAACCATTACTACAACC | TgAaaCcattactacAaCC | 14 |
| 1109 | 1109_34 | TGAAACCATTACTACAACC | TGaaaccattactacaACC | 43 |
| 1109 | 1109_35 | TGAAACCATTACTACAACC | TgAAaCcattactacaACC | 15 |
| 1109 | 1109_36 | TGAAACCATTACTACAACC | TgaAACcattactacaaCC | 15 |
| 1109 | 1109_37 | TGAAACCATTACTACAACC | TGaAaCcattactacaaCC | 16 |
| 1109 | 1109_38 | TGAAACCATTACTACAACC | TGaaaCcattactacaaCC | 38 |
| 1109 | 1109_39 | TGAAACCATTACTACAACC | TgAAACcattactacaaCC | 14 |
| 1109 | 1109_40 | TGAAACCATTACTACAACC | TgAAaCcattactacaaCC | 16 |
| 1109 | 1109_41 | TGAAACCATTACTACAACC | TgaAaCcattactacaaCC | 28 |
| 1109 | 1109_42 | TGAAACCATTACTACAACC | TgaaACcattactacaaCC | 28 |
| 1122 | 1122_44 | AATCTTATTTACATCTTCC | AatcttatttacaTCTtCC | 65 |
| 1122 | 1122_45 | AATCTTATTTACATCTTCC | AatcTtatttacAtCttCC | 38 |
| 1122 | 1122_46 | AATCTTATTTACATCTTCC | AatcTtatttacaTcTTCC | 34 |
| 1122 | 1122_47 | AATCTTATTTACATCTTCC | AAtCttatttacAtcTtCC | 10 |
| 1122 | 1122_48 | AATCTTATTTACATCTTCC | AAtcTtatttacATcTtCC | 35 |
| 1122 | 1122_49 | AATCTTATTTACATCTTCC | AatCttatttacAtcTtCC | 10 |
| 1122 | 1122_50 | AATCTTATTTACATCTTCC | AAtCttatttacAtcttCC | 11 |
| 1122 | 1122_51 | AATCTTATTTACATCTTCC | AAtctTatttacatCTtCC | 9 |
| 1122 | 1122_52 | AATCTTATTTACATCTTCC | AatcTTatttacAtcTtCC | 12 |
| 1122 | 1122_53 | AATCTTATTTACATCTTCC | AatctTatttacatCTtCC | 8 |
| 1122 | 1122_54 | AATCTTATTTACATCTTCC | AaTcTtatttacatcTTCC | 4 |
| 1122 | 1122_55 | AATCTTATTTACATCTTCC | AAtcttatttacAtcTtCC | 27 |
| 1122 | 1122_56 | AATCTTATTTACATCTTCC | AAtCtTatttacAtcttCC | 5 |
| 1122 | 1122_57 | AATCTTATTTACATCTTCC | AAtcTTatttacatcttCC | 14 |
| 1122 | 1122_58 | AATCTTATTTACATCTTCC | AaTCttatttacatcttCC | 13 |
| 1122 | 1122_59 | AATCTTATTTACATCTTCC | AATcttatttacatCttCC | 6 |
| 1122 | 1122_60 | AATCTTATTTACATCTTCC | AAtcTtatttacatCttCC | 10 |
| 1122 | 1122_61 | AATCTTATTTACATCTTCC | AAtcTTatttacatcTtCC | 6 |
| 1122 | 1122_62 | AATCTTATTTACATCTTCC | AatCtTatttacatcTtCC | 3 |
| 1122 | 1122_63 | AATCTTATTTACATCTTCC | AATCttatttacaTcttCC | 5 |
| 1122 | 1122_64 | AATCTTATTTACATCTTCC | AatCttatttacatcTtCC | 7 |
| 1122 | 1122_65 | AATCTTATTTACATCTTCC | AatCttatttacatcttCC | 32 |
| 1122 | 1122_66 | AATCTTATTTACATCTTCC | AatcttatttacatcTTCC | 19 |
| 1122 | 1122_67 | AATCTTATTTACATCTTCC | AATCttatttacatcTtCC | 3 |
| 1122 | 1122_68 | AATCTTATTTACATCTTCC | AATcTtatttacatcTtCC | 4 |
| 1122 | 1122_69 | AATCTTATTTACATCTTCC | AAtCTtatttacatcTtCC | 3 |
| 1122 | 1122_70 | AATCTTATTTACATCTTCC | AAtCtTatttacatcTtCC | 3 |
| 1122 | 1122_71 | AATCTTATTTACATCTTCC | AAtcTtatttacatcTtCC | 13 |
| 1122 | 1122_72 | AATCTTATTTACATCTTCC | AaTCttatttacatcTtCC | 5 |
| 1122 | 1122_73 | AATCTTATTTACATCTTCC | AatCTtatttacatcTtCC | 5 |
| 1122 | 1122_74 | AATCTTATTTACATCTTCC | AatctTatttacatcTtCC | 10 |
| 1122 | 1122_75 | AATCTTATTTACATCTTCC | AAtCTtatttacatctTCC | 3 |
| 1122 | 1122_76 | AATCTTATTTACATCTTCC | AAtCttatttacatctTCC | 5 |
| 1122 | 1122_77 | AATCTTATTTACATCTTCC | AaTCttatttacatctTCC | 5 |
| 1122 | 1122_78 | AATCTTATTTACATCTTCC | AatCTtatttacatctTCC | 4 |
| 1122 | 1122_79 | AATCTTATTTACATCTTCC | AAtCTtatttacatcttCC | 7 |
| 1122 | 1122_80 | AATCTTATTTACATCTTCC | AAtCtTatttacatcttCC | 5 |
| 1122 | 1122_81 | AATCTTATTTACATCTTCC | AatCtTatttacatcttCC | 8 |
| 1109 | 1109_43 | TGAAACCATTACTACAACC | TgAAaccattacTAcAaCC | 18 |
| 1109 | 1109_44 | TGAAACCATTACTACAACC | TgAaAccattacTacAaCC | 27 |
| 1109 | 1109_45 | TGAAACCATTACTACAACC | TgaAaCcattacTacAaCC | 65 |
| 1109 | 1109_46 | TGAAACCATTACTACAACC | TgAaaccattacTacaACC | 25 |
| 1109 | 1109_47 | TGAAACCATTACTACAACC | TgaAaccattacTacaACC | 35 |
| 1109 | 1109_48 | TGAAACCATTACTACAACC | TgaaAccattacTacaACC | 48 |
| 1109 | 1109_49 | TGAAACCATTACTACAACC | TgaAaCcattacTacaaCC | 44 |
| 1109 | 1109_50 | TGAAACCATTACTACAACC | TgaAaccattacTaCaaCC | 34 |
| 1109 | 1109_51 | TGAAACCATTACTACAACC | TGaaaccattacTacaACC | 29 |
| 1109 | 1109_52 | TGAAACCATTACTACAACC | TgAAaccattacTacaACC | 23 |
| 1109 | 1109_53 | TGAAACCATTACTACAACC | TgaaaCcattacTaCaaCC | 39 |
| 1109 | 1109_54 | TGAAACCATTACTACAACC | TGaaaccattactaCaaCC | 33 |
| 1109 | 1109_55 | TGAAACCATTACTACAACC | TgAaAccattactaCaaCC | 29 |
| 1109 | 1109_56 | TGAAACCATTACTACAACC | TGaaAccattactacAACC | 16 |
| 1109 | 1109_57 | TGAAACCATTACTACAACC | TGaaAccattactacAaCC | 18 |
| 1109 | 1109_58 | TGAAACCATTACTACAACC | TgAaACcattactacaaCC | 12 |
| 1109 | 1109_59 | TGAAACCATTACTACAACC | TgAaaccattactaCAaCC | 13 |
| 1109 | 1109_60 | TGAAACCATTACTACAACC | TgaaAccattactACaaCC | 36 |
| 1109 | 1109_61 | TGAAACCATTACTACAACC | TGaaaccattactAcaACC | 34 |
| 1109 | 1109_62 | TGAAACCATTACTACAACC | TgAaaCcattactACaaCC | 43 |
| 1109 | 1109_63 | TGAAACCATTACTACAACC | TGaAAccattactaCaaCC | 19 |
| 1109 | 1109_64 | TGAAACCATTACTACAACC | TGaaaCcattactACaaCC | 29 |
| 1109 | 1109_65 | TGAAACCATTACTACAACC | TGaAaccattactAcaaCC | 40 |
| 1109 | 1109_66 | TGAAACCATTACTACAACC | TgaAAccattactAcAACC | 14 |
| 1109 | 1109_67 | TGAAACCATTACTACAACC | TGaAaccattactAcAaCC | 14 |
| 1109 | 1109_68 | TGAAACCATTACTACAACC | TGaaaCcattactAcAaCC | 27 |
| 1109 | 1109_69 | TGAAACCATTACTACAACC | TgAaaCcattactAcAACC | 31 |
| 1109 | 1109_70 | TGAAACCATTACTACAACC | TgAaAccattactAcAaCC | 24 |
| 1109 | 1109_71 | TGAAACCATTACTACAACC | TgaaACcattactacAACC | 10 |
| 1109 | 1109_72 | TGAAACCATTACTACAACC | TGAaaccattactacAaCC | 11 |
| 1109 | 1109_73 | TGAAACCATTACTACAACC | TgaAACcattactAcAaCC | 34 |
| 1109 | 1109_74 | TGAAACCATTACTACAACC | TGaAaCcattactacaACC | 15 |
| 1109 | 1109_75 | TGAAACCATTACTACAACC | TGaaACcattactacaaCC | 14 |
| 1109 | 1109_76 | TGAAACCATTACTACAACC | TGaAaccattactaCaaCC | 22 |
| 1109 | 1109_77 | TGAAACCATTACTACAACC | TgaAAccattactaCaaCC | 30 |
| 1109 | 1109_78 | TGAAACCATTACTACAACC | TgaaAccattactaCaaCC | 50 |
| 1109 | 1109_79 | TGAAACCATTACTACAACC | TgaAACcattactacAaCC | 9 |
| 1109 | 1109_80 | TGAAACCATTACTACAACC | TGaAaccattactacaaCC | 31 |
| 1109 | 1109_81 | TGAAACCATTACTACAACC | TgAaaCcattactacaaCC | 31 |

In the oligonucleotide compound column, capital letters represent beta-D-oxy LNA nucleosides, LNA cytosines are 5-methyl cytosine, lower case letters are DNA nucleosides, and all internucleoside linkages are phosphorothioate.

### Example 5: Testing in vitro efficacy of LNA oligonucleotides in iCell® GlutaNeurons at 25 μM

### Materials and Methods:

An oligonucleotide screen was performed in a human cell line using selected LNA oligonucleotides from the previous examples.

The iCell® GlutaNeurons derived from human induced pluripotent stem cell were purchased from the vendor listed in Table 2, and were maintained as recommended by the supplier in a humidified incubator at 37° C. with 5% CO₂. For the screening assays, cells were seeded in 96 multi well plates in media recommended by the supplier (see Table 2 in the Materials and Methods section). The number of cells/well was optimized (Table 2).

Cells were grown for 7 days before addition of the oligonucleotide in concentration of 25 μM (dissolved in medium). 4 days after addition of the oligonucleotide, the cells were harvested.

RNA extraction and qPCR was performed as described for “Example 1”

Primer assays for ATXN3 and house keeping gene were:

ATXN3 primer assay (Assay ID: N/A, Item Name:
Hs.PT.58.39355049):
(SEQ ID NO: 1128)
Forward primer: GTTTCTAAAGACATGGTCACAGC
(SEQ ID NO: 1129)
Reverse: CTATCAGGACAGAGTTCACATCC
(SEQ ID NO: 1030)
Probe: 56-FAM/AAAGGCCAG/ZEN/CCACCAGTTCAGG/3IABkFQ/
TBP primer assay (Assay ID: N/A, Item name:
Hs.PT.58v.39858774
(SEQ ID NO: 1131)
Probe: 5′-/5HEX/TGA TCT TTG/ZEN/CAG TGA CCC
AGC ATC A/3IABkFQ/-3′
(SEQ ID NO: 1132)
Primer 1: 5′-GCT GTT TAA CTT CGC TTC CG-3′
(SEQ ID NO: 1133)
Primer 2: 5′-CAG CAA CTT CCT CAA TTC CTT G-3′

### Results:

The relative ATXN3 mRNA expression levels were determined as % of control (medium-treated cells) i.e. the lower the value the larger the inhibition.

The compounds tested and the target knock-down data is presented in Table 7.

### Example 6: Determination of EC50 values of LNA gapmers targeting ATXN3

### Materials and Methods:

Values for EC50 (concentration at which half effect on target knockdown is observed) was determined for the cell lines SK-N-AS, A431 and iPSCs (iCell® GlutaNeurons). The following oligoconcentrations were used:
SK-N-AS: 50 μM—half log dilution (3.16 fold)—8 steps including blank control
A431: 50 μM—half log dilution (3.16 fold)—8 steps including blank control
iPCS: 10 μM—10 fold dilution—8 steps including blank control

The cells were treated with oligo, lysed and analysed as indicated in previous examples.

### Results:

The compounds tested and their EC50 values is shown in table 7.

### Example 7: In vitro toxicity evaluation

### Materials and Methods:

The criterion for selection of oligonucleotides assessed in the various safety assays is based on the magnitude and frequency of signals obtained. Safety assays used were: Caspase activation, hepatotoxicity, nephrotoxicity toxicity and immunotoxicity assays. The signals obtained in the individual in vitro safety assays result in a score (0-safe, 0.5 borderline toxicity, 1-mild toxicity, 2-medium toxicity and 3-severe toxicity) and are summarized into a cumulative score for each sequence (See table 7), providing an objective ranking of compounds. As reported in the references provided, the signal strength is a measure of risk for in vivo toxicity based on validation of the assays using in vivo relevant reference molecules

In vitro toxicity assays were performed as described in the following references: Caspase activation assay: Dieckmann et al., Molecular Therapy: Nucleic Acids Vol. 10 Mar. 2018, pp 45-54.

Hepatotoxicity toxicity assay: Sewing et al., Methods in Molecular Biology Oligonucleotide-Based Therapies MIMB, volume 2036, pp 249-259 2019, Sewing et al., PLOS ONE DOI:10.1371/journal.pone.0159431 Jul. 21, 2016.

Nephrotoxicity toxicity assay: Moisan et al., Mol Ther Nucleic Acids. 2017 Mar. 17; 6:89-105. doi: 10.1016/j.omtn.2016.11.006. Epub 2016 Dec. 10.

Immunotoxicity: Sewing et al., PLoS One. 2017 Nov. 6;12(11):e0187574. doi: 10.1371/journal.pone.0187574. eCollection 2017.

As part of the screening cascade 1170 compounds were evaluated in the cell lines SK-N-AS and A431 where compound efficacy was evaluated (Tables 4-6). Of these, 50 of the most effective compounds were evaluated for caspase activation of which 18 underwent further evaluation in the descried in the three other in vitro tox assays (cumulative score is shown in Table 7).

### Results:

Conclusively, 8 compounds were identified as being highly effective and potent in vitro, and with a low or absent toxicity in the 4 in vitro assays these compounds were therefore selected for evaluated in transgenic mice expressing human ATNX3 pre-mRNA: Compounds #1856_1, 1813_1, 1812_1, 1809_2, 1607_1, 1122_62, 1122_67 and 1122_33.

TABLE 7
Data obtained from examples 5, 6 & 7

| | | | | | HiPCS, Maximal |
|---|---|---|---|---|---|
| | Total | SK-N-AS | A-431 | HiPSC | efficacy at 25 μM |
| | tox | EC50 | EC50 | EC50 | (% remaining |
| CMPID | score | (μM) | (μM) | (μM) | ATXN3 transcript) |
| 1856_1 | 1.5 | 0.53 | 0.22 | 0.23 | 2.87 |
| 1806_2 | 2 | 0.35 | 0.19 | 0.03 | 0.91 |
| 1888_1 | — | 0.72 | 0.54 | — | |
| 1813_1 | 2 | 0.24 | 0.08 | 0.04 | 1.85 |
| 1640_1 | — | 1.50 | 0.19 | — | |
| 1812_1 | 1.5 | 0.20 | 0.09 | 0.09 | 0.59 |
| 1117_2 | — | 0.73 | 0.57 | — | |
| 1810_1 | — | 0.36 | 0.14 | — | |
| 1809_2 | 1.25 | 0.22 | 0.09 | 0.05 | 1.44 |
| 1489_1 | — | 1.16 | 0.30 | — | |
| 1867_1 | — | 0.54 | 0.50 | — | |
| 1893_1 | — | 0.95 | 0.34 | 0.41 | 4 |
| 1906_1 | — | 0.36 | 0.57 | 0.04 | 2.55 |
| 1214_1 | — | 1.05 | 0.38 | — | |
| 1213_1 | — | 1.01 | 0.38 | — | |
| 1423_1 | — | 0.75 | 0.23 | 0.03 | 3.58 |
| 1790_1 | — | 0.42 | 0.47 | — | |
| 1605_1 | — | 0.47 | 0.17 | — | |
| 1607_1 | 2.5 | 0.32 | 0.25 | 0.08 | 4.46 |
| 1805_1 | — | 0.75 | 0.23 | — | |
| 1806_1 | — | 0.45 | 0.20 | 0.04 | 1.3 |
| 1809_1 | 3 | 0.24 | 0.20 | 0.02 | 1.81 |
| 1808_1 | 2 | 0.26 | 0.22 | 0.06 | 1.4 |
| 1625_1 | 0.5 | 0.94 | 0.25 | 0.66 | 7.16 |
| 1122_54 | — | 0.62 | 0.15 | — | |
| 1122_16 | — | 0.30 | 0.15 | — | |
| 1122_17 | — | 0.33 | 0.17 | 0.11 | 1.07 |
| 1122_62 | 0.5 | 0.21 | 0.10 | 0.03 | 3.53 |
| 1122_19 | — | 0.28 | 0.24 | — | |
| 1122_23 | — | 0.54 | 0.18 | 0.05 | 0.59 |
| 1122_67 | 0 | 0.29 | 0.10 | 0.01 | 0.52 |
| 1122_68 | — | 0.28 | 0.13 | 0.01 | |
| 1122_69 | — | 0.27 | 0.12 | — | |
| 1122_70 | — | 0.20 | 0.10 | — | |
| 1122_27 | 1 | 0.23 | 0.12 | 0.03 | 0.55 |
| 1122_72 | 0.5 | 0.25 | 0.15 | 0.06 | 2.28 |
| 1122_28 | 1 | 0.20 | 0.12 | 0.01 | 0.37 |
| 1122_29 | — | 0.19 | 0.09 | 0.02 | 1.6 |
| 1122_73 | — | 0.29 | 0.18 | 0.04 | 1.59 |
| 1122_75 | 1 | 0.44 | 0.12 | 0.03 | 2 |
| 1122_76 | — | 0.33 | 0.19 | — | |
| 1122_77 | 1 | 0.30 | 0.20 | 0.04 | 1.97 |
| 1122_78 | — | 0.29 | 0.18 | 0.02 | 1.91 |
| 1122_33 | 1.25 | 0.18 | 0.10 | 0.02 | 1.84 |
| 1122_37 | — | 0.25 | 0.13 | 0.03 | 0.89 |
| 1122_80 | — | 0.33 | 0.17 | — | |
| 1122_41 | — | 0.24 | 0.16 | 0.01 | 0.47 |
| 1109_22 | — | 0.90 | 0.23 | 0.11 | 8.41 |
| 1109_32 | 0 | 0.75 | 0.17 | 0.09 | 3.49 |
| 1109_79 | — | 1.48 | 0.20 | — | |

### Example 8: In vivo transgenic mouse study

### Materials and Methods:

Animal Care

In vivo activity and tolerability of the compounds were tested in 10-13 week old B6;CBA-Tg(ATXN3*)84.2Cce/IbezJ male and female mice (JAX®. W, Mice, The Jackson Laboratory) housed 3-5 per cage. The mice are transgenic mice which express the human ATXN3 pre-mRNA sequence, with 84 CAG repeats motif, an allele which is associated with MJD in humans). Animals were held in colony rooms maintained at constant temperature (22±2° C.) and humidity (40+80%) and illuminated for 12 hours per day (lights on at 0600 hours). All animals had ad libitum access to food and water throughout the studies. All procedures are performed in accordance with the respective Swiss regulations and approved by the Cantonal Ethical Committee for Animal Research.

Administration Route—Intra-cisterna magna injections.

The compounds were administered to mice by intra cisterna magna (ICM) injections. Prior to ICM injection the animals received 0.05 mg/kg Buprenorphine dosed sc as analgesia. For the ICM injection animals were placed in isofluran. Intracerebroventricular injections were performed using a Hamilton micro syringe with a FEP catheter fitted with a 36 gauge needle. The skin was incised, muscles retracted and the atlanto-occipital membrane exposed. Intracerebroventricular injections were performed using a Hamilton micro syringe with a catheter fitted with a 36 gauge needle. The 4 microliter bolus of test compound or vehicle was injected over 30 seconds. Muscles were repositioned and skin closed with 2-3 sutures. Animals were placed in a varm environment until they recovered from the procedure.

Two independent experiments were performed with groups of different compounds as shown in Table 8A.

TABLE 8A

| Compound ID | Dose, gg | Time-point | Group Size |
|---|---|---|---|
| Saline only | 0 | 4 wk | 6 |
| 1856_1 | 250 | 4 wk | 8 |
| 1813_1 | 250 | 4 wk | 8 |
| 1812_1 | 250 | 4 wk | 8 |
| 1809_2 | 250 | 4 wk | 8 |
| 1607_1 | 250 | 4 wk | 8 |
| 1122_62 | 250 | 4 wk | 8 |
| 1122_67 | 250 | 4 wk | 8 |
| 1122_33 | 250 | 4 wk | 8 |

Tolerability Results:

All compounds were found to be tolerated up to the 4 weeks timepoint. Acute toxicity was measured by monitoring the animal's behavior as described in WO2016/126995 (see example 9). Sub-acute toxicity was measured by monitoring the body weight of each animal during the time course of the experiment, with >5% weight reduction indicative of sub-acute toxicity. In some groups 1 or 2 animals did show some distress after the ICM administration and were euthanized, but this was likely to be due to the procedure rather than a adverse toxicity of any of the compounds. All eight compounds were therefore considered to be well tolerated in vivo.

4 weeks post administration, the animals were sacrificed, and tissues from the cortex, midbrain, cerebellum, hippocampus pons/medulla and striatum were collected weighed and snap frozen in liquid N2 directly after sampling. Samples were stored on dry ice until storage at −80° C.

Analysis of in vivo samples. Description of tissue preparation for content measurement and qPCR.

Mouse tissue samples were homogenized in the MagNA Pure LC RNA Isolation Tissue Lysis Buffer (Roche, Indianapolis, Ind.) using a Qiagen TissueLyzer II. The homogenates were incubated for 30 minutes at room temperature for complete lysis. After lysis the homogenates were centrifuged for 3 minutes at 13000 rpm and the supernatant used for analysis. Half was set aside for bioanalysis and for the other half, RNA extraction was continued directly.

Oligo Content Analysis

For bioanalysis, the samples were diluted 10-50 fold for oligo content measurements with a hybridization ELISA method. A biotinylated LNA-capture probe and a digoxigenin-conjugated LNA-detection probe (both 35 nM in 5×SSCT, each complementary to one end of the LNA oligonucleotide to be detected) was mixed with the diluted homogenates or relevant standards, incubated for 30 minutes at RT and then added to a streptavidine-coated ELISA plates (Nunc cat. no. 436014).

The plates were incubated for 1 hour at RT, washed in 2×SSCT (300 mM sodium chloride, 30 mM sodium citrate and 0.05% v/v Tween-20, pH 7.0) The captured LNA duplexes were detected using an anti-DIG antibodies conjugated with alkaline phosphatase (Roche Applied Science cat. No. 11093274910) and an alkaline phosphatase substrate system (Blue Phos substrate, KPL product code 50-88-00). The amount of oligo complexes was measured as absorbance at 615 nm on a Biotek reader.

Data was normalized to the tissue weight and expressed as nM of oligo.

mRNA Analysis

RNA was purified from 350 μL of supernatant using the MagNA Pure 96 instrument using the kit Cellular RNA Large Volume Kit (Roche, Indianapolis, Ind.). RNA samples were normalized to 2 ng/μL in RNase-Free water and stored at −20° C. until further use.

For one-step qPCR (cDNA synthesis and qPCR), each sample was run in duplicates with four probe sets (IDT, Leuven, Belgium) run in duplex.

To each reaction 44 of previously diluted RNA, 0.54 of water and 5.54 of TaqMan MasterMix was added. Plates were centrifuged and heat-chocked at 90° C. for 40sek followed by a short incubation on ice before analyzing the samples using qPCR (Incubation at 50° C. for 15 minutes and 90° C. for 3 minutes followed by 40 cycles at 95° C. for 5 sec and 60° C. for 45 sec). Assay probes are described below.

Data was analyzed using the relative standard curve method where each is first normalized to the housekeeping gene (RPL4) and then expressed as percent of untreated control animals.

qPCR assays for in vivo studies:
Human ATXN3, qPR assay: (ATXN3_exon_8-9(1)
PrimeTime ® XL qPCR Assay (IDT).
qPCR probe and primers:
(SEQ ID NO: 1134)
Probe: 5′-/56-FAM/CTCCGCAGG/ZEN/GCT ATTCAGCT AAGT/
31ABkFQ/-3′
(SEQ ID NO: 1135)
Primer 1: 5′-AGT AAGATTTGT ACCTGATGTCTGT-3′
(SEQ ID NO: 1136)
Primer 2: 5′-CATGGAAGATGAGGAAGCAGAT-3′
House keeping gene used:
Mouse RPL4, qPCR assay (Mm.PT.58.17609218)
PrimeTime ® XL qPCR Assay (IDT).
qPCR probe and primers:
(SEQ ID NO: 1090)
Probe: 5′-/5HEX/CTG AAC AGC/ZEN/CTC CTT GGT CTT
CTT GTA/3IABkFQ/-3′
(SEQ ID NO: 1091)
Primer 1: 5′-CTT GCC AGC TCT CAT TCT CTG-3′
(SEQ ID NO: 1092)
Primer 2: 5′- TGG TGG TTG AAG ATA AGG TTG A-3′

### Results:

The results are shown in Table 8B.

All compounds tested gave efficacious target inhibition in the tissues tested and were tolerated at the doses tested. Compound 1122_33 across the compounds tested has either the best or second ranked highest specific activity (lower EC50) in all tissues, followed by 1122_62 and 1122_67.

Compounds 1122_67, 1607_1, 1813_1 and 1122_33 provided high efficacy in vivo in all tissues tested, illustrating a remarkable consistent inhibition of ATXN3 expression across the brain tissues tested. Based on an accumulative rank score compound 1122_67 was consistently either the best or second ranked compound in terms of efficacy of ATXN3 knock down in the tissues tested.

### Example 9: Testing in vitro efficacy of LNA oligonucleotides and Reference Compounds in a time course, dose range experiment in human iPSC-derived neurons

### Materials and Methods:

Compounds used: 1122_67 and 1813_1 & the following reference compounds disclosed in WO2019/217708, as referenced by the Compound ID numbers used in WO2019/217708: 1100673, 1101657, 1102130, 1103014 & 1102987. Compounds 1100673, 1101657, 1102130 are highlighted in WO2019/217708 as providing potent in vivo inhibition, compounds 1103014 and 1102987 were not evaluated in vivo in WO2019/217708, but are included as reference compounds due to the sequence similarity to compound 1122_67 (1103014) and 1813_1 (1102987).

The iCell® GlutaNeurons cells were prepared and maintained as described in Example 5 & Table 2. Cells were grown for 7 days before addition of the oligonucleotide in concentration of 0-10 μM (dissolved in medium).

Cells were harvested at 4 days, 6 days, 9 days, 12 days and 20 days after oligo treatment, and RNA extraction and qPCR was performed as described for “Example 1”, using the ATXN3 primar assay described in example 5. The relative ATXN3 mRNA expression levels were determined as % of control (medium-treated cells) i.e. the lower the value the larger the inhibition.

### Results:

The results are shown in Table 9.

TABLE 9

| | EC50 in hiPSC-derived neurons, nM | | | | |
|---|---|---|---|---|---|
| Compound | Day 4 | Day 6 | Day 9 | Day 12 | Day20 |
| 1122_67 | 7.2 | 1.3 | 1.4 | 1.1 | 1.1 |
| 1813_1 | 23 | 6.3 | 10 | 8.9 | 7.7 |
| 1100673 | 110 | 27 | 30 | 34 | 44 |
| 1101657 | 515 | 204 | 69 | 90 | 73 |
| 1102130 | 315 | 164 | 390 | 101 | 133 |
| 1103014 | 662 | 64 | 435 | 98 | 369 |
| 1102987 | 944 | 305 | 135 | 391 | 200 |

Compounds 1122_67 and 1813_1 were remarkably more potent than the 5 reference compounds, with compound 1122_67 being the most potent compound at all time points and both 1122_67 and 1813_1 gave a remarkably effective and long lasting inhibition of ATXN3 mRNA.

### Example 10: Comparative In vivo transgenic mouse study

### Materials and Methods:

A further in vivo study was performed at Charles River Laboratories Den Bosch B.V., Groningen, NL, using compound 1122_67 and 1813_1, and reference compound 1100673 (WO2019/217708). The study used male and female B6;CBA-Tg(ATXN3*)84.2Cce/IbezJ mice with the compounds administered via intracisternal (ICM) administration. At two timepoints after compound administration, 1 or 4 weeks, animals were euthanized and terminal plasma samples and tissues were collected.

Animal Care

In vivo activity and tolerability of the compounds were tested in 62 B6;CBA-Tg(ATXN3*)84.2Cce/IbezJ male and female mice (JAX® Mice, The Jackson Laboratory) at the age between 7-10 weeks. Following arrival, animals were housed in groups up to 5 in individually vented cages (IVC, 40×20×16 cm) in a temperature (22±2° C.) and humidity (55±15%) controlled environment on a 12 hour light cycle (07.00-19.00 h). Males and females were kept in separate cages. Standard diet (SDS Diets, RM1 PL) and domestic quality mains water were available ad libitum. If required, animals received soaked chow and/or Royal Canin in addition to Standard diet as part of pamper care. The experiments were conducted in strict accordance with the Guide for the Care and Use of Laboratory Animals (National Research Council 2011) and were in accordance with European Union directive 2010/63 and the Dutch law. The in vivo experiment described was performed at Charles River Laboratories Den Bosch B.V. location Groningen (Groningen, the Netherlands).

Administration Route-Intra-cisterna Magna injections.

The compounds were administered to mice by intra cisterna magna (ICM) injections. Mice were anesthetized using isoflurane (2.5-3% and 500 mL/min 02). Before surgery, Finadyne (1 mg/kg, s.c.) was administered for analgesia during surgery and the post-surgical recovery period. A mixture of bupivacaine and epinephrine was applied to the incision site and periost of the skull for local analgesia.

Animals were placed in a stereotaxic frame (Kopf instruments, USA) and an incision made at the back of the head towards the neck. Then, the skin was spread and the coordinates marked prior to drilling a hole in the occipital bone of the skull, where a cannula was placed. Next, the compounds were injected into the cisterna magna (ICM). A volume of 4 μL of the assigned test item was injected over 30 seconds. After injection, the needle and cannula were held in place for 30 seconds to ensure no back flow occurred. The cannula was then retracted, the hole was covered with skin and the incision was closed by sutures.

Animals were placed in a warm environment until recovered from the procedure.

Compound 1122_67 was administered at a single dose of 90, 150 or 250 μg, and compound 1813_1 was administered at a single dose of 150 μg or 250 μg. The reference compound 1100673 was administered at a single dose of 250 μg only.

From three days prior to ICM injections, up to one week after administration, animal's weight was registered daily. Animal's weight was monitored and registered at least twice a week for the rest of the experiment.

At the end of the experiment, on day 8 or 29 (1 or 4 weeks), the animals were euthanized by Euthasol® overdose. Terminal plasma was collected in Li-Hep tubes. Terminal tissues were harvested from the animals and were dissected on a chilled surface. Half of the tissue samples were stored in 2.0 mL Safe-Lock tubes, PCR clean, pre-weighted and precooled. Immediately after collection, samples were weighed and flash frozen in liquid N2 prior to storage at −80° C. The other half was fixed in 4% PFA for 72 hours and subsequently transferred to 70% ethanol awaiting shipment. Tissue dissection and collection was performed, collecting tissue from a range of tissues: Midbrain, Cortex, Striatum, Hippocampus, Cerebellum, Brainstem, and spinal cord (Cervical, Thoracic & Lumbar).

Tolerability Results:

Acute toxicity was measured by monitoring the animal's behavior as described in WO2016/126995 (see example 9). Chronic toxicity was measured by monitoring the body weight of each animal during the time course of the experiment, with >5% weight reduction indicative of chronic toxicity. In some groups 1 or 2 animals did show some distress after the ICM administration and were euthanized, but this was likely to be due to the nature of the surgical procedure rather than a adverse toxicity of any of the compounds.

There were signs of acute toxicity at the 250 μg dose of 1813_1 in 3 mice, leading to early euthanisation of this group of animals. Otherwise all compounds were found to be tolerated up to the 4 weeks timepoint.

After 4 weeks the animals were euthanised and brain and CNS tissue collected: Spinal cord, cortex, striatum, hippocampus, midbrain, brainstem and cerebellum as well as liver and kidney was collected in liquid nitrogen for drug concentration analysis an ATAXN3 mRNA analysis at 1 or 4 weeks following dosing.

Analysis of in vivo samples: Description of tissue preparation for content measurement and qPCR was performed as per Example 8. The EC50 was calculated, and maximum KD achieved recorded this data is provided in Table 10.

### Results:

Compound 1122_67 was the most effective compound in all brain tissues tested and gave an excellent effective knock-down in all brain tissues tested, indicating good bio-distribution to all key tissues (1813_1 was as effective as 1122_67 in spinal cord, brainstem and midbrain). Notably compound 1122_67 gave highly effective knock-down in cerebellum, a tissue which the reference compound 1100673 was notably less effective. A further key observation at the after 4 weeks of treatment is that the efficacy of 1122_67 was even further improved as compared to the 1 week timepoint in all brain tissues. Notably, the efficacy of the reference compound, 1100673 was notably lower at the 4 week stage vs. the 1 week timepoint, particularly in key cerebellum and cortex tissues. The long duration of action and high potency of 1122_67 indicates that this compound should require a less frequent administration in a therapeutic setting.

### Example 11: Compound Stability to SVPD

### Materials and Methods:

3′—exonuclease snake venom phosphodiesterase I (SVP) (Art. No. LS003926, Lot. No. 58H18367) was purchased by Worthington Biochemical Corp. (Lakewood, N.Y., USA). The reaction mix for the 3′—exonuclease snake venom phosphodiesterase I (SVP) assay consisted of 50 mM TRIS/HCl pH 8 buffer, 10 mM MgCl2, 30 U CIP (NEB, Ipswich, Mass., USA), 0.02 U SVP and the oligonucleotide compound. The stability of the ASOs against SVPD was determined by performing the nuclease assays over a one day time course. In each reaction mix an amount about 0.2 mg/mL ASO in a totaly volume of 150 μl was used.

The incubation period of 24 h at 37° C. was performed on an autosampler, the SVPD and reactions and the ASO stabilities were monitored in time intervals by an UHPLC system equipped with a diode-array detector and coupled with electrospray ionization-time of flight mass spectrometry (ESI-ToF-MS). To generate the t=0 h time point, the enzyme was added into the reaction mix, directly before the first injection. Further injections took place at regular intervals over a period of 24 hours.

Compounds tested, 1122_67, 1813_1 and the reference compounds 1100673, 1101657, 1102130, 1103014, and 1102987.

### Results:

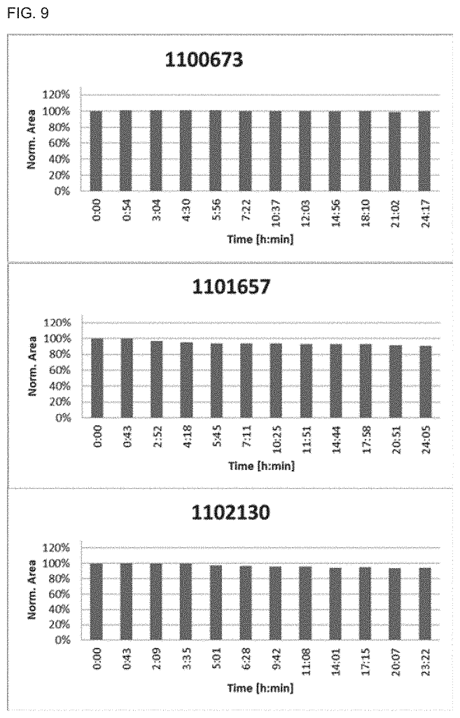

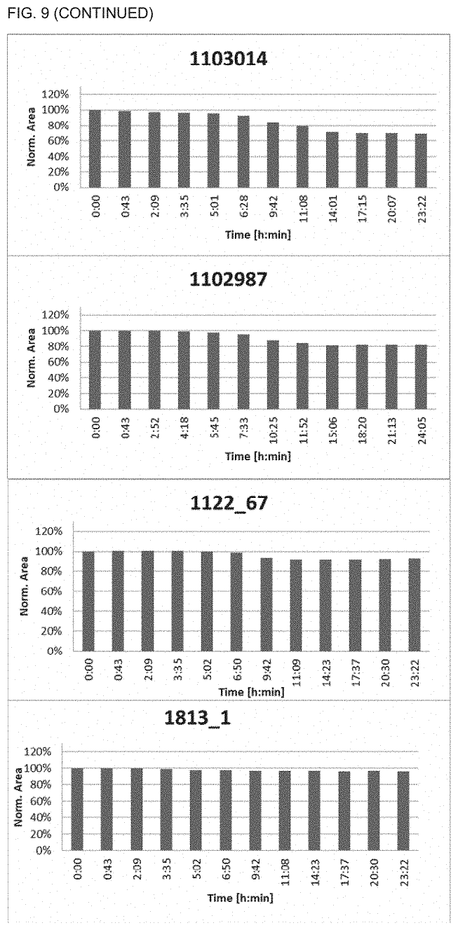

The data is illustrated in FIG. 9. Whilst the three highlighted reference compounds from WO2019/217708 and the 1122_67 and 1813_1 compounds had good stability in the SVPD assay, the 2 reference compounds from WO2019/217708 with the closest sequence to 1122_67 and 1813_1, compounds 1103014 and 1102987 were notably more vulnerable to SVPD degradation as compared to 1122_67 and 1813_1.

### Example 12: WT and polyQ Ataxin 3 protein levels in human SCA3 patient derived fibroblasts treated with selected oligonucleotides (ASO)

### Materials and Methods:

This experiment was performed to investigate the efficacy of effiacty of knock down of the LNA oligonucleotides, 1122_67 and 1122_33, as compared to the prior art compounds, 1100673 and 1102130 in SCA3 patient derived fibroblasts, allowing for an assessment of the efficacy on the disease causing ataxin3 allele and the ataxin3 WT allele.

Cell line used for the ASO treatment, human SCA3 patient derived fibroblasts (GM06153 Coriell Institute). One hundred thousand cells were seeded per well in a 24 well plate with a total volume of 1 ml. ASOs were added immediately after to a final concentration of 10 μM (gymnotic uptake). After 4 days of incubation at, cells were washed twice with PBS, and harvested in 200 μl RIPA buffer (Thermo Scientific, Pierce).

Western blots were performed on the capillary-based immunoassay platform (WES, ProteinSimple) using a WES 12-230 kDa Wes Separation Module. Cell lysate were diluted 10×in Sample load buffer (ProteinSimple) prior loading on the cartridge. Primary antibody for Ataxin 3 (rabbit monoclonal antibody, prod. #702788 from Invitrogen) and for HPRT (rabbit monoclonal antibody, cat. #Ab109021 from Abcam). Both antibodies were used in 1/100 dilutions. Goat anti-rabbit HRP conjugate (Part. #DM-001, ProteinSimple) was used as secondary antibody.

Compass software (ProteinSimple) was for quantification of the protein bands.

### Results:

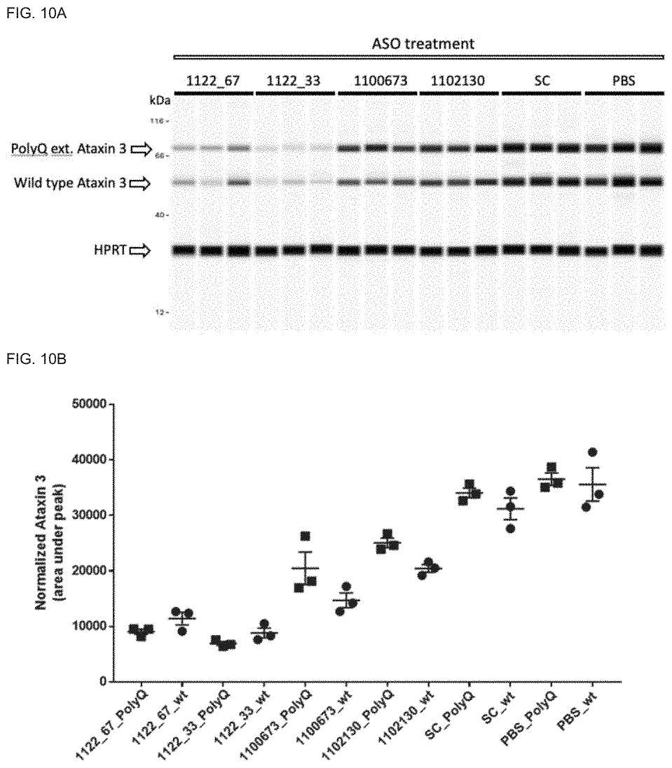

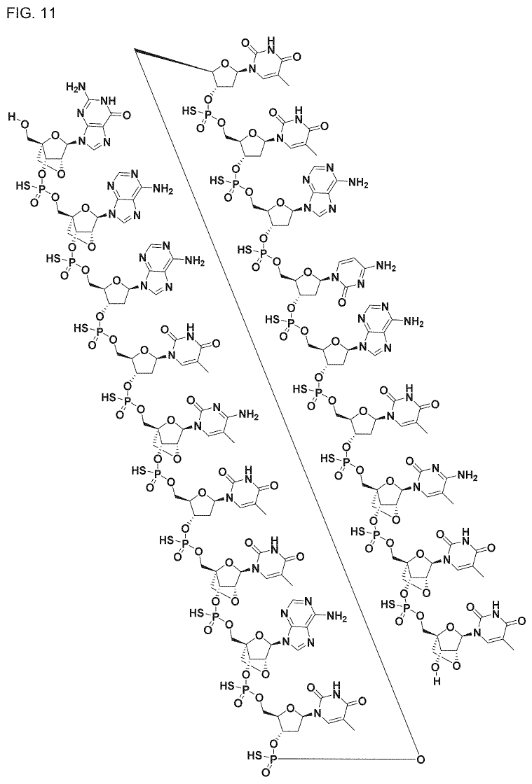

To show an efficient KD of both the wild type as well as the polyQ extended Ataxin 3 protein, GM06153 cells were treated with 10 uM of ASO for four days prior to protein analysis on the WES. Ataxin 3 antibody recognize both isoforms, and the intensity (area under peak) was normalized to the protein input based on the signal from HPRT. As seen from the FIGS. 10A and B, we observe that upon treatment with 1122_67 and 1122_33, there is an increased reduction in the polyQ extended Ataxin 3 compared to the wild type Ataxin 3. This trend is not observed for the other ASOs (Scrambled control, 1100673 or 1102130) where we observe a higher amount of the polyQ extended Ataxin 3, compared to the wild type Ataxin 3. A higher activity on the disease causing polyQ extended Ataxin 3 than the WT Ataxin 3 is preferable as it allows a selective reduction of the disease causing allele.

### Example 13: Redesign library

### Materials and Methods:

Compound ID Nos. 1122_67 and 1122_33 were used as parents in a redesign library. The compounds in the redesign library differed from the parent compounds and each other in intemucleoside linkages and/or the nucleosides used. In general, nucleosides at specific positions were varied between LNA, DNA and nucleoside analogs with different modifications in the 2′ position in the ribose. The intemucleoside linkages were otherwise phosphorothioate linkages. In some compounds, the chirality was controlled by use of phosphorothioate linkages providing for sterodefined backbones. The modifications employed are illustrated below.

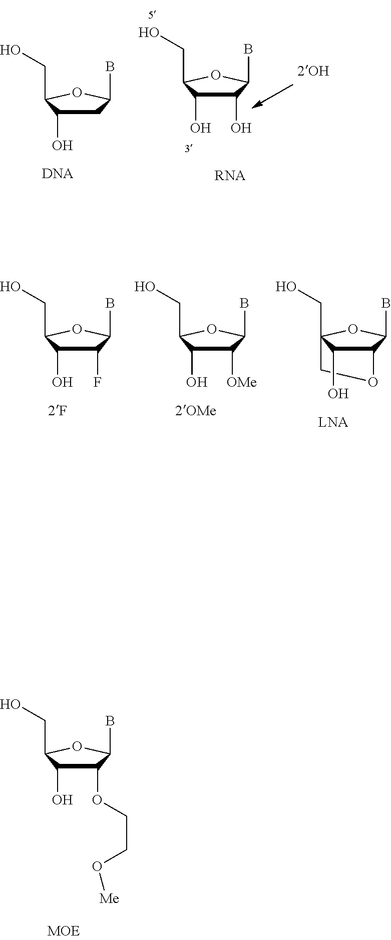

The compounds of the redesign library are listed in Table 11, where the structure of each compound is described by the hierarchical editing language for macromolecules (HELM) (for details, see Zhang et al., Chem. Inf. Model. 2012, 52, 10, 2796-2806) using the following HELM annotation keys:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[sP] is a phosphorothioate internucleoside linkage (stereo undefined)
[ssP] is a stereodefined Sp phosphorothioate internucleoside linkage
[mR](G) is a 2′-O-methyl guanine nucleoside,
[mR](U) is a 2′-O-methyl uracil nucleoside,
[mR](A) is a 2′-O-methyl adenine nucleoside,
[mR](C) is a 2′-O-methyl cytosine nucleoside,
[MOE](G) is a 2′-O-methoxyethyl guanine nucleoside,
[MOE](T) is a 2′-O-methoxyethyl thymine nucleoside,
[MOE](A) is a 2′-O-methoxyethyl adenine nucleoside,
[MOE]([5meC]) is a 2′-O-methoxyethyl 5-methyl cytosine nucleoside,
[fR](G) is a 2′-fluoro guanine nucleoside,
[fR](U) is a 2′-fluoro uracil nucleoside,
[fR](A) is a 2′-fluoro adenine nucleoside,
[fR](C) is a 2′-fluoro cytosine nucleoside.

### Results:

In Table 11, a compound with a compound ID number “1122_” or “1816_” is a modified version of SEQ ID NO:1122 or SEQ ID NO:1816, respectively.

TABLE 11
Compound and HELM table

| CMPIDNO | HELM |
|---|---|
| 1122_82 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[MOE]([5meC])[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_83 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[mR](U)[sP]•[LR]([5meC])[sP]• |
| | [LR]([5meC]) |
| 1122_84 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[mR](U)[sP]•[LR](T)[sP]•[LR]([5meC])[sP]• |
| | [LR]([5meC]) |
| 1122_85 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[mR](U)[sP]•[dR](C)[sP]•[dR](T)[sP]•[LR](T)[sP]•[LR]([5meC])[sP]• |
| | [LR]([5meC]) |
| 1122_86 | [LR](A)[sP]•[dR](A)[sP]•[dR](T)[sP]•[LR]([5meC])[sP]•[dR](T)[sP]•[mR](U)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_87 | [LR](A)[sP]•[dR](A)[sP]•[dR](T)[sP]•[LR]([5meC])[sP]•[mR](U)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]• [dR](T)[sP]•[LR](T)[sP]•[LR]([5meC])[sP]• |
| | [LR]([5meC]) |
| 1122_88 | [LR](A)[sP]•[dR](A)[sP]•[mR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]• [dR](T)[sP]•[LR](T)[sP]•[LR]([5meC])[sP]• |
| | [LR]([5meC]) |
| 1122_89 | [LR](A)[sP]•[dR](A)[sP]•[dR](T)[sP]•[LR]([5meC])[sP]•[dR](T)[sP]•[LR](T)[sP]• |
| | [dR](A)[sP]•[mR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_90 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[mR](U)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_91 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[mR](U)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_92 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[MOE]([5meC])[sP]•[dR](T)[sP]•[LR](T)[sP]• |
| | [LR]([5meC])[sP]•[LR]([5meC]) |
| 1122_93 | [MOE](A)[sP]•[MOE](A)[sP]•[MOE](T)[sP]•[MOE]([5meC])[sP]•[MOE](T)[sP]• |
| | [MOE](T)[sP]•[MOE](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[MOE]([5meC])[sP]•[MOE](T)[sP]•[MOE](T)[sP]• |
| | [MOE]([5meC])[sP]•[MOE]([5meC]) |
| 1122_94 | [MOE](A)[sP]•[MOE](A)P•[MOE](T)P•[MOE]([5meC])P•[MOE](T)P•[MOE](T)P•[MOE](A)[sP]• |
| | [dR](T)[sP]•[dR](T)[sP]•[dR](T)[sP]•[dR](A)[sP]•[dR](C)[sP]•[dR](A)[sP]• |
| | [dR](T)[sP]•[MOE]([5meC])P•[MOE](T)P•[MOE](T)[sP]•[MOE]([5meC])[sP]•[MOE]([5meC]) |
| 1122_95 | [LR](A)[sP]•[mR](A)[sP]•[mR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_96 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[LR]([5meC])[sP]•[LR](T)[sP]•[dR](T)[sP]• |
| | [LR]([5meC])[sP]•[LR]([5meC]) |
| 1122_97 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[LR]([5meC])[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_98 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[MOE]([5meC])[sP]•[LR](T)[sP]•[dR](T)[sP]•[LR]([5meC])[sP]• |
| | [LR]([5meC]) |
| 1122_99 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[MOE]([5meC])[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_100 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[mR](U)[sP]•[LR]([5meC])[sP]• |
| | [LR]([5meC]) |
| 1122_101 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[mR](U)[sP]•[dR](T)[sP]•[LR]([5meC])[sP]• |
| | [LR]([5meC]) |
| 1122_102 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[mR](U)[sP]•[dR](C)[sP]•[LR](T)[sP]•[dR](T)[sP]•[LR]([5meC])[sP]• |
| | [LR]([5meC]) |
| 1122_103 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[mR](U)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_104 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[mR](U)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_105 | [LR](A)[sP]•[LR](A)[sP]•[LR](T)[sP]•[LR]([5meC])[sP]•[dR](T)[sP]•[dR](T)[sP]• |
| | [dR](A)[sP]•[mR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_106 | [LR](A)[sP]•[LR](A)[sP]•[LR](T)[sP]•[LR]([5meC])[sP]•[mR](U)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_107 | [LR](A)[sP]•[LR](A)[sP]•[mR](U)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_108 | [LR](A)[sP]•[mR](A)[sP]•[mR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_109 | [LR](A)[sP]•[LR](A)[sP]•[LR](T)[sP]•[LR]([5meC])[sP]•[dR](T)[sP]•[mR](U)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_110 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[LR]([5meC])[sP]•[LR](T)[sP]•[dR](T)[sP]• |
| | [LR]([5meC])[sP][LR]([5meC]) |
| 1122_111 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[LR]([5meC])[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_112 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[MOE[LR]([5meC]) |
| 1122_113 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[MOE]([5meC])[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_114 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[mR](U)[sP]•[LR]([5meC])[sP]• |
| | [LR]([5meC]) |
| 1122_115 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[mR](U)[sP]•[dR](T)[sP]•[LR]([5meC])[sP]• |
| | [LR]([5meC]) |
| 1122_116 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[mR](U)[sP]•[dR](C)[sP]•[LR](T)[sP]•[dR](T)[sP]•[LR]([5meC])[sP]• |
| | [LR]([5meC]) |
| 1122_117 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[mR](U)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_118 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[mR](U)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_119 | [LR](A)[sP]•[LR](A)[sP]•[dR](T)[sP]•[LR]([5meC])[sP]•[dR](T)[sP]•[dR](T)[sP]• |
| | [LR](A)[sP]•[mR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_120 | [LR](A)[sP]•[LR](A)[sP]•[dR](T)[sP]•[LR]([5meC])[sP]•[dR](T)[sP]•[mR](U)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_121 | [LR](A)[sP]•[LR](A)[sP]•[dR](T)[sP]•[LR]([5meC])[sP]•[mR](U)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_122 | [LR](A)[sP]•[LR](A)[sP]•[mR](U)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_123 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[LR]([5meC])[sP]•[dR](T)[sP]•[LR](T)[sP]•[LR]([5meC])[sP]• |
| | [LR]([5meC]) |
| 1122_124 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[LR]([5meC])[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_125 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_126 | [LR](A)[sP]•[LR](A)[sP]•[dR](T)[sP]•[LR]([5meC])[sP]•[dR](T)[sP]•[dR](T)[sP]• |
| | [LR](A)[ssP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_127 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[ssP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_128 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[ssP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_129 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[ssP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_130 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[ssP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_131 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[ssP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_132 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[ssP]•[dR](T)[sP]•[dR](C)[sP]•[LR](T)[sP]•[dR](T)[sP]•[LR]([5meC])[sP]• |
| | [LR]([5meC]) |
| 1122_133 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[ssP]•[dR](C)[sP]•[LR](T)[sP]•[dR](T)[sP]•[LR]([5meC])[sP]• |
| | [LR]([5meC]) |
| 1122_134 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[ssP]•[LR](T)[sP]•[dR](T)[sP]•[LR]([5meC])[sP]• |
| | [LR]([5meC]) |
| 1122_135 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[ssP]•[dR](T)[sP]•[LR]([5meC])[sP]• |
| | [LR]([5meC]) |
| 1122_136 | [LR](A)[sP]•[LR](A)[sP]•[LR](T)[sP]•[LR]([5meC])[ssP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_137 | [LR](A)[sP]•[LR](A)[sP]•[LR](T)[sP]•[LR]([5meC])[sP]•[dR](T)[ssP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_138 | [LR](A)[sP]•[LR](A)[sP]•[LR](T)[sP]•[LR]([5meC])[sP]•[dR](T)[sP]•[dR](T)[ssP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_139 | [LR](A)[sP]•[LR](A)[sP]•[LR](T)[sP]•[LR]([5meC])[sP]•[dR](T)[sP]•[dR](T)[sP]• |
| | [dR](A)[ssP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_140 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[ssP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_141 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[ssP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_142 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[ssP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_143 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[ssP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_144 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[ssP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_145 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[ssP]•[dR](T)[sP]•[dR](C)[sP]•[LR](T)[sP]•[dR](T)[sP]•[LR]([5meC])[sP]• |
| | [LR]([5meC]) |
| 1122_146 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[ssP]•[dR](C)[sP]•[LR](T)[sP]•[dR](T)[sP]•[LR]([5meC])[sP]• |
| | [LR]([5meC]) |
| 1122_147 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[ssP]•[LR](T)[sP]•[dR](T)[sP]•[LR]([5meC])[sP]• |
| | [LR]([5meC]) |
| 1122_148 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[ssP]•[dR](T)[sP]•[LR]([5meC])[sP]• |
| | [LR]([5meC]) |
| 1122_149 | [LR](A)[sP]•[dR](A)[sP]•[dR](T)[sP]•[LR]([5meC])[sP]•[dR](T)[sP]•[LR](T)[ssP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_150 | [LR](A)[sP]•[dR](A)[sP]•[dR](T)[sP]•[LR]([5meC])[sP]•[dR](T)[sP]•[LR](T)[sP]• |
| | [dR](A)[ssP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_151 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[ssP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_152 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[ssP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_153 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[ssP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_154 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[ssP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_155 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[ssP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_156 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[ssP]•[dR](T)[sP]•[dR](C)[sP]•[dR](T)[sP]•[LR](T)[sP]•[LR]([5meC])[sP]• |
| | [LR]([5meC]) |
| 1122_157 | [LR](A)[sP]•[dR](A)[sP]•[dR](T)[sP]•[LR]([5meC])[sP]•[dR](T)[sP]•[LR](T)[ sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[ssP]•[dR](C)[sP]•[dR](T)[sP]•[LR](T)[sP]•[LR]([5meC])[sP]• |
| | [LR]([5meC]) |
| 1122_158 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[ssP]•[dR](T)[sP]•[LR](T)[sP]•[LR]([5meC])[sP]• |
| | [LR]([5meC]) |
| 1122_159 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[ssP]•[LR](T)[sP]•[LR]([5meC])[sP]• |
| | [LR]([5meC]) |
| 1122_160 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[ssP]•[LR]([5meC])[sP]• |
| | [LR]([5meC]) |
| 1122_161 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[MOE](T)[sP]•[dR](C)[sP]•[dR](T)[sP]•[LR](T)[sP]•[LR]([5meC])[sP]• |
| | [LR]([5meC]) |
| 1122_162 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [MOE](A)[sP]•[dR](T)[sP]•[dR](C)[sP]•[dR](T)[sP]•[LR](T)[sP]•[LR]([5meC])[sP]• |
| | [LR]([5meC]) |
| 1122_163 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[MOE](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_164 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[MOE](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_165 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[MOE](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_166 | [LR](A)[sP]•[dR](A)[sP]•[dR](T)[sP]•[LR]([5meC])[sP]•[dR](T)[sP]•[LR](T)[sP]• |
| | [dR](A)[sP]•[MOE](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_167 | [LR](A)[sP]•[dR](A)[sP]•[dR](T)[sP]•[LR]([5meC])[sP]•[dR](T)[sP]•[LR](T)[sP]• |
| | [MOE](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_168 | [LR](A)[sP]•[dR](A)[sP]•[dR](T)[sP]•[LR]([5meC])[sP]•[dR](T)[sP]•[MOE](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_169 | [LR](A)[sP]•[dR](A)[sP]•[dR](T)[sP]•[LR]([5meC])[sP]•[MOE](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_170 | [LR](A)[sP]•[dR](A)[sP]•[dR](T)[sP]•[MOE]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_171 | [LR](A)[sP]•[dR](A)[sP]•[MOE](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_172 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[mR](C)[sP]•[dR](T)[sP]•[LR](T)[sP]•[LR]([5meC])[sP]• |
| | [LR]([5meC]) |
| 1122_173 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [mR](A)[sP]•[dR](T)[sP]•[dR](C)[sP]•[dR](T)[sP]•[LR](T)[sP]•[LR]([5meC])[sP]• |
| | [LR]([5meC]) |
| 1122_174 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[mR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_175 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[mR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_176 | [LR](A)[sP]•[dR](A)[sP]•[dR](T)[sP]•[LR]([5meC])[sP]•[dR](T)[sP]•[LR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_177 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[LR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_178 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[LR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_179 | [LR](A)[sP]•[dR](A)[sP]•[dR](T)[sP]•[LR]([5meC])[sP]•[dR](T)[sP]•[LR](T)[sP]• |
| | [dR](A)[sP]•[LR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_180 | [LR](A)[sP]•[dR](A)[sP]•[dR](T)[sP]•[LR]([5meC])[sP]•[dR](T)[sP]•[LR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_181 | [LR](A)[sP]•[dR](A)[sP]•[dR](T)[sP]•[LR]([5meC])[sP]•[LR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_182 | [LR](A)[sP]•[dR](A)[sP]•[LR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_183 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[MOE]([5meC])[sP]• |
| | [LR]([5meC]) |
| 1122_184 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[MOE](T)[sP]•[LR]([5meC])[sP]• |
| | [LR]([5meC]) |
| 1122_185 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[LR](T)[sP]•[LR]([5meC])[sP]• |
| | [LR]([5meC]) |
| 1122_186 | [LR](A)[sP]•[LR](A)[sP]•[LR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_188 | [MOE](A)[sP]•[MOE](A)[sP]•[MOE](T)[sP]•[MOE]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]•[dR](A)[sP]• |
| | [dR](T)[sP]•[dR](C)[sP]•[MOE](T)[sP]•[dR](T)[sP]•[MOE]([5meC])[sP]•[MOE]([5meC]) |
| 1122_189 | [MOE](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [MOE]([5meC]) |
| 1122_190 | [MOE](A)[sP]•[MOE](A)P•[MOE](T)P•[MOE]([5meC])[sP]•[dR](T)[sP]•[dR ](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[dR](T)[sP]•[MOE]([5meC])[sP]• |
| | [MOE]([5meC]) |
| 1122_191 | [LR](A)[sP]•[fR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_192 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[fR](C)[sP]•[LR](T)[sP]•[dR](T)[sP]•[LR]([5meC])[sP]• |
| | [LR]([5meC]) |
| 1122_193 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [fR](A)[sP]•[dR](T)[sP]•[dR](C)[sP]•[LR](T)[sP]•[dR](T)[sP]•[LR]([5meC])[sP]• |
| | [LR]([5meC]) |
| 1122_194 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[fR](C)[sP]• |
| | [dR](A)[sP]•[dR](T)[sP]•[dR](C)[sP]•[LR](T)[sP]•[dR](T)[sP]•[LR]([5meC])]sP]• |
| | [LR]([5meC]) |
| 1122_195 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[fR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_196 | [LR](A)[sP]•[LR](A)[sP]•[LR](T)[sP]•[LR]([5meC])[sP]•[dR](T)[sP]•[dR](T)[sP]• |
| | [fR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_197 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[LR](T)[sP]•[LR]([5meC])[sP]• |
| | [LR]([5meC]) |
| 1122_198 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[LR](T)[sP]•[dR](C)[sP]•[LR](T)[sP]•[dR](T)[sP]•[LR]([5meC])[sP]• |
| | [LR]([5meC]) |
| 1122_199 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [LR](A)[sP]•[dR](T)[sP]•[dR](C)[sP]•[LR](T)[sP]•[dR](T)[sP]•[LR]([5meC])[sP]• |
| | [LR]([5meC]) |
| 1122_200 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[LR](A)[sP]•[dR](C)[sP]• |
| | [dR](A)[sP]•[dR](T)[sP]•[dR](C)[sP]•[LR](T)[sP]•[dR](T)[sP]•[LR]([5meC])]sP]• |
| | [LR]([5meC]) |
| 1122_201 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[LR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_202 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[LR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])]sP]• |
| | [LR]([5meC]) |
| 1122_203 | [LR](A)[sP]•[LR](A)[sP]•[LR](T)[sP]•[LR]([5meC])[sP]•[dR](T)[sP]•[dR](T)[sP]• |
| | [dR](A)[sP]•[LR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])]sP]• |
| | [LR]([5meC]) |
| 1122_204 | [LR](A)[sP]•[LR](A)[sP]•[LR](T)[sP]•[LR]([5meC])[sP]•[LR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_205 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[MOE]([5meC])[sP]• |
| | [LR]([5meC]) |
| 1122_206 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[MOE](T)[sP]•[LR]([5meC])[sP]• |
| | [LR]([5meC]) |
| 1122_207 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[dR](T)[sP]•[LR]([5meC])[sP]• |
| | [LR]([5meC]) |
| 1122_208 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[MOE](T)[sP]•[dR](C)[sP]•[LR](T)[sP]•[dR](T)[sP]•[LR]([5meC])[sP]• |
| | [LR]([5meC]) |
| 1122_209 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [MOE](A)[sP]•[dR](T)[sP]•[dR](C)[sP]•[LR](T)[sP]•[dR](T)[sP]•[LR]([5meC])[sP]• |
| | [LR]([5meC]) |
| 1122_210 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[MOE](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_211 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[MOE](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_212 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[MOE](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_213 | [LR](A)[sP]•[LR](A)[sP]•[LR](T)[sP]•[LR]([5meC])[sP]•[dR](T)[sP]•[dR](T)[sP]• |
| | [dR](A)[sP]•[MOE](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_214 | [LR](A)[sP]•[LR](A)[sP]•[LR](T)[sP]•[LR]([5meC])[sP]•[dR](T)[sP]•[dR](T)[sP]• |
| | [MOE](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_215 | [LR](A)[sP]•[LR](A)[sP]•[LR](T)[sP]•[LR]([5meC])[sP]•[dR](T)[sP]•[MOE](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_216 | [LR](A)[sP]•[LR](A)[sP]•[LR](T)[sP]•[LR]([5meC])[sP]•[MOE](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_217 | [LR](A)[sP]•[LR](A)[sP]•[LR](T)[sP]•[MOE]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_218 | [LR](A)[sP]•[LR](A)[sP]•[MOE](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_219 | [LR](A)[sP]•[MOE](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_220 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[mR](C)[sP]•[LR](T)[sP]•[dR](T)[sP]•[LR]([5meC])[sP]• |
| | [LR]([5meC]) |
| 1122_221 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [mR](A)[sP]•[dR](T)[sP]•[dR](C)[sP]•[LR](T)[sP]•[dR](T)[sP]•[LR]([5meC])[sP]• |
| | [LR]([5meC]) |
| 1122_222 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[mR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_223 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[mR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_224 | [LR](A)[sP]•[LR](A)[sP]•[LR](T)[sP]•[LR]([5meC])[sP]•[dR](T)[sP]•[dR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_225 | [LR](A)[sP]•[mR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_226 | [MOE](A)[sP]•[dR](A)[sP]•[dR](T)[sP]•[MOE]([5meC])[sP]•[dR](T)[sP]•[MOE](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[MOE](T)[sP]•[MOE]([5meC])[sP]• |
| | [MOE]([5meC]) |
| 1122_227 | [MOE](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [MOE]([5meC]) |
| 1122_228 | [MOE](A)[sP]•[LR](A)[sP]•[LR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_229 | [LR](A)[sP]•[fR](A)[sP]•[dR](T)[sP]•[LR]([5meC])[sP]•[dR](T)[sP]•[LR](T)[ sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC] )[sP]• |
| | [LR]([5meC]) |
| 1122_230 | [LR](A)[sP]•[LR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_231 | [LR](A)[sP]•[MOE](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_232 | [LR](A)[sP]•[mR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_233 | [LR](A)[sP]•[MOE](A)P•[MOE](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_234 | [LR](A)[sP]•[LR](A)[sP]•[LR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_235 | [LR](A)[sP]•[MOE](A)[sP]•[MOE](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_236 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[fR](C)[sP]•[dR](T)[sP]•[LR](T)[sP]•[LR]([5meC])[sP]• |
| | [LR]([5meC]) |
| 1122_237 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [fR](A)[sP]•[dR](T)[sP]•[dR](C)[sP]•[dR](T)[sP]•[LR](T)[sP]•[LR]([5meC])[sP]• |
| | [LR]([5meC]) |
| 1122_238 | [MOE](A)[sP]•[dR](A)[sP]•[dR](T)[sP]•[MOE]([5meC])[sP]•[dR](T)[sP]•[MOE](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[dR](T)[sP]•[MOE]([5meC])[sP]• |
| | [MOE]([5meC]) |
| 1122_239 | [MOE](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [MOE]([5meC]) |
| 1122_240 | [MOE](A)[sP]•[LR](A)[sP]•[LR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_241 | [LR](A)[sP]•[fR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_242 | [LR](A)[sP]•[MOE](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_243 | [LR](A)[sP]•[mR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_244 | [LR](A)[sP]•[MOE](A)P•[MOE](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_245 | [LR](A)[sP]•[LR](A)[sP]•[LR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_247 | [LR](A)[sP]•[MOE](A)[sP]•[MOE](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_248 | [MOE](A)[sP]•[MOE](A)[sP]•[dR](T)[sP]•[MOE]([5meC])[sP]•[MOE](T)[sP]•[dR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[MOE](T)[sP]•[MOE]([5meC])[sP]• |
| | [MOE]([5meC]) |
| 1122_249 | [MOE](A)[sP]•[LR](A)[sP]•[dR](T)[sP]•[LR]([5meC])[sP]•[LR](T)[sP]•[dR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [MOE]([5meC]) |
| 1122_250 | [MOE](A)[sP]•[MOE](A)[sP]•[dR](T)[sP]•[MOE]([5meC])P•[MOE](T)[sP]•[dR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[MOE](T)[sP]•[MOE]([5meC])[sP]• |
| | [MOE]([5meC]) |
| 1122_251 | [MOE](A)[sP]•[MOE](A)[sP]•[dR](T)[sP]•[MOE]([5meC])[sP]•[dR](T)[sP]•[dR](T)[sP]• |
| | [MOE](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[dR](T)[sP]•[MOE]([5meC])[sP]• |
| | [MOE]([5meC]) |
| 1122_252 | [MOE](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [MOE]([5meC]) |
| 1122_253 | [LR](A)[sP]•[fR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_254 | [LR](A)[sP]•[LR](A)[sP]•[dR](T)[sP]•[LR]([5meC])[sP]•[dR](T)[sP]•[dR](T)[sP]• |
| | [fR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_255 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[fR](C)[sP]•[LR](T)[sP]•[dR](T)[sP]•[LR]([5meC])[sP]• |
| | [LR]([5meC]) |
| 1122_256 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [fR](A)[sP]•[dR](T)[sP]•[dR](C)[sP]•[LR](T)[sP]•[dR](T)[sP]•[LR]([5meC])[sP]• |
| | [LR]([5meC]) |
| 1122_257 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[fR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_258 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[fR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_259 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[LR](T)[sP]•[LR]([5meC])[sP]• |
| | [LR]([5meC]) |
| 1122_260 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[LR](T)[sP]•[dR](C)[sP]•[LR](T)[sP]•[dR](T)[sP]•[LR]([5meC])[sP]• |
| | [LR]([5meC]) |
| 1122_261 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [LR](A)[sP]•[dR](T)[sP]•[dR](C)[sP]•[LR](T)[sP]•[dR](T)[sP]•[LR]([5meC])[sP]• |
| | [LR]([5meC]) |
| 1122_262 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[LR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_263 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[LR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_264 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[LR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_265 | [LR](A)[sP]•[LR](A)[sP]•[dR](T)[sP]•[LR]([5meC])[sP]•[dR](T)[sP]•[dR](T)[sP]• |
| | [LR](A)[sP]•[LR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_266 | [LR](A)[sP]•[LR](A)[sP]•[dR](T)[sP]•[LR]([5meC])[sP]•[dR](T)[sP]•[LR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_267 | [LR](A)[sP]•[LR](A)[sP]•[dR](T)[sP]•[LR]([5meC])[sP]•[LR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_268 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[MOE]([5meC])[sP]• |
| | [LR]([5meC]) |
| 1122_269 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[MOE](T)[sP]•[LR]([5meC])[sP]• |
| | [LR]([5meC]) |
| 1122_270 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[MOE](T)[sP]•[dR](T)[sP]•[LR]([5meC])[sP]• |
| | [LR]([5meC]) |
| 1122_271 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[MOE](T)[sP]•[dR](C)[sP]•[LR](T)[sP]•[dR](T)[sP]•[LR]([5meC])[sP]• |
| | [LR]([5meC]) |
| 1122_272 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [MOE](A)[sP]•[dR](T)[sP]•[dR](C)[sP]•[LR](T)[sP]•[dR](T)[sP]•[LR]([5meC])[sP]• |
| | [LR]([5meC]) |
| 1122_273 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[MOE](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_274 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[MOE](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_275 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[MOE](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_276 | [LR](A)[sP]•[LR](A)[sP]•[dR](T)[sP]•[LR]([5meC])[sP]•[dR](T)[sP]•[dR](T)[sP]• |
| | [LR](A)[sP]•[MOE](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_277 | [LR](A)[sP]•[LR](A)[sP]•[dR](T)[sP]•[LR]([5meC])[sP]•[dR](T)[sP]•[dR](T)[sP]• |
| | [MOE](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_278 | [LR](A)[sP]•[LR](A)[sP]•[dR](T)[sP]•[LR]([5meC])[sP]•[dR](T)[sP]•[MOE](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_279 | [LR](A)[sP]•[LR](A)[sP]•[dR](T)[sP]•[LR]([5meC])[sP]•[MOE](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_280 | [LR](A)[sP]•[LR](A)[sP]•[dR](T)[sP]•[MOE]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_281 | [LR](A)[sP]•[LR](A)[sP]•[MOE](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_282 | [LR](A)[sP]•[MOE](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_283 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[mR](C)[sP]•[LR](T)[sP]•[dR](T)[sP]•[LR]([5meC])[sP]• |
| | [LR]([5meC]) |
| 1122_284 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [mR](A)[sP]•[dR](T)[sP]•[dR](C)[sP]•[LR](T)[sP]•[dR](T)[sP]•[LR]([5meC])[sP]• |
| | [LR]([5meC]) |
| 1122_285 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[mR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_286 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[mR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_287 | [LR](A)[sP]•[LR](A)[sP]•[dR](T)[sP]•[LR]([5meC])[sP]•[dR](T)[sP]•[dR](T)[sP]• |
| | [mR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_288 | [LR](A)[sP]•[mR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_289 | [LR](A)[sP]•[LR](A)[sP]•[LR](T)[sP]•[fR](C)[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_290 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[fR](C)[sP]• |
| | [LR]([5meC]) |
| 1122_291 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[mR](C)[sP]• |
| | [LR]([5meC]) |
| 1122_292 | [LR](A)[sP]•[LR](A)[sP]•[LR](T)[sP]•[mR](C)[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_293 | [LR](A)[sP]•[dR](A)[sP]•[dR](T)[sP]•[fR](C)[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_294 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[fR](C)[sP]• |
| | [LR]([5meC]) |
| 1122_295 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[mR](C)[sP]• |
| | [LR]([5meC]) |
| 1122_296 | [LR](A)[sP]•[dR](A)[sP]•[dR](T)[sP]•[mR](C)[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_297 | [LR](A)[sP]•[LR](A)[sP]•[dR](T)[sP]•[fR](C)[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_298 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[fR](C)[sP]• |
| | [LR]([5meC]) |
| 1122_299 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[mR](C)[sP]• |
| | [LR]([5meC]) |
| 1122_300 | [LR](A)[sP]•[LR](A)[sP]•[dR](T)[sP]•[mR](C)[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_301 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[fR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_302 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[fR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_303 | [LR](A)[sP]•[dR](A)[sP]•[dR](T)[sP]•[LR]([5meC])[sP]•[dR](T)[sP]•[LR](T)[sP]• |
| | [fR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_304 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[LR](T)[sP]•[LR]([5meC])[sP]• |
| | [LR]([5meC]) |
| 1122_305 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[LR](T)[sP]•[dR](C)[sP]•[dR](T)[sP]•[LR](T)[sP]•[LR]([5meC])[sP]• |
| | [LR]([5meC]) |
| 1122_306 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [LR](A)[sP]•[dR](T)[sP]•[dR](C)[sP]•[dR](T)[sP]•[LR](T)[sP]•[LR]([5meC])[sP]• |
| | [LR]([5meC]) |
| 1122_307 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[LR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_308 | [LR](A)[sP]•[fR](A)[sP]•[fR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_309 | [LR](A)[sP]•[LR](A)[sP]•[fR](U)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_310 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[fR](U)[sP]•[dR](T)[sP]•[LR]([5meC])[sP]• |
| | [LR]([5meC]) |
| 1122_311 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[fR](U)[sP]•[LR]([5meC])[sP]• |
| | [LR]([5meC]) |
| 1122_312 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[fR](U)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_313 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[fR](U)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_314 | [LR](A)[sP]•[LR](A)[sP]•[LR](T)[sP]•[LR]([5meC])[sP]•[dR](T)[sP]•[dR](T)[sP]• |
| | [dR](A)[sP]•[fR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_315 | [LR](A)[sP]•[LR](A)[sP]•[LR](T)[sP]•[LR]([5meC])[sP]•[dR](T)[sP]•[fR](U)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_316 | [LR](A)[sP]•[fR](A)[sP]•[fR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_317 | [LR](A)[sP]•[dR](A)[sP]•[dR](T)[sP]•[LR]([5meC])[sP]•[dR](T)[sP]•[fR](U)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_318 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[fR](U)[sP]•[LR]([5meC])[sP]• |
| | [LR]([5meC]) |
| 1122_319 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[fR](U)[sP]•[LR](T)[sP]•[LR]([5meC])[sP]• |
| | [LR]([5meC]) |
| 1122_320 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR[(T)[sP]•[dR[(T)[sP]•[dR](A)[sP]•[dR](C)[sP]• |
| | [dR](A)[sP]•[fR](U)[sP]•[dR](C)[sP]•[dR](T)[sP]•[LR](T)[sP]•[LR]([5meC])[sP]• |
| | [LR]([5meC]) |
| 1122_321 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[dR](T)[sP]•[fR](U)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_322 | [LR](A)[sP]•[dR](A)[sP]•[dR](T)[sP]•[LR]([5meC])[sP]•[dR](T)[sP]•[LR](T)[sP]• |
| | [dR](A)[sP]•[dR](T)[sP]•[fR](U)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_323 | [LR](A)[sP]•[dR](A)[sP]•[dR](T)[sP]•[LR]([5meC])[sP]•[dR](T)[sP]•[LR](T)[sP]• |
| | [dR](A)[sP]•[fR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_324 | [LR](A)[sP]•[dR](A)[sP]•[dR](T)[sP]•[LR]([5meC])[sP]•[fR](U)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_325 | [LR](A)[sP]•[dR](A)[sP]•[fR](U)[sP]•[LR]([5meC])[sP]•[dR](T)[sP]•[LR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[dR](T)[sP]•[LR](T)[sP]•[LR]([5meC])[sP]• |
| | [LR]([5meC]) |
| 1122_326 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[fR](U)[sP]•[dR](T)[sP]•[LR]([5meC])[sP]• |
| | [LR]([5meC]) |
| 1122_327 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[fR](U)[sP]•[LR]([5meC])[sP]• |
| | [LR]([5meC]) |
| 1122_328 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[fR](U)[sP]•[dR](C)[sP]•[LR](T)[sP]•[dR](T)[sP]•[LR]([5meC])[sP]• |
| | [LR]([5meC]) |
| 1122_329 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[dR](T)[sP]•[fR](U)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_330 | [LR](A)[sP]•[LR](A)[sP]•[dR](T)[sP]•[LR]([5meC])[sP]•[dR](T)[sP]•[dR](T)[sP]• |
| | [LR](A)[sP]•[dR](T)[sP]•[fR](U)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_331 | [LR](A)[sP]•[LR](A)[sP]•[dR](T)[sP]•[LR]([5meC])[sP]•[dR](T)[sP]•[dR](T)[sP]• |
| | [LR](A)[sP]•[fR](U)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_332 | [LR](A)[sP]•[LR](A)[sP]•[dR](T)[sP]•[LR]([5meC])[sP]•[dR](T)[sP]•[fR](U)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_333 | [LR](A)[sP]•[LR](A)[sP]•[dR](T)[sP]•[LR]([5meC])[sP]•[fR](U)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_334 | [LR](A)[sP]•[LR](A)[sP]•[fR](U)[sP]•[LR]([5meC])[sP]•[dR](T)[sP]•[dR](T)[sP]• |
| | [LR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_335 | [LR](A)[sP]•[LR](A)[sP]•[LR](T)[sP]•[LR]([5meC])[sP]•[fR](U)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[dR](T)[sP]•[dR](C)[sP]•[LR](T)[sP]•[dR](T)[sP]•[LR]([5meC])[sP]• |
| | [LR]([5meC]) |
| 1122_336 | [LR](A)[sP]•[LR](A)[sP]•[LR](T)[sP]•[LR]([5meC])[sP]•[dR](T)[sP]•[dR](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[fR](U)[sP]•[dR](C)[sP]•[LR](T)[sP]•[dR](T)[sP]•[LR]([5meC])[sP]• |
| | [LR]([5meC]) |
| 1816_2 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [LR]([5meC])[sP]•[dR](A)[sP]•[dR](T)[sP]•[LR]([5meC])[sP]•[LR](T)[sP]•[LR](T) |
| 1816_3 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [MOE]([5meC])[sP]•[dR](A)[sP]•[dR](T)[sP]•[LR]([5meC])[sP]•[LR](T)[sP]•[LR](T) |
| 1816_4 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[mR](U)[sP]•[LR](T) |
| 1816_5 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[mR](U)[sP]•[LR]([5meC])[sP]•[LR](T)[sP]•[LR](T) |
| 1816_6 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[mR](U)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_7 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[mR](U)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_8 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[mR](U)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_9 | [LR](G)[sP]•[LR](A)[sP]•[dR](A)[sP]•[dR](T)[sP]•[LR]([5meC])[sP]•[dR](T)[sP]• |
| | [mR](U)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_10 | [LR](G)[sP]•[LR](A)[sP]•[dR](A)[sP]•[dR](T)[sP]•[LR]([5meC])[sP]•[mR](U)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_11 | [LR](G)[sP]•[LR](A)[sP]•[dR](A)[sP]•[mR](U)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_12 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_13 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[ssP]• |
| | [dR](C)[sP]•[dR](A)[sP]•[dR](T)[sP]•[LR]([5meC])[sP]•[LR](T)[sP]•[LR](T) |
| 1816_14 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[ssP]•[dR](A)[sP]•[dR](T)[sP]•[LR]([5meC])[sP]•[LR](T)[sP]•[LR](T) |
| 1816_15 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[ssP]•[dR](T)[sP]•[LR]([5meC])[sP]•[LR](T)[sP]•[LR](T) |
| 1816_16 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[ssP]•[LR]([5meC])[sP]•[LR](T)[sP]•[LR](T) |
| 1816_17 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[ssP]•[LR](T)[sP]•[LR](T) |
| 1816_18 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[ssP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_19 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[ssP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_20 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[ssP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_21 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[ssP]•[dR](A)[sP]• |
| | [dR](C)[sP]•[dR](A)[sP]•[dR](T)[sP]•[LR]([5meC])[sP]•[LR](T)[sP]•[LR](T) |
| 1816_22 | [MOE](G)[sP]•[MOE](A)[sP]•[dR](A)[sP]•[dR](T)[sP]•[MOE]([5meC])[sP]•[dR](T)[sP]• |
| | [MOE](T)[sP]•[MOE](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[MOE]([5meC])[sP]•[MOE](T)[sP]•[MOE](T) |
| 1816_23 | [MOE](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[MOE](T) |
| 1816_24 | [MOE](G)[sP]•[MOE](A)[sP]•[MOE](A)[sP]•[MOE](T)[sP]•[MOE]([5meC])[sP]• |
| | [MOE](T)[sP]•[MOE](T)[sP]•[dR](A)[sP]•[dR](T)[sP]•[dR](T)[sP]•[dR](T)[sP]• |
| | [dR](A)[sP]•[dR](C)[sP]•[dR](A)[sP]•[MOE](T)[sP]•[MOE]([5meC])[sP]•[MOE](T)[sP]• |
| | [MOE](T) |
| 1816_25 | [MOE](G)[sP]•[MOE](A)[sP]•[dR](A)[sP]•[dR](T)[sP]•[MOE]([5meC])[sP]•[dR](T)[sP]• |
| | [MOE](T)P•[MOE](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[MOE]([5meC])[sP]•[MOE](T)[sP]•[MOE](T) |
| 1816_26 | [MOE](G)[sP]•[MOE](A)P•[MOE](A)P[MOE](T)P•[MOE]([5meC])P•[MOE](T)P•[MOE](T)[sP]• |
| | [dR](A)[sP]•[dR](T)[sP]•[dR](T)[sP]•[dR](T)[sP]•[dR](A)[sP]•[dR](C)[sP]• |
| | [dR](A)[sP]•[MOE](T)P•[MOE]([5meC])[sP]•[MOE](T)[sP]•[MOE](T) |
| 1816_27 | [LR](G)[sP]•[fR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_28 | [LR](G)[sP]•[LR](A)[sP]•[dR](A)[sP]•[dR](T)[sP]•[LR]([5meC])[sP]•[dR](T)[sP]• |
| | [LR](T)[sP]•[fR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_29 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[fR](A)[sP]•[dR](T)[sP]•[LR]([5meC])[sP]•[LR](T)[sP]•[LR](T) |
| 1816_30 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [fR](C)[sP]•[dR](A)[sP]•[dR](T)[sP]•[LR]([5meC])[sP]•[LR](T)[sP]•[LR](T) |
| 1816_31 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[fR](A)[sP]• |
| | [dR](C)[sP]•[dR](A)[sP]•[dR](T)[sP]•[LR]([5meC])[sP]•[LR](T)[sP]•[LR](T) |
| 1816_32 | [LR](G)[sP]•[LR](A)[sP]•[fR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_33 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[LR](T)[sP]•[LR]([5meC])[sP]•[LR](T)[sP]•[LR](T) |
| 1816_34 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[LR](A)[sP]•[dR](T)[sP]•[LR]([5meC])[sP]•[LR](T)[sP]•[LR](T) |
| 1816_35 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[LR](A)[sP]• |
| | [dR](C)[sP]•[dR](A)[sP]•[dR](T)[sP]•[LR]([5meC])[sP]•[LR](T)[sP]•[LR](T) |
| 1816_36 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[LR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_37 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[LR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_38 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[LR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_39 | [LR](G)[sP]•[LR](A)[sP]•[dR](A)[sP]•[dR](T)[sP]•[LR]([5meC])[sP]•[LR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_40 | [LR](G)[sP]•[LR](A)[sP]•[dR](A)[sP]•[LR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_41 | [LR](G)[sP]•[LR](A)[sP]•[LR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_42 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[MOE](T)[sP]•[LR](T) |
| 1816_43 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[MOE]([5meC])[sP]•[LR](T)[sP]•[LR](T) |
| 1816_44 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[MOE](T)[sP]•[LR]([5meC])[sP]•[LR](T)[sP]•[LR](T) |
| 1816_45 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[MOE](A)[sP]•[dR](T)[sP]•[LR]([5meC])[sP]•[LR](T)[sP]•[LR](T) |
| 1816_46 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[MOE](A)[sP]• |
| | [dR](C)[sP]•[dR](A)[sP]•[dR](T)[sP]•[LR]([5meC])[sP]•[LR](T)[sP]•[LR](T) |
| 1816_47 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[MOE](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_48 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[MOE](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_49 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[MOE](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_50 | [LR](G)[sP]•[LR](A)[sP]•[dR](A)[sP]•[dR](T)[sP]•[LR]([5meC])[sP]•[dR](T)[sP]• |
| | [LR](T)[sP]•[MOE](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_51 | [LR](G)[sP]•[LR](A)[sP]•[dR](A)[sP]•[dR](T)[sP]•[LR]([5meC])[sP]•[dR](T)[sP]• |
| | [MOE](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_52 | [LR](G)[sP]•[LR](A)[sP]•[dR](A)[sP]•[dR](T)[sP]•[LR]([5meC])[sP]•[MOE](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_53 | [LR](G)[sP]•[LR](A)[sP]•[dR](A)[sP]•[dR](T)[sP]•[MOE]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_54 | [LR](G)[sP]•[LR](A)[sP]•[dR](A)[sP]•[MOE](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_55 | [LR](G)[sP]•[LR](A)[sP]•[MOE](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_56 | [LR](G)[sP]•[MOE](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_57 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[mR](A)[sP]•[dR](T)[sP]•[LR]([5meC])[sP]•[LR](T)[sP]•[LR](T) |
| 1816_58 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [mR](C)[sP]•[dR](A)[sP]•[dR](T)[sP]•[LR]([5meC])[sP]•[LR](T)[sP]•[LR](T) |
| 1816_59 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[mR](A)[sP]• |
| | [dR](C)[sP]•[dR](A)[sP]•[dR](T)[sP]•[LR]([5meC])[sP]•[LR](T)[sP]•[LR](T) |
| 1816_60 | [LR](G)[sP]•[LR](A)[sP]•[dR](A)[sP]•[dR](T)[sP]•[LR]([5meC])[sP]•[dR](T)[sP]• |
| | [LR](T)[sP]•[mR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_61 | [LR](G)[sP]•[LR](A)[sP]•[mR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_62 | [LR](G)[sP]•[mR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_63 | [LR](G)[sP]•[LR](A)[sP]•[dR](A)[sP]•[dR](T)[sP]•[fR](C)[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_64 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[fR](C)[sP]•[LR](T)[sP]•[LR](T) |
| 1816_65 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[mR](C)[sP]•[LR](T)[sP]•[LR](T) |
| 1816_66 | [LR](G)[sP]•[LR](A)[sP]•[dR](A)[sP]•[dR](T)[sP]•[mR](C)[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_67 | [LR](G)[sP]•[LR](A)[sP]•[dR](A)[sP]•[dR](T)[sP]•[LR]([5meC])[sP]•[dR](T)[sP]• |
| | [fR](U)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_68 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[fR](U)[sP]•[LR](T) |
| 1816_69 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[fR](U)[sP]•[LR]([5meC])[sP]•[LR](T)[sP]•[LR](T) |
| 1816_70 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[dR](T)[sP]•[fR](U)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_71 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[dR](T)[sP]•[fR](U)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_72 | [LR](G)[sP]•[LR](A)[sP]•[dR](A)[sP]•[dR](T)[sP]•[LR]([5meC])[sP]•[dR](T)[sP]• |
| | [LR](T)[sP]•[LR](A)[sP]•[fR](U)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_73 | [LR](G)[sP]•[LR](A)[sP]•[dR](A)[sP]•[dR](T)[sP]•[LR]([5meC])[sP]•[fR](U)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |
| 1816_74 | [LR](G)[sP]•[LR](A)[sP]•[dR](A)[sP]•[fR](U)[sP]•[LR]([5meC])[sP]•[dR](T)[sP]• |
| | [LR](T)[sP]•[LR](A)[sP]•[dR](T)[sP]•[dR](T)[sP]•[dR](T)[sP]•[dR](A)[sP]• |
| | [dR](C)[sP]•[dR](A)[sP]•[dR](T)[sP]•[LR]([5meC])[sP]•[LR](T)[sP]•[LR](T) |

### Example 14: Testing in vitro efficacy of LNA oligonucleotides in iCell® GlutaNeurons at 1.25 μM and 62.5 nM

### Materials and Methods:

The compounds of the redesign library described in Table 11 of Example 13 were evaluated for potency in human iPSC cells using two concentrations; 1.25 μM and 62.5 nM, comparing the effect on the ATXN3 transcript and the KCNB2 transcript at both concentrations.

The iCell GlutaNeuron cells were prepared and maintained essentially as described in Example 5 & Table 2. 96-well cell culture plates were coated with Poly-L-Ornithine (0.01%) (Sigma-P4957), 100μl/well for 4 hours. Rinsed 3 times with PBS and coated with Laminin (Roche Diagnostic, 11243217001) 0.5 mg/ml diluted 1:500 in PBS overnight at 4 degrees Celsius. The cells were treated and maintained as per recommendation by the vendor using the provided protocol: iCell® GlutaNeurons, User's Guide, Document ID: X1005, Version 1.2, Cellular Dynamics, Fujifilm; available at https: address cdn.stemcell.com/media/files/manual/MADX1005-icell_glutaneurons_users_guide.pdf (accessed on e.g. 10 Nov. 2020). Compounds were added to the cells from pre-dilution plates (compound diluted in PBS) to reach the desired final concentration. RNA purification and qPCR were performed as described in Example 2; however, using the qPCR assays described below for analysis.

Human KCNB2 pre-mRNA using the qPCR assay:
“Hs.PT.58.39309562”, PrimeTime ® XL qPCR Assay
(Integrated DNA Technologies (IDT),
Leuven, Belgium)
(SEQ ID NO: 1989)
Probe: 5′-/56-FAM/AGA AAC CTA/ZEN/ACT CAT CAG TGG
CTG CAA/3IABkFQ/-3′
(SEQ ID NO: 1990)
Primer 1: 5′-GAA CAG GAT AGA CAC GAT GGC-3′
(SEQ ID NO: 1991)
Primer 2: 5′-AGA GAC TAT GCG AGA GCG A-3′
Human ATXN3 pre-mRNA using the qPCR assay: costum
design “(ATXN3_exon_8-9(1)”, PrimeTime ®
XL qPCR Assay (IDT).
(SEQ ID NO: 1134)
Probe: 5′-/56-FAM/CTCCGCAGG/ZEN/GCT ATTCAGCT
AAGT/31ABkFQ/-3′
(SEQ ID NO: 1135)
Primer 1: 5′-AGT AAGATTTGT ACCTGATGTCTGT-3′
(SEQ ID NO: 1136)
Primer 2: 5′-CATGGAAGATGAGGAAGCAGAT-3′
Human TBP pre-mRNA using the qPCR assay:
“Hs.PT.58v.39858774”, PrimeTime ® XL
qPCR Assay (IDT)
(SEQ ID NO: 1131)
Probe: 5′-/5HEX/TGA TCT TTG/ZEN/CAG TGA CCC AGC
ATC A/3IABkFQ/-3′
(SEQ ID NO: 1132)
Primer 1: 5′-GCT GTT TAA CTT CGC TTC CG-3′
(SEQ ID NO: 1133)
Primer 2: 5′-CAG CAA CTT CCT CAA TTC CTT G-3′

### Results:

The results from this screen is presented in Table 12 as the level of remaining transcript with values given in percent (%) relative to untreated cells, i.e. low level means efficient knockdown. This was done for each of the applied concentrations for each of the two target genes (ATXN3 and KCNB2). Most of the tested compounds showed efficacious knockdown of the ATXN3 transcript at both concentrations used. The effect on the KCNB2 transcript was variable.

TABLE 12

| | 1.25 uM (% | 62.5 nM (% | 1.25 uM (% | 62.5 nM (% |
|---|---|---|---|---|
| | ATXN3 | ATXN3 | KCNB2 | KCNB2 |
| CMP ID | mRNA | mRNA | mRNA | mRNA |
| NO | remaining) | remaining) | remaining) | remaining) |
| 1122_33 | 14.84 | 41.69 | 66.57 | 88.67 |
| 1122_67 | 15.03 | 39.41 | 9.93 | 59.09 |
| 1122_82 | 18.29 | 50.93 | 17.61 | 73.26 |
| 1122_83 | 18.12 | 54.07 | 84.73 | 96.23 |
| 1122_84 | 12.59 | 40.95 | 27.66 | 74.71 |
| 1122_85 | 15.13 | 40.70 | 23.47 | 82.11 |
| 1122_86 | 16.64 | 53.29 | 75.13 | 113.41 |
| 1122_87 | 13.46 | 35.67 | 19.90 | 69.29 |
| 1122_88 | 12.85 | 34.25 | 24.41 | 68.38 |
| 1122_89 | 16.45 | 46.10 | 38.00 | 75.09 |
| 1122_90 | 18.52 | 50.88 | 60.64 | 93.90 |
| 1122_91 | 17.83 | 45.26 | 35.19 | 86.04 |
| 1122_92 | 15.49 | 41.46 | 21.27 | 68.79 |
| 1122_93 | 65.98 | 89.20 | 90.58 | 103.35 |
| 1122_94 | 63.83 | 92.70 | 82.15 | 98.30 |
| 1122_95 | 15.40 | 35.25 | 6.65 | 49.08 |
| 1122_96 | 16.51 | 44.63 | 2.69 | 48.45 |
| 1122_97 | 37.79 | 65.35 | 42.78 | 75.62 |
| 1122_98 | 14.34 | 38.27 | 0.91 | 42.75 |
| 1122_99 | 33.48 | 58.08 | 25.62 | 69.29 |
| 1122_100 | 12.21 | 31.92 | 0.85 | 44.41 |
| 1122_101 | 15.41 | 46.59 | 60.45 | 90.56 |
| 1122_102 | 15.62 | 41.71 | 16.98 | 62.88 |
| 1122_103 | 14.11 | 37.53 | 2.61 | 46.87 |
| 1122_104 | 18.01 | 47.10 | 36.56 | 81.02 |
| 1122_105 | 18.20 | 43.65 | 19.99 | 74.46 |
| 1122_106 | 16.64 | 36.77 | 7.75 | 55.14 |
| 1122_107 | 14.84 | 41.59 | 32.47 | 69.47 |
| 1122_108 | 12.89 | 32.26 | 8.10 | 72.49 |
| 1122_109 | 15.35 | 35.47 | 2.01 | 57.70 |
| 1122_110 | 19.02 | 45.56 | 0.72 | 45.61 |
| 1122_111 | 55.00 | 73.98 | 36.41 | 88.13 |
| 1122_112 | 16.38 | 40.70 | 0.77 | 50.17 |
| 1122_113 | 29.42 | 56.92 | 17.68 | 60.51 |
| 1122_114 | 13.63 | 33.67 | 0.43 | 42.62 |
| 1122_115 | 18.86 | 46.13 | 57.49 | 98.41 |
| 1122_116 | 31.73 | 59.42 | 28.44 | 86.09 |
| 1122_117 | 26.55 | 55.77 | 10.27 | 67.46 |
| 1122_118 | 49.24 | 68.77 | 52.70 | 88.13 |
| 1122_119 | 19.51 | 44.68 | 9.36 | 60.99 |
| 1122_120 | 16.50 | 40.36 | 2.16 | 51.59 |
| 1122_121 | 16.61 | 40.45 | 5.55 | 56.34 |
| 1122_123 | 15.49 | 38.63 | 0.95 | 52.77 |
| 1122_124 | 28.90 | 54.57 | 17.31 | 68.48 |
| 1122_125 | 15.75 | 41.51 | 12.29 | 63.50 |
| 1122_126 | 15.68 | 44.30 | 9.96 | 63.90 |
| 1122_127 | 14.91 | 45.71 | 9.92 | 55.07 |
| 1122_128 | 18.12 | 46.12 | 20.98 | 79.79 |
| 1122_129 | 15.62 | 44.07 | 9.19 | 69.22 |
| 1122_130 | 14.90 | 43.91 | 8.27 | 68.50 |
| 1122_131 | 17.75 | 46.92 | 20.03 | 67.67 |
| 1122_132 | 17.93 | 41.86 | 8.22 | 72.39 |
| 1122_133 | 18.11 | 47.91 | 14.60 | 78.23 |
| 1122_134 | 17.42 | 48.52 | 14.02 | 61.73 |
| 1122_135 | 14.62 | 46.43 | 3.71 | 52.73 |
| 1122_136 | 16.28 | 35.03 | 8.48 | 56.82 |
| 1122_137 | 14.92 | 37.45 | 12.42 | 65.30 |
| 1122_138 | 14.40 | 37.58 | 14.48 | 63.64 |
| 1122_139 | 15.06 | 38.30 | 14.21 | 66.30 |
| 1122_140 | 14.40 | 37.78 | 18.42 | 63.01 |
| 1122_141 | 16.19 | 37.64 | 16.31 | 64.03 |
| 1122_142 | 15.90 | 40.17 | 15.61 | 61.47 |
| 1122_143 | 14.70 | 103.24 | 12.58 | 109.05 |
| 1122_144 | 15.85 | 38.70 | 24.26 | 63.50 |
| 1122_145 | 14.15 | 36.53 | 13.02 | 67.51 |
| 1122_146 | 15.95 | 38.11 | 20.70 | 69.38 |
| 1122_147 | 15.08 | 40.89 | 19.48 | 62.04 |
| 1122_148 | 17.05 | 37.47 | 15.74 | 57.67 |
| 1122_149 | 15.43 | 35.40 | 43.92 | 78.04 |
| 1122_150 | 14.10 | 34.07 | 43.11 | 82.00 |
| 1122_151 | 15.65 | 37.73 | 48.08 | 92.95 |
| 1122_152 | 13.89 | 33.97 | 32.02 | 83.04 |
| 1122_153 | 13.97 | 35.53 | 40.47 | 82.39 |
| 1122_154 | 13.75 | 34.07 | 35.44 | 86.80 |
| 1122_155 | 14.15 | 43.77 | 43.08 | 83.80 |
| 1122_156 | 15.13 | 37.99 | 44.64 | 87.46 |
| 1122_157 | 14.34 | 40.19 | 57.37 | 88.72 |
| 1122_158 | 13.68 | 35.71 | 42.68 | 80.61 |
| 1122_159 | 15.47 | 40.55 | 57.30 | 86.78 |
| 1122_160 | 13.51 | 38.20 | 36.47 | 80.75 |
| 1122_161 | 15.52 | 42.53 | 10.78 | 64.30 |
| 1122_162 | 16.08 | 46.05 | 43.50 | 90.89 |
| 1122_163 | 17.28 | 45.49 | 42.75 | 86.76 |
| 1122_164 | 33.44 | 63.29 | 64.52 | 94.34 |
| 1122_165 | 29.00 | 63.38 | 67.97 | 84.55 |
| 1122_166 | 16.50 | 50.60 | 37.71 | 85.58 |
| 1122_167 | 12.92 | 42.08 | 37.91 | 76.46 |
| 1122_168 | 17.21 | 53.77 | 74.23 | 98.46 |
| 1122_169 | 13.00 | 41.35 | 17.57 | 69.24 |
| 1122_170 | 15.62 | 48.53 | 71.05 | 92.86 |
| 1122_171 | 12.95 | 35.94 | 10.80 | 59.51 |
| 1122_172 | 14.15 | 40.39 | 30.00 | 76.26 |
| 1122_173 | 16.04 | 48.13 | 50.42 | 95.73 |
| 1122_174 | 16.06 | 46.62 | 27.28 | 75.84 |
| 1122_175 | 17.19 | 41.23 | 44.35 | 89.61 |
| 1122_176 | 12.72 | 38.58 | 24.65 | 74.88 |
| 1122_177 | 43.57 | 68.03 | 56.40 | 78.38 |
| 1122_178 | 18.39 | 48.85 | 7.77 | 60.36 |
| 1122_179 | 12.21 | 36.54 | 0.66 | 39.24 |
| 1122_180 | 11.41 | 29.30 | 2.53 | 50.10 |
| 1122_181 | 11.39 | 29.60 | 1.37 | 45.39 |
| 1122_182 | 11.91 | 33.43 | 1.00 | 38.58 |
| 1122_183 | 15.53 | 53.20 | 70.04 | 89.74 |
| 1122_184 | 18.29 | 50.25 | 71.87 | 94.73 |
| 1122_185 | 12.42 | 38.65 | 16.98 | 64.32 |
| 1122_186 | 15.43 | 35.73 | 0.60 | 38.92 |
| 1122_188 | 95.10 | 91.87 | 98.34 | 92.17 |
| 1122_189 | 13.17 | 45.93 | 42.56 | 81.86 |
| 1122_190 | 88.24 | 96.08 | 101.92 | 93.17 |
| 1122_191 | 17.43 | 47.23 | 35.22 | 84.24 |
| 1122_192 | 14.50 | 41.74 | 2.75 | 47.15 |
| 1122_193 | 17.94 | 45.59 | 16.35 | 70.52 |
| 1122_194 | 19.14 | 42.96 | 2.51 | 44.63 |
| 1122_195 | 15.03 | 41.04 | 6.84 | 58.60 |
| 1122_196 | 14.86 | 39.28 | 0.98 | 47.63 |
| 1122_197 | 14.80 | 39.13 | 0.62 | 45.40 |
| 1122_198 | 23.68 | 54.40 | 20.87 | 71.13 |
| 1122_199 | 22.98 | 50.51 | 18.55 | 67.76 |
| 1122_200 | 23.58 | 50.62 | 37.86 | 81.81 |
| 1122_201 | 19.40 | 46.35 | 7.15 | 59.27 |
| 1122_202 | 18.20 | 46.98 | 5.78 | 54.72 |
| 1122_203 | 17.69 | 45.67 | 2.10 | 48.19 |
| 1122_204 | 13.49 | 37.08 | 0.50 | 43.42 |
| 1122_205 | 115.04 | 44.88 | 5.19 | 83.93 |
| 1122_206 | 12.85 | 35.68 | 1.05 | 52.13 |
| 1122_207 | 16.58 | 45.34 | 58.08 | 97.54 |
| 1122_208 | 17.59 | 47.72 | 13.12 | 61.52 |
| 1122_209 | 18.16 | 44.97 | 35.65 | 72.69 |
| 1122_210 | 17.36 | 47.09 | 30.36 | 68.94 |
| 1122_211 | 19.96 | 46.56 | 20.87 | 72.34 |
| 1122_212 | 24.09 | 54.75 | 49.10 | 83.35 |
| 1122_213 | 18.37 | 48.15 | 27.49 | 74.55 |
| 1122_214 | 14.29 | 39.30 | 15.92 | 66.14 |
| 1122_215 | 15.37 | 38.56 | 2.04 | 41.18 |
| 1122_216 | 15.01 | 42.66 | 7.02 | 58.95 |
| 1122_217 | 17.30 | 46.90 | 32.59 | 82.10 |
| 1122_218 | 13.77 | 42.95 | 25.43 | 71.68 |
| 1122_219 | 12.87 | 37.59 | 17.95 | 52.45 |
| 1122_220 | 13.52 | 38.61 | 1.85 | 48.48 |
| 1122_221 | 18.36 | 45.47 | 29.00 | 75.06 |
| 1122_222 | 23.95 | 54.80 | 19.25 | 67.91 |
| 1122_223 | 19.96 | 48.20 | 33.50 | 74.16 |
| 1122_224 | 68.09 | 95.88 | 81.06 | 104.47 |
| 1122_225 | 31.31 | 66.62 | 53.44 | 90.70 |
| 1122_226 | 102.33 | 93.40 | 96.03 | 101.72 |
| 1122_227 | 17.68 | 49.92 | 63.44 | 98.06 |
| 1122_228 | 12.94 | 28.36 | 0.52 | 40.67 |
| 1122_229 | 13.82 | 36.60 | 25.36 | 83.32 |
| 1122_230 | 13.78 | 29.77 | 1.16 | 53.35 |
| 1122_231 | 13.75 | 34.55 | 14.04 | 67.82 |
| 1122_232 | 12.79 | 33.53 | 16.70 | 73.24 |
| 1122_233 | 11.52 | 28.88 | 0.67 | 39.96 |
| 1122_234 | 12.10 | 29.35 | 0.12 | 30.89 |
| 1122_235 | 10.75 | 31.37 | 1.35 | 45.12 |
| 1122_236 | 15.54 | 36.00 | 19.38 | 78.46 |
| 1122_237 | 14.85 | 38.11 | 20.56 | 76.35 |
| 1122_238 | 98.98 | 100.70 | 93.56 | 109.21 |
| 1122_239 | 18.93 | 56.23 | 59.59 | 93.71 |
| 1122_240 | 14.80 | 38.69 | 0.90 | 42.40 |
| 1122_241 | 15.37 | 46.65 | 20.38 | 66.87 |
| 1122_242 | 15.24 | 42.10 | 13.87 | 68.39 |
| 1122_243 | 15.73 | 44.21 | 14.67 | 66.50 |
| 1122_244 | 14.11 | 40.27 | 0.86 | 39.99 |
| 1122_245 | 13.14 | 33.83 | 0.38 | 39.46 |
| 1122_247 | 12.86 | 33.71 | 0.90 | 43.55 |
| 1122_248 | 90.91 | 95.53 | 106.59 | 91.46 |
| 1122_249 | 14.22 | 41.84 | 40.90 | 87.00 |
| 1122_250 | 90.31 | 100.03 | 100.46 | 97.45 |
| 1122_251 | 102.82 | 102.41 | 98.63 | 99.26 |
| 1122_252 | 16.50 | 48.66 | 38.13 | 85.83 |
| 1122_253 | 17.79 | 48.73 | 31.96 | 79.20 |
| 1122_254 | 16.26 | 46.83 | 26.68 | 70.97 |
| 1122_255 | 16.23 | 39.36 | 1.22 | 48.88 |
| 1122_256 | 18.48 | 44.91 | 7.37 | 62.07 |
| 1122_257 | 18.72 | 43.81 | 1.85 | 49.52 |
| 1122_258 | 28.90 | 59.55 | 22.76 | 80.32 |
| 1122_259 | 13.02 | 35.53 | 0.27 | 37.49 |
| 1122_260 | 39.03 | 64.19 | 25.97 | 75.30 |
| 1122_261 | 25.96 | 54.58 | 4.80 | 54.40 |
| 1122_262 | 64.09 | 79.15 | 48.38 | 86.81 |
| 1122_263 | 41.44 | 64.92 | 14.48 | 60.49 |
| 1122_264 | 31.93 | 53.80 | 8.86 | 57.82 |
| 1122_265 | 21.30 | 46.83 | 1.90 | 48.70 |
| 1122_266 | 12.74 | 38.89 | 0.38 | 39.26 |
| 1122_267 | 13.34 | 34.60 | 0.35 | 35.53 |
| 1122_268 | 21.75 | 52.36 | 45.71 | 88.45 |
| 1122_269 | 15.62 | 34.21 | 0.99 | 42.01 |
| 1122_270 | 18.23 | 48.08 | 55.25 | 92.41 |
| 1122_271 | 29.49 | 58.90 | 12.49 | 74.08 |
| 1122_272 | 22.71 | 53.62 | 17.96 | 86.69 |
| 1122_273 | 30.84 | 63.97 | 39.74 | 79.91 |
| 1122_274 | 25.06 | 55.33 | 14.72 | 72.24 |
| 1122_275 | 46.36 | 77.28 | 49.33 | 91.76 |
| 1122_276 | 22.07 | 52.96 | 22.13 | 75.39 |
| 1122_277 | 24.76 | 52.41 | 2.80 | 104.85 |
| 1122_278 | 16.09 | 39.55 | 1.05 | 53.39 |
| 1122_279 | 16.97 | 41.64 | 2.59 | 53.82 |
| 1122_280 | 19.16 | 47.62 | 53.88 | 89.20 |
| 1122_281 | 15.33 | 35.15 | 1.21 | 49.25 |
| 1122_282 | 18.74 | 46.47 | 19.18 | 70.60 |
| 1122_283 | 14.21 | 38.75 | 0.92 | 49.39 |
| 1122_284 | 21.36 | 50.04 | 17.88 | 77.20 |
| 1122_285 | 24.58 | 53.16 | 11.30 | 69.32 |
| 1122_286 | 26.61 | 59.28 | 32.18 | 85.20 |
| 1122_287 | 19.11 | 46.43 | 40.68 | 87.49 |
| 1122_288 | 17.38 | 46.11 | 29.23 | 83.51 |
| 1122_289 | 22.65 | 50.00 | 18.22 | 80.28 |
| 1122_290 | 15.73 | 46.26 | 46.94 | 97.94 |
| 1122_291 | 17.59 | 47.83 | 57.89 | 97.49 |
| 1122_292 | 18.87 | 45.34 | 51.24 | 76.10 |
| 1122_293 | 19.91 | 50.35 | 80.98 | 93.18 |
| 1122_294 | 15.68 | 44.72 | 63.82 | 89.72 |
| 1122_295 | 17.72 | 46.95 | 72.65 | 97.04 |
| 1122_296 | 18.22 | 45.33 | 84.64 | 65.09 |
| 1122_297 | 21.12 | 52.34 | 55.13 | 90.63 |
| 1122_298 | 19.04 | 49.85 | 43.05 | 83.36 |
| 1122_299 | 21.00 | 53.98 | 52.09 | 88.91 |
| 1122_300 | 21.03 | 56.31 | 63.83 | 96.36 |
| 1122_301 | 15.75 | 41.51 | 15.41 | 71.45 |
| 1122_302 | 15.98 | 41.88 | 33.75 | 82.19 |
| 1122_303 | 14.92 | 33.22 | 16.97 | 73.97 |
| 1122_304 | 12.17 | 33.01 | 0.96 | 54.41 |
| 1122_305 | 17.66 | 38.97 | 1.49 | 58.34 |
| 1122_306 | 18.52 | 42.72 | 3.38 | 58.51 |
| 1122_307 | 42.52 | 62.86 | 49.15 | 83.10 |
| 1122_308 | 15.75 | 46.33 | 18.59 | 81.21 |
| 1122_309 | 18.69 | 46.63 | 43.91 | 88.58 |
| 1122_310 | 21.63 | 49.10 | 64.20 | 92.07 |
| 1122_311 | 15.23 | 36.71 | 0.82 | 55.64 |
| 1122_312 | 16.28 | 40.30 | 14.72 | 71.61 |
| 1122_313 | 17.13 | 42.41 | 26.14 | 78.29 |
| 1122_314 | 17.15 | 44.16 | 17.44 | 74.43 |
| 1122_315 | 15.87 | 37.68 | 3.87 | 56.29 |
| 1122_316 | 12.46 | 40.91 | 15.99 | 79.86 |
| 1122_317 | 19.92 | 51.35 | 75.13 | 91.76 |
| 1122_318 | 19.72 | 55.90 | 84.62 | 96.07 |
| 1122_319 | 14.89 | 40.90 | 30.49 | 84.12 |
| 1122_320 | 14.12 | 38.83 | 35.60 | 80.47 |
| 1122_321 | 16.55 | 47.64 | 39.44 | 85.96 |
| 1122_322 | 19.89 | 46.85 | 51.06 | 83.91 |
| 1122_323 | 16.91 | 39.33 | 31.59 | 85.79 |
| 1122_324 | 13.32 | 34.73 | 23.93 | 76.58 |
| 1122_325 | 13.64 | 36.88 | 34.23 | 82.54 |
| 1122_326 | 17.03 | 47.80 | 65.51 | 93.50 |
| 1122_327 | 13.18 | 34.45 | 0.61 | 41.05 |
| 1122_328 | 28.85 | 51.75 | 33.40 | 73.68 |
| 1122_329 | 18.98 | 45.02 | 9.68 | 55.71 |
| 1122_330 | 26.09 | 52.37 | 29.65 | 77.91 |
| 1122_331 | 19.50 | 48.30 | 9.77 | 63.78 |
| 1122_332 | 17.96 | 54.05 | 7.57 | 72.56 |
| 1122_333 | 18.01 | 45.82 | 5.15 | 54.92 |
| 1122_334 | 16.51 | 45.70 | 5.62 | 58.84 |
| 1122_335 | 15.28 | 35.40 | 5.27 | 59.31 |
| 1122_336 | 18.18 | 45.30 | 24.99 | 75.94 |
| 1816_2 | 60.44 | 75.00 | 72.19 | 101.12 |
| 1816_3 | 32.49 | 64.72 | 81.52 | 99.97 |
| 1816_4 | 20.62 | 53.39 | 86.99 | 94.28 |
| 1816_5 | 28.44 | 63.88 | 87.90 | 101.86 |
| 1816_6 | 22.90 | 52.87 | 82.34 | 95.59 |
| 1816_7 | 80.07 | 88.95 | 93.22 | 101.92 |
| 1816_8 | 43.52 | 67.76 | 96.84 | 104.02 |
| 1816_9 | 21.44 | 54.71 | 92.27 | 103.41 |
| 1816_10 | 17.09 | 49.44 | 76.99 | 95.59 |
| 1816_11 | 21.06 | 53.06 | 78.28 | 98.39 |
| 1816_12 | 20.06 | 55.59 | 84.79 | 102.08 |
| 1816_13 | 17.14 | 48.05 | 93.50 | 100.40 |
| 1816_14 | 19.55 | 59.40 | 90.51 | 105.28 |
| 1816_15 | 22.72 | 59.25 | 101.19 | 106.47 |
| 1816_16 | 24.44 | 62.31 | 96.03 | 102.54 |
| 1816_17 | 22.53 | 55.69 | 91.49 | 101.21 |
| 1816_18 | 21.46 | 53.93 | 85.50 | 99.48 |
| 1816_19 | 21.31 | 54.40 | 87.10 | 95.06 |
| 1816_20 | 23.72 | 53.87 | 92.47 | 105.09 |
| 1816_21 | 19.76 | 50.72 | 89.60 | 105.93 |
| 1816_22 | 95.15 | 104.15 | 102.13 | 103.05 |
| 1816_24 | 91.75 | 95.00 | 98.28 | 102.02 |
| 1816_25 | 56.04 | 88.36 | 86.52 | 109.26 |
| 1816_26 | 102.21 | 101.78 | 105.12 | 108.00 |
| 1816_27 | 24.15 | 67.01 | 92.61 | 99.83 |
| 1816_28 | 21.76 | 57.39 | 101.49 | 98.46 |
| 1816_29 | 30.53 | 72.76 | 99.75 | 98.54 |
| 1816_30 | 24.86 | 57.27 | 74.75 | 84.15 |
| 1816_31 | 41.00 | 72.35 | 106.54 | 101.47 |
| 1816_32 | 21.68 | 53.64 | 83.45 | 100.28 |
| 1816_33 | 36.10 | 74.71 | 78.48 | 109.62 |
| 1816_34 | 43.48 | 68.78 | 90.47 | 99.53 |
| 1816_35 | 78.05 | 89.09 | 93.93 | 96.53 |
| 1816_36 | 81.65 | 87.07 | 91.10 | 97.06 |
| 1816_37 | 62.87 | 82.62 | 96.63 | 102.82 |
| 1816_38 | 34.50 | 62.63 | 88.71 | 98.47 |
| 1816_39 | 17.06 | 43.55 | 68.41 | 91.25 |
| 1816_40 | 22.42 | 59.75 | 58.94 | 93.82 |
| 1816_41 | 24.34 | 60.50 | 78.94 | 94.74 |
| 1816_42 | 17.21 | 51.49 | 88.87 | 106.71 |
| 1816_43 | 15.45 | 48.67 | 93.79 | 96.40 |
| 1816_44 | 27.69 | 62.14 | 89.86 | 98.76 |
| 1816_45 | 28.43 | 62.53 | 93.60 | 104.83 |
| 1816_46 | 38.65 | 95.31 | 99.48 | 95.34 |
| 1816_47 | 39.25 | 73.69 | 85.32 | 99.46 |
| 1816_48 | 72.88 | 95.79 | 95.86 | 105.23 |
| 1816_49 | 42.05 | 72.73 | 95.26 | 95.07 |
| 1816_50 | 24.87 | 61.64 | 93.95 | 95.41 |
| 1816_51 | 24.13 | 58.48 | 94.87 | 86.45 |
| 1816_52 | 19.07 | 46.64 | 61.73 | 103.50 |
| 1816_53 | 24.65 | 59.39 | 95.95 | 103.39 |
| 1816_54 | 18.52 | 50.37 | 75.25 | 100.65 |
| 1816_55 | 21.38 | 55.81 | 80.93 | 97.54 |
| 1816_56 | 23.72 | 74.56 | 81.95 | 94.12 |
| 1816_57 | 31.08 | 63.23 | 97.13 | 97.12 |
| 1816_58 | 26.25 | 60.06 | 82.44 | 91.63 |
| 1816_59 | 38.54 | 74.45 | 96.86 | 99.10 |
| 1816_60 | 21.27 | 53.40 | 102.79 | 95.99 |
| 1816_61 | 22.84 | 55.21 | 84.32 | 108.50 |
| 1816_62 | 28.18 | 66.34 | 89.02 | 100.92 |
| 1816_63 | 22.52 | 58.32 | 99.09 | 104.39 |
| 1816_64 | 17.21 | 48.69 | 101.88 | 109.05 |
| 1816_65 | 18.02 | 48.71 | 97.59 | 108.12 |
| 1816_66 | 24.48 | 59.70 | 98.74 | 91.48 |
| 1816_67 | 25.08 | 60.20 | 102.17 | 102.41 |
| 1816_68 | 18.20 | 52.03 | 93.53 | 107.27 |
| 1816_69 | 28.58 | 64.76 | 100.03 | 95.56 |
| 1816_70 | 32.32 | 65.70 | 93.94 | 104.28 |
| 1816_71 | 47.83 | 78.25 | 89.25 | 109.07 |
| 1816_72 | 35.33 | 62.27 | 91.64 | 107.72 |
| 1816_73 | 20.55 | 98.77 | 85.68 | 103.65 |
| 1816_74 | 26.71 | 60.10 | 87.66 | 99.83 |

### Example 15: In vitro toxicity evaluation caspase assays

### Materials and Methods:

Based on the results in Table 12, the compounds most potent in targeting ATXN3 with the least effect on KCNB2 were selected for toxicity evaluation in two caspase activation assays (see Dieckmann et al., Molecular Therapy: Nucleic Acids Vol. 10 Mar. 2018, pp 45-54); respectively conducted in HepG2 cells (“HEPG2”) and 3T3 cells (“3T3”), Results:

The results are shown in Table 13. Using the same scoring criteria as in Example 7, the vast majority of the compounds were found safe in the toxicity assessment. In the “HEPG2” assay, compound 1816_52 had a score of 0.5, compounds 1816_30 and 1816_40 a score of 1, and compound 1816_39 a score of 3. In the “313” assay, the compounds with a score of 0.5 were: 1816_11, 1816_74, and 1816_30; the compounds with a score of I were: 1816_54 and 1816_10; the compound with a score of two was: 1816_52; and the compounds with a score of three were: 1816_40 and 1816_39.

TABLE 13

| | HepG2 | 3T3 | EC50 | EC50 | EC50 ratio |
|---|---|---|---|---|---|
| | Assay | Assay | ATXN3 | KCNB2 | (KCNB2/ |
| CMPIDNO | score | score | (nM) | (nM) | ATXN3) |
| 1122_33 | 0 | 0 | 103.7 | 2330 | 22.5 |
| 1122_67 | 0 | 0 | 71.09 | 155.40 | 2.20 |
| 1122_91 | 0 | 0 | 87.73 | 673.50 | 7.68 |
| 1122_107 | 0 | 0 | 89.93 | 789.00 | 8.77 |
| 1122_125 | 0 | 0 | 81.85 | 98.95 | 1.2 |
| 1122_144 | 0 | 0 | 103.10 | 515.60 | 5.00 |
| 1122_146 | 0 | 0 | 93.11 | 490.10 | 5.26 |
| 1122_149 | 0 | 0 | 62.77 | 766.80 | 12.22 |
| 1122_150 | 0 | 0 | 89.36 | 808.50 | 9.05 |
| 1122_151 | 0 | 0 | 75.71 | 654.80 | 8.65 |
| 1122_152 | 0 | 0 | 66.09 | 662.20 | 10.02 |
| 1122_153 | 0 | 0 | 84.38 | 1208.00 | 14.32 |
| 1122_154 | 0 | 0 | 64.22 | 820.00 | 12.77 |
| 1122_155 | 0 | 0 | 75.16 | 1303.00 | 17.34 |
| 1122_156 | 0 | 0 | 55.26 | 736.10 | 13.32 |
| 1122_157 | 0 | 0 | 71.27 | 1658.00 | 23.26 |
| 1122_158 | 0 | 0 | 53.18 | 1217.00 | 22.88 |
| 1122_159 | 0 | 0 | 92.93 | 1472.00 | 15.84 |
| 1122_160 | 0 | 0 | 57.47 | 791.50 | 13.77 |
| 1122_163 | 0 | 0 | 98.81 | 564.40 | 5.71 |
| 1122_167 | 0 | 0 | 87.59 | 763.60 | 8.72 |
| 1122_172 | 0 | 0 | 67.13 | 465.70 | 6.94 |
| 1122_175 | 0 | 0 | 127.90 | 1277.00 | 9.98 |
| 1122_218 | 0 | 0 | 87.76 | 311.80 | 3.55 |
| 1122_294 | 0 | 0 | 93.12 | 2338.00 | 25.11 |
| 1122_296 | 0 | 0 | 114.80 | 8032.00 | 69.97 |
| 1122_302 | 0 | 0 | 104.10 | 682.80 | 6.56 |
| 1122_313 | 0 | 0 | 90.50 | 418.70 | 4.63 |
| 1122_319 | 0 | 0 | 70.04 | 408.20 | 5.83 |
| 1122_320 | 0 | 0 | 87.66 | 364.50 | 4.16 |
| 1122_323 | 0 | 0 | 99.59 | 720.60 | 7.24 |
| 1122_325 | 0 | 0 | 90.83 | 585.40 | 6.45 |
| 1816_4 | 0 | 0 | 200.90 | 248307.00 | 1235.97 |
| 1816_6 | 0 | 0 | 261.00 | 26826.00 | 102.78 |
| 1816_9 | 0 | 0 | 224.00 | 3280.00 | 14.64 |
| 1816_10 | 0 | 1 | — | — | — |
| 1816_11 | 0 | 0.5 | — | — | — |
| 1816_12 | 0 | 0 | 110 | 27238 | 247.6 |
| 1816_13 | 0 | 0 | 129.30 | 36344.00 | 281.08 |
| 1816_14 | 0 | 0 | 181.10 | 2262.00 | 12.49 |
| 1816_15 | 0 | 0 | 159.80 | 18449.00 | 115.45 |
| 1816_17 | 0 | 0 | 208.90 | 33758.00 | 161.60 |
| 1816_18 | 0 | 0 | 138.40 | 322538.00 | 2330.48 |
| 1816_19 | 0 | 0 | 180.40 | 107214.00 | 594.31 |
| 1816_20 | 0 | 0 | 299.50 | 279468.00 | 933.12 |
| 1816_21 | 0 | 0 | 203.00 | 16057.00 | 79.10 |
| 1816_28 | 0 | 0 | 141.10 | 10000.00 | 70.87 |
| 1816_30 | 1 | 0.5 | — | — | — |
| 1816_32 | 0 | 0 | 170.80 | 13406.00 | 78.49 |
| 1816_39 | 3 | 3 | — | — | — |
| 1816_40 | 1 | 3 | — | — | — |
| 1816_41 | 0 | 0 | 154.50 | 12106.00 | 78.36 |
| 1816_42 | 0 | 0 | 134.60 | 63672.00 | 473.05 |
| 1816_43 | 0 | 0 | 89.79 | 569489.00 | 6342.45 |
| 1816_51 | 0 | 0 | 239.70 | 9150.00 | 38.17 |
| 1816_52 | 0.5 | 2 | — | — | — |
| 1816_53 | 0 | 0 | 392.80 | 35864.00 | 91.30 |
| 1816_54 | 0 | 1 | — | — | — |
| 1816_55 | 0 | 0 | 209.00 | 7219.00 | 34.54 |
| 1816_58 | 0 | 0 | 259.00 | 23941.00 | 92.44 |
| 1816_60 | 0 | 0 | 167.10 | 67695.00 | 405.12 |
| 1816_61 | 0 | 0 | 165.20 | 22618.00 | 136.91 |
| 1816_63 | 0 | 0 | 263.00 | 19355.00 | 73.59 |
| 1816_64 | 0 | 0 | 127.00 | 5929.00 | 46.69 |
| 1816_65 | 0 | 0 | 99.75 | 36592.00 | 366.84 |
| 1816_66 | 0 | 0 | 351.30 | 3822.00 | 10.88 |
| 1816_67 | 0 | 0 | — | — | — |
| 1816_68 | 0 | 0 | 177.80 | 112276.00 | 631.47 |
| 1816_74 | 0 | 0.5 | — | — | — |

### Example 16: Determination of EC50 values for ATXN3 and KCNB2 in iCell® Glutaneurons

### Materials and Methods:

Compounds were assessed in an EC50 determination in iCell Glutaneurons including compound 1122_67 and 1122:33. The experimental setup was the same as described in Example 14, except that the following compound concentrations were used (nM): 31.6; 10; 3.2; 1; 0.32; 0.1; 0.03; 0.01 (8-step half-log).

### Results:

The resulting EC50 values for ATXN3 and KCNB2 as well as the resulting ratio between the EC50 values (KCNB2/ATXN3) are shown in Table 13. From the data generated, it was observed that the compounds showed an increased ratio between the determined EC50 values for KCNB2 and ATXN3 as compared to compound 1122_67. The potency of the compounds on ATXN3 knockdown was relatively maintained for most compounds while it was decreased (higher EC50 value) for all compounds when focusing on the potency on KCNB2 knockdown.

Based on the data in Table 12 (double point determination) and Table 13 (caspase in vitro toxicity; EC50 for ATXN3 knock down and the ratio between KCNB2 and ATXN3 EC50 values). 23 compounds (shown in Table 14 and in FIG. 12) were selected based on their safety (caspase scores above 0 were discontinued), high potency and efficacy in ATXN3 inhibition, selectivity (i.e., high ratio between KCNB2/ATXN3 EC50 values) as well as their chemical diversity.

The base sequence, sugar sequence and backbone sequence features of the selected compounds are shown in Table 14, using the HELM-dictionary shown below (see Example 13 for more detailed HELM annotations).

| Base sequence | Sugar sequence | Backbone sequence |
|---|---|---|
| A: (A) | D: [dR] | S: [ssP] |
| C: (C) | F: [fR] | X: [sP] |
| E: (5meC) | L: [LR] | |
| G: (G) | M: [MOE] | |
| T: (T) | O: [mR] | |
| U: (U) | | |

TABLE 14
Selected compounds

| CMPIDNO | Base sequence | Sugar sequence | Backbone sequence | FIG. |
|---|---|---|---|---|
| 1122_91 | AATETTATTUACATCTTEE | LDDLDLDDDODDDDDDLLL | XXXXXXXXXXXXXXXXXX | 12A |
| 1122_107 | AAUETTATTTACATCTTEE | LLOLDDDDDDDDDDDLDLL | XXXXXXXXXXXXXXXXXX | 12B |
| 1122_154 | AATETTATTTACATCTTEE | LDDLDLDDDDDDDDDDLLL | XXXXXXXXXXSXXXXXXX | 12C |
| 1122_155 | AATETTATTTACATCTTEE | LDDLDLDDDDDDDDDDLLL | XXXXXXXXXXXSXXXXXX | 12D |
| 1122_156 | AATETTATTTACATCTTEE | LDDLDLDDDDDDDDDDLLL | XXXXXXXXXXXXSXXXXX | 12E |
| 1122_157 | AATETTATTTACATCTTEE | LDDLDLDDDDDDDDDDLLL | XXXXXXXXXXXXXSXXXX | 12F |
| 1122_158 | AATETTATTTACATCTTEE | LDDLDLDDDDDDDDDDLLL | XXXXXXXXXXXXXSXXXX | 12G |
| 1122_167 | AATETTATTTACATCTTEE | LDDLDLMDDDDDDDDDLLL | XXXXXXXXXXXXXXXXXX | 12H |
| 1122_172 | AATETTATTTACATCTTEE | LDDLDLDDDDDDDDODLLL | XXXXXXXXXXXXXXXXXX | 12I |
| 1122_175 | AATETTATTTACATCTTEE | LDDLDLDDDDODDDDDLLL | XXXXXXXXXXXXXXXXXX | 12J |
| 1122_294 | AATETTATTTACATCTTCE | LDDLDLDDDDDDDDDDLFL | XXXXXXXXXXXXXXXXXX | 12K |
| 1122_296 | AATCTTATTTACATCTTEE | LDDODLDDDDDDDDDDLLL | XXXXXXXXXXXXXXXXXX | 12L |
| 1816_13 | GAATETTATTTACATETT | LLDDLDLLDDDDDDDLLL | XXXXXXXXXXXSXXXXX | 12M |
| 1816_15 | GAATETTATTTACATETT | LLDDLDLLDDDDDDDLLL | XXXXXXXXXXXXXSXXX | 12N |
| 1816_28 | GAATETTATTTACATETT | LLDDLDLFDDDDDDDLLL | XXXXXXXXXXXXXSXXX | 12O |
| 1816_41 | GAATETTATTTACATETT | LLLDLDLLDDDDDDDLLL | XXXXXXXXXXXXXXXXX | 12P |
| 1816_42 | GAATETTATTTACATETT | LLDDLDLLDDDDDDDLML | XXXXXXXXXXXXXXXXX | 12Q |
| 1816_43 | GAATETTATTTACATETT | LLDDLDLLDDDDDDDMLL | XXXXXXXXXXXXXXXXX | 12R |
| 1816_60 | GAATETTATTTACATETT | LLDDLDLODDDDDDDLLL | XXXXXXXXXXXXXXXXX | 12S |
| 1816_61 | GAATETTATTTACATETT | LLODLDLLDDDDDDDLLL | XXXXXXXXXXXXXXXXX | 12T |
| 1816_64 | GAATETTATTTACATCTT | LLDDLDLLDDDDDDDFLL | XXXXXXXXXXXXXXXXX | 12U |
| 1816_65 | GAATETTATTTACATCTT | LLDDLDLLDDDDDDDOLL | XXXXXXXXXXXXXXXXX | 12V |
| 1816_68 | GAATETTATTTACATEUT | LLDDLDLLDDDDDDDLFL | XXXXXXXXXXXXXXXXX | 12W |

TABLE 8B

| | Cortex | | Midbrain | | Cerebellum | | Hippocampus | | Pons/medulla | | Striatum | |
|---|---|---|---|---|---|---|---|---|---|---|---|---|
| | | Max | | Max | | Max | | Max | | Max | | Max |
| | EC50 | efficacy (% | EC50 | efficacy (% | EC50 | efficacy (% | EC50 | efficacy (% | EC50 | efficacy (% | EC50 | efficacy (% |
| Compounds | (nM) | remaining) | (nM) | remaining) | (nM) | remaining) | (nM) | remaining) | (nM) | remaining) | (nM) | remaining) |
| 1856_1 | 251 | 33 | 77 | 20 | 434 | 49 | 202 | 41 | — | 24 | 103 | 27 |
| 1813_1 | 260 | 22 | 93 | 20 | 347 | 47 | 279 | 30 | — | 22 | 89 | 18 |
| 1812_1 | 307 | 52 | 156 | 28 | 603 | 50 | 233 | 35 | — | 26 | 184 | 32 |
| 1809_2 | 134 | 57 | 153 | 34 | 511 | 50 | 111 | 46 | — | 21 | 93 | 29 |
| 1607_1 | 193 | 40 | 89 | 17 | 120 | 42 | 81 | 21 | — | 15 | 63 | 26 |
| 1122_62 | 125 | 56 | 74 | 26 | 226 | 16 | 86 | 46 | — | 19 | 54 | 36 |
| 1122_67 | 125 | 23 | 79 | 14 | 261 | 27 | 146 | 22 | — | 13 | 88 | 19 |
| 1122_33 | 102 | 47 | 38 | 16 | 166 | 35 | 79 | 24 | — | 17 | 63 | 29 |

TABLE 10

| | | Cortex (A1) | | Cerebellum | | Brainstem | | Midbrain | | Striatum | |
|---|---|---|---|---|---|---|---|---|---|---|---|
| | | EC50 | Max KD | EC50 | Max KD | EC50 | Max KD | EC50 | Max KD | EC50 | Max KD |
| Tissue | | (nM) | observed | (nM) | observed | (nM) | observed | (nM) | observed | (nM) | observed |
| 1 week of | 1122_67 | 242 | 88% | 833 | 74% | 196 | 87% | 165 | 89% | 148 | 77% |
| treatment | 1813_1 | 278 | 61% | 966 | 57% | 377 | 85% | 183 | 90% | 118 | 51% |
| | 1100673 | 391 | 67% | 2012 | 48% | 769 | 79% | 279 | 81% | 331 | 69% |
| 4 week of | 1122_67 | 100 | 92% | 365 | 81% | 81 | 93% | 94 | 95% | 46 | 89% |
| treatment | 1813_1 | ND | ND | ND | ND | ND | ND | ND | ND | ND | ND |
| | 1100673 | 199 | 49% | 1229 | 33% | 419 | 72% | 129 | 74% | 130 | 35% |

| | | | | Spinal cord, | | Spinal cord, | | Spinal cord, | |
|---|---|---|---|---|---|---|---|---|---|
| | | Hippocampus | | cervical | | thoracic | | lumbar | |
| | | EC50 | Max KD | EC50 | Max KD | EC50 | Max KD | EC50 | Max KD |
| Tissue | | (nM) | observed | (nM) | observed | (nM) | observed | (nM) | observed |
| 1 week of | 1122_67 | 243 | 75% | 41 | 89% | 39 | 90% | 54 | 89% |
| treatment | 1813_1 | 341 | 63% | 45 | 90% | 36 | 92% | 48 | 91% |
| | 1100673 | 516 | 66% | 83 | 83% | 51 | 83% | 68 | 82% |
| 4 week of | 1122_67 | 89 | 92% | 16 | 93% | Imprecise | 93% | 18 | 93% |
| treatment | 1813_1 | ND | ND | ND | ND | ND | ND | ND | ND |
| | 1100673 | 329 | 52% | 48 | 83% | Imprecise | 84% | 56 | 84% |

TABLE 15
Particular antisense oligonucleotide variants of SEQ ID NO: 1122
Options for nucleobases, sugar modifications and internucleoside linkages for particular antisense
oligonucleotides according to the invention. The options from which the nucleoside at each specific
residue # in SEQ ID NO: 1122 can be chosen is shown in each line indicated by that residue #,
starting from the 5′-end. The selection of a specific nucleobase (“Nucleobase options for residue #”)
and a sugar modification (“Sugar modification options for residue #”) defines the nucleoside at
that residue #. The options for the internucleoside linkage between the nucleoside at a specific
residue # and the next residue, starting from the 5′-end, is shown in the column entitled
“Backbone modification options for internucleoside linkage #”.

| | | | | | | | | | | | | | Sugar | | | | | | | | | | | | Backbone | | | | | | | | | | | |
|---|---|---|---|---|---|---|---|---|---|---|---|---|---|---|---|---|---|---|---|---|---|---|---|---|---|---|---|---|---|---|---|---|---|---|---|---|
| Residue # | Nucleobase options | | | | | | | | | | | | modification options | | | | | | | | | | | | modification options for | | | | | | | | | | | |
| in SEQ ID | for residue # | | | | | | | | | | | | for residue # | | | | | | | | | | | | internucleoside linkage # | | | | | | | | | | | |
| NO: 1122 | A: (A); C: (C); E: (5meC); | | | | | | | | | | | | D: [dR]; F: [fR]; | | | | | | | | | | | | (from 5′-end) | | | | | | | | | | | |
| (from 5′-end) | G: (G); T: (T); U: (U) | | | | | | | | | | | | L: [LR]; M: [MOE]; O: [mR] | | | | | | | | | | | | S: [ssP]; X: [sP] | | | | | | | | | | | |
| 1 | A | A | A | A | A | A | A | A | A | A | A | A | L | L | L | L | L | L | L | L | L | L | L | L | X | X | X | X | X | X | X | X | X | X | X | X |
| 2 | A | A | A | A | A | A | A | A | A | A | A | A | D | L | D | D | D | D | D | D | D | D | D | D | X | X | X | X | X | X | X | X | X | X | X | X |
| 3 | T | U | T | T | T | T | T | T | T | T | T | T | D | O | D | D | D | D | D | D | D | D | D | D | X | X | X | X | X | X | X | X | X | X | X | X |
| 4 | E | E | E | E | E | E | E | E | E | E | E | C | L | L | L | L | L | L | L | L | L | L | L | O | X | X | X | X | X | X | X | X | X | X | X | X |
| 5 | T | T | T | T | T | T | T | T | T | T | T | T | D | D | D | D | D | D | D | D | D | D | D | D | X | X | X | X | X | X | X | X | X | X | X | X |
| 6 | T | T | T | T | T | T | T | T | T | T | T | T | L | D | L | L | L | L | L | L | L | L | L | L | X | X | X | X | X | X | X | X | X | X | X | X |
| 7 | A | A | A | A | A | A | A | A | A | A | A | A | D | D | D | D | D | D | D | M | D | D | D | D | X | X | X | X | X | X | X | X | X | X | X | X |
| 8 | T | T | T | T | T | T | T | T | T | T | T | T | D | D | D | D | D | D | D | D | D | D | D | D | X | X | X | X | X | X | X | X | X | X | X | X |
| 9 | T | T | T | T | T | T | T | T | T | T | T | T | D | D | D | D | D | D | D | D | D | D | D | D | X | X | X | X | X | X | X | X | X | X | X | X |
| 10 | U | T | T | T | T | T | T | T | T | T | T | T | O | D | D | D | D | D | D | D | D | D | D | D | X | X | X | X | X | X | X | X | X | X | X | X |
| 11 | A | A | A | A | A | A | A | A | A | A | A | A | D | D | D | D | D | D | D | D | D | O | D | D | X | X | S | X | X | X | X | X | X | X | X | X |
| 12 | C | C | C | C | C | C | C | C | C | C | C | C | D | D | D | D | D | D | D | D | D | D | D | D | X | X | X | S | X | X | X | X | X | X | X | X |
| 13 | A | A | A | A | A | A | A | A | A | A | A | A | D | D | D | D | D | D | D | D | D | D | D | D | X | X | X | X | S | X | X | X | X | X | X | X |
| 14 | T | T | T | T | T | T | T | T | T | T | T | T | D | D | D | D | D | D | D | D | D | D | D | D | X | X | X | X | X | S | X | X | X | X | X | X |
| 15 | C | C | C | C | C | C | C | C | C | C | C | C | D | D | D | D | D | D | D | D | O | D | D | D | X | X | X | X | X | X | S | X | X | X | X | X |
| 16 | T | T | T | T | T | T | T | T | T | T | T | T | D | L | D | D | D | D | D | D | D | D | D | D | X | X | X | X | X | X | X | X | X | X | X | X |
| 17 | T | T | T | T | T | T | T | T | T | T | T | T | L | D | L | L | L | L | L | L | L | L | L | L | X | X | X | X | X | X | X | X | X | X | X | X |
| 18 | E | E | E | E | E | E | E | E | E | E | C | E | L | L | L | L | L | L | L | L | L | L | F | L | X | X | X | X | X | X | X | X | X | X | X | X |
| 19 | E | E | E | E | E | E | E | E | E | E | E | E | L | L | L | L | L | L | L | L | L | L | L | L | | | | | | | | | | | | |

TABLE 16
Particular antisense oligonucleotide variants of SEQ ID NO: 1816
Options for nucleobases, sugar modifications and internucleoside linkages
for particular antisense oligonucleotides according to the invention.
The options from which the nucleoside at each specific residue # in SEQ ID NO: 1122
can be chosen is shown in each line indicated by that residue #, starting from the 5′-end.
The selection of a specific nucleobase (“Nucleobase options for residue #”) and a sugar modification
(“Sugar modification options for residue #”) defines the nucleoside at that residue #.
The options for the internucleoside linkage between the nucleoside at a specific residue #
and the next residue, starting from the 5′-end, is shown in the column entitled
“Backbone modification options for internucleoside linkage #”.

| | Nucleobase options for | | | | | | | | | | | Sugar modification options | | | | | | | | | | | Backbone modification | | | | | | | | | | |
|---|---|---|---|---|---|---|---|---|---|---|---|---|---|---|---|---|---|---|---|---|---|---|---|---|---|---|---|---|---|---|---|---|---|
| Residue # in | residue # | | | | | | | | | | | for residue # | | | | | | | | | | | options for residue # | | | | | | | | | | |
| SEQ ID NO: 1816 | A: (A); C: (C); E: (5meC); | | | | | | | | | | | D: [dR]; F:[fR]; L: [LR]; | | | | | | | | | | | (from 5′-end) | | | | | | | | | | |
| (from 5′-end) | G: (G); T: (T); U: (U) | | | | | | | | | | | M: [MOE]; O: [mR] | | | | | | | | | | | S: [ssP]; X: [sP] | | | | | | | | | | |
| 1 | G | G | G | G | G | G | G | G | G | G | G | L | L | L | L | L | L | L | L | L | L | L | X | X | X | X | X | X | X | X | X | X | X |
| 2 | A | A | A | A | A | A | A | A | A | A | A | L | L | L | L | L | L | L | L | L | L | L | X | X | X | X | X | X | X | X | X | X | X |
| 3 | A | A | A | A | A | A | A | A | A | A | A | D | D | D | L | D | D | D | O | D | D | D | X | X | X | X | X | X | X | X | X | X | X |
| 4 | T | T | T | T | T | T | T | T | T | T | T | D | D | D | D | D | D | D | D | D | D | D | X | X | X | X | X | X | X | X | X | X | X |
| 5 | E | E | E | E | E | E | E | E | E | E | E | L | L | L | L | L | L | L | L | L | L | L | X | X | X | X | X | X | X | X | X | X | X |
| 6 | T | T | T | T | T | T | T | T | T | T | T | D | D | D | D | D | D | D | D | D | D | D | X | X | X | X | X | X | X | X | X | X | X |
| 7 | T | T | T | T | T | T | T | T | T | T | T | L | L | L | L | L | L | L | L | L | L | L | X | X | X | X | X | X | X | X | X | X | X |
| 8 | A | A | A | A | A | A | A | A | A | A | A | L | L | F | L | L | L | O | L | L | L | L | X | X | X | X | X | X | X | X | X | X | X |
| 9 | T | T | T | T | T | T | T | T | T | T | T | D | D | D | D | D | D | D | D | D | D | D | X | X | X | X | X | X | X | X | X | X | X |
| 10 | T | T | T | T | T | T | T | T | T | T | T | D | D | D | D | D | D | D | D | D | D | D | X | X | X | X | X | X | X | X | X | X | X |
| 11 | T | T | T | T | T | T | T | T | T | T | T | D | D | D | D | D | D | D | D | D | D | D | X | X | X | X | X | X | X | X | X | X | X |
| 12 | A | A | A | A | A | A | A | A | A | A | A | D | D | D | D | D | D | D | D | D | D | D | S | X | X | X | X | X | X | X | X | X | X |
| 13 | C | C | C | C | C | C | C | C | C | C | C | D | D | D | D | D | D | D | D | D | D | D | X | X | X | X | X | X | X | X | X | X | X |
| 14 | A | A | A | A | A | A | A | A | A | A | A | D | D | D | D | D | D | D | D | D | D | D | X | S | X | X | X | X | X | X | X | X | X |
| 15 | T | T | T | T | T | T | T | T | T | T | T | D | D | D | D | D | D | D | D | D | D | D | X | X | X | X | X | X | X | X | X | X | X |
| 16 | E | E | E | E | E | E | E | E | C | C | E | L | L | L | L | L | M | L | L | F | O | L | X | X | X | X | X | X | X | X | X | X | X |
| 17 | T | T | T | T | T | T | T | T | T | T | U | L | L | L | L | M | L | L | L | L | L | F | X | X | X | X | X | X | X | X | X | X | X |
| 18 | T | T | T | T | T | T | T | T | T | T | T | L | L | L | L | L | L | L | L | L | L | L | | | | | | | | | | | |

### Example 17: Transgenic mouse study to assess PK/PD of redesigned compounds

### Materials and Methods:

The study was largely performed as described in Example 8. In brief, transgenic animals (B6;CBA-Tg(ATXN3*)84.2Cce/IbezJ male and female mice 8 per group (JAX® Mice, The Jackson Laboratory)) of 10-12 weeks of age were included in the study. The animals were injected with 150 μg of compound in 4 μl into the cisterna magna (ICM). The animals were sacrificed after 4 weeks and selected tissues were obtained for evaluation of oligo content as well as knockdown of target transcript. Analysis methods were as described in Example 8.

### Results:

The results are presented in Table 17 below.

TABLE 17
Efficacy results.

| | Cortex | | Midbrain | | Cerebellum | | Pons/medulla | |
|---|---|---|---|---|---|---|---|---|
| | | Max | | Max | | Max | | Max |
| Compound ID | EC50 | efficacy (% | EC50 | efficacy (% | EC50 | efficacy (% | EC50 | efficacy (% |
| NO | (nM) | remaining) | (nM) | remaining) | (nM) | remaining) | (nM) | remaining) |
| 1122_91 | 894 | 77 | 69 | 52 | 616 | 63 | 134 | 41 |
| 1122_107 | 1385 | 35 | 73 | 20 | 388 | 22 | 95 | 21 |
| 1816_28 | 1791 | 71 | 442 | 62 | 1682 | 79 | 664 | 70 |
| 1816_42 | 8587 | 86 | 14763 | 73 | 1990 | 76 | 598 | 69 |
| 1122_156 | 812 | 69 | 75 | 43 | 330 | 56 | 105 | 40 |
| 1816_65 | 1633 | 73 | 453 | 55 | 1147 | 73 | 1053 | 59 |

*ND Value was not possible to determine due to the available data

Table 17 shows the readout from the study described in Example 17. An EC50 value was calculated for each compound in each of the obtained brain regions. Also, the maximally obtained target knockdown was highlighted within each group/tissue showing the remaining percentage of target mRNA relative to the saline control group.

The outcome of the evaluation resulted in compounds with attractive profiles in terms of efficacy and potency, i.e. with a low value for EC50 and a low value in the column for “max efficacy (% remaining)”. A most attractive compound based in these criteria is 1122_107 with best efficacy across all tissues and EC50 values in the lowest half of all the measured values. Another attractive compound based on efficacy and potency is 1122_156.

### Example 18: Comparative transgenic mouse study evaluation of tolerability

### Materials and Methods:

A further in vivo study was conducted to evaluate tolerability of test compounds. The study was largely performed as described in Example 10. In short, wild type animals (C57BL/6J) of around 7 weeks were included in the study. Animals, all female, arrived from The Jackson Laboratory, Bar Harbor, Me., USA were used. The mice were injected with the compounds via intracisternal (ICM) administration. After a period of 4 weeks, animals were euthanized and selected tissues were collected. The animals were injected with a dose 300 μg compound in WT animals.

Functional Observation Battery (FOB) scores from the animals were obtained On Day 1 at 1, 4, and 24±2 hours post dosing, then again prior to scheduled termination.

The FOB (functional observation Battery) score

The FOB score is a non-invasive tool describing various neurobehavioral and activity related parameters.

### Procedure:

Technicians were blinded to group belongings. Animals were observed for at least 1 minute in their home cage to record scores of the below mentioned parameters. Body temperature was recorded using a rectal thermometer.

The evaluation, except body temperature, was performed while the animal remained in its home cage; the open-field testing box will not be used.

| | | |
|---|---|---|
| Activity: | Excitability: | Autonomic: |
| Arousal/Alertness | Convulsions | Palpebral |
| Stereotypy | | Closure/Ptosis |
| Posture/Body Carriage | | Erected Fur |
| Neuromuscular: | Physiological: | |
| Gait/Mobility | Respiration | |
| Tremor | Body Temperature | |

Based on the evaluation of the animals they were scored as being either mild (in same range as saline control group), moderate or severely impacted by the administration of compound.

### Administration of Compound

Dose Route: Stereotaxic intracerebroventricular (ICV) injection. Dose volume was 10 μl. The dose was administered using a 10 μL Hamilton syringe with attached 22 gauge needle over one injection at a target of 1 μL/sec.

### Results:

The list of compounds is provided in Table 18 listing the compound ID number, the injected amount of test compound and a rating (mild, moderate or severe) of the in life observations, with mild meaning no or only few signs of adverse events, and severe meaning severe observation in the group with potential of premature takedown of animals in the group.

TABLE 18
Tolerability results.

| | | In life tolerability | No of | Premature |
|---|---|---|---|---|
| Compound ID | Dose | observations | animals* | termination |
| Saline | — | Mild | 6 | 0 |
| 1122_91 | 300 μg | Moderate | 7 | 0 |
| 1122_107 | 300 μg | Moderate | 7 | 0 |
| 1122_172 | 300 μg | Moderate | 7 | 0 |
| 1816_28 | 300 μg | Severe | 3 | 1 |
| 1816_42 | 300 μg | Severe | 3 | 0 |
| 1816_43 | 300 μg | Severe | 3 | 1 |
| 1122_154 | 300 μg | Moderate | 6 | 0 |
| 1122_156 | 300 μg | Mild | 6 | 0 |
| 1813_15 | 300 μg | Severe | 3 | 1 |
| 1605_4 | 300 μg | Mild | 6 | 0 |
| 1816_65 | 300 μg | Severe | 3 | 1 |
| 1605_2 | 300 μg | Mild | 6 | 0 |
| 1816_64 | 300 μg | Severe | 3 | 3 |

*For humane reasons 3 animals were dosed and depending on tolerability additional 3-4 animals were dosed on the following day.

Compound ID NO: 1813_15 has the following structure (HELM notation):

| | |
|---|---|
| 1813_15 | [LR]([5meC])[sP]•[LR](T)[sP]•[LR](G)[sP]•[dR](T)[sP]•[dR](A)[sP]•[mR](C)[sP]• |
| | [mR](A)[sP]•[dR](C)[sP]•[dR](T)[sP]•[dR](T)[sP]•[dR](T)[sP]•[dR](T)[sP]• |
| | [dR](A)[sP]•[dR](C)[sP]•[dR](A)[sP]•[LR](T)[sP]•[LR](T)[sP]•[LR]([5meC]) |

From Table 18, different outcomes relating to acute tolerability were observed for the different compounds, with some compounds showing “mild” signs (like the saline group animals) including compound ID NOs 1122_156, 1605_4 and 1605_2, some compounds showing “moderate” signs like compound ID NOs 1122_107 and 1816_64, and other compounds again showing “severe” signs like compound ID NOs 1816_43 and 1816_64.

A histopathological evaluation (macroscopic and microscopic) of the mice was conducted following termination of the animals. The aim of the evaluation was to identify compound related toxicological events, both of subacute and late onset nature. The evaluation included the pathologist's assessment of possible compound related neuronal changes based and hematoxylin and eosin stains and by Fluoro-Jade stain to assess signs of neuronal degeneration.

### Example 19: In vitro duration of action evaluation of LNA oligonucleotides in a time course, dose range experiment in human iPSC-derived neurons

### Materials and Methods:

The compounds listed in Table 19 and Table 20 were submitted to an in vitro evaluation of potency and efficacy in a time-course experimental setup. The study was largely performed as described in Example 9, with different time points and compounds.

The iCell® GlutaNeuron cells were prepared and maintained essentially as described in Example 5 & Table 2. 96-well cell culture plates were coated with Poly-L-Ornithine (0.01%) (Sigma-P4957), 100 μl/well for 4 hours, rinsed 3 times with PBS and coated with Laminin (Roche Diagnostic, 11243217001) 0.5 mg/ml diluted 1:500 in PBS overnight at 4 degrees Celsius. The cells were treated and maintained as per recommendation by the vendor using the provided protocol: iCell® GlutaNeurons, User's Guide, Document ID: X1005, Version 1.2, Cellular Dynamics, Fujifilm; available at https address cdn.stemcell.com/media/files/manual/MADX1005-icell_glutaneurons_users_guide.pdf (accessed on e.g. 10 Nov. 2020). Cells were grown for 7 days before addition of the oligonucleotide. Compounds were added to the cells from pre-dilution plates (compound diluted in PBS) to reach the desired final concentration. The concentrations used were an 8-step halflog with the following concentrations (nM): 31.6; 10; 3.2; 1; 0.32; 0.1; 0.03; 0.01.

Compounds used are listed in Table 19 and Table 20.

The following primers were used to assess the level of knockdown of the target mRNA of hATXN3.

Human ATXN3 pre-mRNA using the qPCR assay: custom
design “(ATXN3_exon_8-9(1)”, PrimeTime ® XL qPCR
Assay (IDT).
(SEQ ID NO: 1134)
Probe: 5′/56-FAM/CTCCGCAGG/ZEN/GCT ATTCAGCT AAGT/
31ABkFQ/-3′
(SEQ ID NO: 1135)
Primer 1: 5′-AGT AAGATTTGT ACCTGATGTCTGT-3′
(SEQ ID NO: 1136)
Primer 2: 5′-CATGGAAGATGAGGAAGCAGAT-3′
Human TBP pre-mRNA using the qPCR assay:
“Hs.PT.58v.39858774”, PrimeTime ® XL
qPCR Assay (IDT)
(SEQ ID NO: 1131)
Probe: 5′-/5HEX/TGA TCT TTG/ZEN/CAG TGA CCC AGC
ATC A/3IABkFQ/-3′
(SEQ ID NO: 1132)
Primer 1: 5′-GCT GTT TAA CTT CGC TTC CG-3′
(SEQ ID NO: 1133)
Primer 2: 5′-CAG CAA CTT CCT CAA TTC CTT G-3′

Cells were harvested at 4 days, 20 days, 29 days, and 40 days after oligo treatment, and RNA extraction and qPCR was performed as described for Example 1, using the ATXN3 primer assay described in Example 5. The relative ATXN3 mRNA expression levels were determined as % of control (medium-treated cells), i.e. the lower the value the larger the inhibition.

### Results:

The results are shown in Table 19 and Table 20. Table 19 presents the potency of the tested compounds (EC50 value (nM)) over the indicated time points post dosing. Table 20 presents the data as percent remaining ATXN3 mRNA relative to PBS treated cells after dosing with 3.204 compound for the indicated time points post dosing.

TABLE 19
EC50 in hiPSC-derived neurons

EC50 in hiPSC-derived neurons, nM

| CMP ID | Day 4 | Day 20 | Day 29 | Day 40 |
|---|---|---|---|---|
| 1122_154 | 56.5 | 42.4 | 89.3 | 314.4 |
| 1122_156 | 53.2 | 33.4 | 50.1 | 88.7 |
| 1816_28 | 128.4 | 653.3 | 926.1 | 1465.0 |
| 1605_4 | 76.7 | 33.6 | 47.3 | 71.8 |
| 1816_65 | 70.9 | 51.0 | 184.2 | 692.3 |
| 1122_91 | 115.5 | 76.9 | 171.6 | 748.0 |
| 1122_107 | 74.6 | 20.7 | 19.0 | 35.1 |
| 1122_172 | 66.1 | 50.1 | 90.4 | 306.1 |
| 1816_42 | 196.0 | 262.0 | 546.3 | 830.7 |
| 1816_43 | 97.2 | 68.5 | 136.1 | 709.8 |
| Negative control | ND | ND | ND | ND |

*ND Value was not possible to determine due to the available data

The calculated EC50 values for each compound for each of the time points are shown in Table 19. From the data it can be seen that there was a wide range of obtained EC50 values for the different compounds at the different time points. Generally there were some compounds showing high EC50 values indicating a low potency which also decreases (higher value) over time. Examples of these are compound ID NOs 1816_28, and 1122_172. On the other hand, there were also compounds which maintained a high potency (low value for EC50) over the duration of 40 days, showing EC50 values below 100 nM. Examples of these compounds are compound ID NOs 1122_156, 1605_4 and 1122_107. A low EC50 value is a beneficial property of a compound because it indicates that a lower amount of compound is required to elicit an effect.

TABLE 20
Efficacy in hiPSC-derived neurons following
addition of 3.2 μM compound.

| | % remaining transcript in hiPSC-derived neurons | | | |
|---|---|---|---|---|
| | after treatment with 3.2 μM compound | | | |
| CMP ID | Day 4 | Day 20 | Day 29 | Day 40 |
| 1122_154 | 6.3 | 4.4 | 11.5 | 19.0 |
| 1122_156 | 6.8 | 5.0 | 7.8 | 16.0 |
| 1816_28 | 13.2 | 29.6 | 32.3 | 50.5 |
| 1605_4 | 8.7 | 3.3 | 2.9 | 4.5 |
| 1816_65 | 7.5 | 8.2 | 13.1 | 22.7 |
| 1122_91 | 10.5 | 8.0 | 15.5 | 32.5 |
| 1122_107 | 8.3 | 4.5 | 4.3 | 6.0 |
| 1122_172 | 7.1 | 4.8 | 8.2 | 22.0 |
| 1816_42 | 9.8 | 15.2 | 23.6 | 46.3 |
| 1816_43 | 7.1 | 7.8 | 9.8 | 20.9 |
| Negative control | 93.5 | 95.1 | 94.1 | 115.8 |

Table 20 show the observed levels of remaining mRNA (% remaining transcript compared to PBS control) in the cells following a treatment of the cells with 3.204 of compound as described above. The evaluation was performed at multiple time points after compound addition. From the table it is clear that many of the compounds were able to maintain a suppression of the level of mRNA over the duration of 40 days following a single exposure to compound. A number of compounds were able to maintain a suppression of more than 90% for the duration of 40 days such as compound ID NOs 1605_4 and 1122_107. Furthermore, compound ID NO 1122_156 showed a high level of knockdown for the duration of 40 days. A high level of knockdown over a long period indicates a long duration of action and potential for a more infrequent administration.

### Example 20: In vivo assessment in transgenic animals of efficacy and duration of action

### Materials and Methods:

A further in vivo study was performed using compound ID NOs 1605_4, 1122_107 and 1122_156. The study used male and female B6;CBA-Tg(ATXN3*)84.2Cce/IbezJ mice with the compounds administered via intra cisterna magna (ICM) administration. At three time points after compound administration, 4, 8 and 11 weeks, animals were euthanized and terminal plasma samples and tissues were collected.

### Animal Care

In vivo activity and tolerability of the compounds were tested in 62 B6;CBATg(ATXN3*)84.2Cce/IbezJ male and female mice (JAX® Mice, The Jackson Laboratory) at the age of 10 weeks. Following arrival, animals were housed in groups up to 5 in individually vented cages (IVC, 38×22×15 cm) in a temperature (22±2° C.) and humidity (55±15%) controlled environment on a 12 hour light cycle (07.00-19.00 h). Males and females were kept in separate cages. Standard diet (SDS Diets, RM1 PL) and domestic quality mains water were available ad libitum. If required, animals received soaked chow and/or Royal Canin in addition to standard diet as part of pamper care. The experiments were conducted in strict accordance with the Guide for the Care and Use of Laboratory Animals (National Research Council 2011) and were in accordance with European Union directive 2010/63 and the Dutch law.

Administration Route-Intra-cisterna Magna injections.

The compounds were administered to mice by intra cisterna magna (ICM) injections. Mice were anesthetized using isoflurane (2.5-3% and 500 mL/min 02). Before surgery, Finadyne (1 mg/kg, s.c.) was administered for analgesia during surgery and the post-surgical recovery period. A mixture of bupivacaine and epinephrine was applied to the incision site and periost of the skull for local analgesia. Animals were placed in a stereotaxic frame (Kopf instruments, USA) and an incision made at the back of the head towards the neck. Then, the skin was spread and the coordinates marked prior to drilling a hole in the occipital bone of the skull, where a cannula was placed. Next, the compounds were injected into the cisterna magna (ICM). A volume of 10 μL of the assigned test item was injected over 30 seconds. After injection, the needle and cannula were held in place for 30 seconds to ensure no back flow occurred. The cannula was then retracted, the hole was covered with skin and the incision was closed by sutures. Animals were placed in a warm environment until recovered from the procedure. The compounds were administered as a single dose as listed in Table 21. Each group contained 4-6 animals.

TABLE 21
Treatment regimes.

| | Dose of compound in 10 μl | Weeks of |
|---|---|---|
| Compound ID NO | volume | treatment |
| 0.9% Saline | — | 4 |
| 1605_4 | 20 μg/μl equals 200 μg | 4, 8, 11 |
| | compound | |
| 1122_107 | 20 μg/μl equals 200 μg | 4, 8, 11 |
| | compound | |
| 1122_156 | 20 μg/μl equals 200 μg | 4, 8,11 |
| | compound | |

At the end of the experiment, after week 4, 8 and 11, the animals were euthanized by Euthasol® overdose. Terminal plasma was collected in Li-Hep tubes. Terminal tissues were harvested from the animals and were dissected on a chilled surface. Half of the tissue samples were stored in 2.0 mL Safe-Lock tubes, PCR clean, pre-weighted and precooled. Immediately after collection, samples were weighed and flash frozen in liquid N2 prior to storage at −80° C. The other half was fixed in 4% PFA for 72 hours and subsequently transferred to 70% ethanol awaiting shipment. Tissue dissection and collection was performed, collecting tissue from a range of tissues: Cortex, Cerebellum, Brainstem, Midbrain and Striatum. There were no signs of acute toxicity of any of the administered compounds and hence no premature termination of animals due to compound related toxicity.

Analysis of in vivo samples: Description of tissue preparation for content measurement and qPCR was performed as per Example 8. The median knockdown of target mRNA achieved was recorded as percent remaining target transcript relative to saline control group—this data is provided in Table 22.

### Results:

TABLE 22
Knock down data for each compound for each time point.

| | | Cortex | Cerebellum | Brainstem | Midbrain | Striatum |
|---|---|---|---|---|---|---|
| | | Median KD | Median KD | Median KD | Median KD | Median KD |
| | | (% remaining | (% remaining | (% remaining | (% remaining | (% remaining |
| Tissue | | transcript) | transcript) | transcript) | transcript) | transcript) |
| 4 week of | 1605_4 - 200 μg | 19 | 34 | 8 | 5 | 34 |
| treatment | 1122_107 - 200 μg | 35 | 31 | 14 | 8 | 41 |
| | 1122_156 - 200 μg | 53 | 44 | 16 | 12 | 53 |
| 8 week of | 1605_4 - 200 μg | 56 | 41 | 22 | 12 | 67 |
| treatment | 1122_107 - 200 μg | 43 | 30 | 25 | 17 | 33 |
| | 1122_156 - 200 μg | 76 | 46 | 46 | 38 | 72 |
| 11 week of | 1605_4 - 200 μg | 65 | 50 | 38 | 21 | 73 |
| treatment | 1122_107 - 200 μg | 82 | 41 | 46 | 39 | 75 |
| | 1122_156 - 200 μg | 99 | 70 | 70 | 75 | 100 |

Overall. it can be seen that the brainstem and the midbrain were the brain areas targeted most efficiently across all compounds when focusing on knock down efficacy.

### Example 21: WT and polyQ Ataxin 3 protein levels in human SCA3 patient derived fibroblasts treated with selected oligonucleotides (ASO)

### Materials and Methods:

Reference compounds 1287095 and 1102579 referred to in this Example correspond to the oligonucleotides disclosed as Compound No. 1287095 in WO2020/172559 A1 and Compound No. 1102579 in WO2019/217708 respectively.

This experiment was performed to investigate the efficacy of knock down of the tested antisense oligonucleotides. The study was largely performed as described in Example 12.

The evaluation was performed in the SCA3 patient derived fibroblasts, allowing for an assessment of the efficacy on the disease causing ataxin3 allele and the ataxin3 WT allele.

The cell line used for the ASO treatment was human SCA3 patient derived fibroblasts (GM06153—Coriell Institute). Twenty thousand cells were seeded per well in a 24 well plate with a total volume of 1 ml. ASOs were added immediately after to a final concentration of 5 μM (gymnotic uptake). After 4 days of incubation, cells were washed twice with PBS, and harvested in 50 μl LDS sample buffer (NuPAGE, Thermo Scientific) with addition of 50 mM fresh DTT.

Western blots were performed on the capillary-based immunoassay platform (WES, ProteinSimple) using a WES 12-230 kDa Wes Separation Module. Cell lysates were diluted 10×in Sample load buffer (ProteinSimple) prior to loading on the cartridge. Primary antibody for Ataxin 3 (rabbit monoclonal antibody, prod. #702788 from Invitrogen) and for HPRT (rabbit monoclonal antibody, cat. #Ab109021 from Abcam). Both antibodies were used in 1/100 dilutions. Goat anti-rabbit HRP conjugate (Part. #DM-001, ProteinSimple) was used as secondary antibody.

Compass software (ProteinSimple) was for quantification of the protein bands.

### Results:

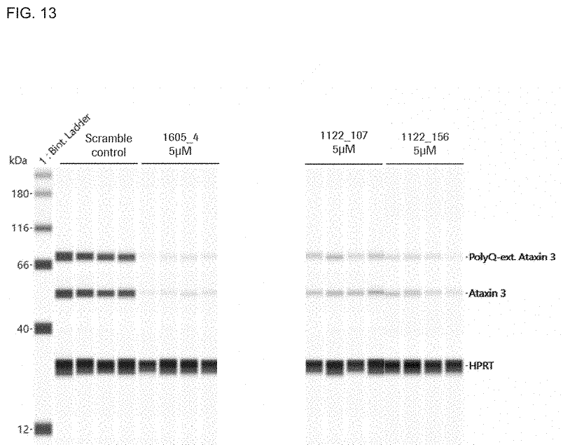

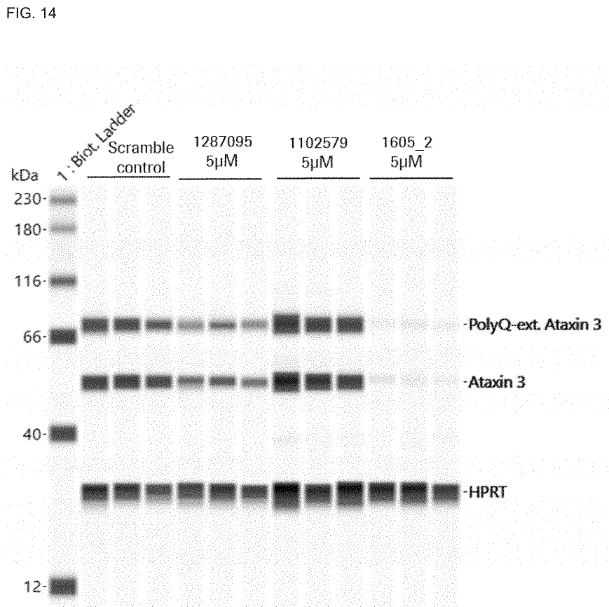

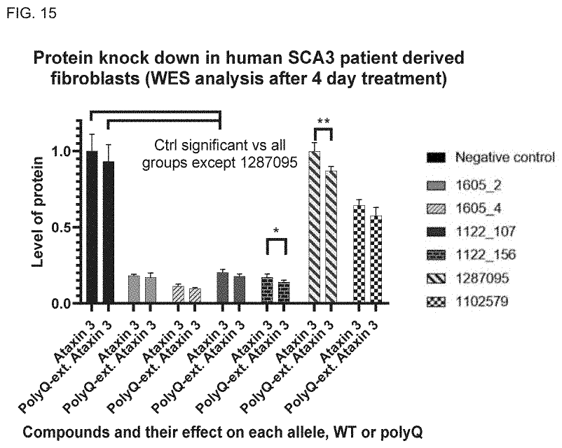

Ataxin 3 antibody recognized both isoforms, and the intensity (area under peak) was normalized to the protein input based on the signal from HPRT. The raw data are shown in FIGS. 13 and 14, and are quantified as described in FIG. 15.

Difference between allele selectivity was evaluated using an ordinary t-test, (unpaired, two-tailed, homoscedastic, calculated using Microsoft Excel, 2016, version 160.5227.1000) for each compound comparing the level of each detected allele. A difference (α=0.05) was observed for compound ID NOs 1287095 and 1122_156.

The effect on protein knock down was evaluated for each allele (e.g. wild type and polyQ expanded allele, individually) and compared to the negative control using an ordinary one-way ANOVA test with Dunnett correction for multiple testing (Calculated using GraphPad Prism, version 8.4.2 (679)). Some level of a decrease in protein from both WT and polyQ allele was observed for all compounds except for compound ID NO 1287095, where no decrease was observed for either allele using an effective concentration of compound of 504.

In summary it can be observed in FIGS. 13 to 15 that compound ID NOs 1605_2, 1605_4, 1122_107 and 1122_156 effectively induce a knockdown of target protein—both wild type and polyQ expanded version—and that compound ID NO 1122_156 additionally has a statically significant increased effect on the polyQ expanded allele. A higher activity on the disease causing polyQ extended Ataxin 3 than the WT Ataxin 3 is preferable as it allows a selective reduction of the disease causing allele. The reference compounds 1287095 and 1102579 did not induce a significant reduction of either of the versions of the target protein under the tested conditions.

This experimental setup also measured the resulting effect on mRNA level following treatment with the compounds shown in FIG. 15. The data are not shown, but the analysis was performed exactly as described in Example 22. The effect of the compounds on the ATXN3 mRNA level followed the same pattern as did the protein levels. This means that when a high level of protein knock down was observed, there was also observed a high level of mRNA knock down and vice versa, as shown in Example 22.

### Example 22: Knock down of WT Ataxin 3 protein levels in human SK-N-AS cell line treated with selected oligonucleotides (ASO)

### Materials and Methods:

This experiment was performed to investigate the efficacy of efficacy of knock down of the tested antisense oligonucleotides. The evaluation was performed in SK-N-AS cells (cell line listed in Table 2), allowing for an assessment of the efficacy on ataxin3 alleles and related protein production. Twenty five thousand cells were seeded per well in a 24 well plate with a total volume of 500 μl. In three replicate wells, ASOs were added immediately after to a final concentration of 5 μM (gymnotic uptake). The reference compounds 1287095 and 1102579 were additionally tested in a concentration of 15 μM. After 4 days of incubation, cells were washed twice with PBS, and harvested in 100 μl LDS sample buffer (NuPAGE, Thermo Scientific) with addition of 50 mM fresh DTT. Western blots were performed on the capillary-based immunoassay platform (WES, ProteinSimple) using a WES 12-230 kDa Wes Separation Module. Cell lysate were diluted 10× in Sample load buffer (ProteinSimple) prior loading on the cartridge. Primary antibody for Ataxin 3 (rabbit monoclonal antibody, prod. #702788 from Invitrogen) and for HPRT (rabbit monoclonal antibody, cat. #Ab109021 from Abcam). Both antibodies were used in 1/100 dilutions. Goat anti-rabbit HRP conjugate (Part. #DM-001, ProteinSimple) was used as secondary antibody.

Compass software (ProteinSimple) was for quantification of the protein bands.

The effects of the above mentioned compounds were also evaluated on transcript level by digital droplet PCR (ddPCR) analysis in a similar setup. Cells were treated similarly to the cells used for protein determination with respect to compound concentrations and time points. The SK-N-AS cell line was used for the ASO treatment, with 10.000 cells seeded per well in a 96 well plate with a total volume of 0.1 ml. ASOs were added immediately after to a final concentration of 5 μM and for compounds 1287095 and 1102579 also with 15 μM (gymnotic uptake). After 4 days of incubation, cells were washed twice with PBS, and harvested in 200 μl RIPA buffer (Thermo Scientific, Pierce). The purified RNA was denatured before cDNA synthesis. cDNA was created using the iScript Advanced cDNA Synthesis Kit for RT-qPCR (Biorad) according to the manufacturer's instructions. Measurements of the expression levels of the target genes was done by droplet digital PCR using the QX200 droplet system (Bio-Rad) together with the QX200 software standard edition.

The following assays were used for the analysis:

qPCR probe and primers set:
PrimeTime ® XL qPCR Assay (IDT)-
ATXN3_exon_8-9(1)
(SEQ ID NO: 1134)
Probe: 5′-/56-FAM/CTCCGCAGG/ZEN/GCT ATTCAGCT
AAGT/31ABkFQ/-3′
(SEQ ID NO: 1135)
Primer 1: 5′-AGT AAGATTTGT ACCTGATGTCTGT-3′
(SEQ ID NO: 1136)
Primer 2: 5′-CATGGAAGATGAGGAAGCAGAT-3′
Reference gene was hHPRT
PrimeTime ® XL qPCR Assay (IDT)-
Hs.PT.58v.45621572
(SEQ ID NO: 1993)
Probe: 5′-/5HEX/AGCCTAAGA/ZEN/TGAGAGTTCAAGTTGAGTT
TGG/3IABkFQ/-3′
(SEQ ID NO: 1994)
Primer 1: 5′-GCGATGTCAATAGGACTCCAG-3′
(SEQ ID NO: 1995)
Primer 2: 5′-TTGTTGTAGGATATGCCCTTGA-3′

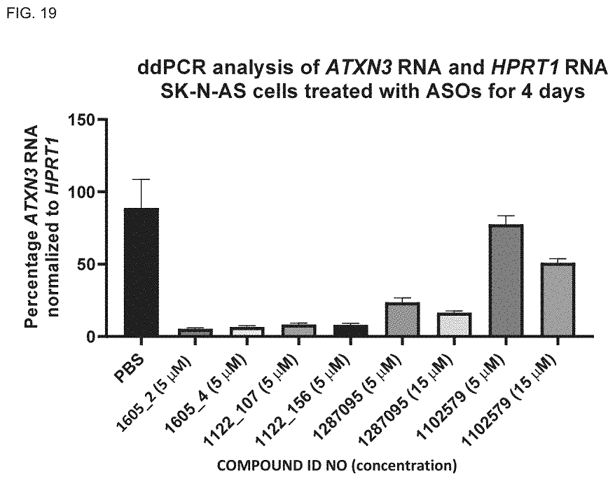

The resulting evaluation of the remaining mRNA level following compound treatment is presented in FIG. 19.

### Results:

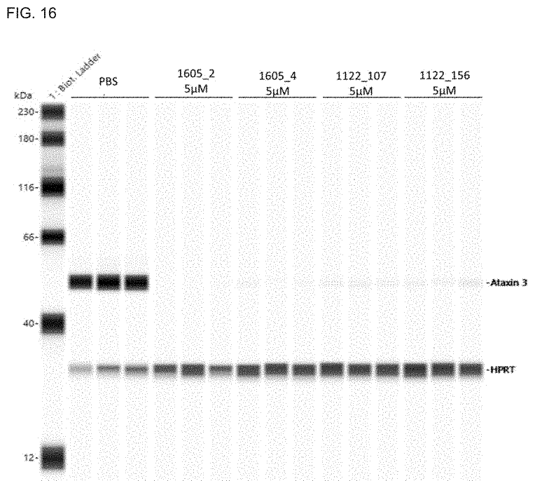

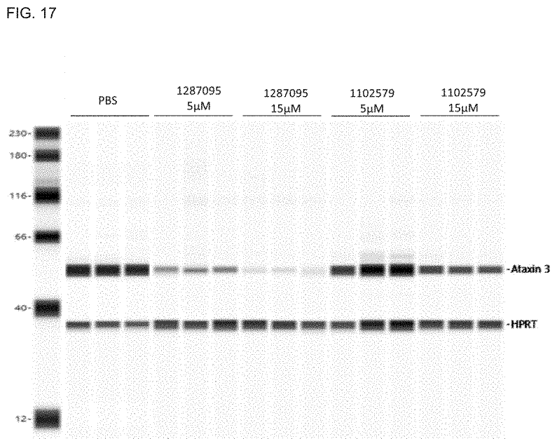

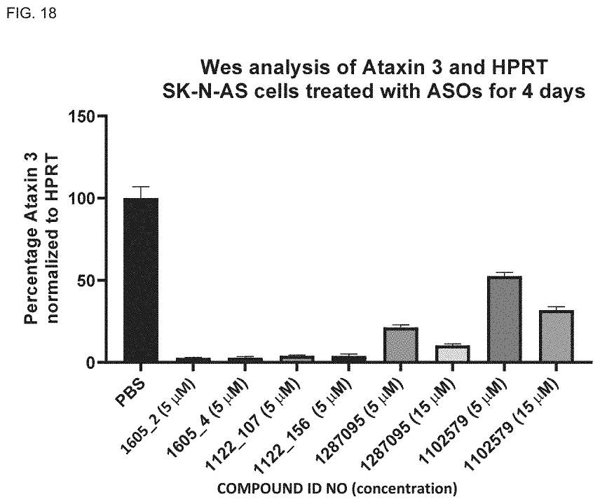

Ataxin 3 antibody recognizes the wild type Ataxin 3 protein expressed by the cells, and the intensity (area under peak) was normalized to the protein input based on the signal from HPRT. The raw data are shown in FIG. 16 and FIG. 17 (Reference compounds including negative and positive controls), and the quantification is shown in graphical format in FIG. 18.

The effect on protein knock down for each of the tested compounds was evaluated by comparison to the negative PBS control using an ordinary one-way ANOVA test with Dunnett correction for multiple testing (Calculated using GraphPad Prism, version 8.4.2 (679)). A decrease in protein levels were observed for all compounds with p-value<0.001 compared to PCR control samples. From the raw WES data (FIG. 16 and FIG. 17) and the quantification (FIG. 18) it is clear that there are pronounced differences in the level of protein knock down between the compounds. The compounds 1605_2, 1605_4, 1122_107 and 1122_156 were very efficacious in knocking down Ataxin 3 protein using the effective concentrations of 504. The reference compounds 1287095 and 1102579 were both less efficacious. Reference compounds 1287095 and 1102579 were unable to induce the same level of effect (level of protein knockdown) as the best performing compounds even when used in a disadvantageously high concentration (15 μM, 3 times higher than the other respective doses of 5 μM).

FIG. 19 shows the efficacy of the compounds with the listed applied concentrations in terms of knockdown of the ATXN3 encoding transcript. The mRNA knock down follows the same pattern as observed for the protein quantification shown in FIG. 18. Again it is observed that the compound ID NOs 1605_2, 1605_4, 1122_107 and 1122_156 are more efficacious at reducing ATXN3 mRNA levels compared to compounds 1287095 and 1102579. Again, a three times higher applied concentration of compounds 1287095 and 1102579 still did not obtain a level of efficacy which was as high as that of the best performing compounds.

1. An antisense oligonucleotide selected from the group consisting of Compound ID Nos. 1122_107, 1122_156, 1122_91, 1122_154, 1122_155, 1122_157, 1122_158, 1122_167, 1122_172, 1122_175, 1122_294, 1122_296, 1816_13, 1816_15, 1816_28, 1816_41, 1816_42, 1816_43, 1816_60, 1816_61, 1816_64, 1816_65, and 1816_68, or a pharmaceutically acceptable salt thereof.

2. An antisense oligonucleotide according to claim 1 of the following chemical annotation:
(a) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[m R]}U_{[sP]}.^{[dR]}(A)_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C)_{[sP]}.^{[LR][5me]}C (SEQ ID NO:1122, wherein residue 10 is U) (Compound ID No. 1122_91);
(b) ^{[LR]}A_{[sP]}.^{[LR]}A_{[sP]}.^{[mR]}U_{[sP]}.^{[LR][5me]}C_{[sP]}.^{[dR]}T_{[sP]}.^{[dR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[LR]}T_{[sP]}.^{[dR]}T_{[sP]}.^{[LR][5me]}C_{[sP]}.^{[LR][5me]}C (SEQ ID NO:1122, wherein residue 3 is U) (Compound ID No. 1122_107);
(c) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[ssP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_154);
(d) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[ssP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_155);
(e) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[ssP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_156);
(f) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[sP]}.^{[dR]}T_{[ssP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_157);
(g) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[d R]}T_{[sP]}.^{[dR]}A_{[sP]}.^{[dR]}C_{[sP]}.^{[dR]}A_{[sP]}.^{[dR]}T_{[sP]}.^{[dR]}C_{[ssP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_158);
(h) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[MOE]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_167);
(i) ^{[LR]}A_{[sP]}.^{[dR]}A_{[sP]}.^{[dR]}T_{[sP]}.^{[LR]}[5meC]_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[ssP]}.^{[dR]}A_{[sP]}.^{[dR]}T_{[sP]}.^{[mR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_172);
(j) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_175);
(k) ^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[fR]}C_{[sP]}.^{[LR][5me]}C (SEQ ID NO:1122) (Compound ID No. 1122_294);
(l) ^{[LR]}A_{[sP]}.^{[dR]}A_{[sP]}.^{[dR]}T_{[sP]}.^{[mR]}C_{[sP]}.^{[dR]}T_{[sP]}.^{[LR]}T_{[sP]}.^{[dR]}A_{[sp]}.^{[dR]}T_{[sP]}.^{[dR]}T_{[sP]}.^{[dR]}T_{[s P]}.^{[dR]}A_{[sP]}.^{[dR]}C_{[sP]}.^{[dR]}A_{[sP]}.^{[dR]}T_{[sP]}.^{[dR]}C_{[sP]}.^{[dR]}T_{[sP]}.^{[LR]}T_{[sP]}.^{[LR][5me]}C_{[sP]}.^{[LR][5me]}C (SEQ ID NO:1122) (Compound ID No. 1122_296);
(m) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[ssP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_13);
(n) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[ssP]}.^{[dR]}T_{[sP]}.^{[LR][5me]}C_{[sP]}.^{[LR]}T_{[sP]}.^{[LR]}T (SEQ ID NO:1816) (Compound ID No. 1816_15);
(o) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[fR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_28);
(p) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_41);
(q) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[MOE]}T_{[sP]}.^{[LR]}T (SEQ ID NO:1816) (Compound ID No. 1816_42);
(r) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[MOE][5me]}C_{[sP]}.^{[LR]}T_{[sP]}.^{[LR]}T (SEQ ID NO:1816) (Compound ID No. 1816_43);
(s) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[mR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_60);
(t) ^{[LR]}G_{[sP]}.^{[LR]}A_{[sP]}.^{[mR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[LR]}T_{[sP]}.^{[LR]}T (SEQ ID NO:1816) (Compound ID No. 1816_61);
(u) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[fR]}C_{[sP]}.^{[LR]}T_{[sP]}.^{[LR]}T (SEQ ID NO:1816) (Compound ID No. 1816_64);
(v) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[mR]}C_{[sP]}.^{[LR]}T_{[sP]}.^{[LR]}T (SEQ ID NO:1816) (Compound ID No. 1816_65); or
(w) ^{[LR]}G_{[sP]}.^{[LR]}A_{[sP]}.^{[dR]}A_{[sP]}.^{[dR]}T_{[sP]}.^{[LR][5me]}C_{[sP]}.^{[dR]}T_{[sP]}.^{[LR]}T_{[sP]}.^{[LR]}A_{[sP]}.^{[dR]}T_{[sP]}.^{[dR]}T_{[sP]}.^{[dR]}T_{[sP]}.^{[dR]}A_{[sP]}.^{[dR]}C_{[sP]}.^{[dR]}A_{[sP]}.^{[dR]}T_{[sP]}.^{[LR][5me]}C_{[sP]}.^{[fR]}U_{[sP]}.^{[LR]}T (SEQ ID NO:1816, wherein residue 17 is U)(Compound ID No. 1816_68),
or is a pharmaceutically acceptable salt thereof, wherein
[LR] is a beta-D-oxy-LNA nucleoside,
[LR][5me]C is a beta-D-oxy-LNA 5-methyl cytosine nucleoside,
[dR] is a DNA nucleoside,
[sP] is a phosphorothioate internucleoside linkage (stereo undefined)
[ssP] is a stereodefined Sp phosphorothioate internucleoside linkage
[mR] is a 2′-O-methyl nucleoside,
[MOE] is a 2′-O-methoxyethyl nucleoside, and
[fR] is a 2′-fluoro nucleoside.

3. The antisense oligonucleotide according to claim 1, which is the antisense oligonucleotide shown in FIG. 12A (Compound ID No. 1122_91); or a pharmaceutically acceptable salt thereof.

4. The antisense oligonucleotide according to claim 1, which is the antisense oligonucleotide shown in FIG. 12B (Compound ID No. 1122_107); or a pharmaceutically acceptable salt thereof.

5. The antisense oligonucleotide according to claim 1, which is the antisense oligonucleotide shown in FIG. 12C (Compound ID No. 1122_154); or a pharmaceutically acceptable salt thereof.

6. The antisense oligonucleotide according to claim 1, which is the antisense oligonucleotide shown in FIG. 12D (Compound ID No. 1122_155); or a pharmaceutically acceptable salt thereof.

7. The antisense oligonucleotide according to claim 1, which is the antisense oligonucleotide shown in FIG. 12E (Compound ID No. 1122_156); or a pharmaceutically acceptable salt thereof.

8. The antisense oligonucleotide according to claim 1, which is the antisense oligonucleotide shown in FIG. 12F (Compound ID No. 1122_157); or a pharmaceutically acceptable salt thereof.

9. The antisense oligonucleotide according to claim 1, which is the antisense oligonucleotide shown in FIG. 12G (Compound ID No. 1122_158); or a pharmaceutically acceptable salt thereof.

10. The antisense oligonucleotide according to claim 1, which is the antisense oligonucleotide shown in FIG. 12H (Compound ID No. 1122_167); or a pharmaceutically acceptable salt thereof.

11. The antisense oligonucleotide according to claim 1, which is the antisense oligonucleotide shown in FIG. 12I (Compound ID No. 1122_172); or a pharmaceutically acceptable salt thereof.

12. The antisense oligonucleotide according to claim 1, which is the antisense oligonucleotide shown in FIG. 12J (Compound ID No. 1122_175); or a pharmaceutically acceptable salt thereof.

13. The antisense oligonucleotide according to claim 1, which is the antisense oligonucleotide shown in FIG. 12K (Compound ID No. 1122_294); or a pharmaceutically acceptable salt thereof.

14. The antisense oligonucleotide according to claim 1, which is the antisense oligonucleotide shown in FIG. 12L (Compound ID No. 1122_296); or a pharmaceutically acceptable salt thereof.

15. The antisense oligonucleotide according to claim 1, which is the antisense oligonucleotide shown in FIG. 12M (Compound ID No. 1816_13); or a pharmaceutically acceptable salt thereof.

16. The antisense oligonucleotide according to claim 1, which is the antisense oligonucleotide shown in FIG. 12N (Compound ID No. 1816_15); or a pharmaceutically acceptable salt thereof.

17. The antisense oligonucleotide according to claim 1, which is the antisense oligonucleotide shown in FIG. 12O (Compound ID No. 1816_28); or a pharmaceutically acceptable salt thereof.

18. The antisense oligonucleotide according to claim 1, which is the antisense oligonucleotide shown in FIG. 12P (Compound ID No. 1816_41); or a pharmaceutically acceptable salt thereof.

19. The antisense oligonucleotide according to claim 1, which is the antisense oligonucleotide shown in FIG. 12Q (Compound ID No. 1816_42); or a pharmaceutically acceptable salt thereof.

20. The antisense oligonucleotide according to claim 1, which is the antisense oligonucleotide shown in FIG. 12R (Compound ID No. 1816_43); or a pharmaceutically acceptable salt thereof.

21. The antisense oligonucleotide according to claim 1, which is the antisense oligonucleotide shown in FIG. 12S (Compound ID No. 1816_60); or a pharmaceutically acceptable salt thereof.

22. The antisense oligonucleotide according to claim 1, which is the antisense oligonucleotide shown in FIG. 12T (Compound ID No. 1816_61); or a pharmaceutically acceptable salt thereof.

23. The antisense oligonucleotide according to claim 1, which is the antisense oligonucleotide shown in FIG. 12U (Compound ID No. 1816_64); or a pharmaceutically acceptable salt thereof.

24. The antisense oligonucleotide according to claim 1, which is the antisense oligonucleotide shown in FIG. 12V (Compound ID No. 1816_65); or a pharmaceutically acceptable salt thereof.

25. The antisense oligonucleotide according to claim 1, which is the antisense oligonucleotide shown in FIG. 12W (Compound ID No. 1816_68); or a pharmaceutically acceptable salt thereof.

26. A conjugate comprising an oligonucleotide according to claim 1, and at least one conjugate moiety covalently attached to said oligonucleotide; or a pharmaceutically acceptable salt thereof.

27. A pharmaceutical composition comprising an oligonucleotide according to claim 1 or a conjugate thereof and a pharmaceutically acceptable diluent, solvent, carrier, salt and/or adjuvant.

28. An in vivo or in vitro method for modulating ATXN3 expression in a target cell which is expressing ATXN3, said method comprising administering an oligonucleotide selected from the group consisting of Compound ID Nos. 1122_91, 1122_107, 1122_154, 1122_155, 1122_156, 1122_157, 1122_158, 1122_167, 1122_172, 1122_175, 1122_294, 1122_296, 1816_13, 1816_15, 1816_28, 1816_41, 1816_42, 1816_43, 1816_60, 1816_61, 1816_64, 1816_65, and 1816_68, a conjugate, a salt, or a pharmaceutical composition thereof in an effective amount to said cell.

29. A method for treating or preventing a disease comprising administering a therapeutically or prophylactically effective amount of an oligonucleotide selected from the group consisting of Compound ID Nos. 1122_91, 1122_107, 1122_154, 1122_155, 1122_156, 1122_157, 1122_158, 1122_167, 1122_172, 1122_175, 1122_294, 1122_296, 1816_13, 1816_15, 1816_28, 1816_41, 1816_42, 1816_43, 1816_60,1816_61,1816_64,1816_65, and 1816_68, a conjugate, a salt, or a pharmaceutical composition thereof to a subject suffering from or susceptible to the disease.

30. The method of claim 29, wherein the disease is spinocerebellar ataxia, such as spinocerebellar ataxia 3, such as Machado-Joseph disease

31. The oligonucleotide, a conjugate, a salt, or a pharmaceutical composition thereof according to claim 1 for use in medicine.

32. The oligonucleotide, a conjugate, a salt, or a pharmaceutical composition thereof according to claim 1 for use in the treatment or prevention of spinocerebellar ataxia, such as spinocerebellar ataxia 3, such as Machado-Joseph disease, (MJD).

33. Use of the oligonucleotide, a conjugate, a salt, or a pharmaceutical composition thereof according to claim 1 for the preparation of a medicament for treatment or prevention of spinocerebellar ataxia, such as spinocerebellar ataxia 3, such as Machado-Joseph disease.
